(19) United States
(12) Patent Application Publication
(10) Pub. No.: US 2020/0321536 A1
(43) Pub. Date: Oct. 8, 2020

(54) COMPOUND, MATERIAL FOR ORGANIC ELECTROLUMINESCENT ELEMENT, ORGANIC ELECTROLUMINESCENT ELEMENT, AND ELECTRONIC DEVICE

(71) Applicant: IDEMITSU KOSAN CO.,LTD., Tokyo (JP)

(72) Inventor: Ryota TAKAHASHI, Sodegaura-shi, Chiba (JP)

(73) Assignee: IDEMITSU KOSAN CO.,LTD., Tokyo (JP)

(21) Appl. No.: 16/753,532

(22) PCT Filed: Oct. 9, 2018
(86) PCT No.: PCT/JP2018/037605

§ 371 Date: Apr. 3, 2020

(30) Foreign Application Priority Data
Oct. 6, 2017 (JP) 2017-196431

(51) Int. Cl. H01L 51/00; C07D 487/06; C09K 11/06

(52) U.S. Cl. CPC H01L 51/0072; C07D 487/06; H01L 51/5012; H01L 51/0061; C09K 11/06

(57) ABSTRACT

Novel compounds for use in the production of organic electroluminescence (EL) devices, organic EL devices comprising the compounds, and electronic devices comprising the organic EL devices are provided. The compound is represented by formula (1):

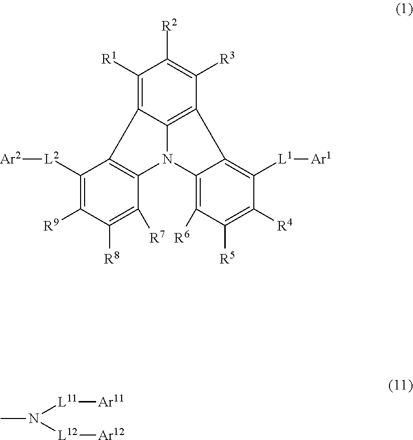

wherein R¹ to R⁹, Ar¹, Ar², Ar¹¹ to Ar¹², L¹ to L², and L¹¹ to L¹² are as defined in the description.

### TECHNICAL FIELD

The present invention relates to compounds, materials for organic electroluminescence device (hereinafter simply referred to as “organic EL device”) comprising the compounds, organic EL devices comprising the compounds, and electronic devices comprising the organic EL devices.

### BACKGROUND ART

An organic EL device generally comprises an anode, a cathode, and an organic layer sandwiched between the anode and the cathode. When a voltage is applied between the electrodes, electrons are injected from the cathode and holes are injected from the anode into a light emitting region. The injected electrons recombine with the injected holes in the light emitting region to form excited states. When the excited states return to the ground state, light is emitted.

Many compounds for use in the production of organic EL devices have been reported. One of these compounds is a compound having an indro[3,2,1-jk]carbazole skeleton.

For example, Patent Literatures 1 to 14 describe the compounds having an indro[3,2,1-jk]carbazole skeleton.

### CITATION LIST

### Patent Literature

Patent Literature 1: U.S. Pat. No. 8,174,002

### Patent Literature 2: WO 2013/077344

Patent Literature 3: U.S. Pat. No. 9,422,287

### Patent Literature 4: JP 2014-073965

### Patent Literature 5: KR 10-2015-0135125

### Patent Literature 6: WO 2016/006925

### Patent Literature 7: CN 104119347

### Patent Literature 8: KR 10-2016-0142792

### Patent Literature 9: WO 2017/022983

### Patent Literature 10: KR 10-2017-0075646

### Patent Literature 11: US 2016/0233435

### Patent Literature 12: US 2015/0287933

### Patent Literature 13: US 2015/0333273

### Patent Literature 14: US 2015/0179942

### SUMMARY OF INVENTION

### Technical Problem

As a result of extensive research of the inventors, it has been found that there is still room to further improve the compounds disclosed in Patent Literatures 1 to 14 so as to provide an organic EL device with an improved performance.

An object of the invention is to provide a novel compound having a high orientation and a material for organic EL device comprising the novel compound. Another object of the invention is to provide an organic EL device comprising the compound and an electronic device comprising the organic EL device.

### Solution to Problem

As a result of extensive research on the compounds having an indro[3,2,1-jk]carbazole skeleton, the inventors have found that two substituents on the specific two positions of the benzene rings in the skeleton are important for improving the orientation of the compound. The present invention is based on this finding.

In an aspect, the present invention provides a compound represented by formula (1) (hereinafter also referred to as “compound (1)”):

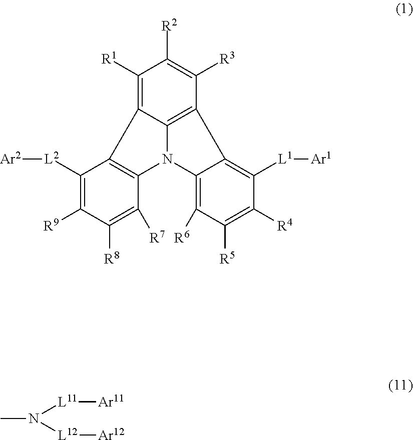

wherein:

R¹ to R⁹ are each independently a hydrogen atom, a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³), a group represented by —N(R¹⁰⁴)(R¹⁰⁵), or a substituted or unsubstituted heterocyclic group having 5 to 50 ring atoms, or

adjacent two selected from R¹ to R³, adjacent two selected from R⁴ to R⁶, or adjacent two selected from R⁷ to R⁹ form a substituted or unsubstituted ring structure;

R¹⁰¹ to R¹⁰⁵ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heterocyclic group having 5 to 50 ring atoms;

Ar¹ and Ar² are each independently a substituted or unsubstituted aryl group having 6 to 50 ring carbon atoms, a substituted or unsubstituted heterocyclic group having 5 to 50 ring atoms, or a group represented by formula (11);

Ar¹¹ and Ar¹² are each independently a substituted or unsubstituted aryl group having 6 to 50 ring carbon atoms or a substituted or unsubstituted heterocyclic group having 5 to 50 ring atoms, and Ar¹¹ and Ar¹² may be bonded to each other via a single bond; and

L¹, L², L¹¹ and L¹² are each independently a single bond, a substituted or unsubstituted arylene group having 6 to 50 ring carbon atoms, or a substituted or unsubstituted divalent heterocyclic group having 5 to 50 ring atoms.

In another aspect, the present invention provides a compound represented by formula (1) (hereinafter also referred to as “compound (1)”):

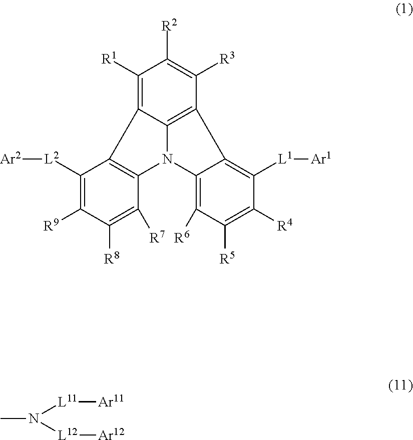

wherein:

R¹ to R⁹ are each independently a hydrogen atom, a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³), a group represented by —N(R¹⁰⁴)(R¹⁰⁵), or a substituted or unsubstituted heterocyclic group having 5 to 50 ring atoms, or

adjacent two selected from R¹ to R³, adjacent two selected from R⁴ to R⁶, or adjacent two selected from R⁷ to R⁹ form a substituted or unsubstituted ring structure;

R¹⁰¹ to R¹⁰⁵ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heterocyclic group having 5 to 50 ring atoms;

Ar¹ and Ar² are each independently a substituted or unsubstituted aryl group having 6 to 50 ring carbon atoms, a substituted or unsubstituted heterocyclic group having 5 to 50 ring atoms, or a group represented by formula (11);

Ar¹¹ and Ar¹² are each independently a substituted or unsubstituted aryl group having 6 to 50 ring carbon atoms or a substituted or unsubstituted heterocyclic group having 5 to 50 ring atoms;

Ar¹¹ and Ar¹² may be bonded to each other via a single bond or may form a substituted or unsubstituted ring structure together with R⁴; and

L¹, L², L¹¹ and L¹² are each independently a single bond, a substituted or unsubstituted arylene group having 6 to 50 ring carbon atoms, or a substituted or unsubstituted divalent heterocyclic group having 5 to 50 ring atoms.

In still another aspect, the present invention provides a material for organic EL device comprising the compound (1).

In still another aspect, the present invention provides an organic EL device comprising a cathode, an anode, and an organic layer disposed between the cathode and the anode, wherein the organic layer comprises a light emitting layer and at least one layer of the organic layer comprises the compound (1).

In still another aspect, the present invention provides an electronic device comprising the organic EL device.

### Advantageous Effects of Invention

An organic EL device produced by using the compound of the invention as a material for organic EL device has an excellent performance. Therefore, the organic EL device comprising the compound of the invention is useful for an electronic device.

### BRIEF DESCRIPTION OF DRAWINGS

FIG. 1 is a schematic cross-sectional view showing the layered structure of an organic electroluminescence device in an embodiment of the invention.

FIG. 2 is a schematic cross-sectional view showing the layered structure of an organic electroluminescence device in another embodiment of the invention.

### DESCRIPTION OF EMBODIMENTS

The term of “XX to YY carbon atoms” referred to by “a substituted or unsubstituted group ZZ having XX to YY carbon atoms” used herein is the number of carbon atoms of the unsubstituted group ZZ and does not include any carbon atom in the substituent of the substituted group ZZ.

The term of “XX to YY atoms” referred to by “a substituted or unsubstituted group ZZ having XX to YY atoms” used herein is the number of atoms of the unsubstituted group ZZ and does not include any atom in the substituent of the substituted group ZZ.

The number of “ring carbon atoms” referred to herein means the number of the carbon atoms included in the atoms which are members forming the ring itself of a compound in which a series of atoms is bonded to form the ring (for example, a monocyclic compound, a fused ring compound, a cross-linked compound, a carbocyclic compound, and a heterocyclic compound). If the ring has a substituent, the carbon atom in the substituent is not included in the ring carbon atom. The same applies to the number of “ring carbon atom” described below, unless otherwise noted. For example, a benzene ring has 6 ring carbon atoms, a naphthalene ring has 10 ring carbon atoms, a pyridinyl group has 5 ring carbon atoms, and a furanyl group has 4 ring carbon atoms. If a benzene ring or a naphthalene ring has, for example, an alkyl substituent, the carbon atom in the alkyl substituent is not counted as the ring carbon atom of the benzene or naphthalene ring. In case of a fluorene ring to which a fluorene substituent is bonded (inclusive of a spirofluorene ring), the carbon atom in the fluorene substituent is not counted as the ring carbon atom of the fluorene ring.

The number of “ring atom” referred to herein means the number of the atoms which are members forming the ring itself (for example, a monocyclic ring, a fused ring, and a ring assembly) of a compound in which a series of atoms is bonded to form the ring (for example, a monocyclic compound, a fused ring compound, a cross-linked compound, a carbocyclic compound, and a heterocyclic compound). The atom not forming the ring (for example, hydrogen atom(s) for saturating the valence of the atom which forms the ring) and the atom in a substituent, if the ring is substituted, are not counted as the ring atom. The same applies to the number of “ring atoms” described below, unless otherwise noted. For example, a pyridine ring has 6 ring atoms, a quinazoline ring has 10 ring atoms, and a furan ring has 5 ring atoms. The hydrogen atom on the ring carbon atom of a pyridine ring or a quinazoline ring and the atom in a substituent are not counted as the ring atom. In case of a fluorene ring to which a fluorene substituent is bonded (inclusive of a spirofluorene ring), the atom in the fluorene substituent is not counted as the ring atom of the fluorene ring.

The atom, the group and the ring structure represented by each symbol in the formulae described herein will be explained below.

### Hydrogen Atom

Hydrogen atom includes isotopes different in the neutron numbers, i.e., light hydrogen (protium), heavy hydrogen (deuterium), and tritium.

### Halogen Atom

The halogen atom is a fluorine atom, a chlorine atom, a bromine atom, or an iodine atom, with a fluorine atom being preferred.

### Alkyl Group

Unless otherwise noted, the alkyl group has 1 to 20, preferably 1 to 10, more preferably 1 to 6, still more preferably 1 to 5, and particularly preferably 1 to 4 carbon atoms.

Examples of the alkyl group include a methyl group, an ethyl group, a n-propyl group, an isopropyl group, a n-butyl group, a s-butyl group, an isobutyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), and a dodecyl group (inclusive of isomeric groups). Examples of the substituted alkyl group include a fluoromethyl group, a 1-fluoroethyl group, a 2-fluoroethyl group, a 2-fluoroisobutyl group, a 1,2-difluoroethyl group, a 1,3-difluoroisopropyl group, a 2,3-difluoro-t-butyl group, a 1,2,3-trifluoropropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, and a 1,2,3-triiodopropyl group.

Unless otherwise noted, the alkyl group is preferably a methyl group, an ethyl group, a n-propyl group, an isopropyl group, a n-butyl group, an isobutyl group, a s-butyl group, a t-butyl group, or a pentyl group (inclusive of isomeric groups), more preferably a methyl group, an ethyl group, a n-propyl group, an isopropyl group, a n-butyl group, an isobutyl group, a s-butyl group, or a t-butyl group, and still more preferably a methyl group, an ethyl group, an isopropyl group, or a t-butyl group.

### Alkenyl Group

Unless otherwise noted, the alkenyl group has 1 to 20, preferably 1 to 10, and more preferably 1 to 6 carbon atoms.

Examples of the alkenyl group include a vinyl group, a 2-propenyl group, a 2-butenyl group, a 3-butenyl group, a 4-pentenyl group, a 2-methyl-2-propenyl group, a 2-methyl-2-butenyl group, and a 3-methyl-2-butenyl group.

### Alkynyl Group

Unless otherwise noted, the alkynyl group has 1 to 20, preferably 1 to 10, and more preferably 1 to 6 carbon atoms.

Examples of the alkynyl group include a 2-propynyl group, a 2-butynyl group, a 3-butynyl group, a 4-pentynyl group, a 5-hexynyl group, a 1-methyl-2-propynyl group, a 1-methyl-2-butynyl group, and a 1,1-dimethyl-2-propynyl group.

### Cycloalkyl Group

Unless otherwise noted, the cycloalkyl group has 3 to 20, preferably 3 to 6, and more preferably 5 or 6 ring carbon atoms.

Examples of the cycloalkyl group include a cyclopropyl group, a cyclobutyl group, a cyclopentyl group, a cyclohexyl group, a 4-methylcyclohexyl group, a cycloheptyl group, a cyclooctyl group, an adamantyl group, and a norbornyl group.

Unless otherwise noted, preferred are a cyclopentyl group and a cyclohexyl group.

### Alkoxy Group

Unless otherwise noted, the alkoxy group has 1 to 20, preferably 1 to 10, and more preferably 1 to 6 carbon atoms.

The alkoxy group is represented by —OR^{A}, wherein R^{A} is an alkyl group or a cycloalkyl group which are selected from the examples of the alkyl group and the cycloalkyl group described above.

Unless otherwise noted, preferred alkoxy groups include a methoxy group, an ethoxy group, a n-propoxy group, an isopropoxy group, and a t-butoxy group.

### Alkylthio Group

Unless otherwise noted, the alkylthio group has 1 to 20, preferably 1 to 10, and more preferably 1 to 6 carbon atoms.

The alkylthio group is represented by —SR^{A}, wherein R^{A} is as defined above.

Unless otherwise noted, the alkylthio group is preferably a methylthio group, an ethylthio group, a n-propylthio group, an isopropylthio group, or a t-butylthio group.

### Aryl Group and Arylene Group

Unless otherwise noted, the aryl group has 6 to 50, preferably 6 to 30, and more preferably 6 to 24 ring carbon atoms.

Examples of the aryl group include a phenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, a 1-naphthyl group, a 2-naphthyl group, an acenaphthylenyl group, a 1-anthryl group, a 2-anthryl group, a 9-anthrylgroup, abenzanthrylgroup, an aceanthrylgroup, a 1-phenanthrylgroup, a 2-phenanthryl group, a 3-phenanthryl group, a 4-phenanthryl group, a 9-phenanthryl group, a 1-naphthacenyl group, a 2-naphthacenyl group, a 9-naphthacenyl group, a benzo[c]phenanthryl group, a phenalenyl group, a fluorenyl group, a picenyl group, a pentaphenyl group, a 1-pyrenyl group, a 2-pyrenyl group, a 4-pyrenyl group, a chrysenyl group, a benzo[g]chrysenyl group, a s-indacenyl group, an as-indacenyl group, a fluoranthenyl group, a benzo[k]fluoranthenyl group, a triphenylenyl group, a benzo[b]triphenylenyl group, and a perylenyl group. Examples of the substituted aryl group include an o-tolyl group, a m-tolyl group, a p-tolyl group, a 2,6-dimethylphenyl group, a p-isopropylphenyl group, a m-isopropylphenyl group, an o-isopropylphenyl group, a p-t-butylphenyl group, a m-t-butylphenyl group, an o-t-butylphenyl group, a (2-phenylpropyl)phenyl group, a 3,4,5-trimethylphenyl group, a 4-methoxyphenyl group, a 4-phenoxyphenyl group, a 3,4-dimethoxyphenyl group, a 3,4,5-trimethoxyphenyl group, a 4′-methylbiphenylyl group, a 4″-t-butyl-p-terphenyl-4-yl group, a 3-methyl-2-naphthyl group, a 4-methyl-1-naphthyl group, a 4-methyl-1-anthryl group, a 9,9-dimethylfluorenyl group, a 9,9-di(4-methylphenyl)fluorenyl group, a 9,9-di(4-isopropylphenyl)fluorenyl group, a 9,9-di(4-t-butylphenyl)fluorenyl group, a 9,9-diphenylfluorenyl group, a 9,9′-spirobifluorenyl group, a 4-(methylsulfanyl)phenyl group, a 4-(phenylsulfanyl)phenyl group, and a N′,N′-dimethyl-N-phenyl group.

Unless otherwise noted, the aryl group is preferably a phenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, a 1-naphthyl group, a 2-naphthyl group, a 1-anthryl group, a 2-anthryl group, a 9-anthryl group, a 1-pyrenyl group, a 2-pyrenyl group, a 4-pyrenyl group, or a fluoranthenyl group, more preferably a phenyl group, a 2-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, or a m-terphenyl-2-yl group, and still more preferably a phenyl group.

Examples of the arylene group include divalent residues derived from the examples of the aryl groups described above. The carbon number and preferred carbon number of the arylene group are the same as those of the aryl group.

### Aralkyl Group

Unless otherwise noted, the aralkyl group has 6 to 50, preferably 6 to 30, and more preferably 6 to 24 ring carbon atoms. Unless otherwise noted, the aralkyl group has 7 to 51, preferably 7 to 30, and more preferably 7 to 20 carbon atoms.

The aralkyl group is represented by —R^{B}Ar^{C}, wherein R^{B} is an alkylene group, for example, a group derived from R^{A} described above by removing one hydrogen atom and Ar^{C} is an aryl group, for example, a group selected from the examples of the aryl group described above.

Unless otherwise noted, the aralkyl group is preferably a benzyl group, a phenethyl group, or a phenylpropyl group and more preferably a benzyl group.

Aryloxy Group Unless otherwise noted, the aryloxy group has 6 to 50, preferably 6 to 25, and more preferably 6 to 18 ring carbon atoms.

The aryloxy group is represented by —OAr^{C}, wherein Ar^{C} is as defined above.

Unless otherwise noted, the aryloxy group is preferably a phenoxy group, a biphenyloxy group, or a terphenyloxy group, more preferably a phenoxy group or a biphenyloxy group, and still more preferably a phenoxy group.

### Arylthio Group

Unless otherwise noted, the arylthio group has 6 to 50, preferably 6 to 25, and more preferably 6 to 18 ring carbon atoms.

The arylthio group is represented by —SAr^{C}, wherein Ar^{C} is as defined above.

Unless otherwise noted, the arylthio group is preferably a phenylthio group, a biphenylthio group, or a terphenylthio group, more preferably a phenylthio group or a biphenylthio group, and still more preferably a phenylthio group.

Group Represented by —Si(R¹⁰¹)(R¹⁰²)(R¹⁰³)

Examples of the group represented by —Si(R¹⁰¹)(R¹⁰²)(R¹⁰³) include a trimethylsilyl group, a triethylsilyl group, a t-butyldimethylsilyl group, a propyldimethylsilyl group, an isopropyldimethylsilyl group, a triphenylsilyl group, a phenyldimethylsilyl group, a t-butyldiphenylsilyl group, and a tritolylsilyl group.

R¹⁰¹ to R¹⁰³ are each independently a hydrogen atom, a substituted or unsubstituted alkyl group having 1 to 20, preferably 1 to 10, and more preferably 1 to 6 carbon atoms, a substituted or unsubstituted cycloalkyl group having 3 to 20, preferably 3 to 6, and more preferably 5 or 6 ring carbon atoms, a substituted or unsubstituted aryl group having 6 to 50, preferably 6 to 25, and more preferably 6 to 18 ring carbon atoms, or a substituted or unsubstituted heterocyclic group having 5 to 50, preferably 5 to 24, and still more preferably 5 to 13 ring atoms.

Group Represented by —N(R¹⁰⁴)(R¹⁰⁵)

Examples of the group represented by —N(R¹⁰⁴)(R¹⁰⁵) include a dimethylamino group, a diethylamino group, a diisopropylamino group, and a diphenylamino group.

R¹⁰⁴ and R¹⁰⁵ are as defined above with respect to R¹⁰¹ to R¹⁰³.

### Heterocyclic Group

Unless otherwise noted, the heterocyclic group has 3 to 50, preferably 5 to 24, and more preferably 5 to 13 ring atoms.

The heterocyclic group has one or more ring hetero atoms that are selected from, for example, a nitrogen atom, an oxygen atom, and a sulfur atom. The free valence of the heterocyclic group is present on a ring carbon atom or a ring hetero atom.

The heterocyclic group is classified into an aliphatic heterocyclic group and an aromatic heterocyclic group. Examples of the aliphatic heterocyclic group include an epoxy group, an oxetanyl group, a tetrahydrofuranyl group, a pyrrolidyl group, a piperidinyl group, and a morpholinyl group. Examples of the aromatic heterocyclic group include a pyrrolyl group, an imidazolyl group, a pyrazolyl group, a triazolyl group, a furyl group, a thienyl group, an oxazolyl group, an isoxazolyl group, an oxadiazolyl group, a thiazolyl group, an isothiazolyl group, a thiadiazolyl group, a pyridyl group, a pyridazinyl group, a pyrimidinyl group, a pyridinyl group, a triazinyl group, an indolyl group, an isoindolyl group, an indolizinyl group, a quinolizinyl group, a quinolinyl group, an isoquinolyl group, a cinnolyl group, a phthalazinyl group, a quinazolinyl group, a quinoxalinyl group, a benzimidazolyl group, an indazolyl group, a phenanthrolinyl group, a phenanthridinyl group, an acridinyl group, a phenazinyl group, a carbazolyl group, a benzocarbazolyl group, a xanthenyl group, a benzofuranyl group, an isobenzofuranyl group, a dibenzofuranyl group, a naphthobenzofuranyl group, a benzothiophenyl group (a benzothienyl group), a dibenzothiophenyl group (a dibenzothienyl group), and a naphthobenzothiophenyl group (a naphthobenzothienyl group).

Unless otherwise noted, the heterocyclic group is preferably a furyl group, a thienyl group, a pyridyl group, a pyridazinyl group, a pyrimidinyl group, a pyrazinyl group, a triazinyl group, a benzofuranyl group, a benzothiophenyl group, a dibenzofuranyl group, a naphthobenzofuranyl group, a dibenzothiophenyl group, a naphtobenzothiophenyl group, a carbazolyl group, or a benzocarbazolyl group.

Examples of the divalent heterocyclic group include divalent residues derived from the examples of the heterocyclic groups described above. The carbon number and preferred carbon number of the divalent heterocyclic group are the same as those of the heterocyclic group.

### Ring Structure

The ring structure is a fused or non-fused, aromatic or aliphatic ring. For example, the ring structure is a substituted or unsubstituted aromatic hydrocarbon ring, a substituted or unsubstituted aliphatic hydrocarbon ring, a substituted or unsubstituted aromatic heterocyclic ring, or a substituted or unsubstituted aliphatic heterocyclic ring.

The ring structure may include a substituted or unsubstituted fused ring comprising aromatic heterocyclic rings or aliphatic rings and a substituted or unsubstituted non-fused ring.

### Aromatic Hydrocarbon Ring

Unless otherwise noted, the aromatic hydrocarbon ring has 6 to 30, preferably 6 to 25, and more preferably 6 to 18 ring carbon atoms.

Examples of the aromatic hydrocarbon ring include a benzene ring, a biphenylene ring, a naphthalene ring, an anthracene ring, a benzanthracene ring, a phenanthrene ring, a benzophenanthrene ring, a phenalene ring, a pyrene ring, a chrysene ring, and a triphenylene ring, with a benzene ring and a naphthalene ring being preferred.

### Aliphatic Hydrocarbon Ring

Unless otherwise noted, the aliphatic hydrocarbon ring has 5 to 30, preferably 6 to 25, more preferably 6 to 18 ring carbon atoms. Examples of the aliphatic hydrocarbon ring include a cyclopentene ring, a cyclopentadiene ring, a cyclohexene ring, a cyclohexadiene ring, and an aliphatic hydrocarbon ring obtained by partially hydrogenating the aromatic hydrocarbon ring described above.

### Heterocyclic Ring

Unless otherwise noted, the aromatic heterocyclic ring has 5 to 30, preferably 6 to 25, and more preferably 6 to 18 ring atoms.

Examples of the aromatic heterocyclic ring include a pyrrole ring, a furan ring, a thiophene ring, a pyridine ring, an imidazole ring, a pyrazole ring, an indole ring, an isoindole ring, a benzofuran ring, an isobenzofuran ring, a benzothiophene ring, a benzimidazole ring, an imidazole ring, a dibenzofuran ring, a naphthobenzofuran ring, a dibenzothiophene ring, a naphthobenzothiophene ring, a carbazole ring, and a benzocarbazole ring.

### Aliphatic Heterocyclic Ring

Unless otherwise noted, the aliphatic heterocyclic ring has 5 to 30, preferably 6 to 25, and more preferably 6 to 18 ring atoms.

Examples of the aliphatic heterocyclic ring include those obtained by partially hydrogenating the aromatic heterocyclic ring described above.

Unless otherwise noted, the optional substituent referred to by “substituted or unsubstituted” and the substituent are selected from a halogen atom, a cyano group, an alkyl group, an alkenyl group, an alkynyl group, a cycloalkyl group, an alkoxy group, an alkylthio group, an aryl group, an aralkyl group, an aryloxy group, an arylthio group, a group represented by —Si(R¹⁰¹)(R¹⁰²)(R¹⁰³), a group represented by —N(R¹⁰⁴)(R¹⁰⁵), a heterocyclic group, a nitro group, a hydroxy group, a carboxyl group, a vinyl group, a carbonyl group having a substituent selected from an alkyl group and an aryl group, a sulfonyl group having a substituent selected from an alkyl group and an aryl group, a di-substituted phosphoryl group having a substituent selected from an alkyl group and an aryl group, a alkylcarbonyloxy group, an arylcarbonyloxy group, an alkylsulfonyloxy group, an arylsulfonyloxy group, and a (meth)acryloyl group.

Adjacent optional substituents may form a substituted or unsubstituted ring structure.

The details of the halogen atom, the alkyl group, the alkenyl group, the alkynyl group, the cycloalkyl group, the alkoxy group, the alkylthio group, the aryl group, the aralkyl group, the aryloxy group, the arylthio group, R¹⁰¹ to R¹⁰⁵, and the heterocyclic group are as described above.

Unless otherwise noted, the optional substituent is preferably a halogen atom, a cyano group, an alkyl group, a cycloalkyl group, an aryl group, or a heterocyclic group.

### Compound

The compound in an aspect of the invention (“compound (1)”) is represented by formula (1):

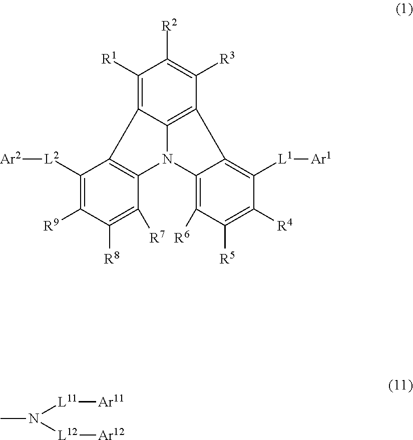

wherein:

R¹ to R⁹ are each independently a hydrogen atom, a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R^{1O1})(R¹⁰²)(R¹⁰³), a group represented by —N(R¹⁰⁴)(R¹⁰⁵), or a substituted or unsubstituted heterocyclic group having 5 to 50 ring atoms, or

adjacent two selected from R¹ to R³, adjacent two selected from R⁴ to R⁶, or adjacent two selected from R⁷ to R⁹ form a substituted or unsubstituted ring structure, wherein adjacent two selected from R¹ to R³ and adjacent two selected from R⁴ to R⁶ may simultaneously form the ring structure;

R¹⁰¹ to R¹⁰⁵ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heterocyclic group having 5 to 50 ring atoms;

Ar¹ and Ar² are each independently a substituted or unsubstituted aryl group having 6 to 50 ring carbon atoms, a substituted or unsubstituted heterocyclic group having 5 to 50 ring atoms, or a group represented by formula (11);

Ar¹¹ and Ar¹² are each independently a substituted or unsubstituted aryl group having 6 to 50 ring carbon atoms or a substituted or unsubstituted heterocyclic group having 5 to 50 ring atoms, and Ar¹¹ and Ar¹² may be bonded to each other via a single bond; and

L¹, L², L¹¹ and L¹² are each independently a single bond, a substituted or unsubstituted arylene group having 6 to 50 ring carbon atoms, or a substituted or unsubstituted divalent heterocyclic group having 5 to 50 ring atoms.

Preferably, Ar¹ and Ar² are each independently a substituted aryl group having 6 to 50 ring carbon atoms. Also preferably, Ar¹ and Ar² are each independently a substituted or unsubstituted aryl group having 10 to 50 ring carbon atoms, a substituted or unsubstituted heterocyclic group having 5 to 50 ring atoms, a group represented by formula (11), or a group represented by formula (21):

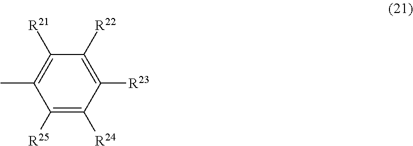

wherein:

R²¹ to R²⁵ are each independently a hydrogen atom, a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³) wherein R¹⁰¹ to R¹⁰³ are as defined above, a group represented by —N(R¹⁰⁴)(R¹⁰⁵) wherein R¹⁰⁴ and R¹⁰⁵ are as defined above, or a substituted or unsubstituted heterocyclic group having 5 to 50 ring atoms, with a cyano group being preferred;

provided that at least one of R²¹ to R²⁵ is a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³) wherein R¹⁰¹ to R¹⁰³ are as defined above, a group represented by —N(R¹⁰⁴)(R¹⁰⁵) wherein R¹⁰⁴ and R¹⁰⁵ are as defined above, or a substituted or unsubstituted heterocyclic group having 5 to 50 ring atoms.

The group represented by formula (11) is preferably a group represented by formula (31) wherein Ar¹¹ and Ar¹² of formula (11) are bonded to each other via a single bond:

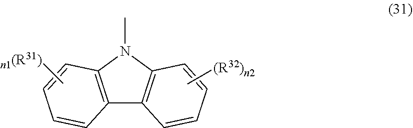

wherein:

R³¹ to R³² are each independently a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³) wherein R¹⁰¹ to R¹⁰³ are as defined above, a group represented by —N(R¹⁰⁴)(R¹⁰⁵) wherein R¹⁰⁴ and R¹⁰⁵ are as defined above, or a substituted or unsubstituted heterocyclic group having 5 to 50 ring atoms, or

adjacent two selected from R³¹ and R³² form a ring structure; and

n1 and n2 are each independently an integer of 0 to 4.

In a preferred embodiment of formula (1), -L¹-Ar¹ and -L²-Ar² are the same. In another preferred embodiment, adjacent two selected from R¹ to R³, adjacent two selected from R⁴ to R⁶, and adjacent two selected from R⁷ to R⁹ do not form a ring structure.

Preferably, R¹ to R⁹ of formula (1) are each independently a hydrogen atom, a cyano group, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, a group represented by —N(R¹⁰⁴)(R¹⁰⁵), or a substituted or unsubstituted heterocyclic group having 5 to 50 ring atoms. In a preferred embodiment, R¹ to R⁹ are all hydrogen atoms.

Preferred embodiments of formula (1) include formulae (2) to (9):

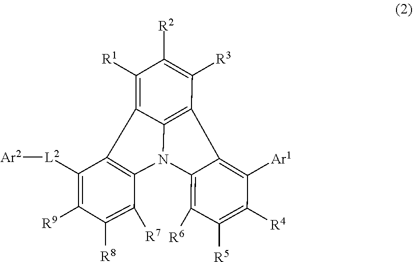

wherein R¹ to R⁹, Ar¹ to Ar² and L² are as defined above;

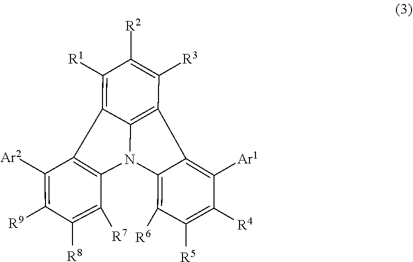

wherein R¹ to R⁹ and Ar¹ to Ar² are as defined above;

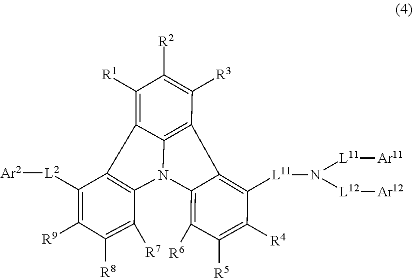

wherein R¹ to R⁹, Ar², Ar¹¹ to Ar¹², L¹ to L², and L¹¹ to L¹² are as defined above;

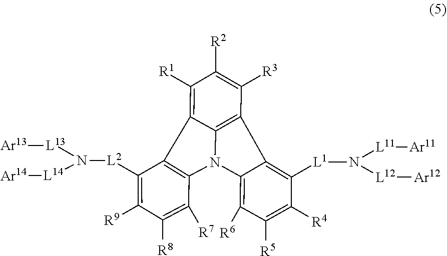

wherein:

R¹ to R⁹, Ar², Ar¹¹ to Ar¹², L¹ to L², and L¹¹ to L¹² are as defined above;

L¹³ to L¹⁴ are as defined above with respect to L¹¹ to L¹²; and

Ar¹³ to Ar¹⁴ are as defined above with respect to Ar¹¹ to Ar¹²;

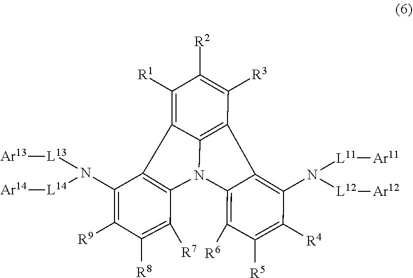

wherein:

R¹ to R⁹, Ar², Ar¹¹ to Ar¹², and L¹¹ to L¹² are as defined above;

L¹³ to L¹⁴ are as defined above with respect to L¹¹ to L¹²; and

Ar¹³ to Ar¹⁴ are as defined above with respect to Ar¹¹ to Ar¹²;

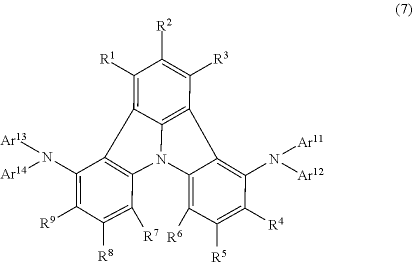

wherein:

R¹ to R⁹, Ar², and Ar¹¹ to Ar¹² are as defined above; and

Ar¹³ to Ar¹⁴ are as defined above with respect to Ar¹¹ to Ar¹²;

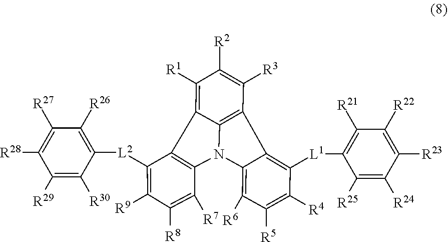

wherein:

R¹ to R⁹, L¹ to L², and R²¹ to R²⁵ are as defined above; and

R²⁶ to R³⁰ are as defined above with respect to R²¹ to R²⁵;

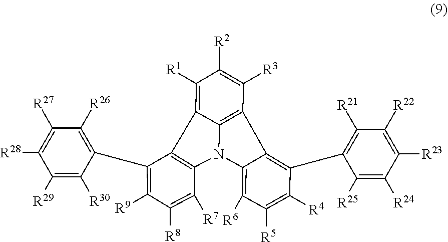

wherein:

R¹ to R⁹ and R²¹ to R²⁵ are as defined above; and

R²⁶ to R³⁰ are as defined above with respect to R²¹ to R²⁵.

In formulae (8) and (9), at least one of R²¹ to R²⁵ is preferably a cyano group and at least one of R²⁶ to R³⁰ is preferably a cyano group. More preferably, R²³ and R²⁸ are both cyano groups.

Preferred embodiments of formula (1) further include formulae (1-1) to (1-7):

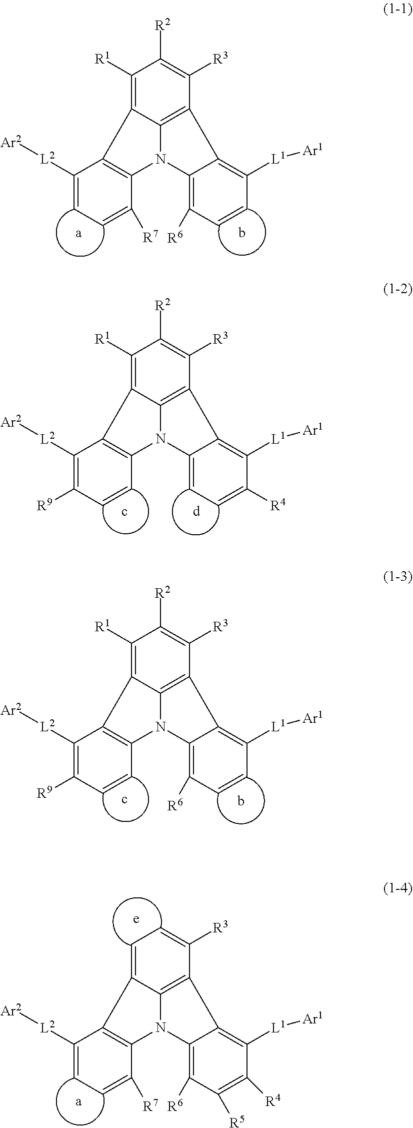

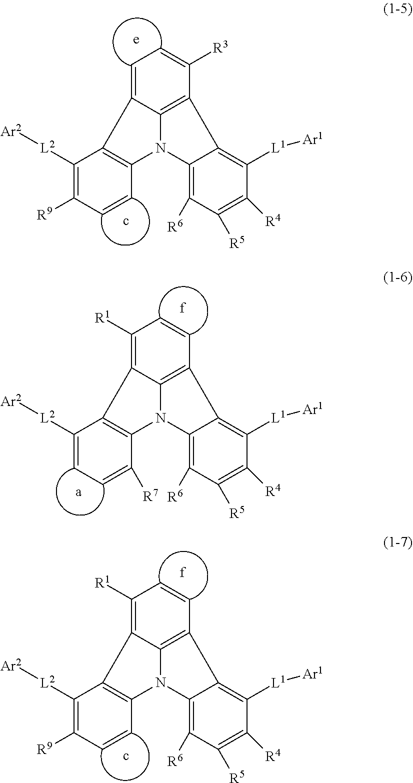

wherein:

R¹ to R⁷, R⁹, L¹, L², Ar¹, and Ar² are as defined above; and

each of rings a to f is a substituted or unsubstituted aromatic hydrocarbon ring having 6 to 30 ring carbon atoms or a substituted or unsubstituted aromatic heterocyclic ring having 5 to 30 ring atoms.

Preferred is formula (1-1) that is more preferably represented by formula (1-1-1):

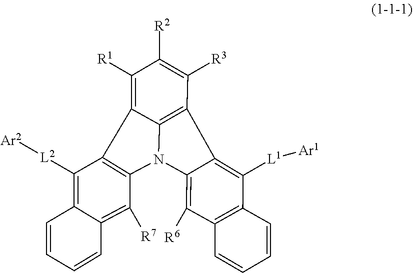

wherein R¹ to R³, R⁶ to R⁷, L¹, L², Ar¹, and Ar² are as defined above.

Preferred embodiments of formula (1) further include formulae (2-1) to (2-3) and (3-1) to (3-4):

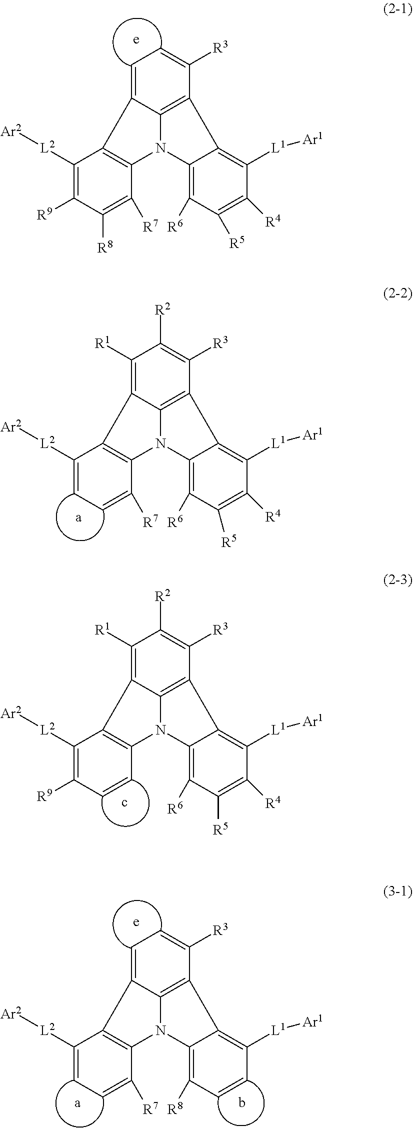

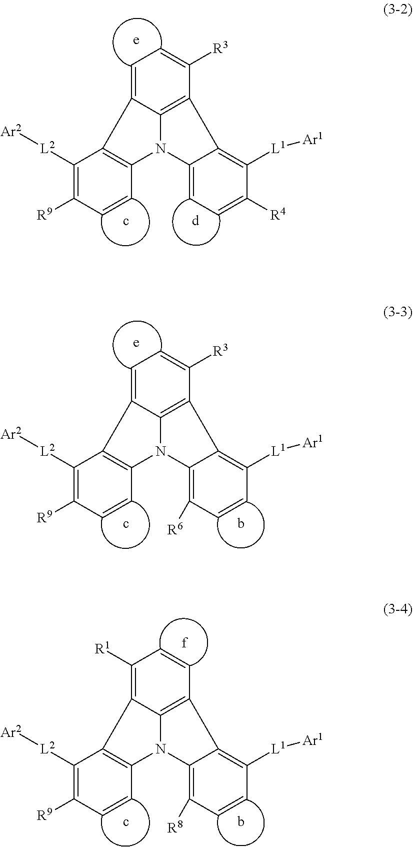

wherein:

R¹ to R⁹, L¹, L², Ar¹, and Ar² are as defined above; and

each of rings a to e is a substituted or unsubstituted aromatic hydrocarbon ring having 6 to 30 ring carbon atoms or a substituted or unsubstituted aromatic heterocyclic ring having 5 to 30 ring atoms.

Each of the rings a to e is preferably represented by any of formulae (41) to (47):

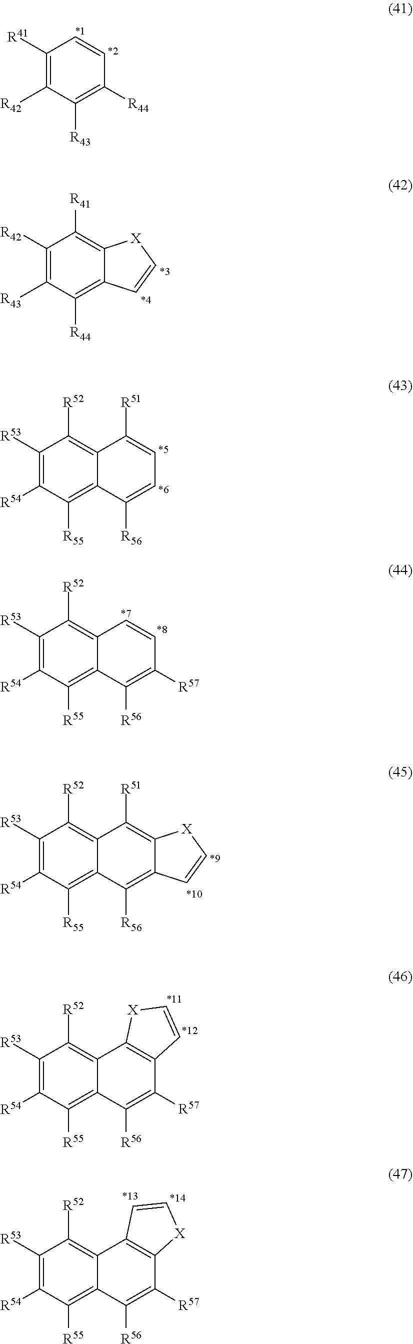

wherein:

*1 and *2, *3 and *4, *5 and *6, *7 and*8,*9 and *10, *11 and *12, and *13 and *14 each represent ring carbon atoms of a benzene ring to which each ring is bonded;

X is selected from C(R⁵⁸)(R⁵⁹), NR⁶⁰, O, and S; and

the definitions, examples, and preferred embodiments of R⁴¹ to R⁴⁴ and R⁵¹ to R⁶⁰ are as described above with respect to R¹ to R⁹.

Examples of the compound (1) are shown below, although not limited thereto.

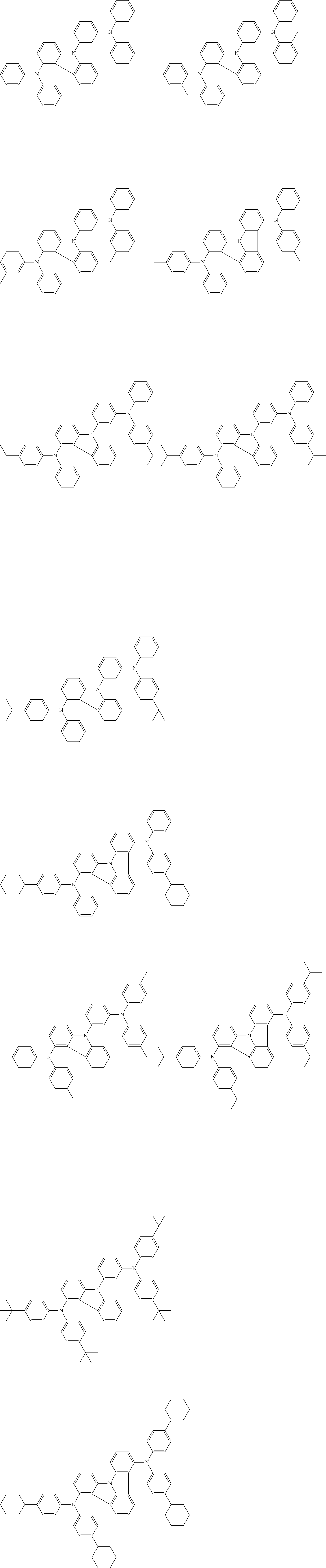

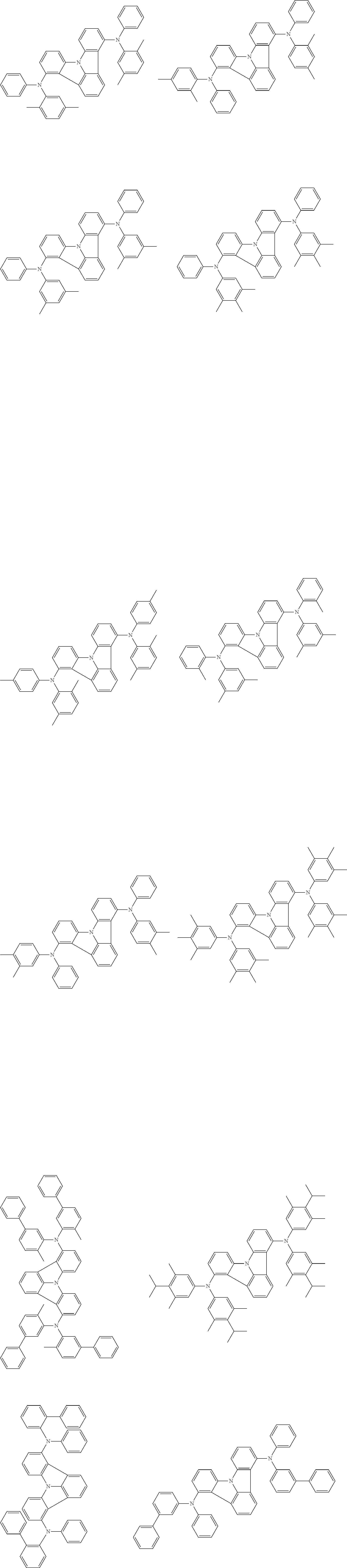

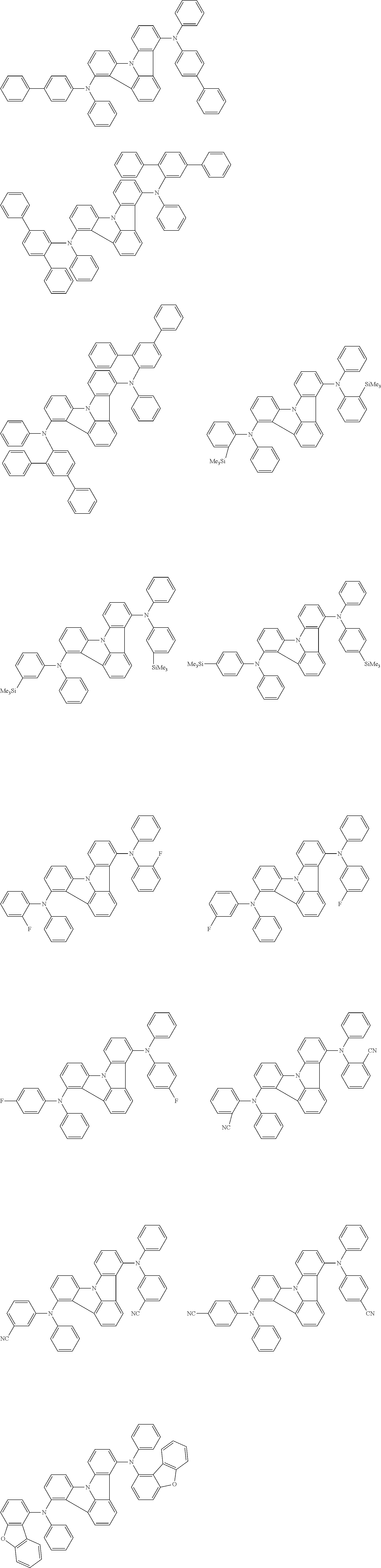

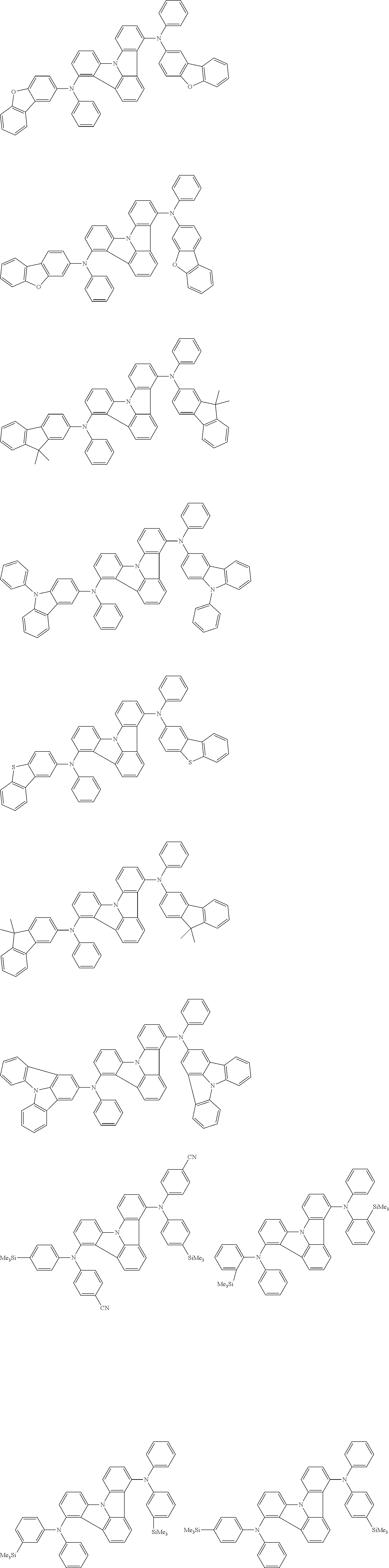

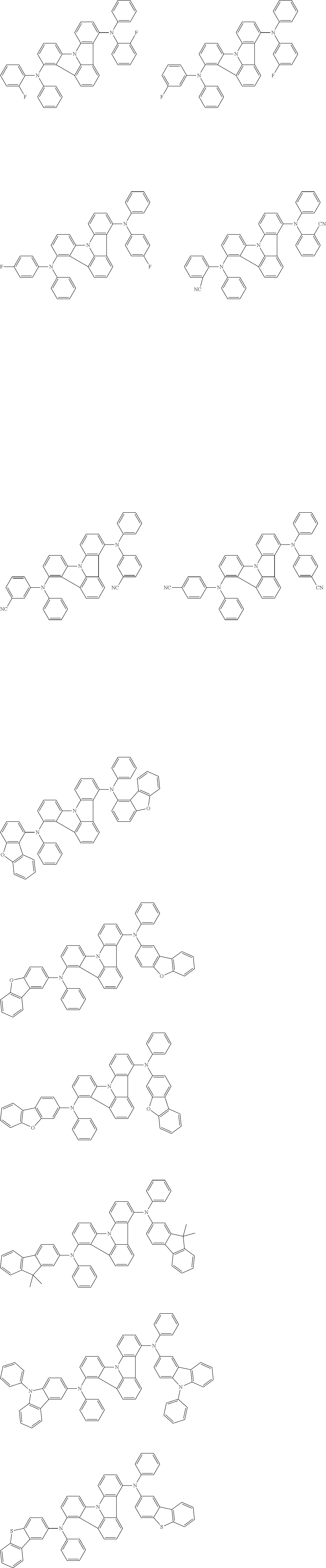

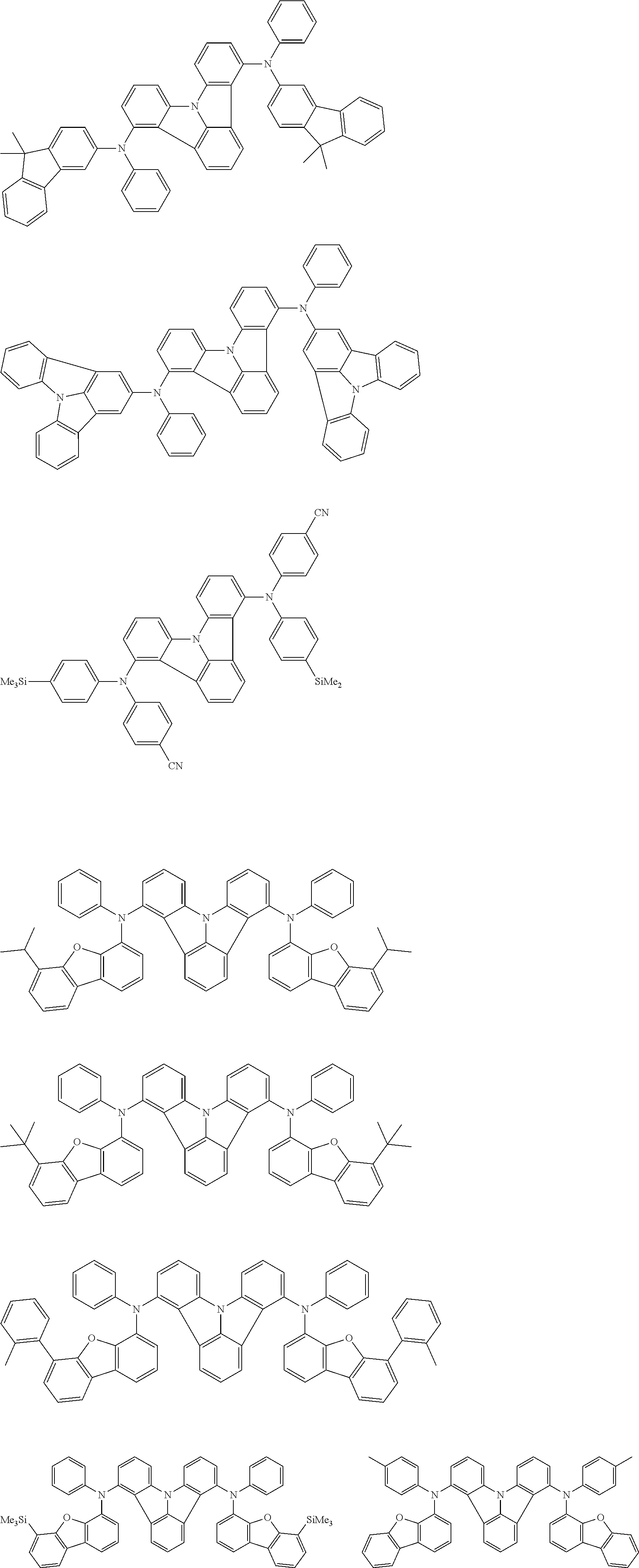

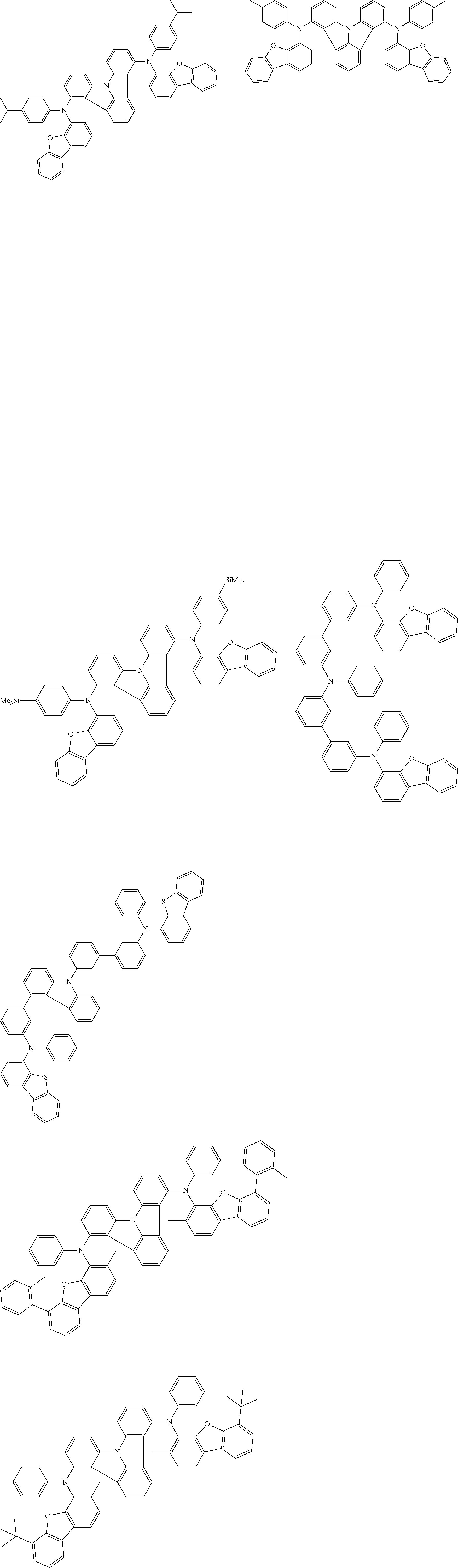

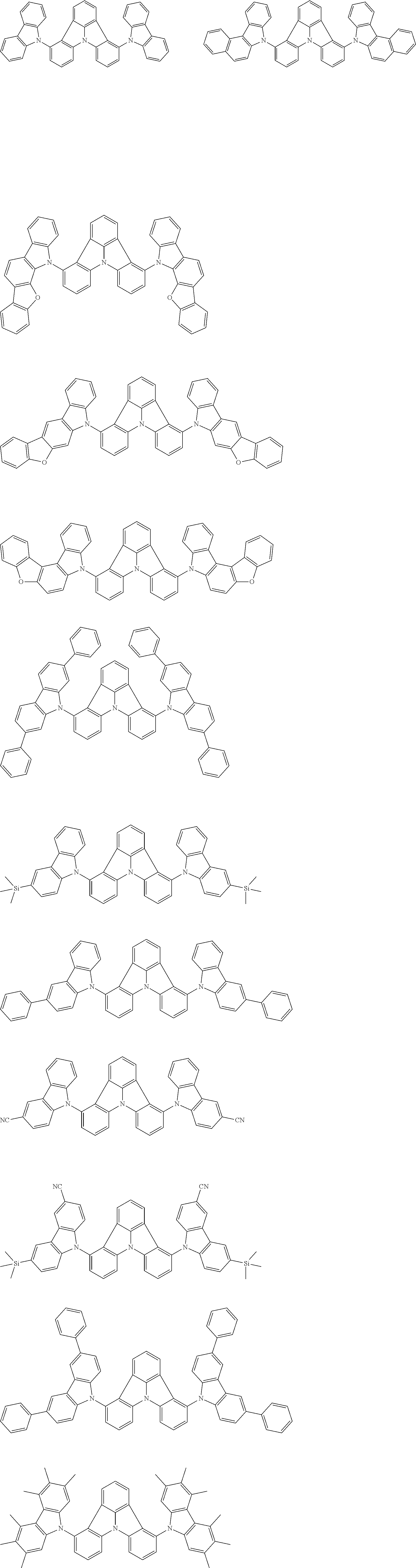

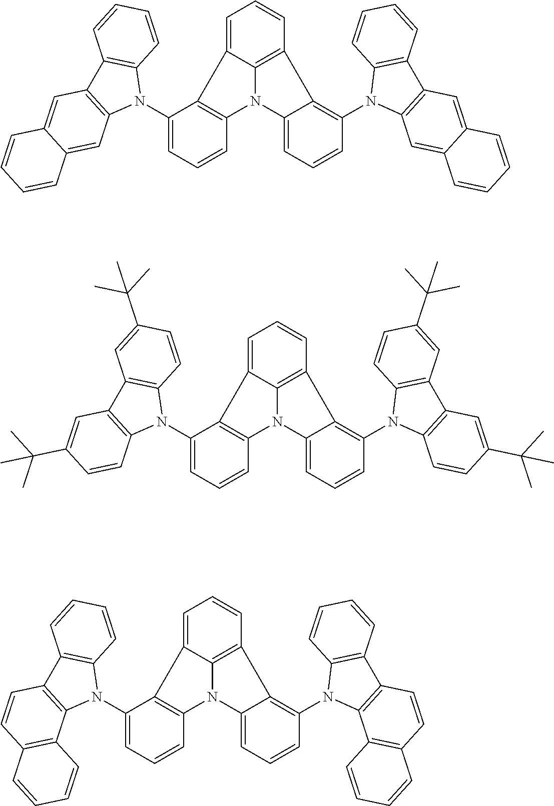 [SMILES: c1ccc2cc3c(cc2c1)c1ccccc1n3-c1cccc2c1c1cccc3c4c(-n5c6ccccc6c6cc7ccccc7cc65)cccc4n2c13 ; CC(C)(C)c1ccc2c(c1)c1cc(C(C)(C)C)ccc1n2-c1cccc2c1c1cccc3c4c(-n5c6ccc(C(C)(C)C)cc6c6cc(C(C)(C)C)ccc65)cccc4n2c13 ; c1ccc2c(c1)ccc1c3ccccc3n(-c3cccc4c3c3cccc5c6c(-n7c8ccccc8c8ccc9ccccc9c87)cccc6n4c35)c21]

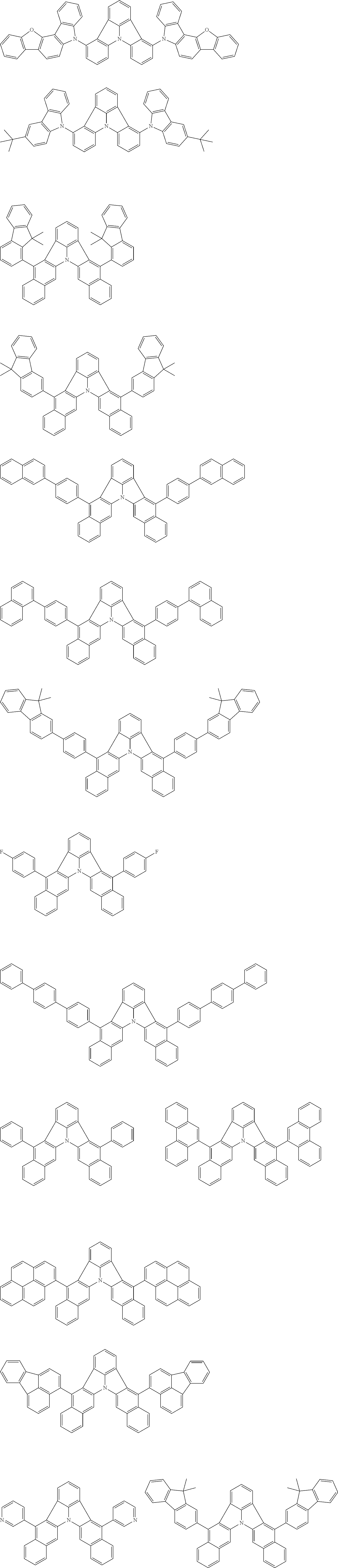

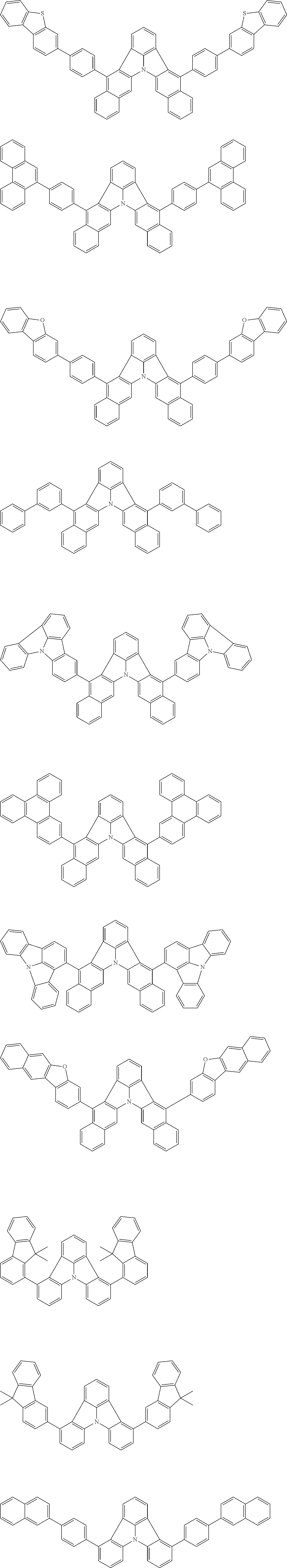

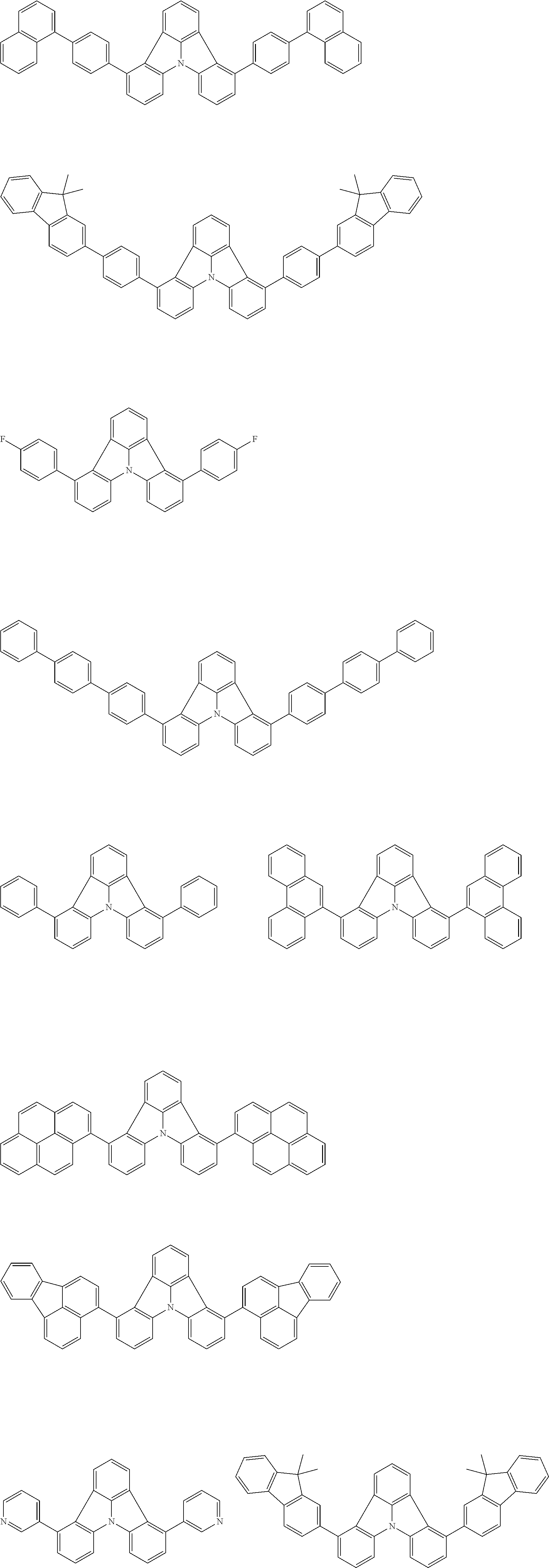

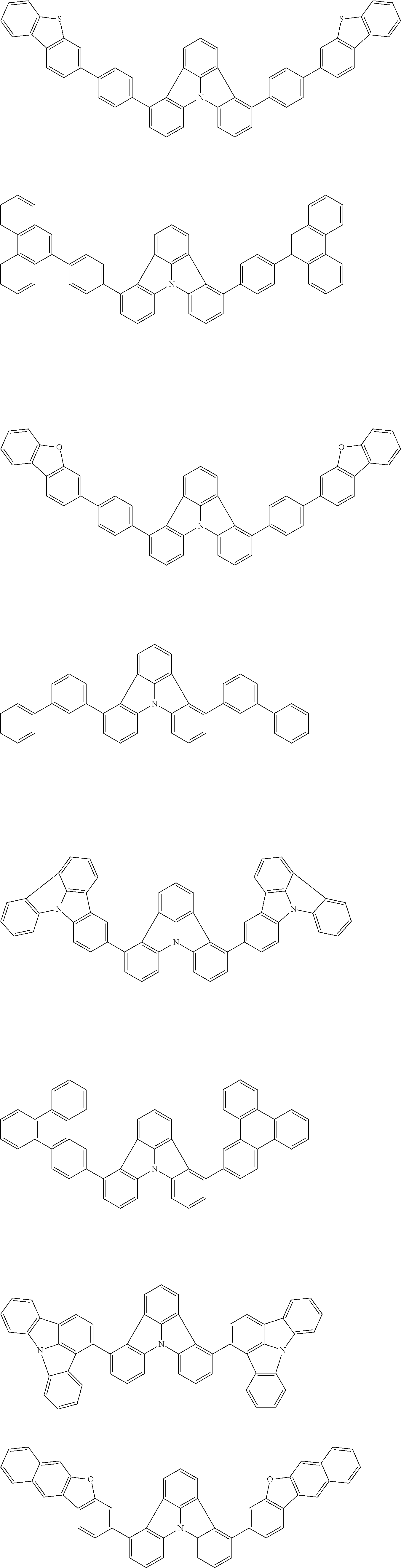

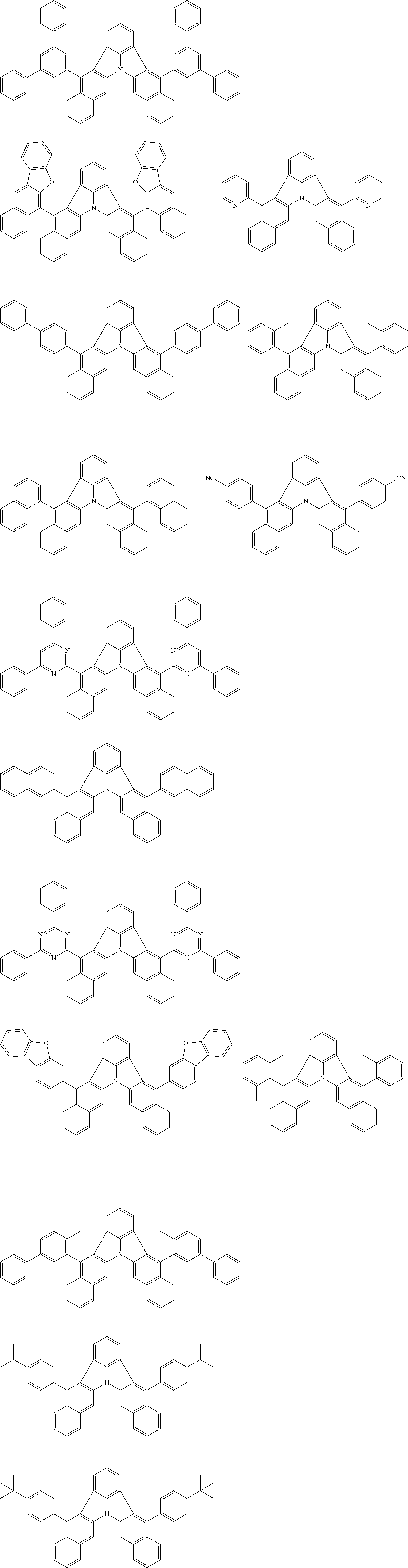

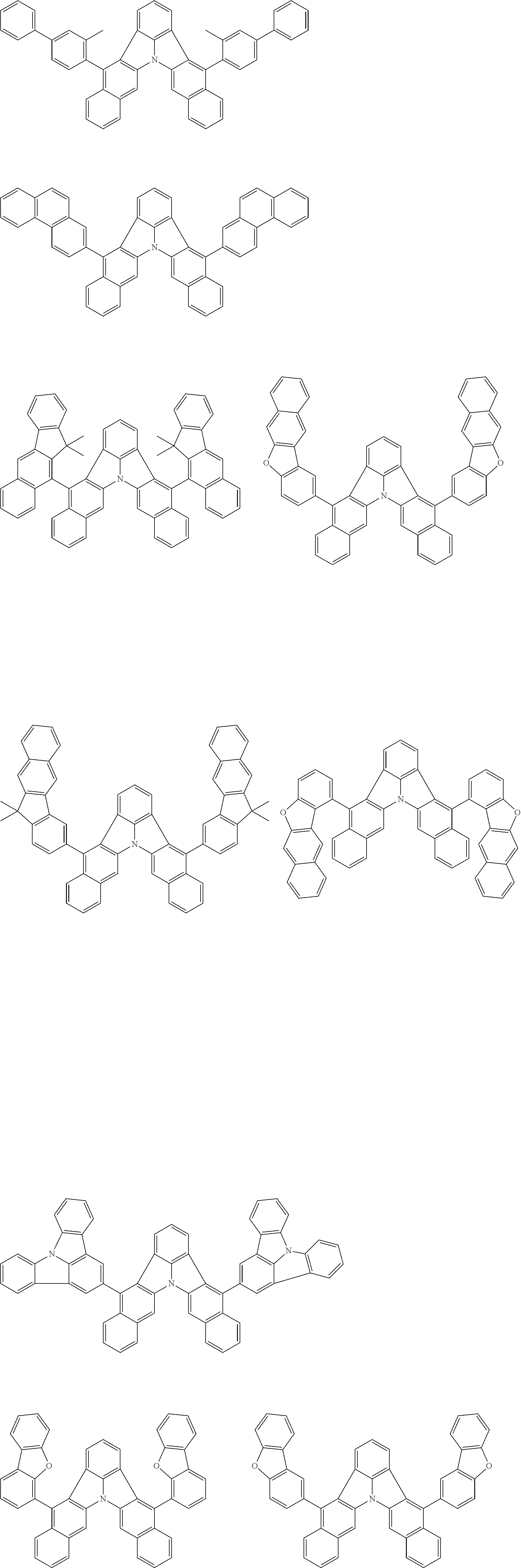

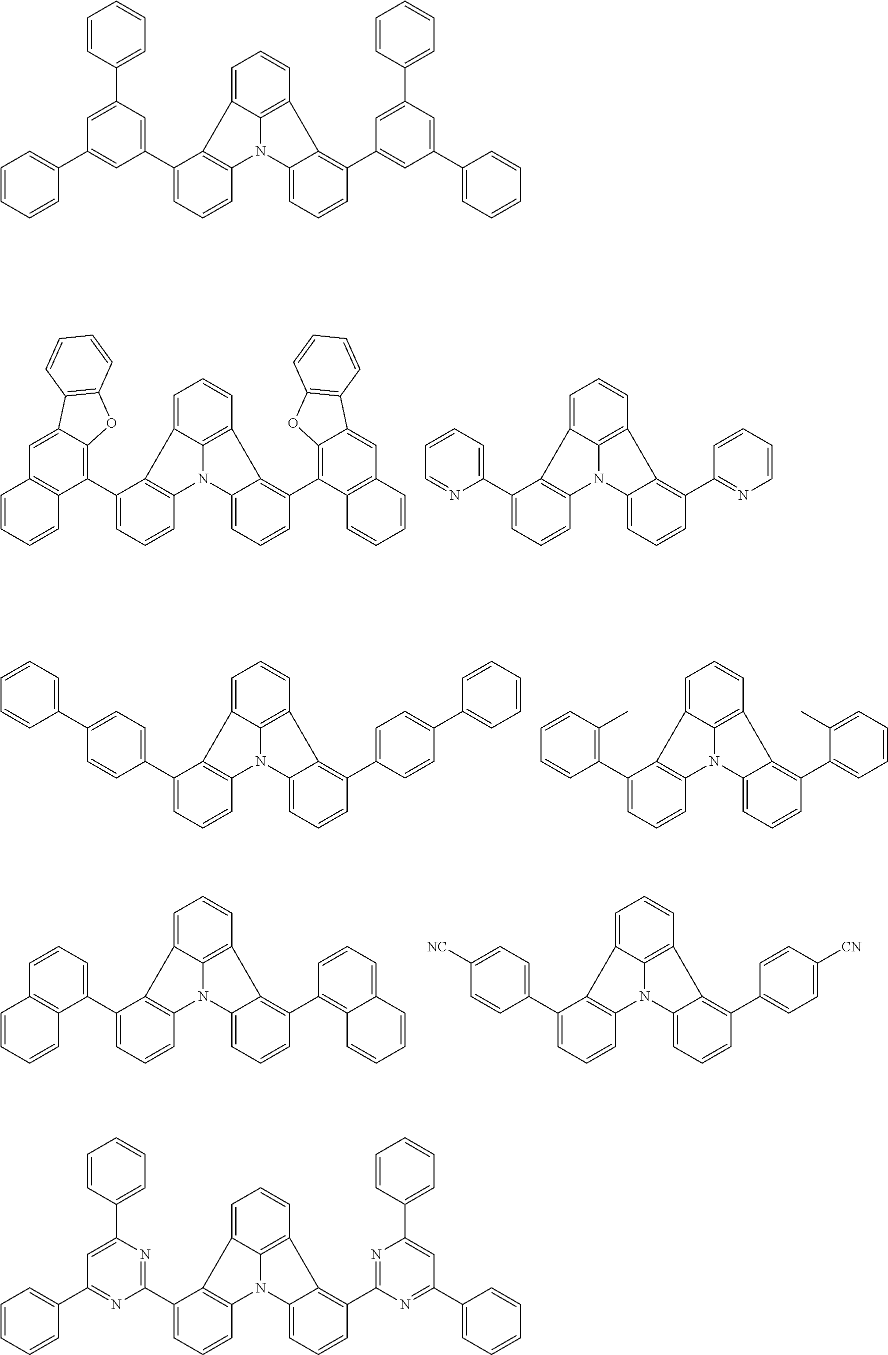

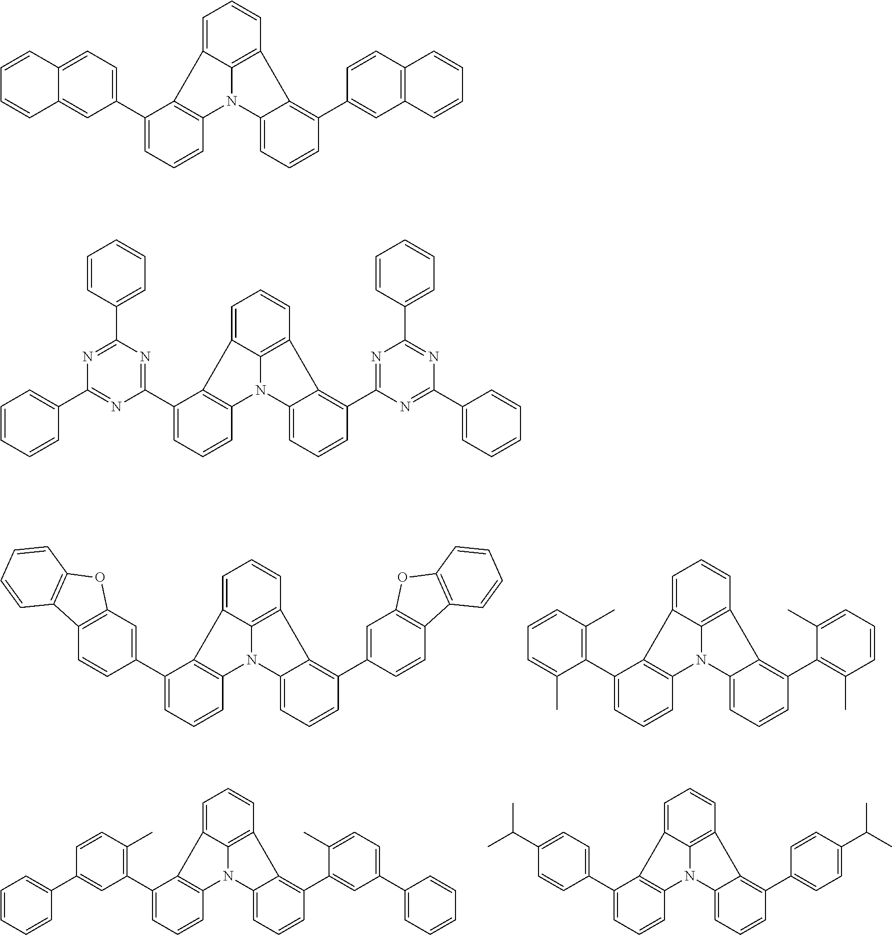 [SMILES: c1ccc2cc(-c3cccc4c3c3cccc5c6c(-c7ccc8ccccc8c7)cccc6n4c35)ccc2c1 ; c1ccc(-c2nc(-c3ccccc3)nc(-c3cccc4c3c3cccc5c6c(-c7nc(-c8ccccc8)nc(-c8ccccc8)n7)cccc6n4c35)n2)cc1 ; c1ccc2c(c1)oc1cc(-c3cccc4c3c3cccc5c6c(-c7ccc8c(c7)oc7ccccc78)cccc6n4c35)ccc12 ; Cc1cccc(C)c1-c1cccc2c1c1cccc3c4c(-c5c(C)cccc5C)cccc4n2c13 ; Cc1ccc(-c2ccccc2)cc1-c1cccc2c1c1cccc3c4c(-c5cc(-c6ccccc6)ccc5C)cccc4n2c13 ; CC(C)c1ccc(-c2cccc3c2c2cccc4c5c(-c6ccc(C(C)C)cc6)cccc5n3c24)cc1]

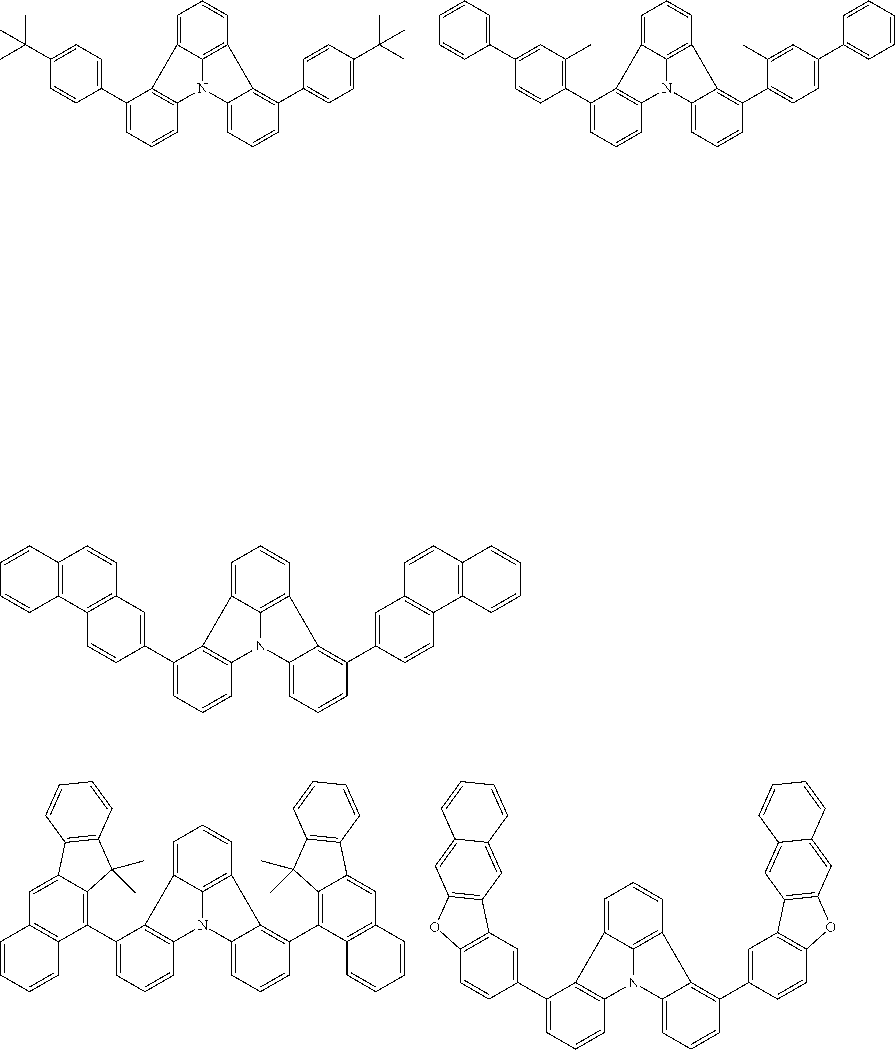

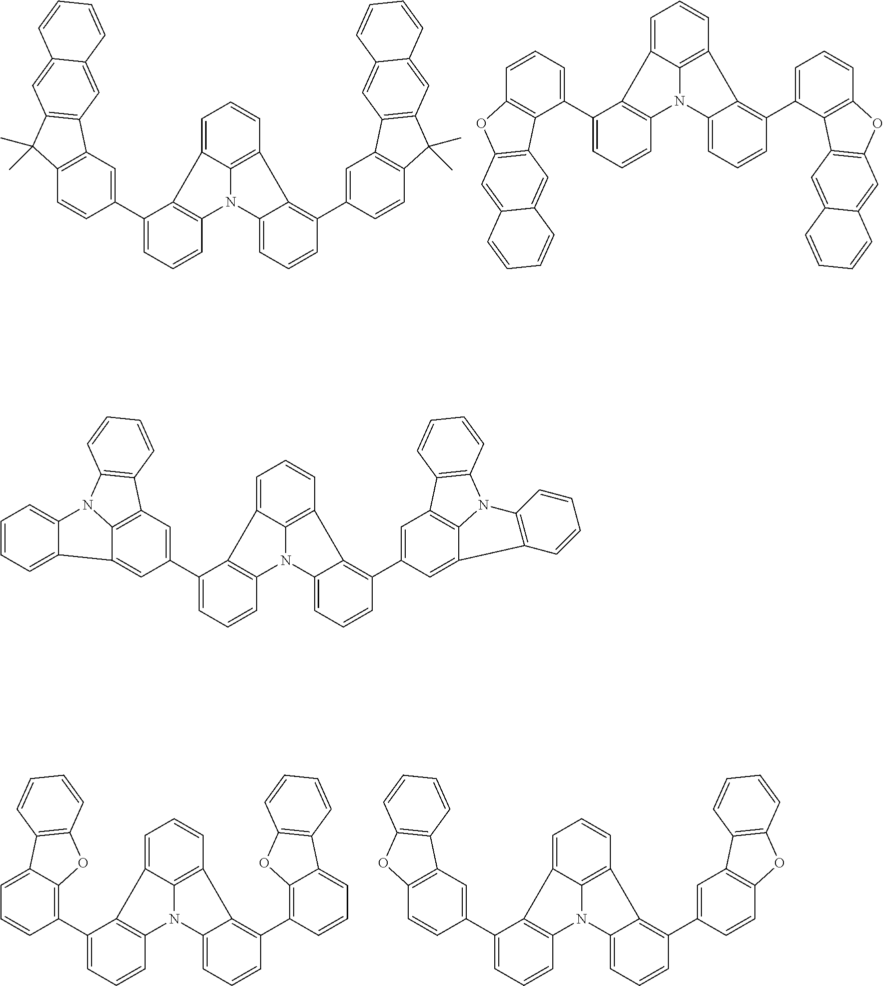

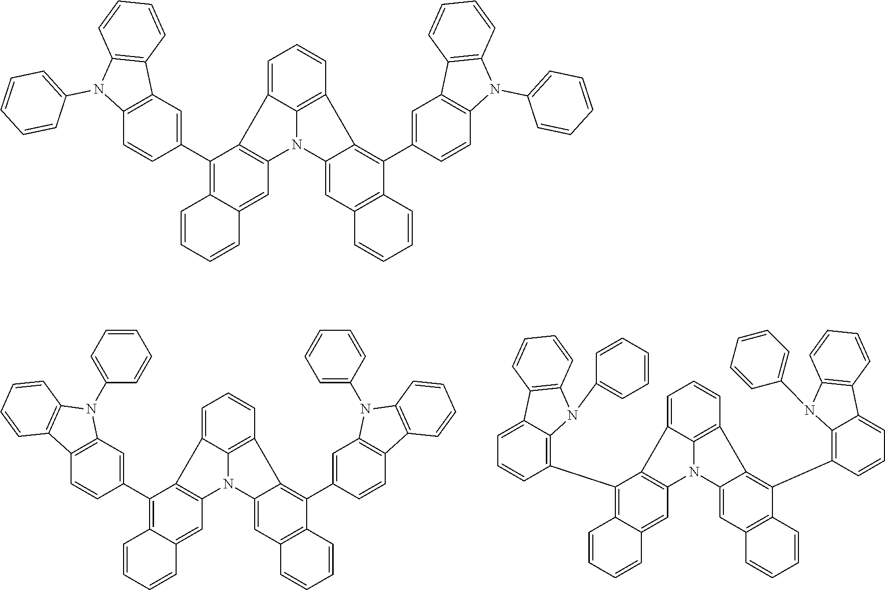 [SMILES: c1ccc(-n2c3ccccc3c3cc(-c4c5ccccc5cc5c4c4cccc6c7c(-c8ccc9c(c8)c8ccccc8n9-c8ccccc8)c8ccccc8cc7n5c46)ccc32)cc1 ; c1ccc(-n2c3ccccc3c3ccc(-c4c5ccccc5cc5c4c4cccc6c7c(-c8ccc9c%10ccccc%10n(-c%10ccccc%10)c9c8)c8ccccc8cc7n5c46)cc32)cc1 ; c1ccc(-n2c3ccccc3c3cccc(-c4c5ccccc5cc5c4c4cccc6c7c(-c8cccc9c%10ccccc%10n(-c%10ccccc%10)c89)c8ccccc8cc7n5c46)c32)cc1]

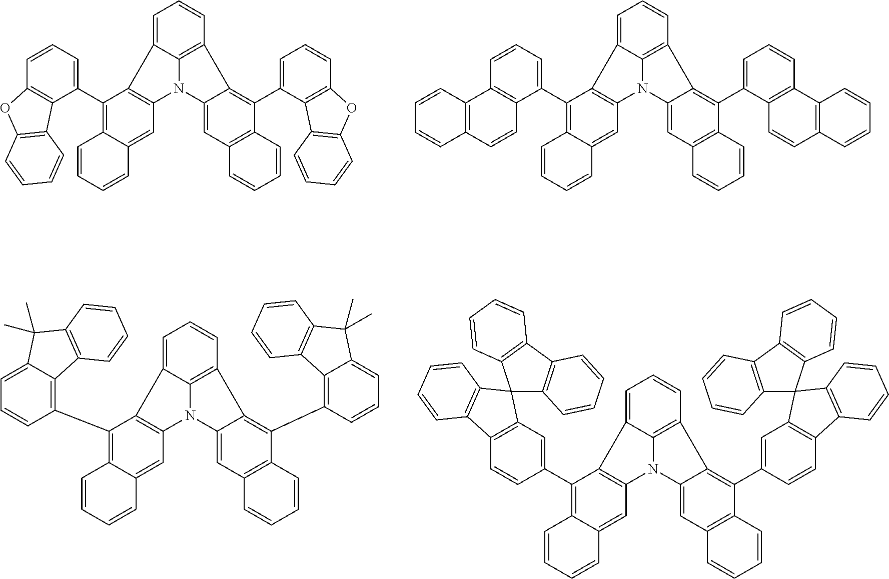

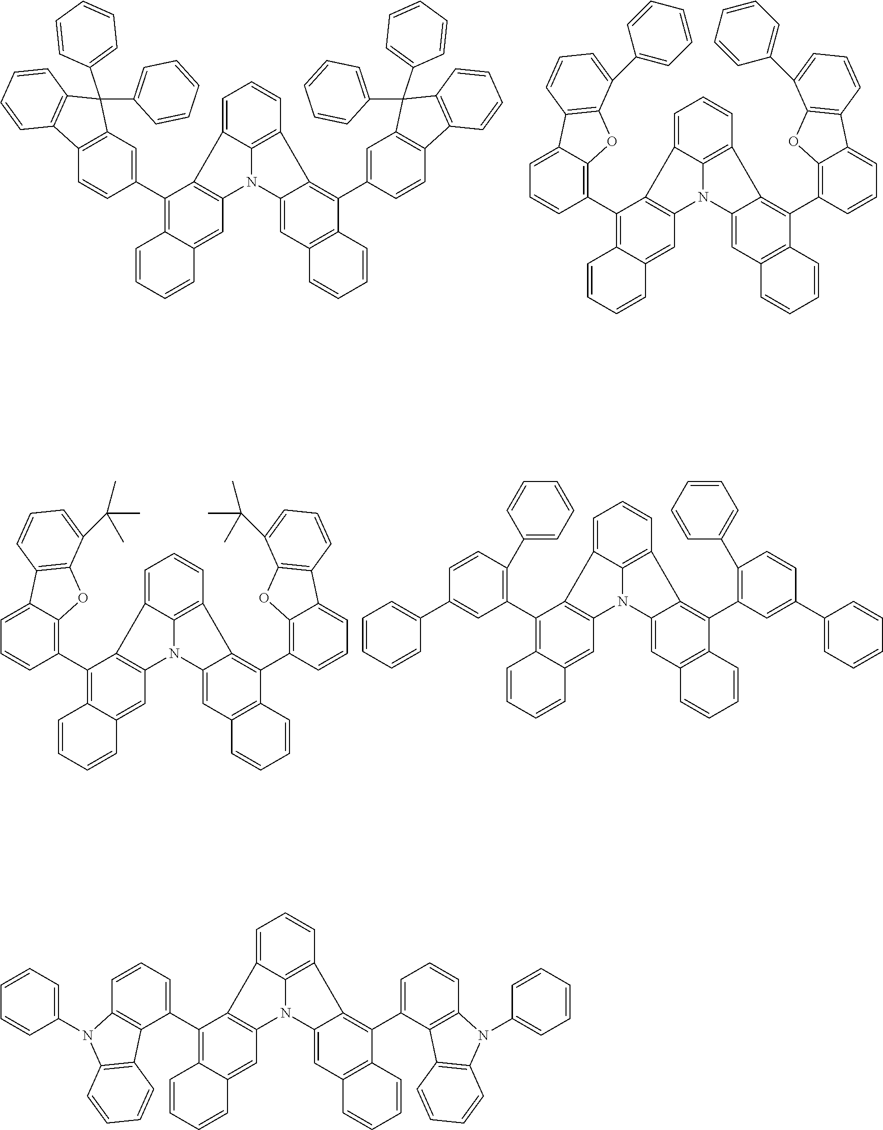

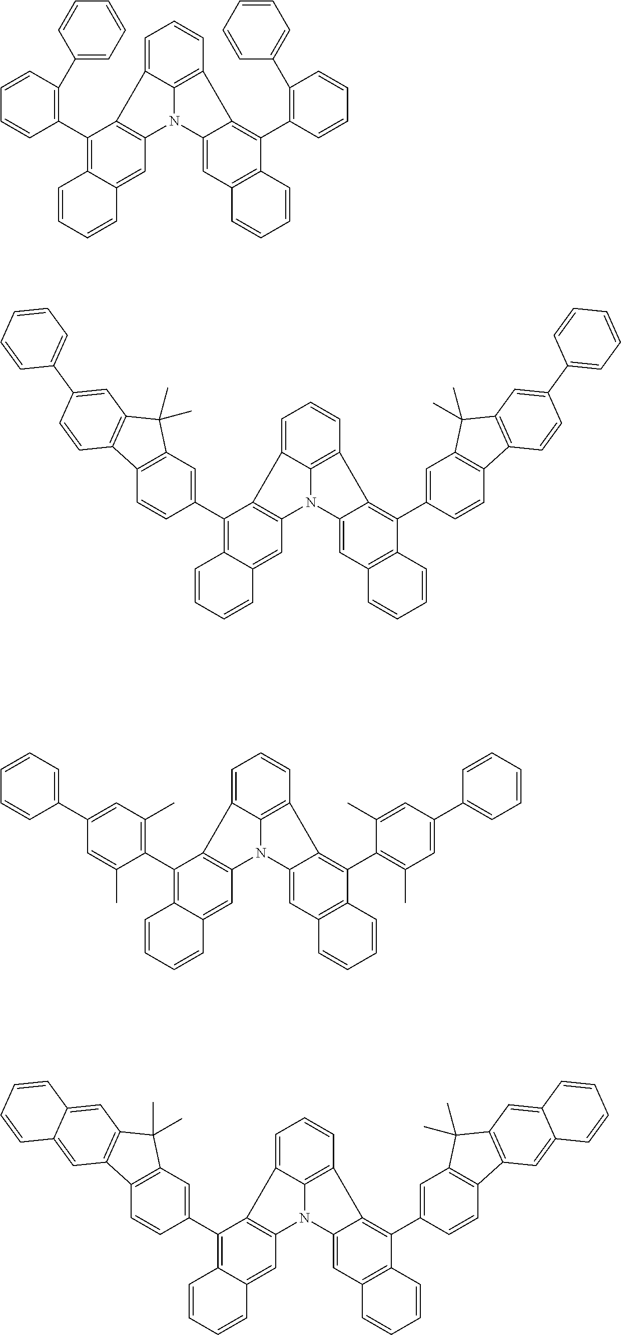 [SMILES: c1ccc(-c2ccccc2-c2c3ccccc3cc3c2c2cccc4c5c(-c6ccccc6-c6ccccc6)c6ccccc6cc5n3c24)cc1 ; CC1(C)c2cc(-c3ccccc3)ccc2-c2ccc(-c3c4ccccc4cc4c3c3cccc5c6c(-c7ccc8c(c7)C(C)(C)c7cc(-c9ccccc9)ccc7-8)c7ccccc7cc6n4c35)cc21 ; Cc1cc(-c2ccccc2)cc(C)c1-c1c2ccccc2cc2c1c1cccc3c4c(-c5c(C)cc(-c6ccccc6)cc5C)c5ccccc5cc4n2c13 ; CC1(C)c2cc(-c3c4ccccc4cc4c3c3cccc5c6c(-c7ccc8c(c7)C(C)(C)c7cc9ccccc9cc7-8)c7ccccc7cc6n4c35)ccc2-c2cc3ccccc3cc21]

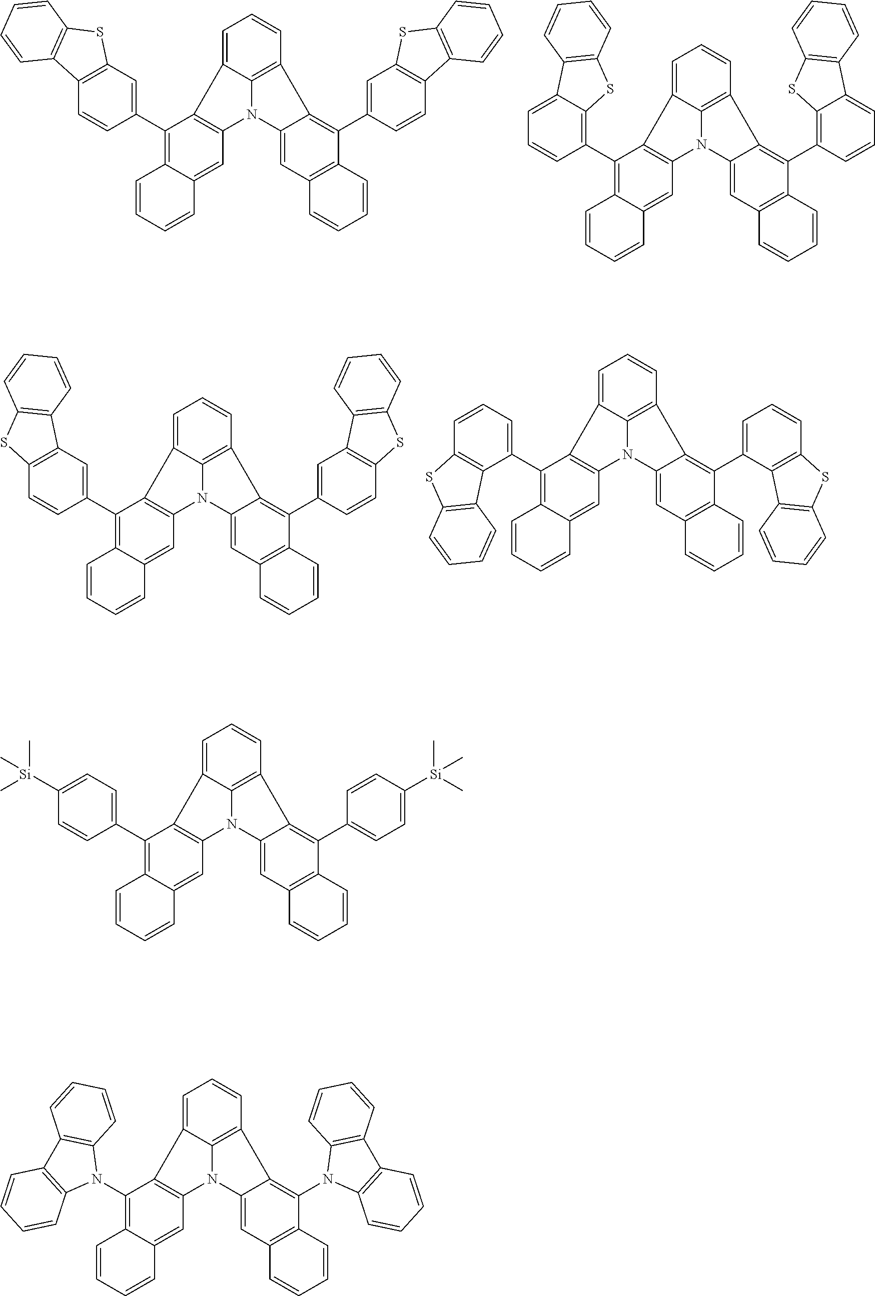

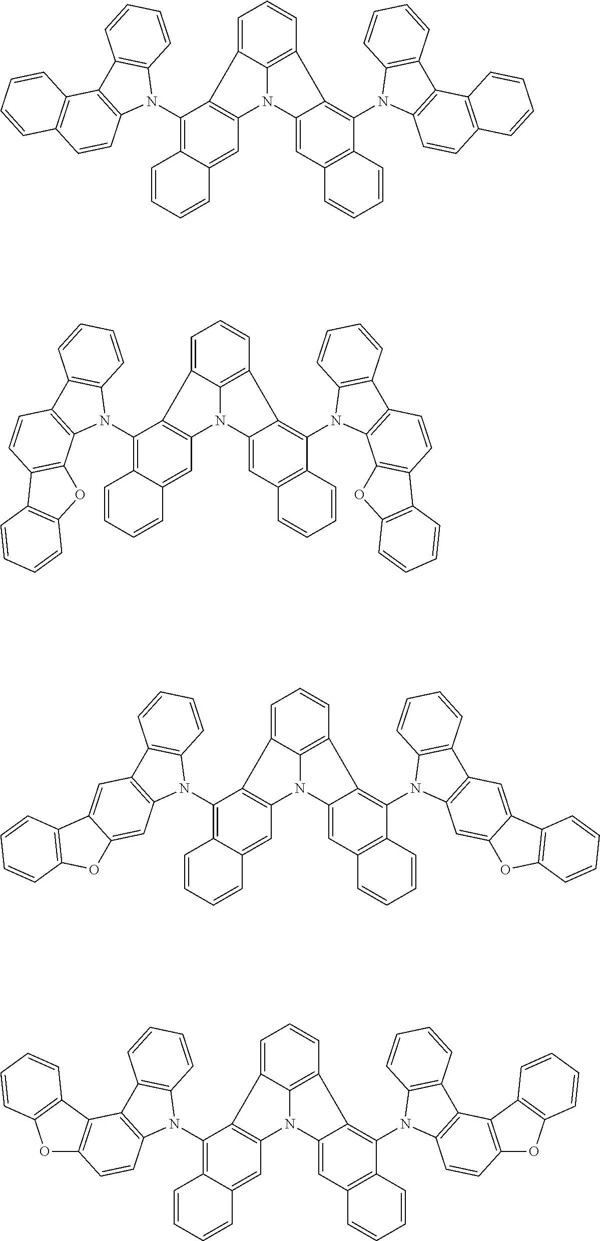 [SMILES: c1ccc2c(-n3c4ccccc4c4c5ccccc5ccc43)c3c4cccc5c6c(-n7c8ccccc8c8c9ccccc9ccc87)c7ccccc7cc6n(c3cc2c1)c45 ; c1ccc2c(-n3c4ccccc4c4ccc5c6ccccc6oc5c43)c3c4cccc5c6c(-n7c8ccccc8c8ccc9c%10ccccc%10oc9c87)c7ccccc7cc6n(c3cc2c1)c45 ; c1ccc2c(-n3c4ccccc4c4cc5c(cc43)oc3ccccc35)c3c4cccc5c6c(-n7c8ccccc8c8cc9c(cc87)oc7ccccc79)c7ccccc7cc6n(c3cc2c1)c45 ; c1ccc2c(-n3c4ccccc4c4c5c(ccc43)oc3ccccc35)c3c4cccc5c6c(-n7c8ccccc8c8c9c(ccc87)oc7ccccc79)c7ccccc7cc6n(c3cc2c1)c45]

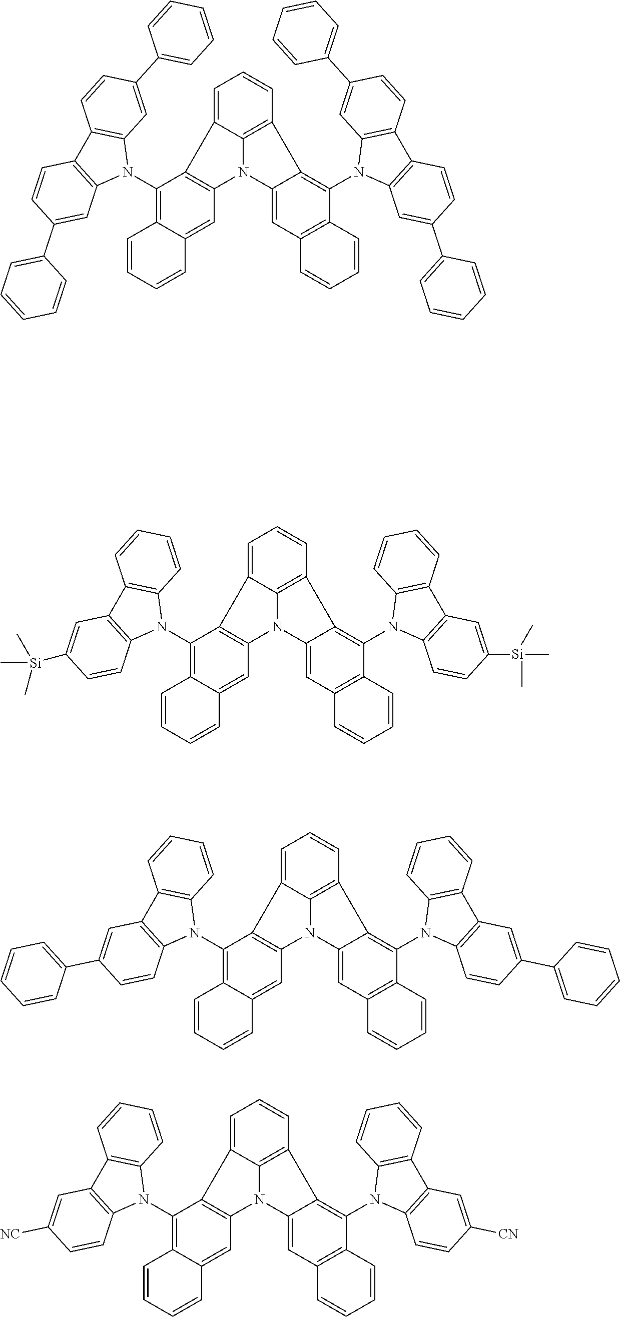

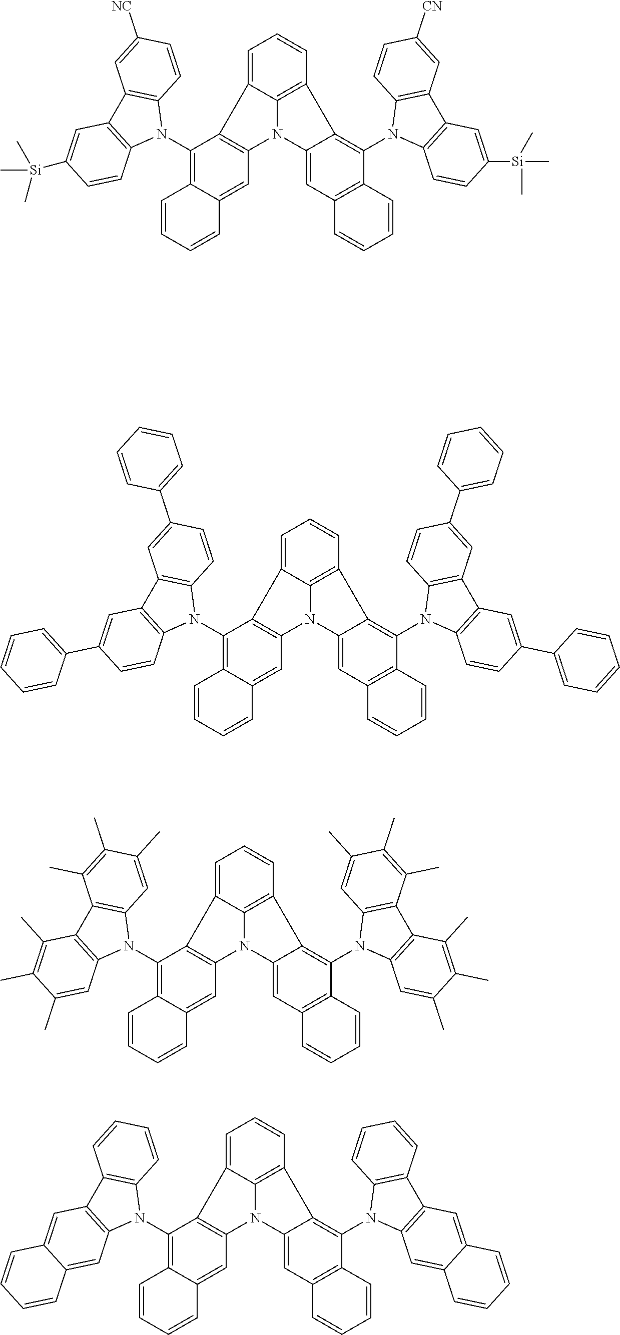

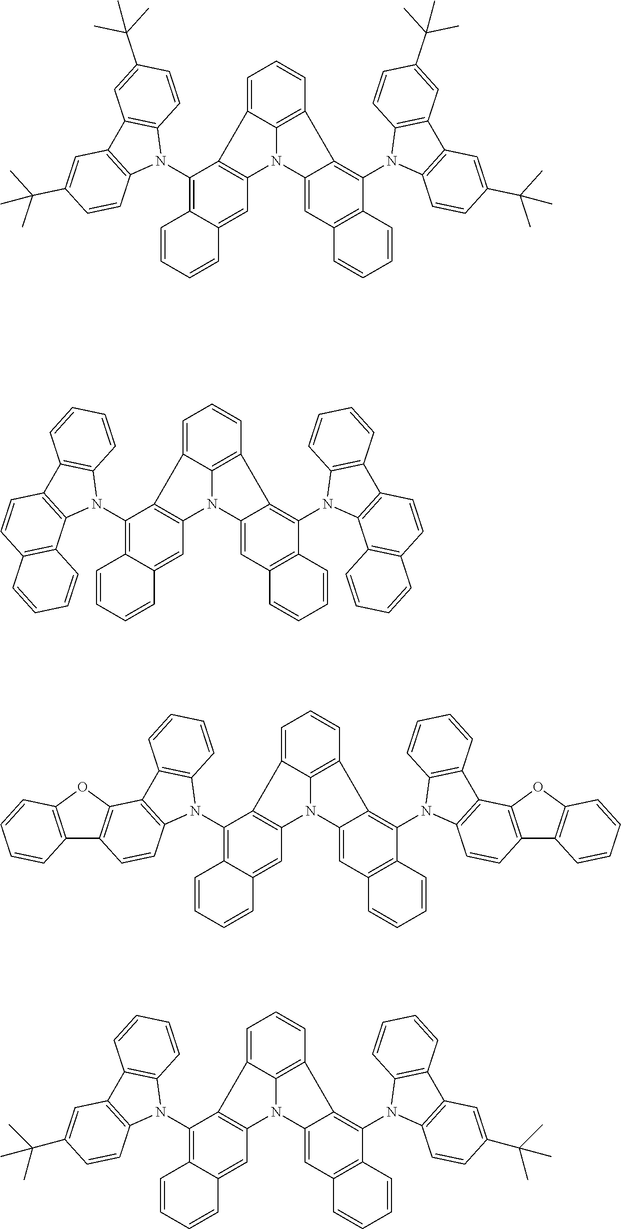

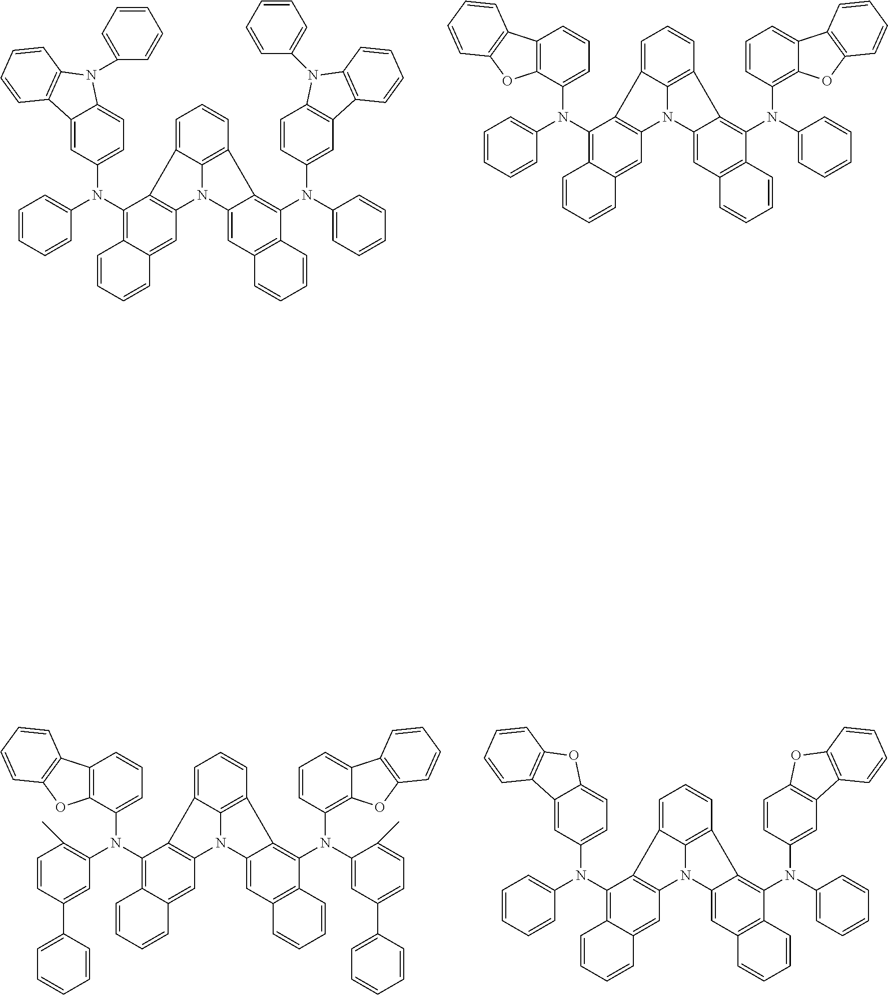 [SMILES: c1ccc(N(c2ccc3c(c2)c2ccccc2n3-c2ccccc2)c2c3ccccc3cc3c2c2cccc4c5c(N(c6ccccc6)c6ccc7c(c6)c6ccccc6n7-c6ccccc6)c6ccccc6cc5n3c24)cc1 ; c1ccc(N(c2cccc3c2oc2ccccc23)c2c3ccccc3cc3c2c2cccc4c5c(N(c6ccccc6)c6cccc7c6oc6ccccc67)c6ccccc6cc5n3c24)cc1 ; Cc1ccc(-c2ccccc2)cc1N(c1cccc2c1oc1ccccc12)c1c2ccccc2cc2c1c1cccc3c4c(N(c5cc(-c6ccccc6)ccc5C)c5cccc6c5oc5ccccc56)c5ccccc5cc4n2c13 ; c1ccc(N(c2ccc3oc4ccccc4c3c2)c2c3ccccc3cc3c2c2cccc4c5c(N(c6ccccc6)c6ccc7oc8ccccc8c7c6)c6ccccc6cc5n3c24)cc1]

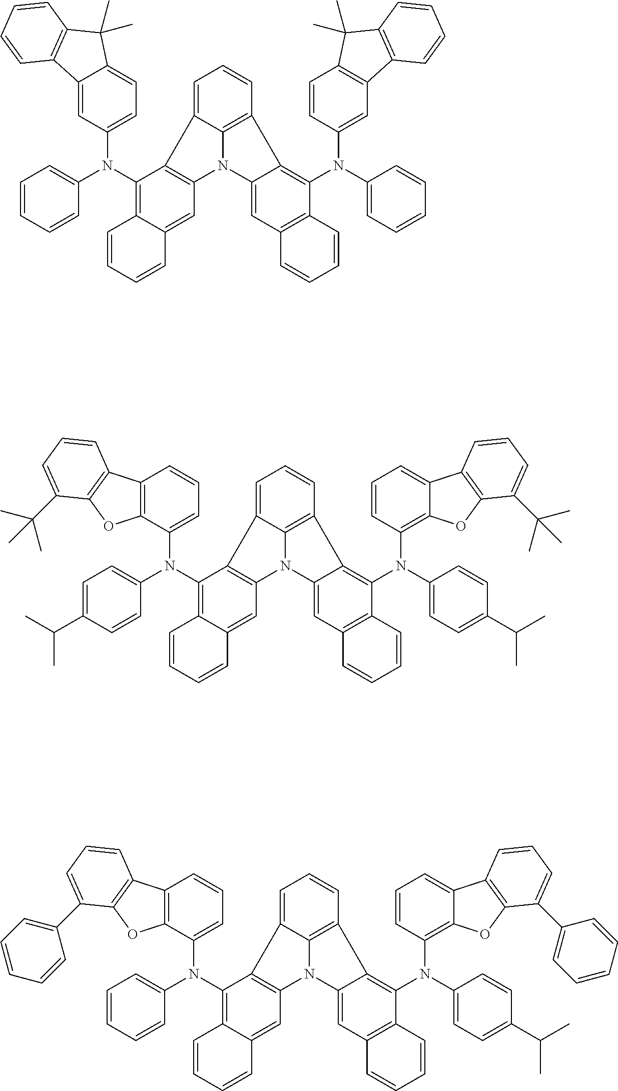 [SMILES: CC1(C)c2ccccc2-c2cc(N(c3ccccc3)c3c4ccccc4cc4c3c3cccc5c6c(N(c7ccccc7)c7ccc8c(c7)-c7ccccc7C8(C)C)c7ccccc7cc6n4c35)ccc21 ; CC(C)c1ccc(N(c2cccc3c2oc2c(C(C)(C)C)cccc23)c2c3ccccc3cc3c2c2cccc4c5c(N(c6ccc(C(C)C)cc6)c6cccc7c6oc6c(C(C)(C)C)cccc67)c6ccccc6cc5n3c24)cc1 ; CC(C)c1ccc(N(c2cccc3c2oc2c(-c4ccccc4)cccc23)c2c3ccccc3cc3c2c2cccc4c5c(N(c6ccccc6)c6cccc7c6oc6c(-c8ccccc8)cccc67)c6ccccc6cc5n3c42)cc1]

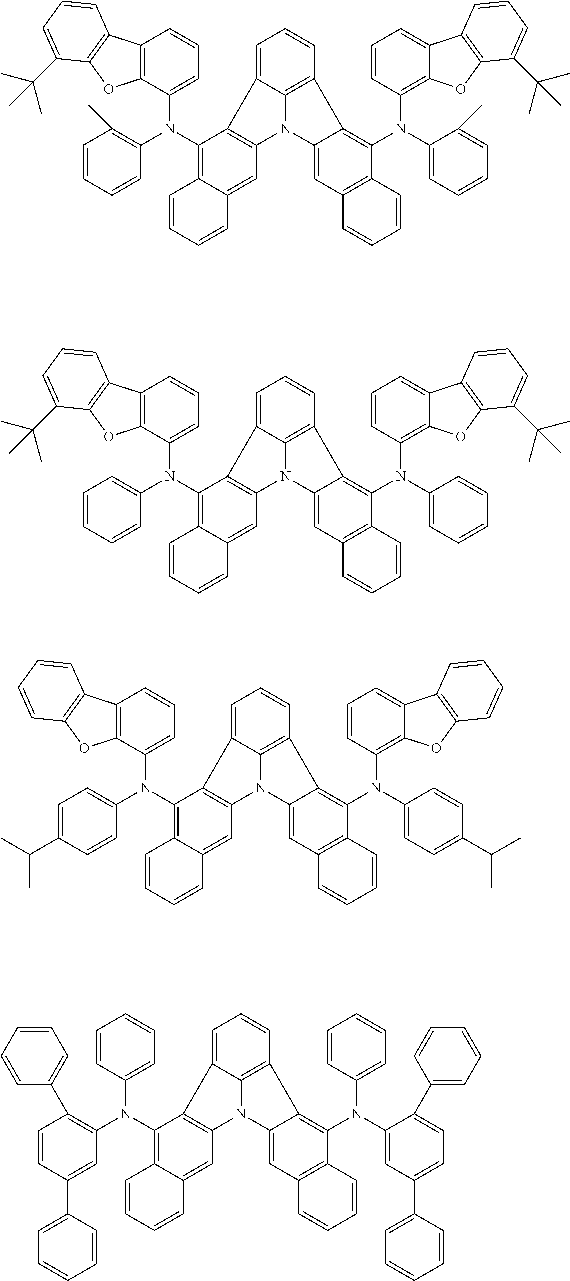

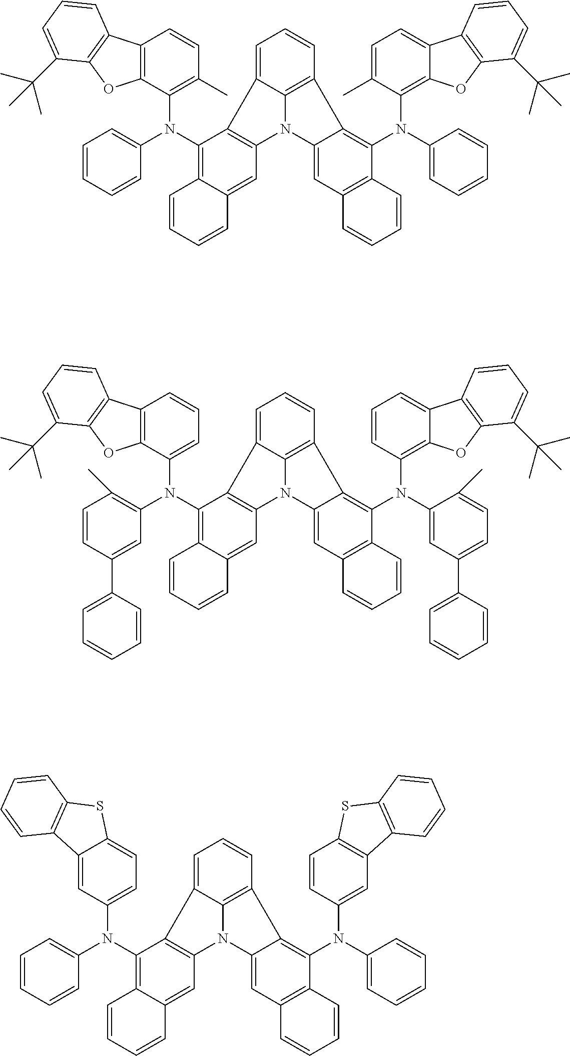

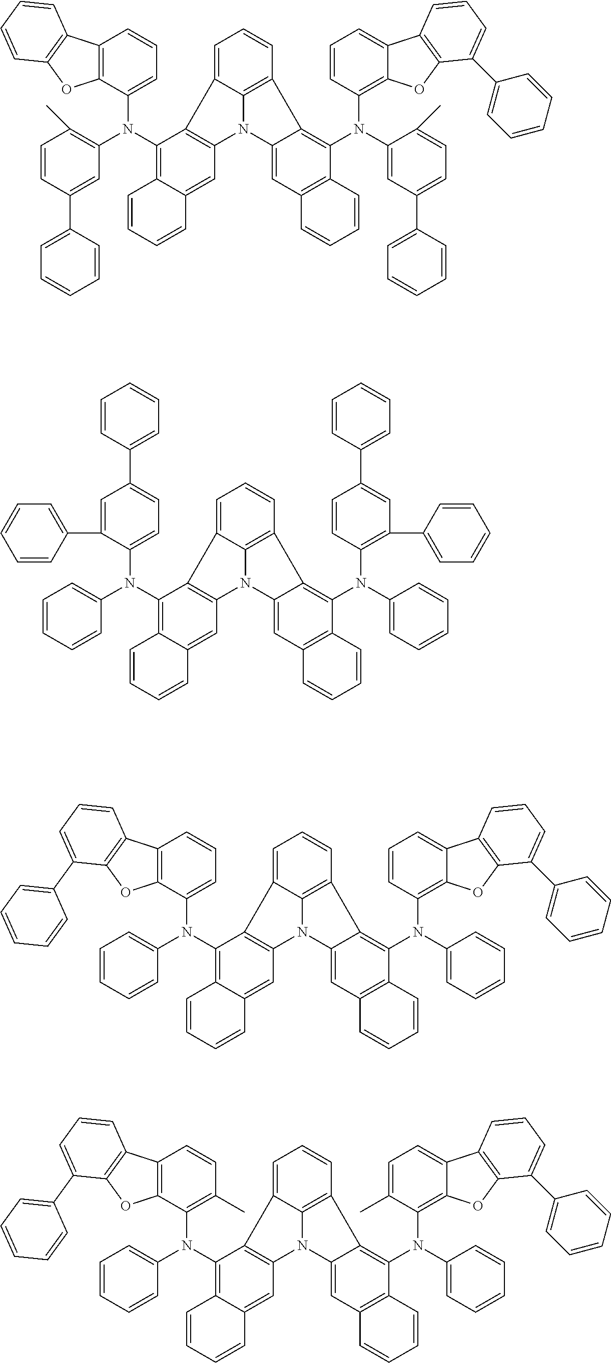

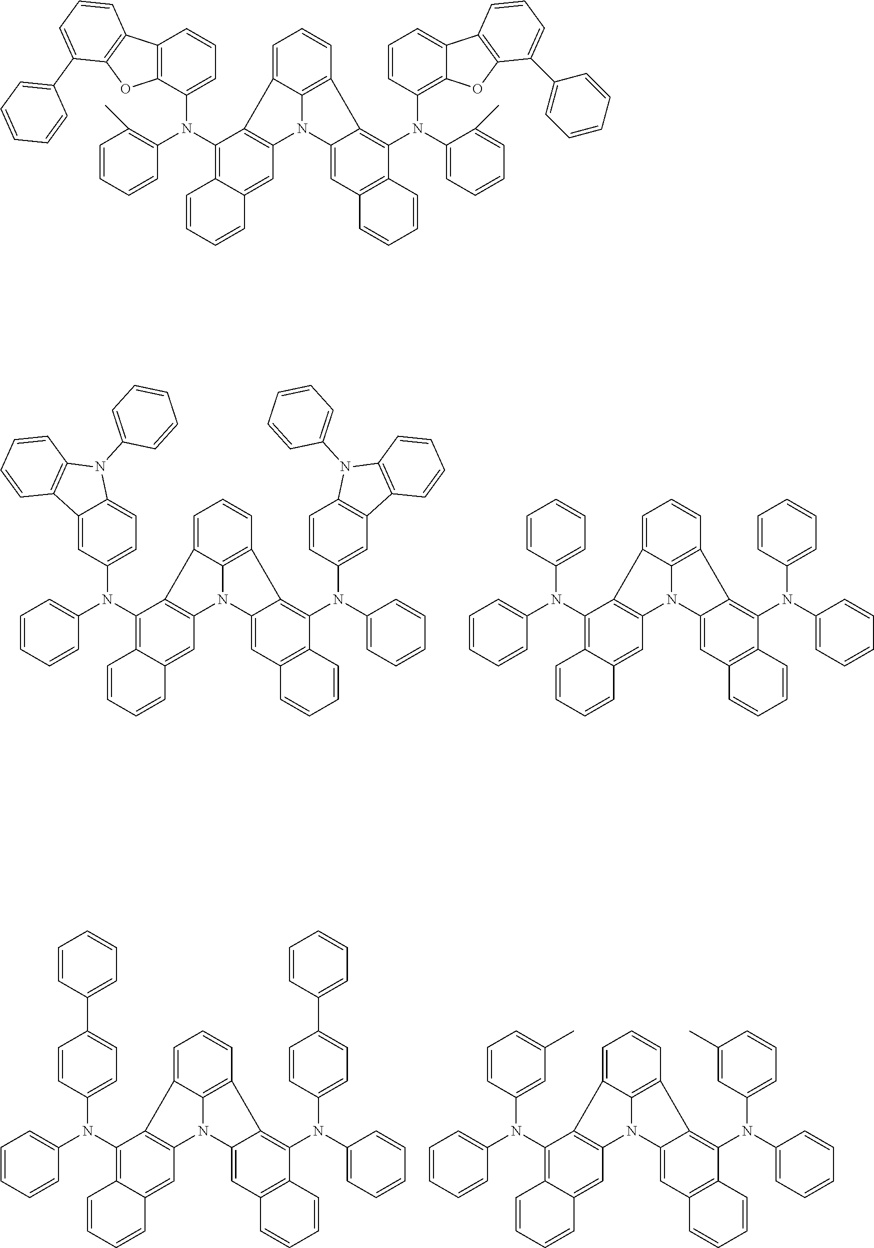

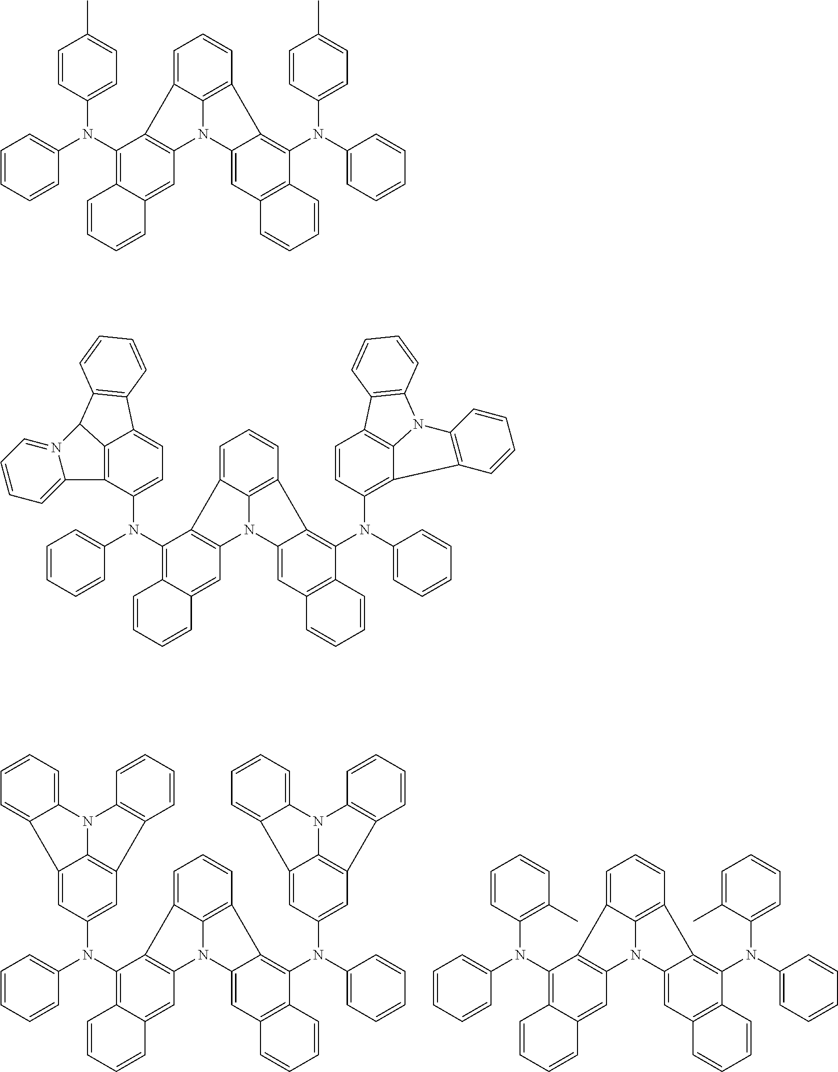

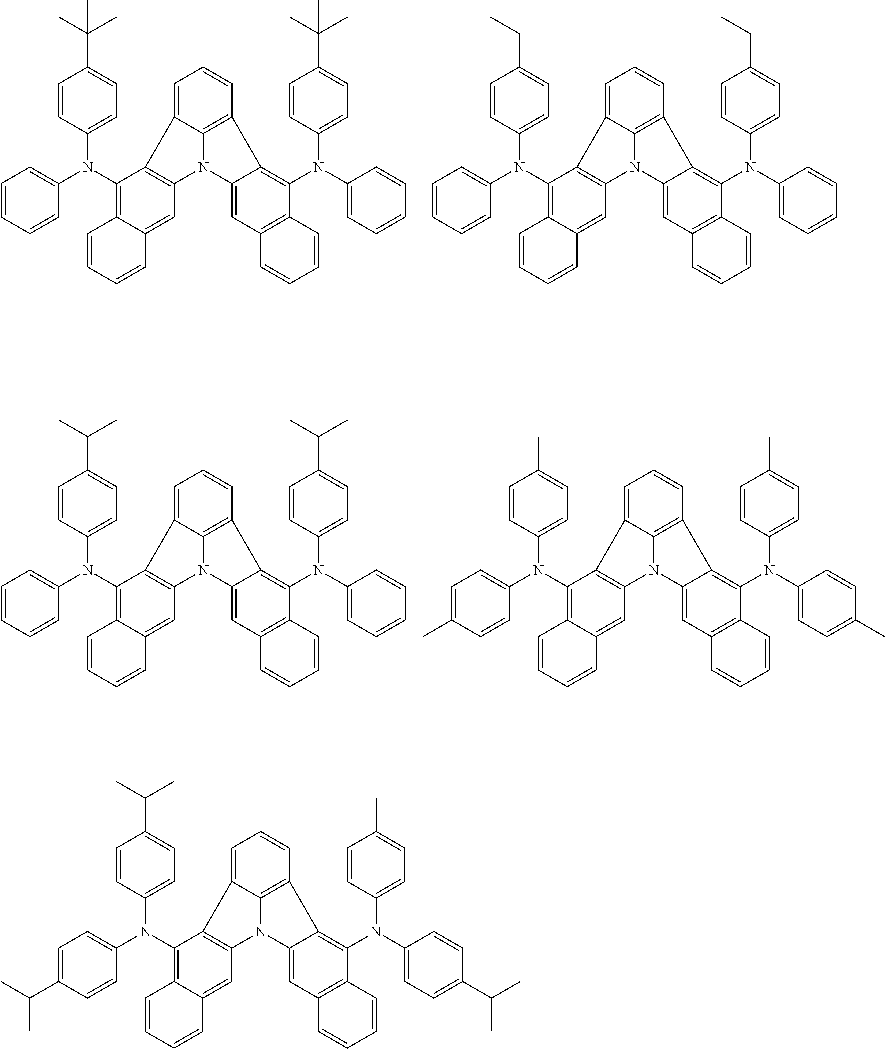

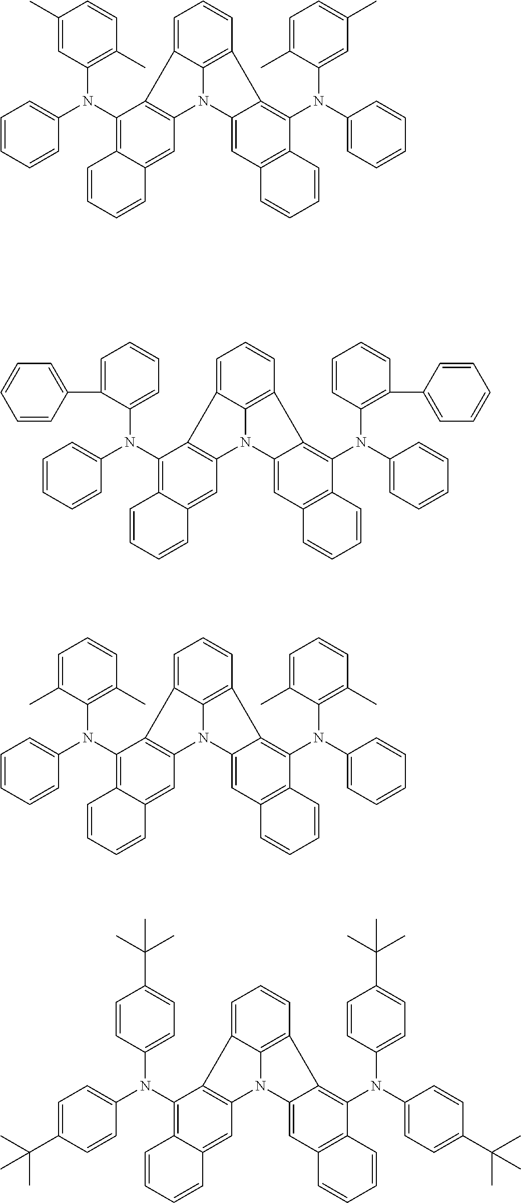 [SMILES: Cc1ccc(C)c(N(c2ccccc2)c2c3ccccc3cc3c2c2cccc4c5c(N(c6ccccc6)c6cc(C)ccc6C)c6ccccc6cc5n3c24)c1 ; c1ccc(-c2ccccc2N(c2ccccc2)c2c3ccccc3cc3c2c2cccc4c5c(N(c6ccccc6)c6ccccc6-c6ccccc6)c6ccccc6cc5n3c24)cc1 ; Cc1cccc(C)c1N(c1ccccc1)c1c2ccccc2cc2c1c1cccc3c4c(N(c5ccccc5)c5c(C)cccc5C)c5ccccc5cc4n2c13 ; CC(C)(C)c1ccc(N(c2ccc(C(C)(C)C)cc2)c2c3ccccc3cc3c2c2cccc4c5c(N(c6ccc(C(C)(C)C)cc6)c6ccc(C(C)(C)C)cc6)c6ccccc6cc5n3c24)cc1]

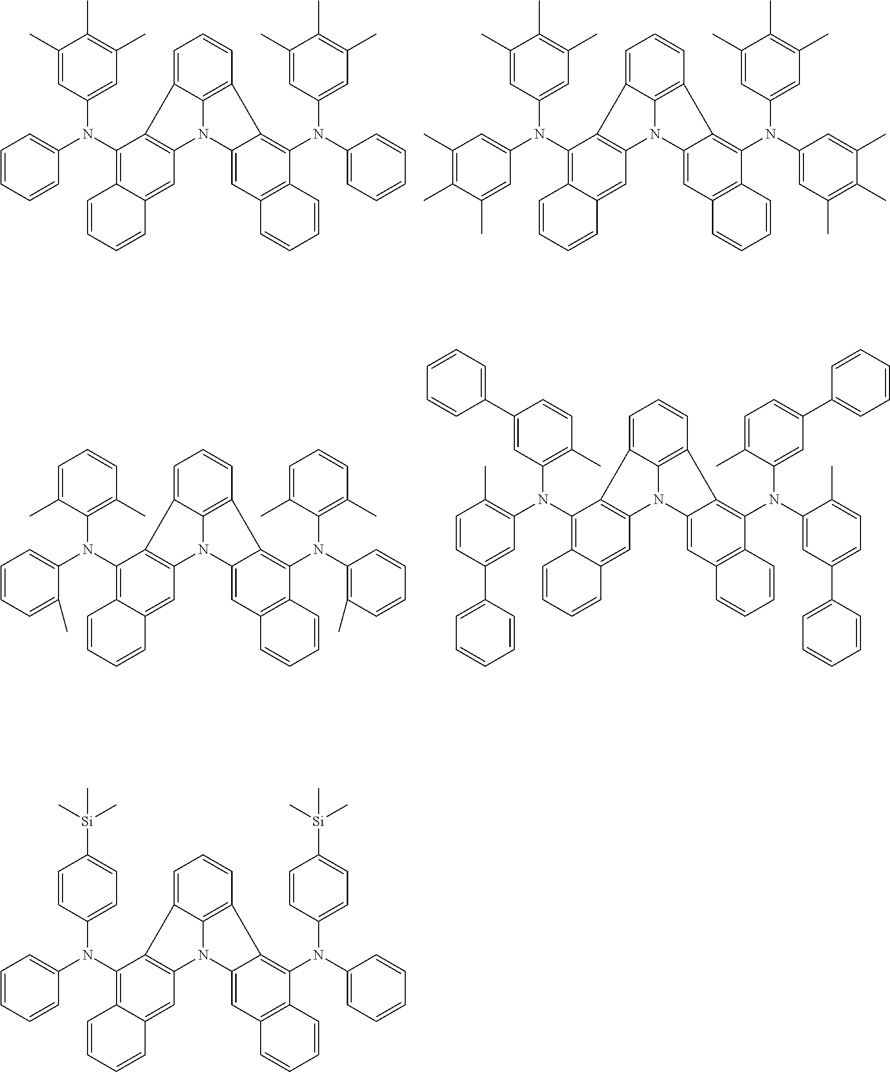

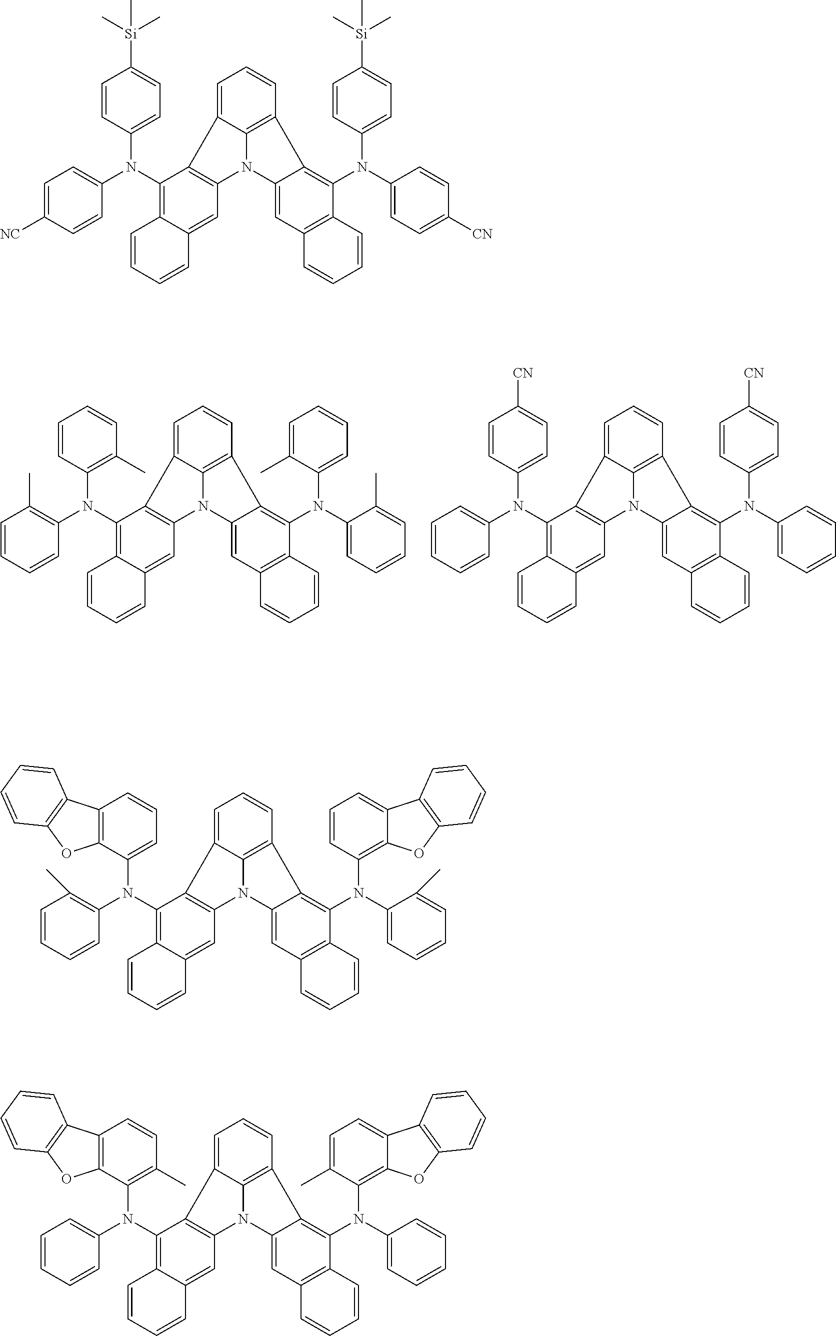

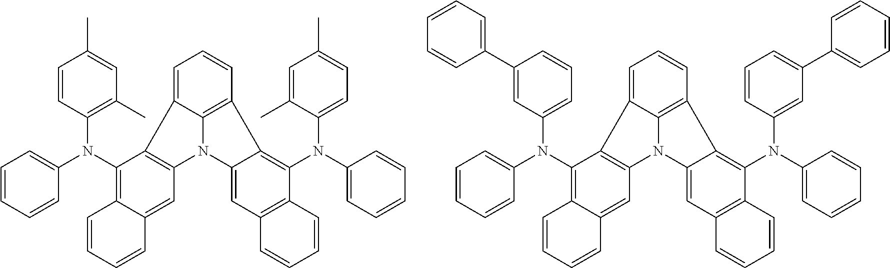 [SMILES: Cc1ccc(N(c2ccccc2)c2c3ccccc3cc3c2c2cccc4c5c(N(c6ccccc6)c6ccc(C)cc6C)c6ccccc6cc5n3c24)c(C)c1 ; c1ccc(-c2cccc(N(c3ccccc3)c3c4ccccc4cc4c3c3cccc5c6c(N(c7ccccc7)c7cccc(-c8ccccc8)c7)c7ccccc7cc6n4c35)c2)cc1]

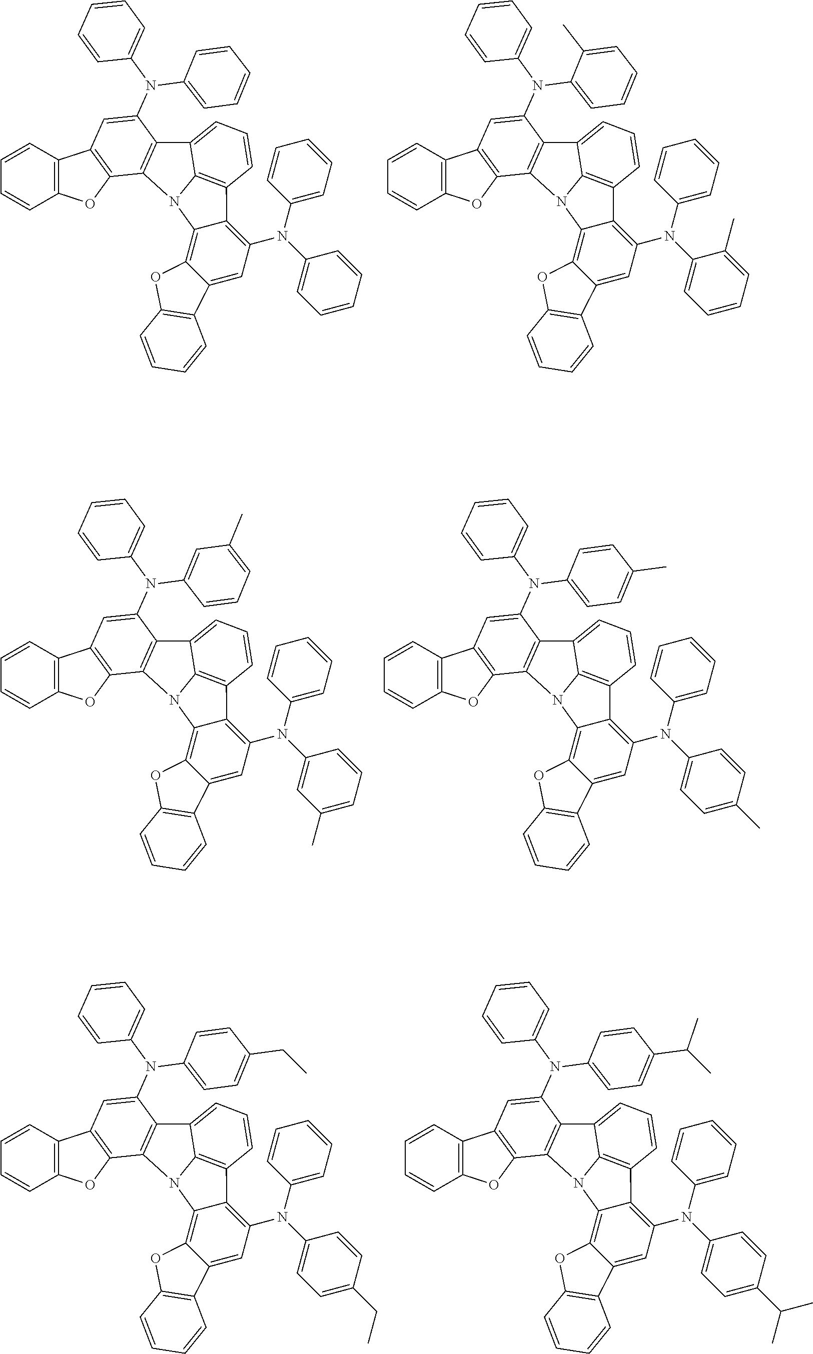

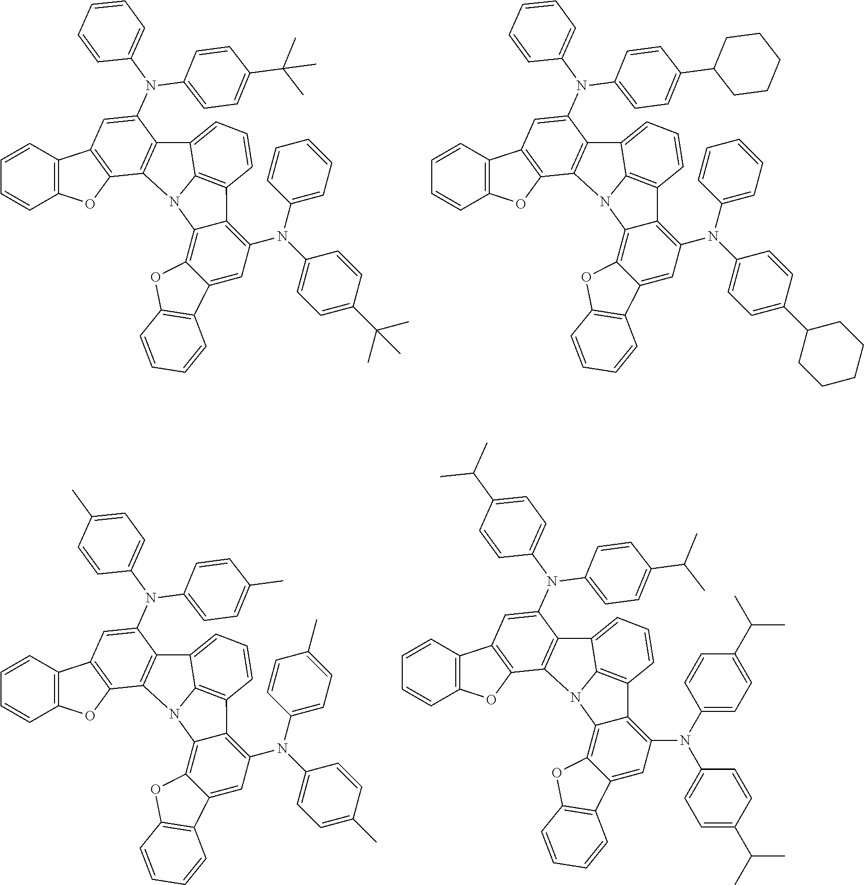 [SMILES: CC(C)(C)c1ccc(N(c2ccccc2)c2cc3c4ccccc4oc3c3c2c2cccc4c5c(N(c6ccccc6)c6ccc(C(C)(C)C)cc6)cc6c7ccccc7oc6c5n3c24)cc1 ; c1ccc(N(c2ccc(C3CCCCC3)cc2)c2cc3c4ccccc4oc3c3c2c2cccc4c5c(N(c6ccccc6)c6ccc(C7CCCCC7)cc6)cc6c7ccccc7oc6c5n3c24)cc1 ; Cc1ccc(N(c2ccc(C)cc2)c2cc3c4ccccc4oc3c3c2c2cccc4c5c(N(c6ccc(C)cc6)c6ccc(C)cc6)cc6c7ccccc7oc6c5n3c24)cc1 ; CC(C)c1ccc(N(c2ccc(C(C)C)cc2)c2cc3c4ccccc4oc3c3c2c2cccc4c5c(N(c6ccc(C(C)C)cc6)c6ccc(C(C)C)cc6)cc6c7ccccc7oc6c5n3c24)cc1]

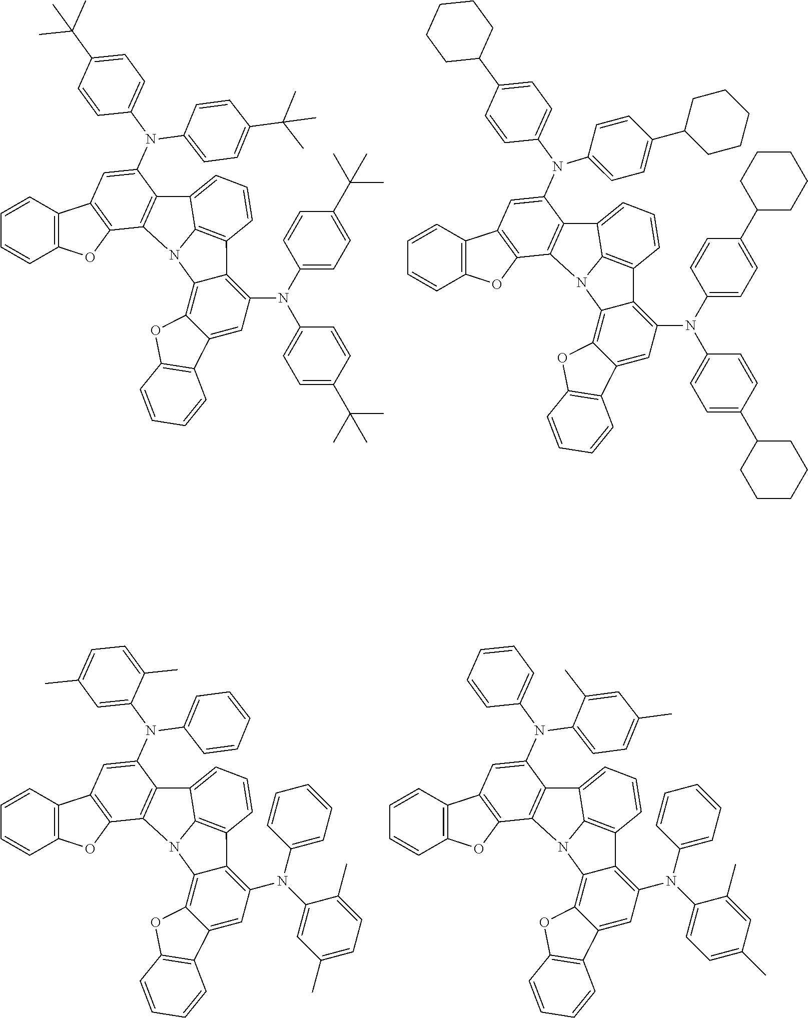 [SMILES: CC(C)(C)c1ccc(N(c2ccc(C(C)(C)C)cc2)c2cc3c4ccccc4oc3c3c2c2cccc4c5c(N(c6ccc(C(C)(C)C)cc6)c6ccc(C(C)(C)C)cc6)cc6c7ccccc7oc6c5n3c24)cc1 ; c1ccc2c(c1)oc1c2cc(N(c2ccc(C3CCCCC3)cc2)c2ccc(C3CCCCC3)cc2)c2c3cccc4c5c(N(c6ccc(C7CCCCC7)cc6)c6ccc(C7CCCCC7)cc6)cc6c7ccccc7oc6c5n(c34)c12 ; Cc1ccc(C)c(N(c2ccccc2)c2cc3c4ccccc4oc3c3c2c2cccc4c5c(N(c6ccccc6)c6cc(C)ccc6C)cc6c7ccccc7oc6c5n3c24)c1 ; Cc1ccc(N(c2ccccc2)c2cc3c4ccccc4oc3c3c2c2cccc4c5c(N(c6ccccc6)c6ccc(C)cc6C)cc6c7ccccc7oc6c5n3c24)c(C)c1]

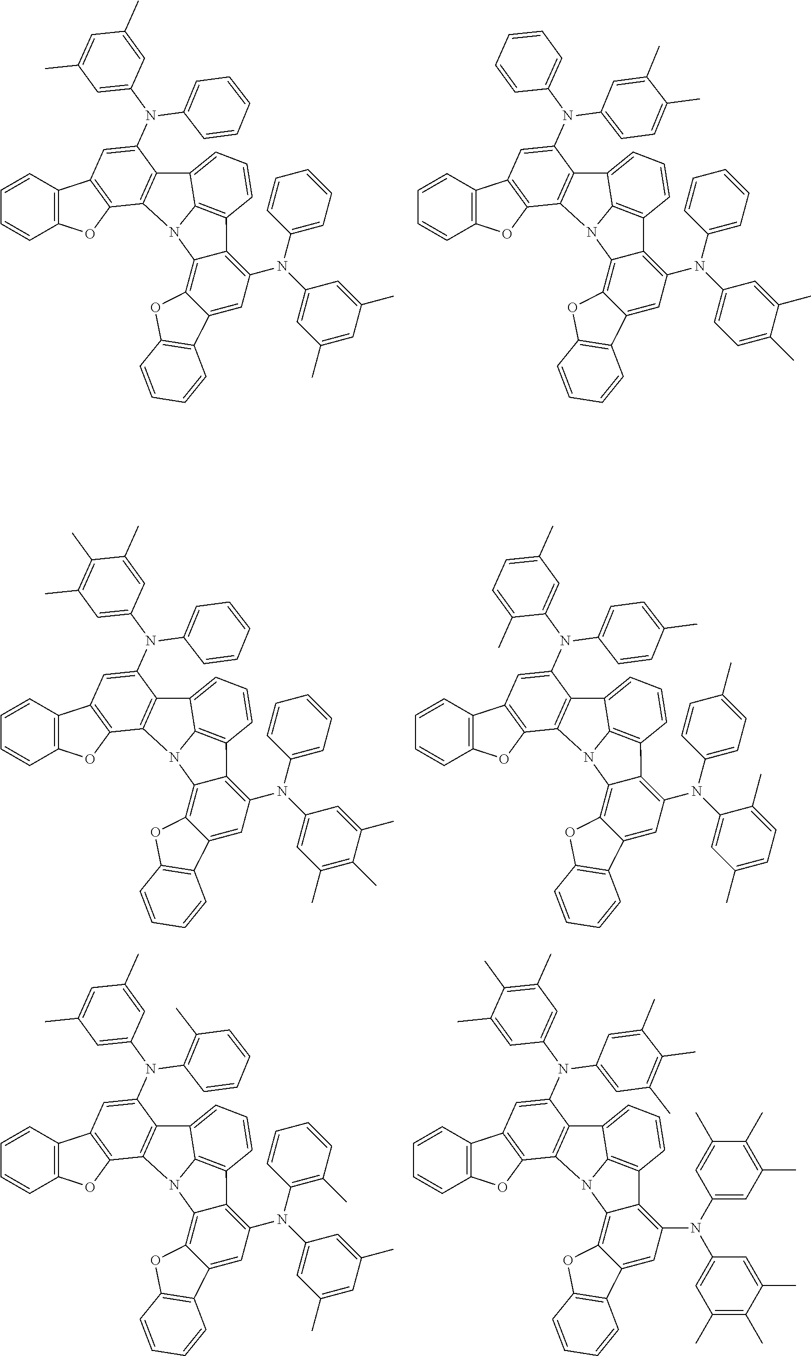

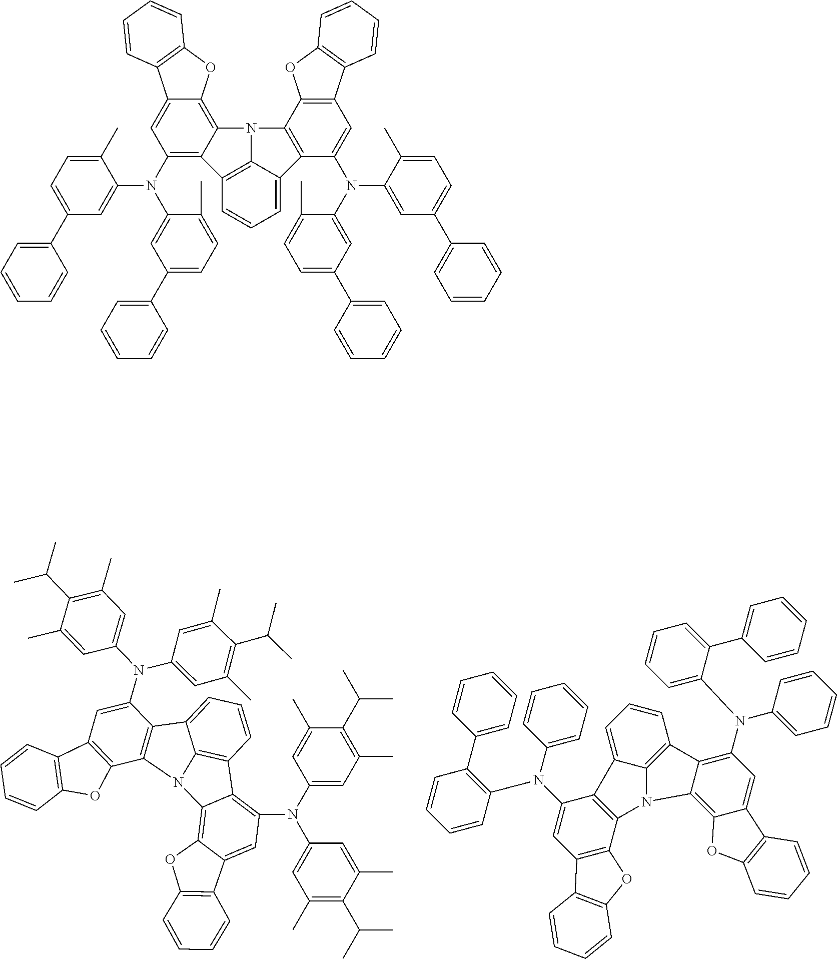 [SMILES: Cc1ccc(-c2ccccc2)cc1N(c1cc(-c2ccccc2)ccc1C)c1cc2c3ccccc3oc2c2c1c1cccc3c4c(N(c5cc(-c6ccccc6)ccc5C)c5cc(-c6ccccc6)ccc5C)cc5c6ccccc6oc5c4n2c13 ; Cc1cc(N(c2cc(C)c(C(C)C)c(C)c2)c2cc3c4ccccc4oc3c3c2c2cccc4c5c(N(c6cc(C)c(C(C)C)c(C)c6)c6cc(C)c(C(C)C)c(C)c6)cc6c7ccccc7oc6c5n3c24)cc(C)c1C(C)C ; c1ccc(-c2ccccc2N(c2ccccc2)c2cc3c4ccccc4oc3c3c2c2cccc4c5c(N(c6ccccc6)c6ccccc6-c6ccccc6)cc6c7ccccc7oc6c5n3c24)cc1]

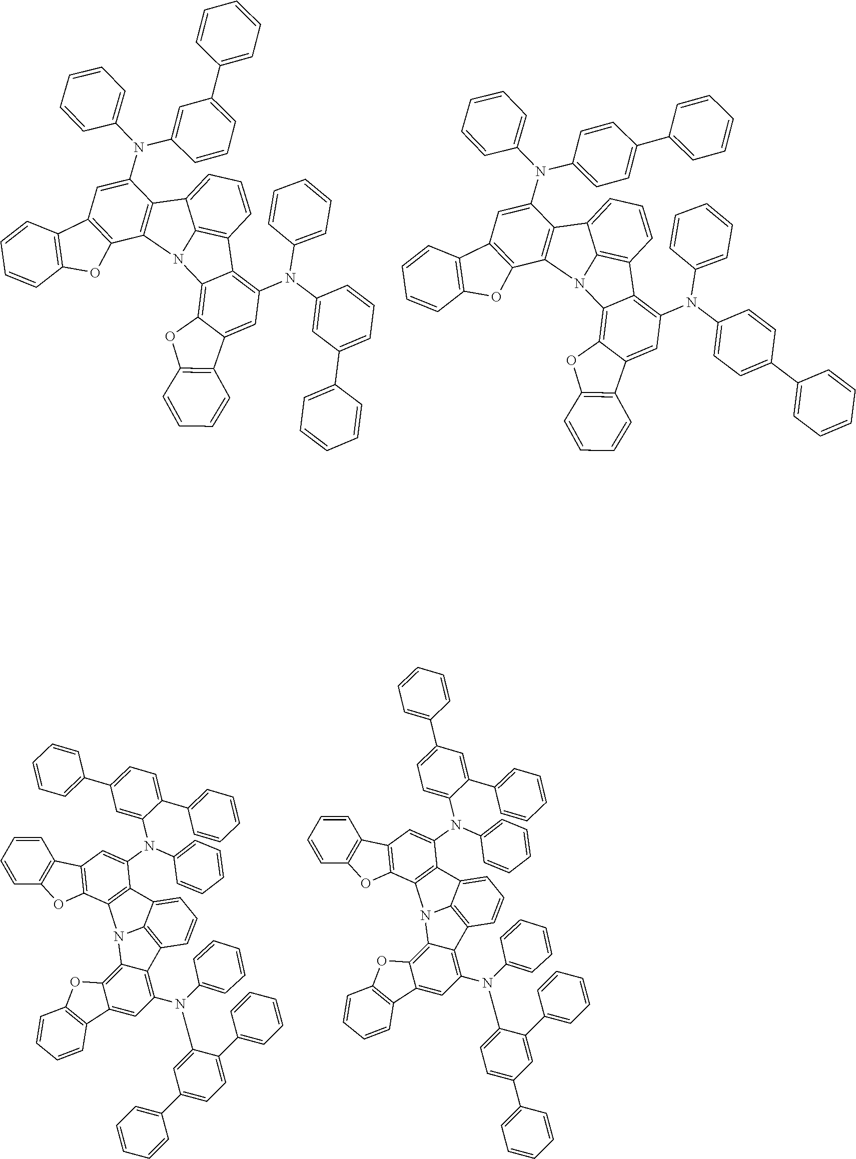

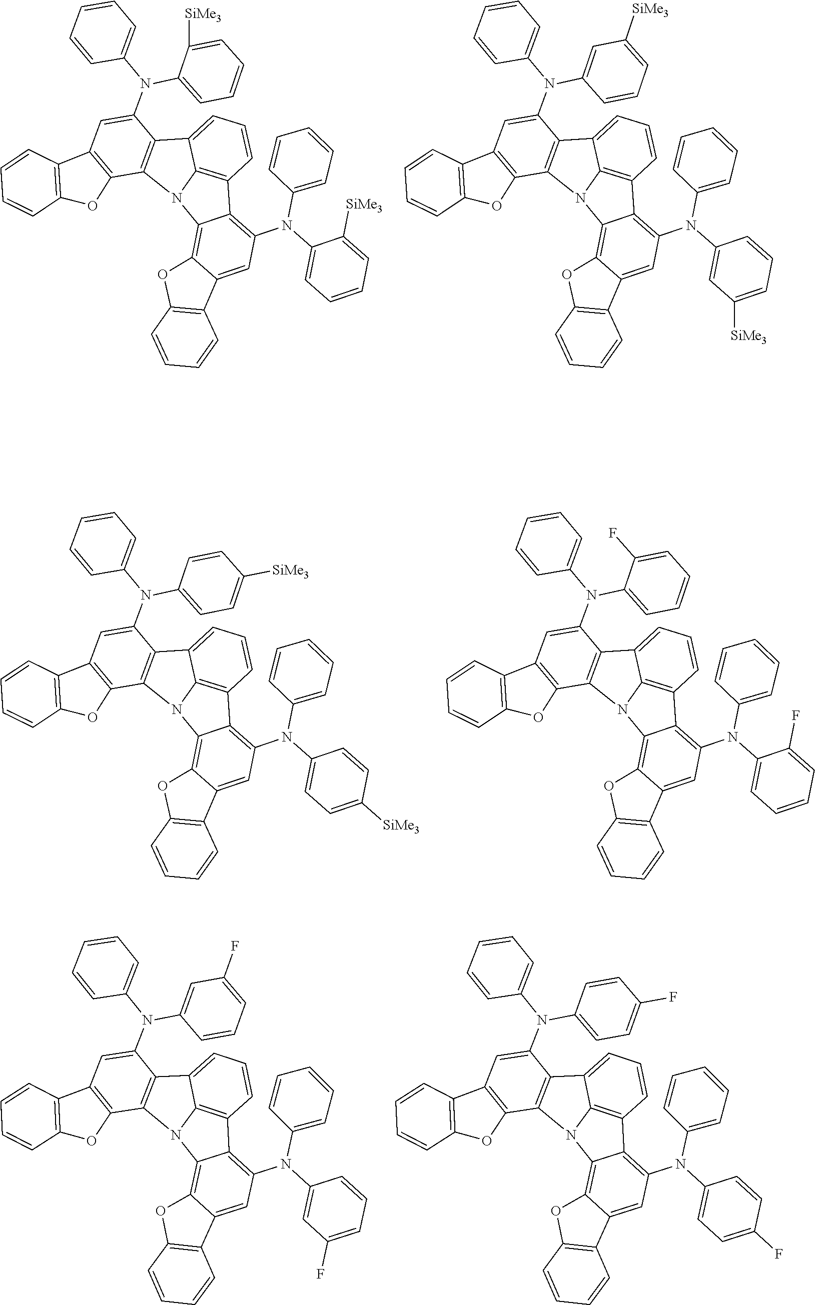

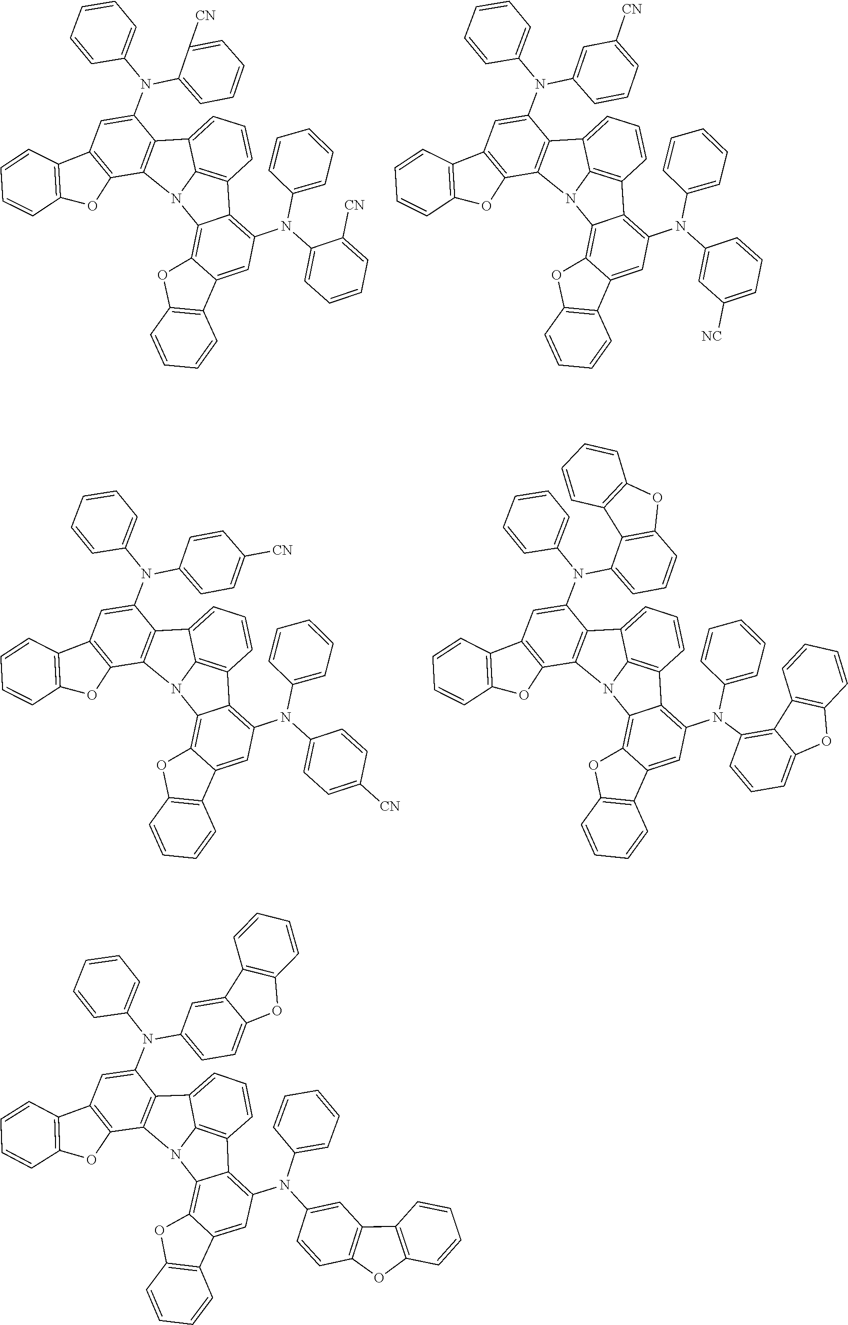

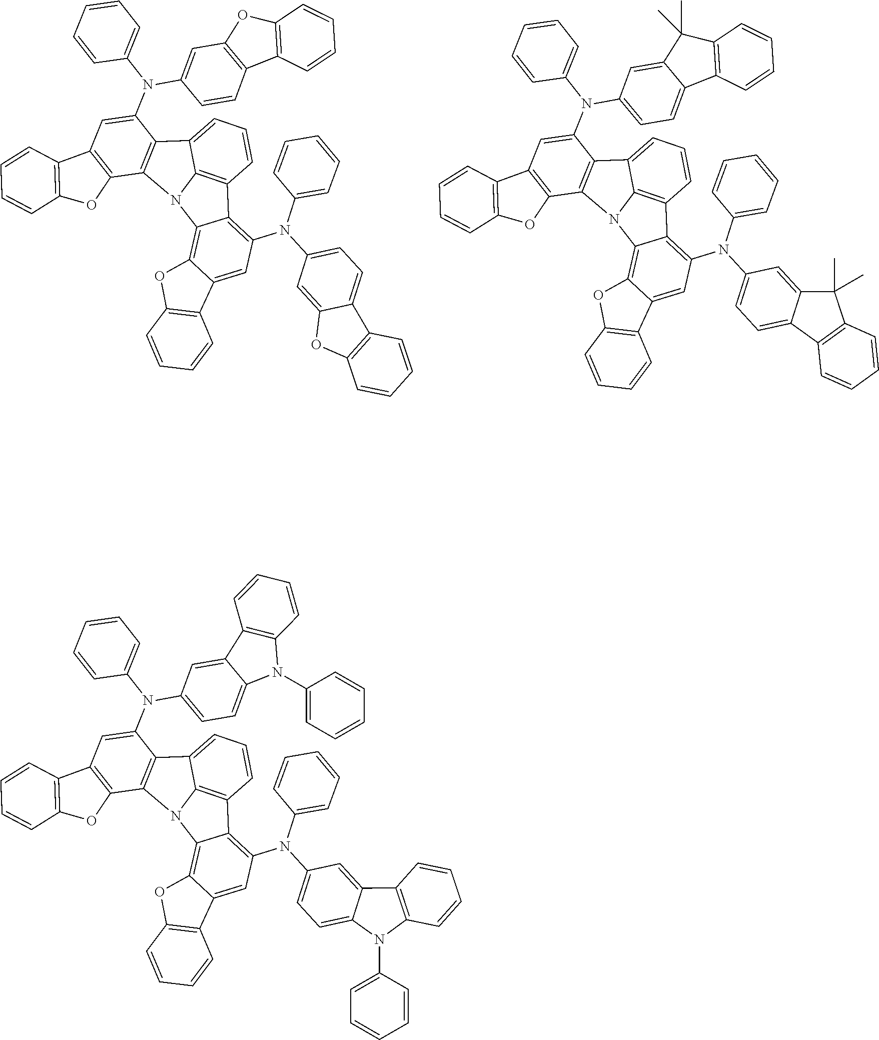 [SMILES: c1ccc(N(c2ccc3c(c2)oc2ccccc23)c2cc3c4ccccc4oc3c3c2c2cccc4c5c(N(c6ccccc6)c6ccc7c(c6)oc6ccccc67)cc6c7ccccc7oc6c5n3c24)cc1 ; CC1(C)c2ccccc2-c2ccc(N(c3ccccc3)c3cc4c5ccccc5oc4c4c3c3cccc5c6c(N(c7ccccc7)c7ccc8c(c7)C(C)(C)c7ccccc7-8)cc7c8ccccc8oc7c6n4c35)cc21 ; c1ccc(N(c2ccc3c(c2)c2ccccc2n3-c2ccccc2)c2cc3c4ccccc4oc3c3c2c2cccc4c5c(N(c6ccccc6)c6ccc7c(c6)c6ccccc6n7-c6ccccc6)cc6c7ccccc7oc6c5n3c24)cc1]

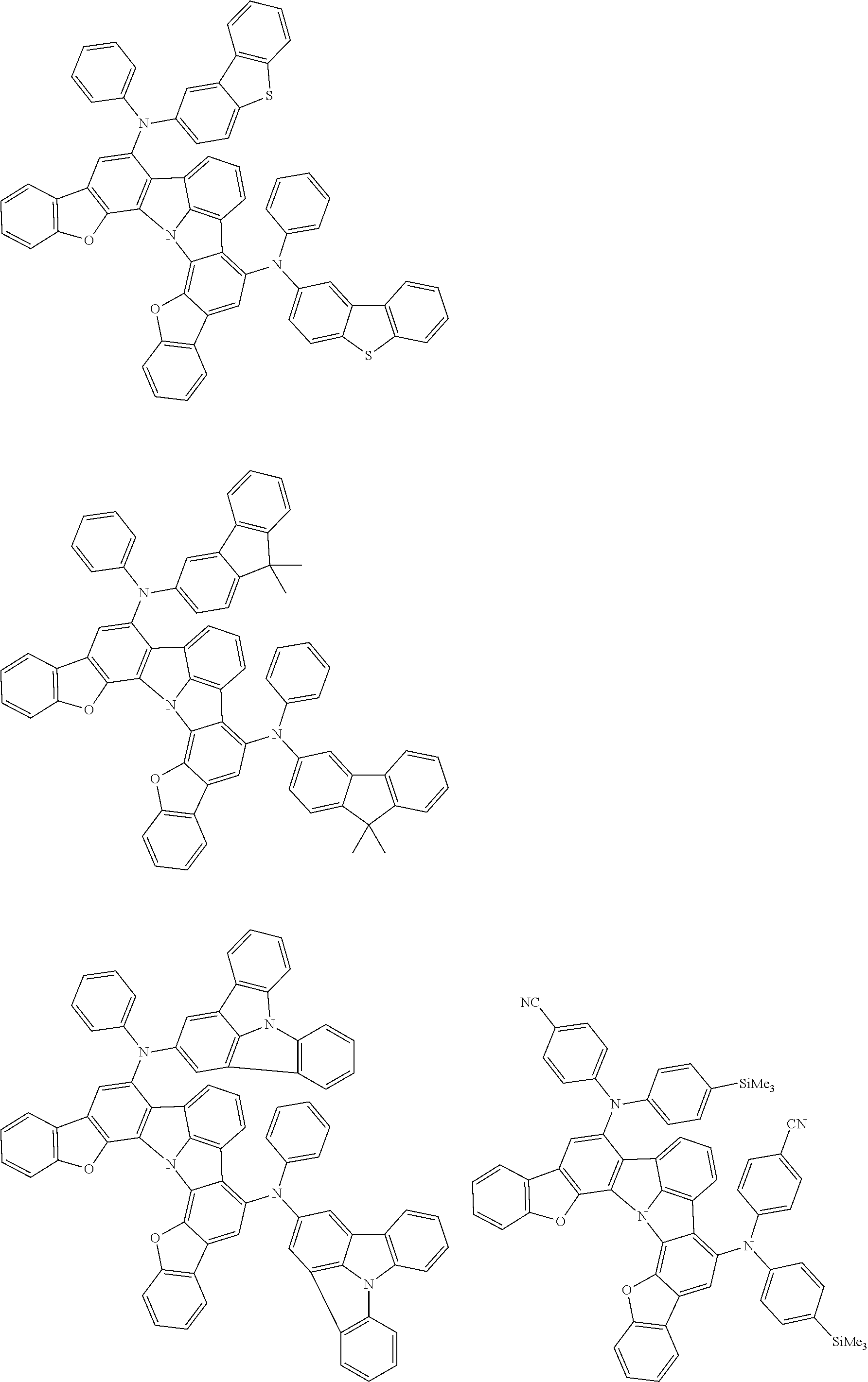

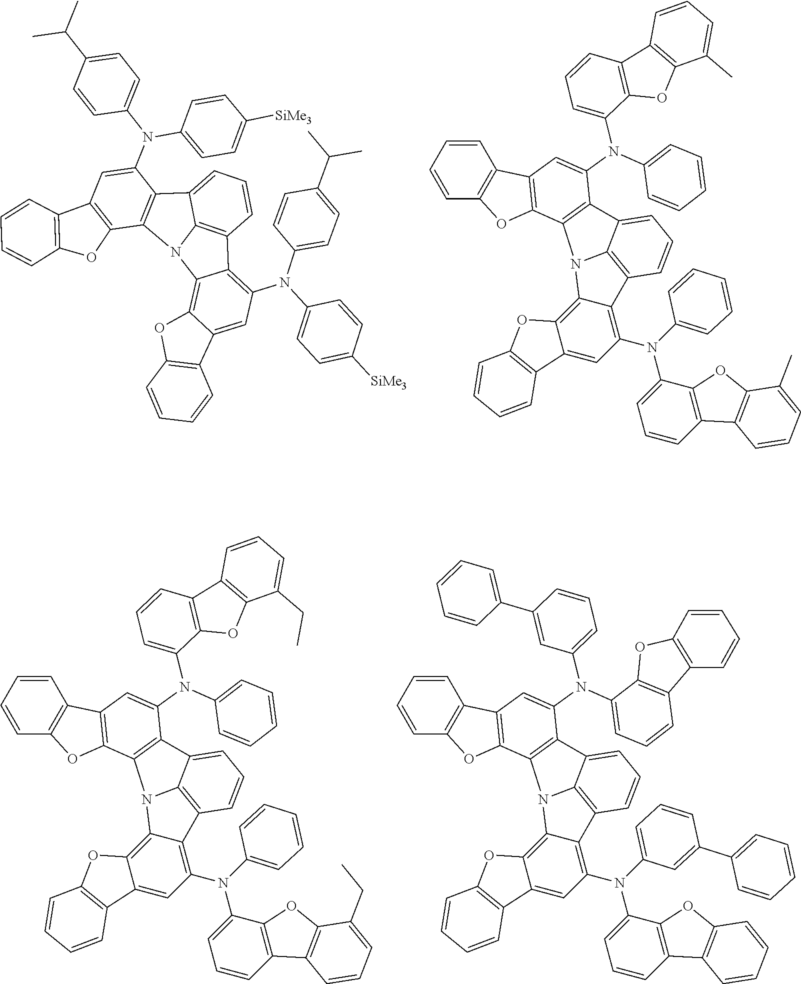

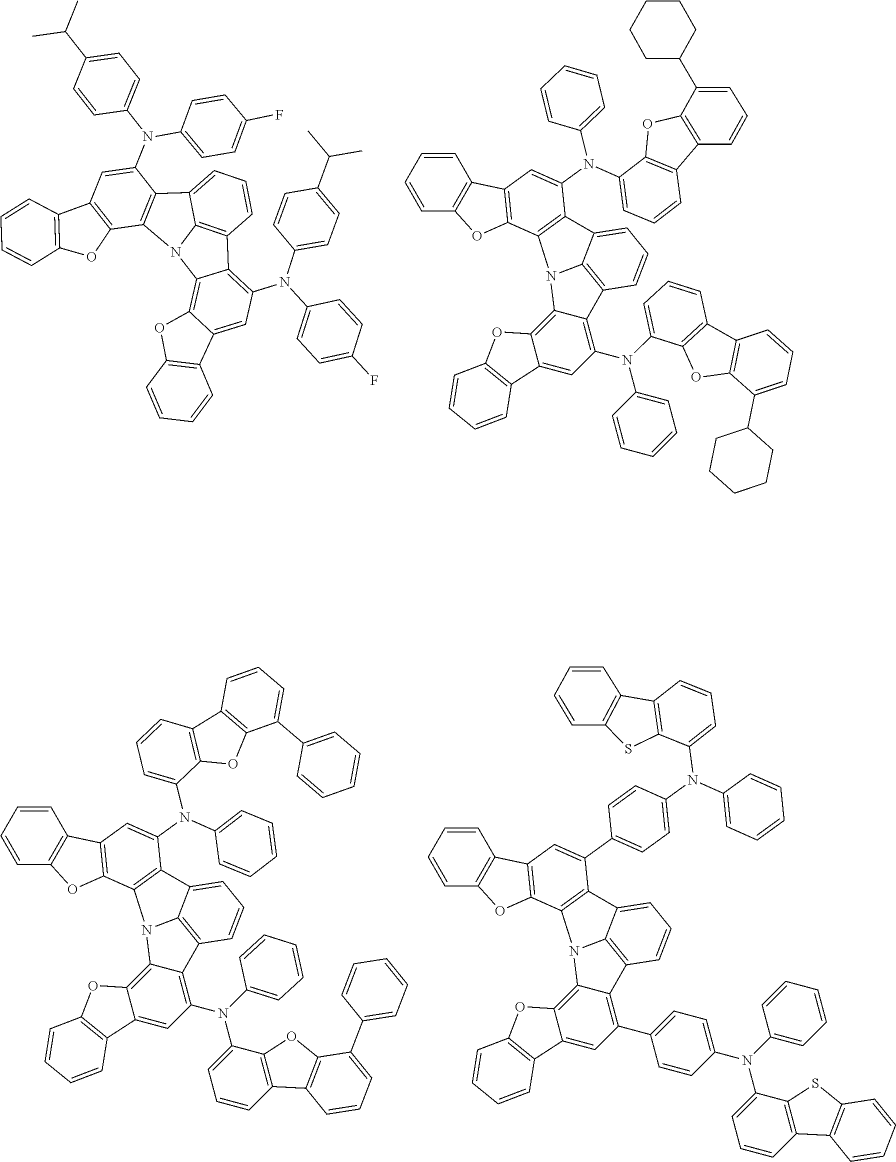

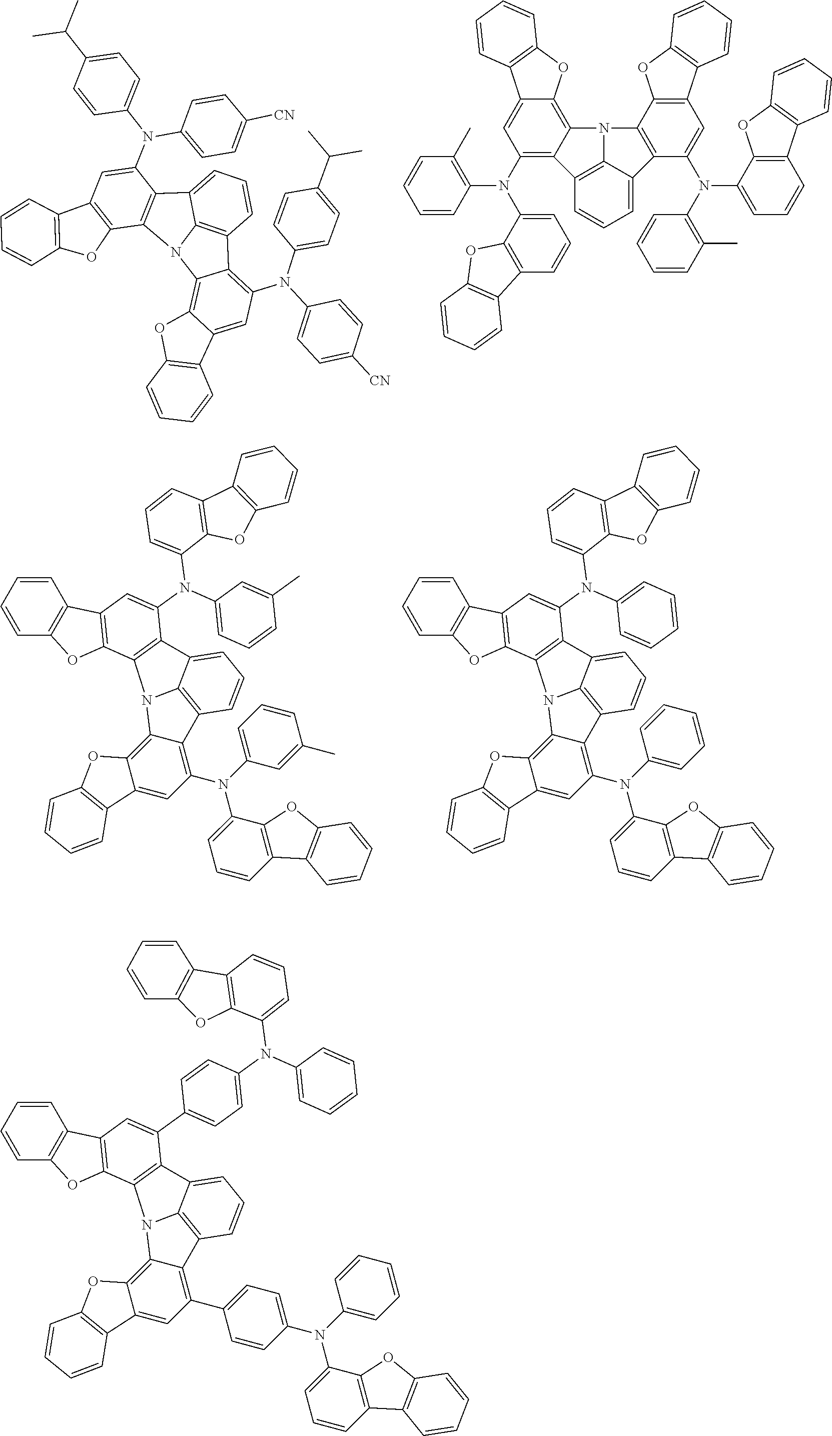

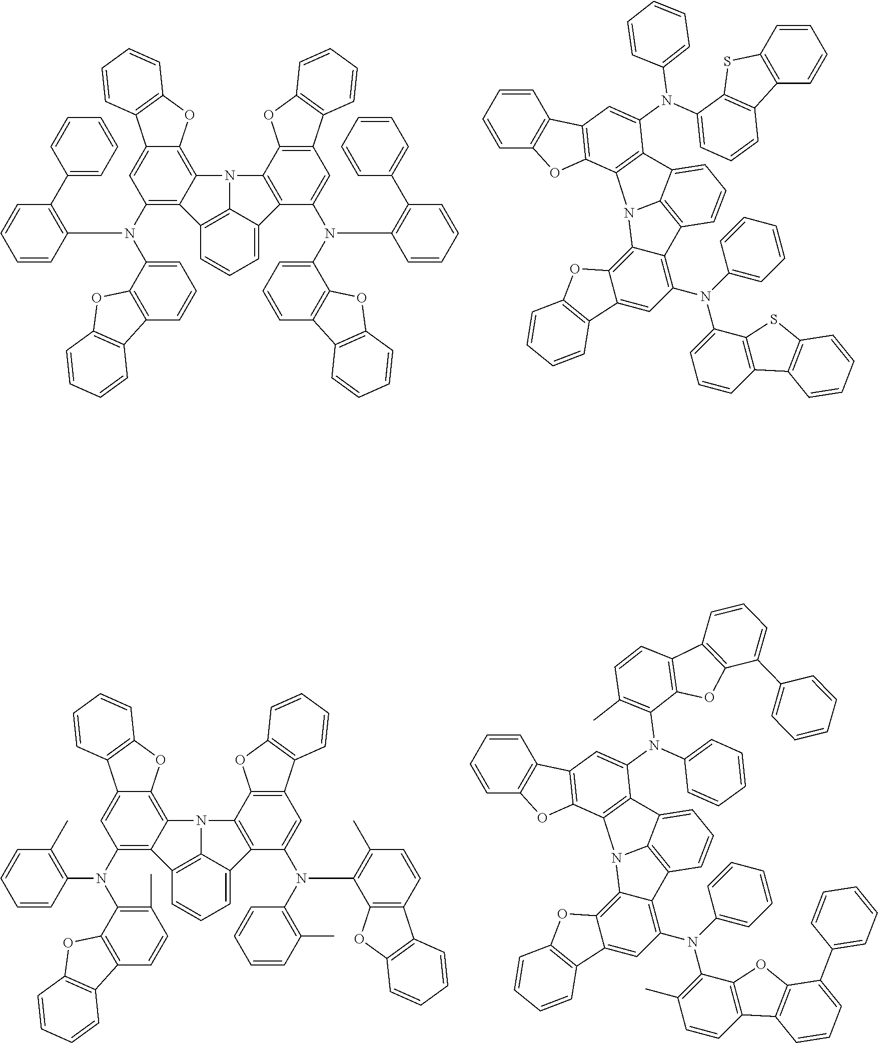

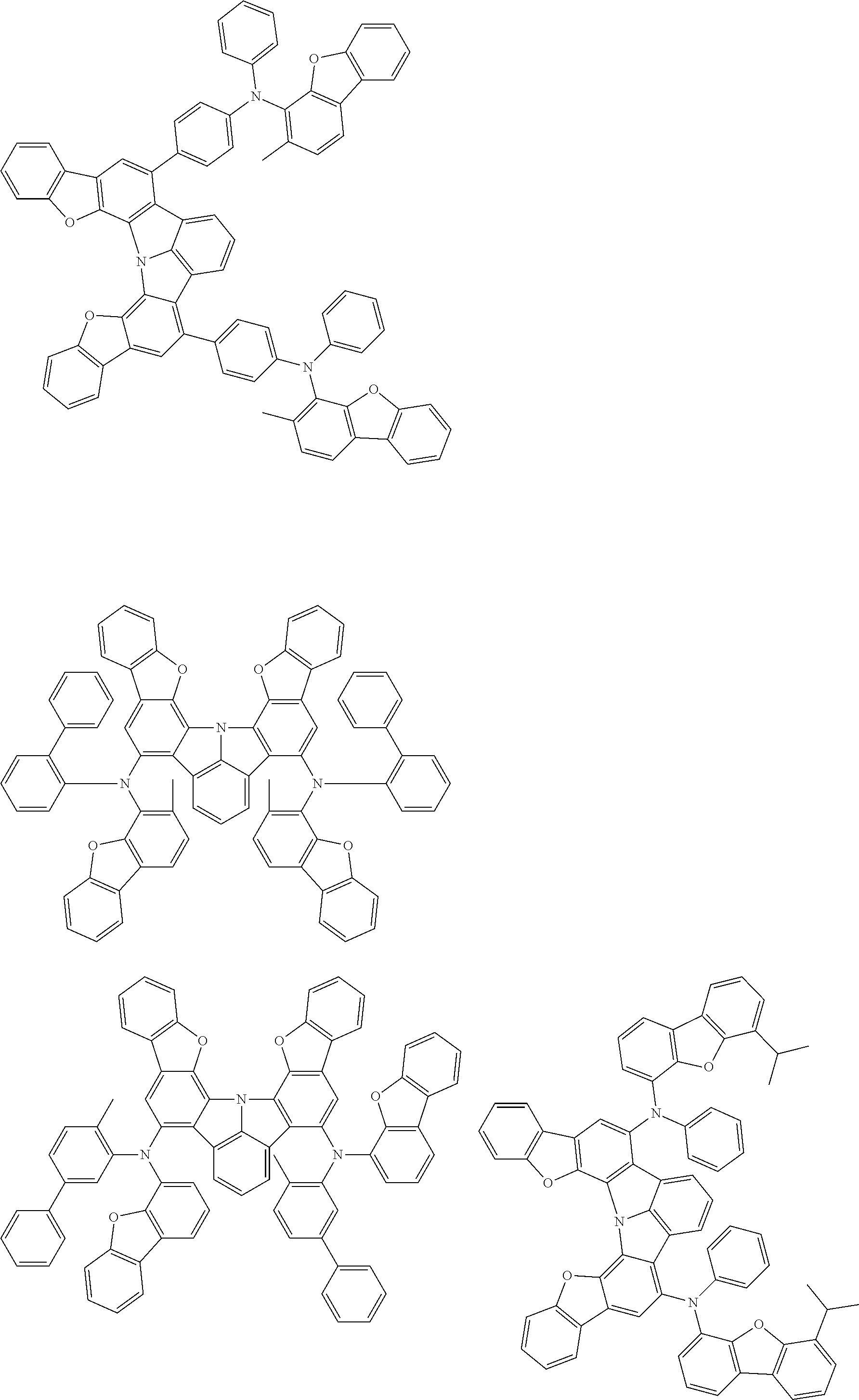

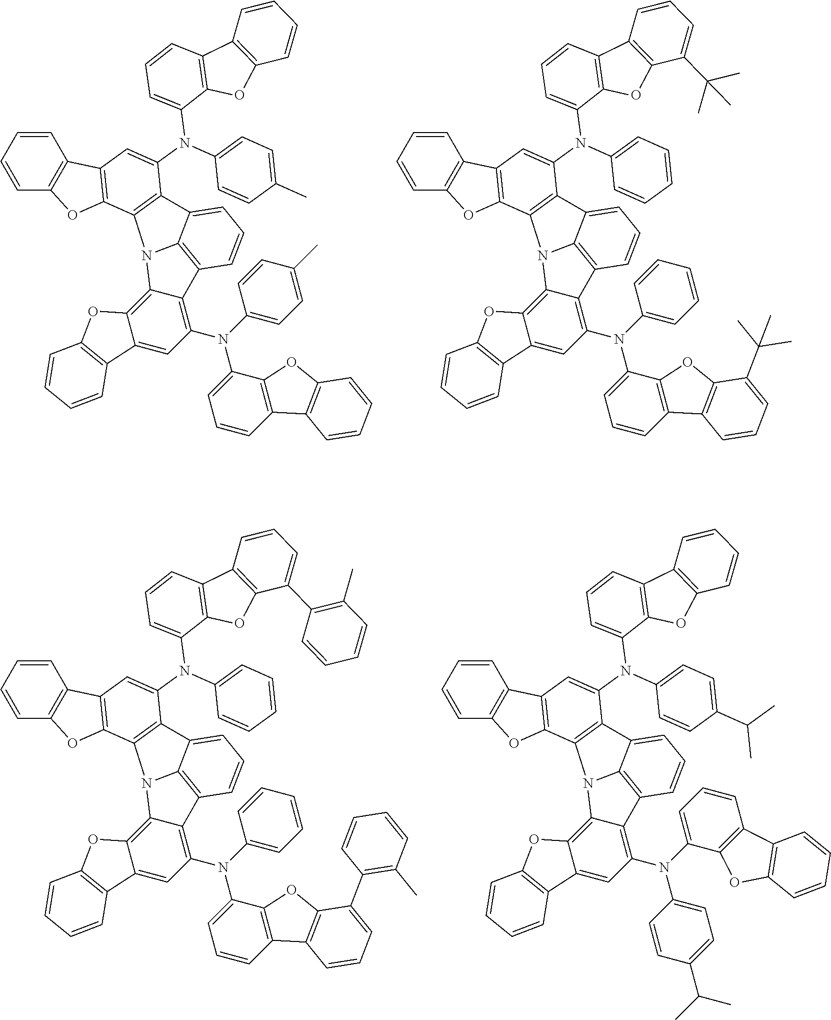

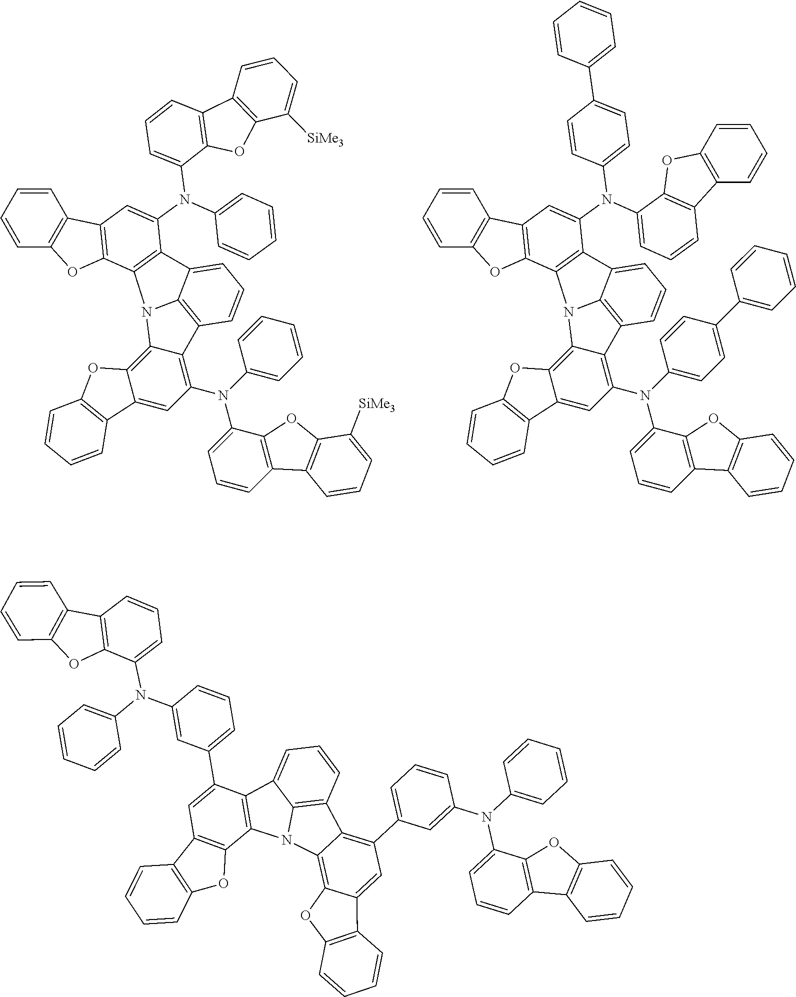 [SMILES: C[Si](C)(C)c1cccc2c1oc1c(N(c3ccccc3)c3cc4c5ccccc5oc4c4c3c3cccc5c6c(N(c7ccccc7)c7cccc8c7oc7c([Si](C)(C)C)cccc78)cc7c8ccccc8oc7c6n4c35)cccc12 ; c1ccc(-c2ccc(N(c3cccc4c3oc3ccccc34)c3cc4c5ccccc5oc4c4c3c3cccc5c6c(N(c7ccc(-c8ccccc8)cc7)c7cccc8c7oc7ccccc78)cc7c8ccccc8oc7c6n4c35)cc2)cc1 ; c1ccc(N(c2cccc(-c3cc4c5ccccc5oc4c4c3c3cccc5c6c(-c7cccc(N(c8ccccc8)c8cccc9c8oc8ccccc89)c7)cc7c8ccccc8oc7c6n4c35)c2)c2cccc3c2oc2ccccc23)cc1]

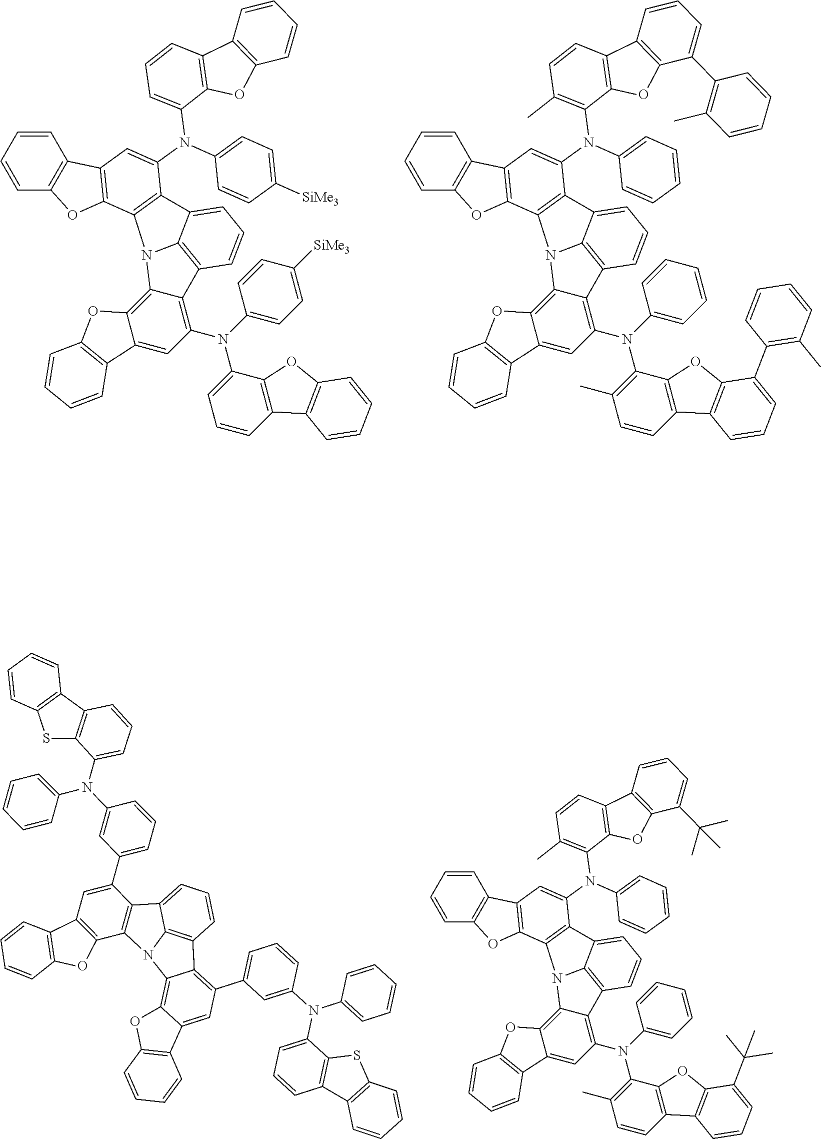

The production method of the compound of the invention is not particularly limited, and the compound can be easily produced by using or modifying a known synthetic reaction while referring to the examples described below.

### Material for Organic EL Device

The material for organic EL device of the invention comprises the compound (1). The content of the compound (1) in the material for organic electroluminescence devices is, but not particularly limited, 1 to 100% by mass, preferably 10 to 100% by mass, more preferably 50 to 100% by mass, still more preferably 80 to 100% by mass, and particularly preferably 90 to 100% by mass.

The material for organic EL device is useful for the production of organic EL devices.

In an embodiment, the compound of the invention is used as a fluorescent dopant material in a light emitting layer of organic EL device.

In view of the emission efficiency, the fluorescence quantum yield (PLQY) and the shape of fluorescence emission spectrum (half width) are important for the dopant material for use in the light emitting layer of organic EL device.

In a full-color display, to obtain an optimum color gamut, the three primary colors, i.e., red, green and blue light or four or more colors, for example, yellow in addition to the three primary colors are taken out after cutting off through a color filter or after amplifying a light of intended wavelength and attenuating light of other wavelengths. Thus, the light of a wavelength other than required is removed, this leading to a loss of energy. Therefore, a material showing an emission spectrum with a sharp shape is advantageous for the efficiency, because the range of wavelength to be cut off is small to reduce the loss of energy.

A material little changing its structure between the ground state and the excited state is considered suitable as a dopant material that shows an emission spectrum with a sharp shape.

The compound of the invention is characterized by the substituents at the specific positions. As a result of the extensive research, the inventors have found that the orientation is improved by the substituents at the specific positions.

For example, when the compound of the invention is used as an emitting material in an embodiment of the invention, the orientation of the emitting material is improved and the emitted light is efficiently taken out, thereby increasing the emission efficiency of the organic EL device.

Generally, it has been known that a rod-shape molecular structure is preferred for improving the orientation. However, it is very difficult to predict the molecular orientation in a vapor-deposited film. Therefore, it is essential to actually measure a vapor-deposited film to specify the substitution positions that increase the orientation.

### Organic EL Device

The organic EL device of the invention comprises a cathode, an anode and an organic layer between the cathode and the anode. The organic layer comprises a light emitting layer and at least one layer of the organic layer comprises the compound (1).

The layered structure of the organic EL device of the invention will be described below.

The organic EL device has an organic layer between a pair of electrodes consisting of a cathode and an anode. The organic layer comprises at least one layer formed from an organic compound. The organic layer may be a stack of two or more layers each formed from an organic compound. The organic layer may further include an inorganic compound.

At least one layer of the organic layer is a light emitting layer. The organic layer may be a single light emitting layer or additionally include another layer that is employed in an organic EL device, for example, a hole transporting region (a hole transporting layer, a hole injecting layer, an electron blocking layer, an exciton blocking layer, etc.) formed between an anode and a light emitting layer, a light emitting layer, a space layer, and an electron transporting region (an electron transporting layer, an electron injecting layer, a hole blocking layer, etc.) formed between a cathode and a light emitting layer, although not particularly limited thereto.

The organic EL device of the invention may be any of a single-color emitting fluorescent or phosphorescent device, a white-emitting fluorescent/phosphorescent hybrid device, a simple-type emitting device having a single emission unit, and a tandem emitting device having two or more emission units.

The “emission unit” referred to herein is the smallest unit for emitting light by the recombination of injected holes and injected electrons, which comprises one or more organic layers wherein at least one layer is a light emitting layer.

The “light emitting layer” referred to herein is a layer having a function of emitting light. The light emitting layer may be a phosphorescent emitting layer or a fluorescent emitting layer and may be a single layer or a multi-layer.

The emission unit may be a stack of layers having two or more phosphorescent emitting layers and fluorescent emitting layers. A space layer may be disposed between two light emitting layers to prevent the diffusion of excitons generated in a phosphorescent emitting layer into a fluorescent emitting layer.

The simple-type organic EL device has a device structure, for example, a structure of anode/emission unit/cathode.

Representative layered structures of the emission unit are shown below, wherein the layer in the parenthesis is optional:

(a) (Hole injecting layer/)Hole transporting layer/Fluorescent emitting layer(/Electron transporting layer/Electron injecting layer)
(b) (Hole injecting layer/)Hole transporting layer/Phosphorescent emitting layer(/Electron transporting layer/Electron injecting layer)
(c) (Hole injecting layer/)Hole transporting layer/First fluorescent emitting layer/Second fluorescent emitting layer(/Electron transporting layer/Electron injecting layer)
(d) (Hole injecting layer/)Hole transporting layer/First phosphorescent emitting layer/Second phosphorescent emitting layer(/Electron transporting layer/Electron injecting layer)
(e) (Hole injecting layer/)Hole transporting layer/Phosphorescent emitting layer/Space layer/Fluorescent emitting layer(/Electron transporting layer/Electron injecting layer)
(f) (Hole injecting layer/)Hole transporting layer/First phosphorescent emitting layer/Second phosphorescent emitting layer/Space layer/Fluorescent emitting layer(/Electron transporting layer/Electron injecting layer)
(g) (Hole injecting layer/)Hole transporting layer/First phosphorescent emitting layer/Space layer/Second phosphorescent emitting layer/Space layer/Fluorescent emitting layer(/Electron transporting layer/Electron injecting layer)
(h) (Hole injecting layer/)Hole transporting layer/Phosphorescent emitting layer/Space layer/First fluorescent emitting layer/Second fluorescent emitting layer(/Electron transporting layer/Electron injecting layer)
(i) (Hole injecting layer/)Hole transporting layer/Electron blocking layer/Fluorescent emitting layer(/Electron transporting layer/Electron injecting layer)
(j) (Hole injecting layer/)Hole transporting layer/Electron blocking layer/Phosphorescent emitting layer(/Electron transporting layer/Electron injecting layer)
(k) (Hole injecting layer/)Hole transporting layer/Exciton blocking layer/Fluorescent emitting layer(/Electron transporting layer/Electron injecting layer)
(l) (Hole injecting layer/)Hole transporting layer/Exciton blocking layer/Phosphorescent emitting layer(/Electron transporting layer/Electron injecting layer)
(m) (Hole injecting layer/)First hole transporting layer/Second hole transporting layer/Fluorescent emitting layer(/Electron transporting layer/Electron injecting layer)
(n) (Hole injecting layer/)First hole transporting layer/Second hole transporting layer/Fluorescent emitting layer(/First electron transporting layer/Second electron transporting layer/Electron injecting layer)
(o) (Hole injecting layer/)First hole transporting layer/Second hole transporting layer/Phosphorescent emitting layer(/Electron transporting layer/Electron injecting layer)
(p) (Hole injecting layer/)First hole transporting layer/Second hole transporting layer/Phosphorescent emitting layer(/First electron transporting layer/Second electron transporting layer/Electron injecting layer)
(q) (Hole injecting layer/)Hole transporting layer/Fluorescent emitting layer/Hole blocking layer(/Electron transporting layer/Electron injecting layer/Electron injecting layer)
(r) (Hole injecting layer/)Hole transporting layer/Phosphorescent emitting layer/Hole blocking layer(/Electron transporting layer/Electron injecting layer)
(s) (Hole injecting layer/)Hole transporting layer/Fluorescent emitting layer/Exciton blocking layer(/Electron transporting layer/Electron injecting layer)
(t) (Hole injecting layer/)Hole transporting layer/Phosphorescent emitting layer/Exciton blocking layer(/Electron transporting layer/Electron injecting layer)

The layered structure of the organic EL device of the invention is not limited to those described above. When a hole injecting layer and a hole transporting layer are provided, the hole injecting layer is preferably formed between the hole transporting layer and the anode. When an electron injecting layer and an electron transporting layer are provided, the electron injecting layer is formed between the electron transporting and the cathode. Each of the hole injecting layer, the hole transporting layer, the electron transporting layer, and the electron injecting layer may be a single layered structure or a multi-layered structure.

The emission colors of phosphorescent emitting layers and the emission colors of a phosphorescent emitting layer and a fluorescent emitting layer may be different. For example, the emission unit (f) may be Hole transporting layer/First phosphorescent emitting layer (red emission)/Second phosphorescent emitting layer (green emission)/Space layer/Fluorescent emitting layer (blue emission)/Electron transporting layer.

An electron blocking layer may be disposed between each light emitting layer and the hole transporting layer or between each light emitting layer and the space layer. Also, a hole blocking layer may be disposed between each light emitting layer and the electron transporting layer. With such an electron blocking layer or a hole blocking layer, electrons and holes are confined in the light emitting layer to increase the charge recombination in the light emitting layer, thereby improving the emission efficiency.

Representative device structure of the tandem-type organic EL device is Anode/First emission unit/Intermediate layer/Second emission unit/Cathode.

The first emission unit and the second emission unit may be independently selected from those described above with respect to the emission unit.

Generally, the intermediate layer is also called an intermediate electrode, an intermediate conductive layer, a charge generation layer, an electron withdrawing layer, a connecting layer, or an intermediate insulating layer. The intermediate layer supplies electrons to the first emission unit and holes to the second emission unit and may be formed by known materials.

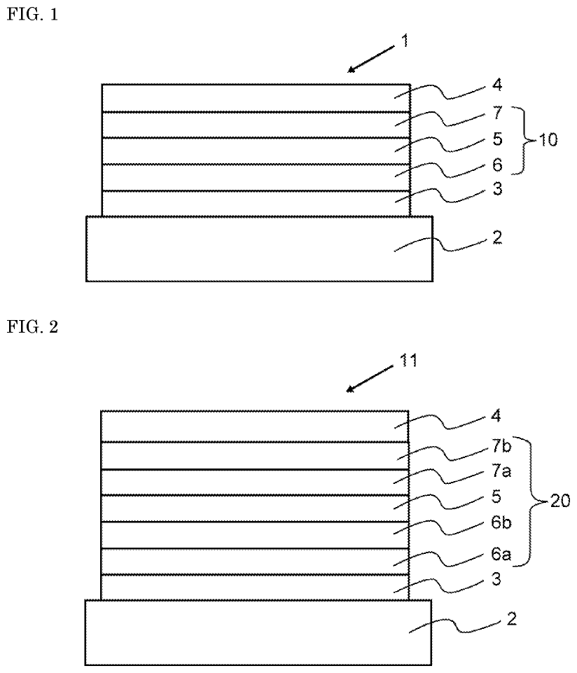

A schematic structure of an example of the organic EL device is shown in FIG. 1, wherein the organic EL device 1 comprises a substrate 2, an anode 3, a cathode 4, and an emission unit 10 (organic layer) disposed between the anode 3 and the cathode 4. The emission unit 10 includes at least one light emitting layer 5.

A hole transporting region 6 (a hole injecting layer, a hole transporting layer, etc.) may be disposed between the light emitting layer 5 and the anode 3, and an electron transporting region 7 (an electron injecting layer, an electron transporting layer, etc.) may be disposed between the light emitting layer 5 and the cathode 4. An electron blocking layer (not shown) may be disposed on the anode 3 side of the light emitting layer 5, and a hole blocking layer (not shown) may be disposed on the cathode 4 side of the light emitting layer 5. With these blocking layers, electrons and holes are confined in the light emitting layer 5 to increase the exciton generation in the light emitting layer 5.

A schematic structure of another example of the organic EL device is shown in FIG. 2. In an emission unit 20 of an organic EL device 11 of FIG. 2, the hole transporting layer in the hole transporting region 6 and the electron transporting layer in the electron transporting region 7, each in the emission unit 10 of the organic EL device 1 of FIG. 1, are made into two-layered structure, respectively. The hole transporting region includes an anode-side first hole transporting layer 6a and a cathode-side second hole transporting layer 6b. The electron transporting region 7 includes an anode-side first electron transporting layer 7a and a cathode-side second electron transporting layer 7b. The other reference signs are the same as those of FIG. 1 and the explanation thereof is omitted here.

The function and material of each layer of the organic EL device will be described below.

### Substrate

The substrate serves as a support for the organic EL device. The substrate is preferably a flat substrate having a transmittance of 50% or more to 400 to 700 nm visible light. The material of the substrate may include glass, quartz, and plastics. The substrate may be a flexible substrate, such as a plastic substrate. Examples of the material for the plastic substrate include polycarbonate, polyarylate, polyether sulfone, polypropylene, polyester, polyvinyl fluoride, polyvinyl chloride, polyimide, and polyethylene naphthalate. An inorganic deposition film is also usable.

### Anode

The anode is formed on the substrate preferably from a metal, an alloy, an electrically conductive compound, and a mixture thereof, each having a large work function, for example, 4.0 eV or more. Examples of the material for the anode include indium oxide-tin oxide (ITO: indium tin oxide), indium oxide-tin oxide doped with silicon or silicon oxide, indium oxide-zinc oxide, indium oxide doped with tungsten oxide and zinc oxide, and graphene. In addition, gold, platinum, nickel, tungsten, chromium, molybdenum, iron, cobalt, copper, palladium, titanium, and a nitride of the above metal (for example, titanium nitride) are also usable.

The anode is formed on the substrate generally by sputtering these materials into a film. For example, a film of indium oxide-zinc oxide is formed by sputtering an indium oxide target doped with 1 to 10% by mass of zinc oxide, and a film of indium oxide doped with tungsten oxide or zinc oxide is formed by sputtering an indium oxide target doped with 0.5 to 5% by mass of tungsten oxide or 0.1 to 1% by mass of zinc oxide.

In addition, a vacuum vapor deposition method, a coating method, an inkjet method, and a spin coating method are usable for forming the anode. For example, a coating method or an inkjet method is usable when a silver paste is used.

A hole injecting layer to be formed in contact with the anode is formed from a material which easily injects holes independently of the work function of the anode. Therefore, the anode can be formed by a known electrode material, for example, a metal, an alloy, an electroconductive compound, or a mixture thereof. Examples thereof include an alkali metal, such as lithium and cesium, an alkaline earth metal, such as magnesium, calcium, and strontium, an alloy thereof, such as magnesium-silver and aluminum-lithium, a rare earth metal, such as europium and ytterbium, and an alloy thereof, each having a small work function.

Hole Injecting Layer The hole injecting layer comprises a material having a high hole injecting ability and has a function of injecting holes from an anode into an organic layer. Examples of the material having a high hole injecting ability include molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, an electron accepting (acceptor) compound, and a high molecular compound, such as an oligomer, a dendrimer, and a polymer, with an aromatic amine compound and an acceptor compound being preferred and an acceptor compound being more preferred.

Examples of the aromatic amine compound include 4,4′,4″-tris(N,N-diphenylamino)triphenylamine (TDATA), 4,4′,4″-tris[N-(3-methylphenyl)-N-phenylamino]triphenylamine (MTDATA), 4,4′-bis[N-(4-diphenylaminophenyl)-N-phenylamino]biphenyl (DPAB), 4,4′-bis(N-{4-[N′-(3-methylphenyl)-N′-phenylamino]phenyl}-N-phenylamino)biphenyl (DNTPD), 1,3,5-tris[N-(4-diphenylaminophenyl)-N-phenylamino]benzene (DPA3B), 3-[N-(9-phenylcarbazole-3-yl)-N-phenylamino]-9-phenylcarbazole (PCzPCA1), 3,6-bis[N-(9-phenylcarbazole-3-yl)-N-phenylamino]-9-phenylcarbazole (PCzPCA2), and 3-[N-(1-naphthyl)-N-(9-phenylcarbazole-3-yl)amino]-9-phenylcarbazole (PCzPCN1).

Preferred examples of the acceptor compound include a heterocyclic derivative having an electron accepting group, a quinone derivative having an electron accepting group, an arylborane derivative, and a heteroarylborane derivative, for example, hexacyanohexaazatriphenylene, 2,3,5,6-tetrafluoro-7,7,8,8-tetracyanoquinodimethane (F₄TCNQ), and 1,2,3-tris[(cyano)(4-cyano-2,3,5,6-tetrafluorophenyl)methylene]cyclopropane.

A hole injecting layer comprising the acceptor compound preferably comprises a matrix material. The matrix material may be selected from a known material for organic EL devices, for example, an electron donating (donor) compound is preferred and an aromatic amine compound is more preferred.

### Hole Transporting Layer

The hole transporting layer comprises a highly hole transporting compound and has a function of transporting holes from an anode into an organic layer.

The highly hole transporting compound other than the compound (1) is preferably a compound having a hole mobility of 10⁻⁶ cm²/(V·s) or more, for example, an aromatic amine compound, a carbazole derivative, an anthracene derivative, and a polymer.

Examples of the aromatic amine compounds include 4,4′-bis[N-(1-naphthyl)-N-phenylamino]biphenyl (NPB), N,N′-bis(3-methylphenyl)-N,N′-diphenyl-[1,1′-biphenyl]-4,4′-diamine (TPD), 4-phenyl-4′-(9-phenylfluorene-9-yl)triphenylamine (BAFLP), 4,4′-bis[N-(9,9-dimethylfluorene-2-yl)-N-phenylamino]biphenyl (DFLDPBi), 4,4′,4″-tris(N,N-diphenylamino)triphenylamine (TDATA), 4,4′,4″-tris[N-(3-methylphenyl)-N-phenylamino]triphenylamine (MTDATA), and 4,4′-bis[N-(spiro-9,9′-bifluorene-2-yl)-N-phenylamino]biphenyl (BSPB).

Examples of the carbazole derivative include 4,4′-di(9-carbazolyl)biphenyl (CBP), 9-[4-(9-carbazolyl)phenyl]-10-phenylanthracene (CzPA), and 9-phenyl-3-[4-(10-phenyl-9-anthryl)phenyl]-9H-carbazole (PCzPA).

Examples of the anthracene derivative include 2-t-butyl-9,10-di(2-naphthyl)anthracene (t-BuDNA), 9,10-di(2-naphthyl)anthracene (DNA), and 9,10-diphenylanthracene (DPAnth).

Examples of the polymer include poly(N-vinylcarbazole) (PVK) and poly(4-vinyltriphenylamine) (PVTPA).

A compound other than those mentioned above is also usable, if its hole transporting ability is higher than its electron transporting ability.

The hole transporting layer may be a single layer or a multi-layer including two or more layers. In a multi-layered structure, a hole transporting layer comprising a highly hole transporting compound that has a larger energy gap is preferably positioned closer to a light emitting layer.

For example, as shown in FIG. 2, the hole transporting layer may be made into a structure having an anode-side first hole transporting layer 6a and a cathode-side second hole transporting layer 6b.

### Light Emitting Layer

The light emitting layer comprises a highly light-emitting material (dopant material). A various kind of materials may be used as the dopant material, for example, a fluorescent emitting compound (a fluorescent dopant) and a phosphorescent emitting compound (a phosphorescent dopant) are usable. The fluorescent emitting compound is a compound capable of emitting light from a singlet excited state and a light emitting layer comprising it is called a fluorescent emitting layer. The phosphorescent emitting compound is a compound capable of emitting light from a triplet excited state and a light emitting layer comprising it is called a phosphorescent emitting layer.

The light emitting layer generally comprises a dopant material and a host material that causes the dopant material to efficiently emit light. The dopant material may be described as a guest material, an emitter material, or a light emitting material in some document. The host material may be described as a matrix material in some documents.

Two or more dopant materials or two or more host materials may be included in a single light emitting layer. Two or more light emitting layers may be included in a device.

In the present invention, a host material is referred to as “a fluorescent host material” when combinedly used with a fluorescent dopant material and as “a phosphorescent host material” when combinedly used with a phosphorescent dopant material. Therefore, the fluorescent host material and the phosphorescent host material are not distinguished from each other merely by the difference in their molecular structures. The phosphorescent host is a material for forming a phosphorescent emitting layer comprising a phosphorescent dopant and does not mean a material that cannot be used as a material for a fluorescent emitting layer. The same applies to the fluorescent host.

The light emitting layer preferably comprises the compound (1), more preferably as a dopant material, and still more preferably as a fluorescent dopant. In addition, the compound (1) is preferably used in a light emitting layer as a dopant for a thermally activated delayed fluorescence (TADF).

The content of the compound (1) as a dopant material in a light emitting layer is not particularly limited and preferably 0.1 to 70% by mass, more preferably 0.1 to 30% by mass, still more preferably 1 to 30% by mass, further more preferably 1 to 20% by mass, and particularly preferably 1 to 10% by mass, in view of sufficient emission and concentration quenching.

### Fluorescent Dopant

Examples of the fluorescent dopant other than the compound (1) include a fused aromatic polycyclic derivative, a styrylamine derivative, a fused amine derivative, a boron-containing compound, a pyrrole derivative, an indole derivative, and a carbazole derivative, with a fused amine derivative and a boron-containing compound being preferred.

Examples of the fused amine derivative include a diaminopyrene derivative, a diaminochrysene derivative, a diaminoanthracene derivative, a diaminofluorene derivative, and a diaminofluorene derivative to which at least one benzofuro skeleton is fused.

Examples of the boron-containing compound include a pyrromethene derivative and a triphenylborane derivative.

Examples of blue fluorescent dopant include a pyrene derivative, a styrylamine derivative, a chrysene derivative, a fluoranthene derivative, a fluorene derivative, a diamine derivative, and a triarylamine derivative, such as N,N′-bis[4-(9H-carbazole-9-yl)phenyl]-N,N′-diphenylstilbene-4,4′-diamine (YGA2S), 4-(9H-carbazole-9-yl)-4′-(10-phenyl-9-anthryl)triphenylamine (YGAPA), and 4-(10-phenyl-9-anthryl)-4′-(9-phenyl-9H-carbazole-3-yl)triphenylamine (PCBAPA).

Examples of green fluorescent dopant include an aromatic amine derivative, such as N-(9,10-diphenyl-2-anthryl)-N,9-diphenyl-9H-carbazole-3-amine (2PCAPA), N-[9,10-bis(1,1′-biphenyl-2-yl)-2-anthryl]-N,9-diphenyl-9H-carbazole-3-amine (2PCABPhA), N-(9,10-diphenyl-2-anthryl)-N,N′,N′-triphenyl-1,4-phenylenediamine (2DPAPA), N-[9,10-bis(1,1′-biphenyl-2-yl)-2-anthryl]-N,N′,N′-triphenyl-1,4-phenylenediamine (2DPABPhA), N-[9,10-bis(1,1′-biphenyl-2-yl)]—N-[4-(9H-carbazole-9-yl)phenyl]-N-phenylanthracene-2-amine (2YGABPhA), and N,N,9-triphenylanthracene-9-amine (DPhAPhA).

Examples of red fluorescent dopant include a tetracene derivative and a diamine derivative, such as N,N,N′,N′-tetrakis(4-methylphenyl)tetracene-5,11-diamine (p-mPhTD) and 7,14-diphenyl-N,N,N′,N′-tetrakis(4-methylphenyl)acenaphtho[1,2-a]fluoranthene-3,10-diamine (p-mPhAFD).

### Phosphorescent Dopant

Examples of the phosphorescent dopant include a phosphorescent heavy metal complex and a phosphorescent rare earth metal complex.

Examples of the heavy metal complex include an iridium complex, an osmium complex, and a platinum complex, with an ortho metallated complex of a metal selected from iridium, osmium, and platinum being preferred.

Examples of the rare earth metal complex include a terbium complex and a europium complex, for example, tris(acetylacetonato)(monophenanthrorine)terbium(III) (Tb(acac)₃(Phen)), tris(1,3-diphenyl-1,3-prop anedionato)(monophenanthrorine)europium(III) (Eu(DBM)₃(Phen)), and tris[1-(2-thenoyl)-3,3,3-trifluoroacetonato](monophenanthrorine)europium(III) (Eu(TTA)₃(Phen)). These rare earth metal complexes are preferred as the phosphorescent dopant, because the rare earth metal ion emits light by the electron transition between different multiple states.

Examples of blue phosphorescent dopant include an iridium complex, an osmium complex, and a platinum complex, such as bis[2-(4′,6′-difluorophenyl)pyridinato-N,C2′]iridium(III) tetrakis(1-pyrazolyl)borato (FIr₆), bis[2-(4′,6′-difluorophenyl)pyridinato-N,C2′]iridium(III) picolinato (FIrpic), bis[2-(3′,5′-bistrifluoromethylphenyl)pyridinato-N,C2′]iridium(III) picolinato (Ir(CF₃ppy)₂(pic)), and bis[2-(4′,6′-difluorophenyl)pyridinato-N,C2′]iridium(III) acetylacetonato (FIracac).

Examples of green phosphorescent dopant include an iridium complex, such as tris(2-phenylpyridinato-N,C2′)iridium(III) (Ir(ppy)₃), bis(2-phenylpyridinato-N,C2′)iridium(III) acetylacetonato (Ir(ppy)₂(acac)), bis(1,2-diphenyl-1H-benzimidazolato)iridium(III) acetylacetonato (Ir(pbi)₂(acac)), and bis(benzo[h]quinolinato)iridium(III) acetylacetonato (Ir(bzq)₂(acac)).

Examples of red phosphorescent dopant include an iridium complex, a platinum complex, a terbium complex, and a europium complex, such as an organometallic complex, such as bis[2-(2′-benzo[4,5-a]thienyl)pyridinato-N,C3′]iridium(III) acetylacetonato (Ir(btp)₂(acac)), bis(1-phenylisoquinolinato-N,C2′)iridium(III) acetylacetonato (Ir(piq)₂(acac)), (acetylacetonato)bis[2,3-bis(4-fluorophenyl)quinoxalinato]iridium(III) (Ir(Fdpq)₂(acac)), and 2,3,7,8,12,13,17,18-octaethyl-21H,23H-porphyrin platinum(II) (PtOEP).

### Host Material

In an embodiment of the invention, an anthracene derivative is preferably used as a host material in a light emitting layer.

The organic EL device in an aspect of the invention preferably comprises the compound described above and the compound represented by formula (10) in at least one layer of the organic layer, for example, in a light emitting layer.

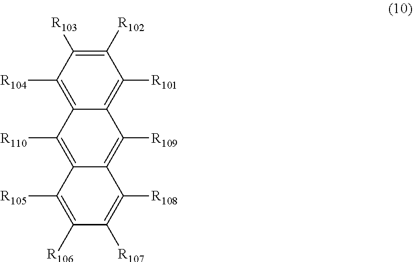

wherein at least one of R₁₀₁ to R₁₁₀ is a group represented by formula (31) and two or more groups represented by formula (31), if present, may be the same or different.

-L₁₀₁-Ar₁₀₁ (31)

wherein:

L₁₀₁ is a single bond, a substituted or unsubstituted arylene group having 6 to 30 ring carbon atoms, or a substituted or unsubstituted divalent heterocyclic group having 5 to 30 ring atoms; and

Ar₁₀₁ is a substituted or unsubstituted aryl group having 6 to 50 ring carbon atoms or a substituted or unsubstituted monovalent heterocyclic group having 5 to 50 ring atoms.

One or more sets of adjacent two selected from R₁₀₁ to R₁₁₀ not representing the group represented by formula (31) form a substituted or unsubstituted, saturated or unsaturated ring or do not form a ring.

R₁₀₁ to R₁₁₀ not representing the group represented by formula (31) and not forming a ring are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a substituted or unsubstituted aryl group having 6 to 50 ring carbon atoms, or a substituted or unsubstituted monovalent heterocyclic group having 5 to 50 ring atoms.

R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted monovalent heterocyclic group having 5 to 50 ring atoms. Two or more R₉₀₁ to R₉₀₇, if present, may be the same or different.

In an embodiment of the invention, the compound (10) is represented by formula (10-1):

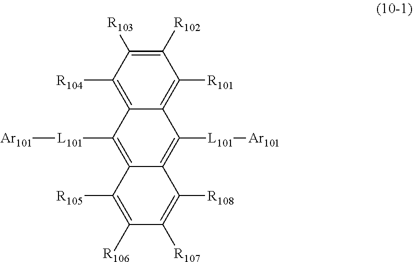

wherein R₁₀₁ to R₁₀₈, L₁₀₁, and Ar₁₀₁ are as defined in formula (10).

In an embodiment of the invention, the compound (10) is represented by formula (10-2):

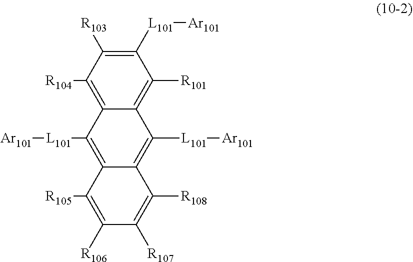

wherein R₁₀₁, R₁₀₃ to R₁₀₈, L₁₀₁, and Ar₁₀₁ are as defined in formula (10).

In an embodiment of the invention, the compound (10) is represented by formula (10-3):

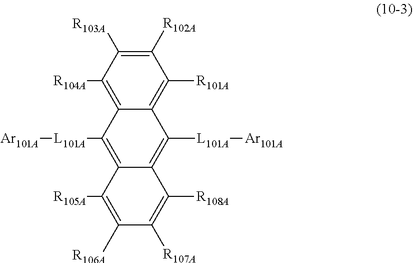

wherein:

R_{101A} to R_{108A} are each independently a hydrogen atom or a substituted or unsubstituted aryl group having 6 to 50 ring carbon atoms;

L_{101A} is a single bond or a substituted or unsubstituted arylene group having 6 to 30 ring carbon atoms and two L_{101A} may be the same or different; and

Ar^{101A} is a substituted or unsubstituted aryl group having 6 to 50 ring carbon atoms and two Ar_{101A} may be the same or different.

In an embodiment of the invention, the compound (10) is represented by formula (10-4):

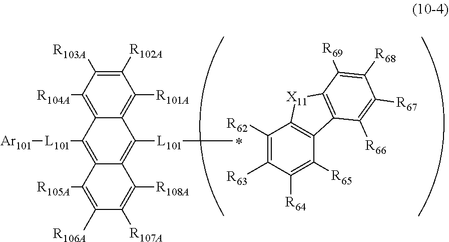

wherein:

L₁₀₁ and Ar₁₀₁ are as defined in formula (10);

R_{101A} to R_{108A} are each independently a hydrogen atom or a substituted or unsubstituted aryl group having 6 to 50 ring carbon atoms;

X₁₁ is O, S, C(R₉₁)(R₉₂), or N(R₆₁)

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms;

R₆₁ is a hydrogen atom, a substituted or unsubstituted alkyl group having 1 to 50 carbon atoms, or a substituted or unsubstituted aryl group having 6 to 50 ring carbon atoms;

at least one of R₆₂ to R₆₉ is a bond bonded to L₁₀₁;

one or more sets of adjacent two selected from R₆₂ to R₆₉ not bonded to L₁₀₁ form a substituted or unsubstituted, saturated or unsaturated ring or do not form a ring; and

R₆₂ to R₆₉ not bonded to L₁₀₁ and not forming the ring are each independently a hydrogen atom, a substituted or unsubstituted alkyl group having 1 to 50 carbon atoms, or a substituted or unsubstituted aryl group having 6 to 50 ring carbon atoms.

In an embodiment of the invention, the compound (10) is represented by formula (10-4A):

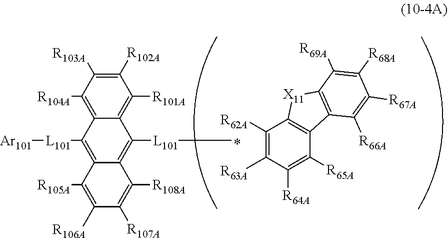

wherein:

L₁₀₁ and Ar₁₀₁ are as defined in formula (10);

R_{101A} to R_{108A} are each independently a hydrogen atom or a substituted or unsubstituted aryl group having 6 to 50 ring carbon atoms;

X₁₁ is O, S, C(R₉₁)(R₉₂), or N(R₆₁)

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms;

R₆₁ is a hydrogen atom, a substituted or unsubstituted alkyl group having 1 to 50 carbon atoms, or a substituted or unsubstituted aryl group having 6 to 50 ring carbon atoms;

one set of adjacent two selected from R_{62A} to R_{69A} forms a ring represented by formula (10-4A-1):

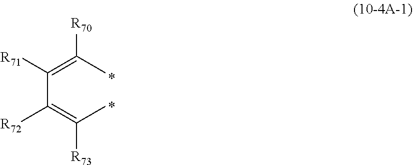

wherein:

two bonds * are bonded to adjacent two selected from R_{62A} to R_{69A};

one of R₇₀ to R₇₃ is a bond bonded to L₁₀₁; and

R₇₀ to R₇₃ not bonded to L₁₀₁ are each independently a hydrogen atom, a substituted or unsubstituted alkyl group having 1 to 50 carbon atoms, or a substituted or unsubstituted aryl group having 6 to 50 ring carbon atoms.

One or more sets of adjacent two selected from R_{62A} to R_{69A} not forming the ring represented by formula (10-4A-1) form a substituted or unsubstituted, saturated or unsaturated ring or do not form a ring.

R_{62A} to R_{69A} not forming the ring represented by formula (10-4A-1) and a substituted or unsubstituted, saturated or unsaturated ring are each independently a hydrogen atom, a substituted or unsubstituted alkyl group having 1 to 50 carbon atoms, or a substituted or unsubstituted aryl group having 6 to 50 ring carbon atoms.

In an embodiment of the invention, the compound (10) is represented by formula (10-6):

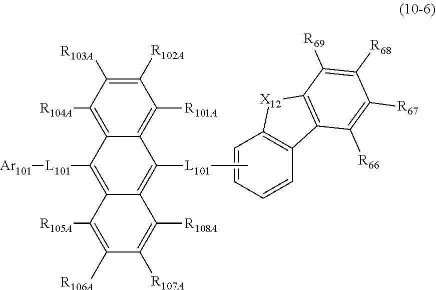

wherein:

L₁₀₁ and Ar₁₀₁ are as defined in formula (10);

R_{101A} to R_{108A} are as defined in formula (10-4);

R₆₆ to R₆₉ are as defined in formula (10-4);

X₁₂ is O, S, or C(R₉₁)(R₉₂); and

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms.

In an embodiment of the invention, the compound represented by formula (10-6) is represented by formula (10-6H):

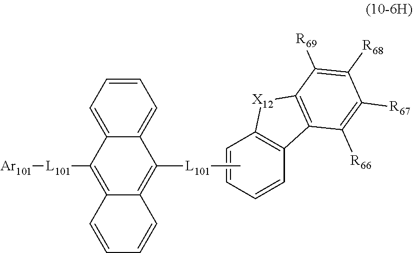

wherein:

L₁₀₁ and Ar₁₀₁ are as defined in formula (10);

R₆₆ to R₆₉ are as defined in formula (10-4);

X₁₂ is O, S, or C(R₉₁)(R₉₂); and

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms.

In an embodiment of the invention, the compound represented by formula (10-6) or (10-6H) is represented by formula (10-6Ha):

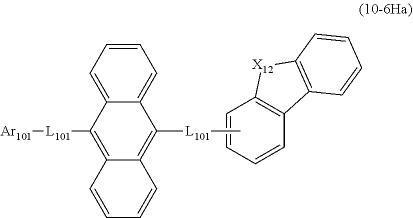

wherein:

L₁₀₁ and Ar₁₀₁ are as defined in formula (10);

X₁₂ is O, S, or C(R₉₁)(R₉₂); and

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms.

In an embodiment of the invention, the compound represented by formula (10-6), (10-6H), or (10-6Ha) is represented by formula (10-6Ha-1) or (10-6Ha-2):

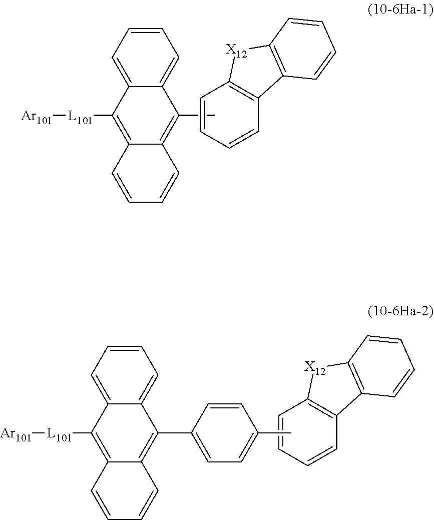

wherein:

L₁₀₁ and Ar₁₀₁ are as defined in formula (10);

X₁₂ is O, S, or C(R₉₁)(R₉₂); and

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms.

In an embodiment of the invention, the compound (10) is represented by formula (10-7):

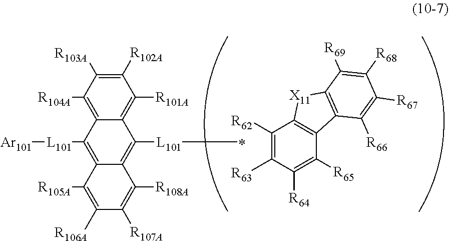

wherein:

L₁₀₁ and Ar₁₀₁ are as defined in formula (10);

R_{101A} to R_{108A} are as defined in formula (10-4);

X₁₁ is as defined in formula (10-4); and

R₆₂ to R₆₉ are as defined in formula (10-4), provided that one set of adjacent two selected from R₆₆ and R₆₇, R₆₇ and R₆₈, and R₆₈ and R₆₉ are bonded to each other to form a substituted or unsubstituted, saturated or unsaturated ring.

In an embodiment of the invention, the compound (10) is represented by formula (10-7H):

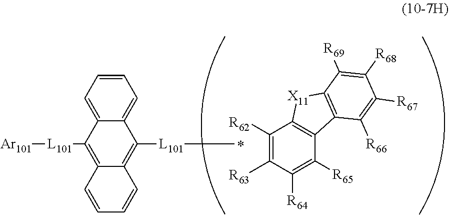

wherein

L₁₀₁ and Ar₁₀₁ are as defined in formula (10);

X₁₁ is as defined in formula (10-4); and

R₆₂ to R₆₉ are as defined in formula (10-4), provided that one set of adjacent two selected from R₆₆ and R₆₇, R₆₇ and R₆₈, and R₆₈ and R₆₉ are bonded to each other to form a substituted or unsubstituted, saturated or unsaturated ring.

In an embodiment of the invention, the compound (10) is represented by formula (10-8):

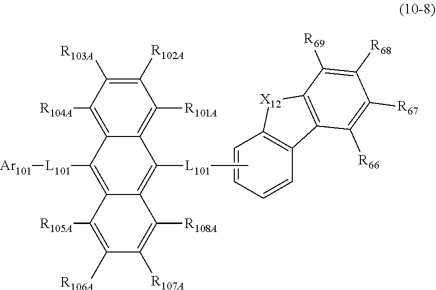

wherein:

L₁₀₁ and Ar₁₀₁ are as defined in formula (10);

R_{101A} to R_{108A} are as defined in formula (10-4);

X₁₂ is O, S, or C(R₉₁)(R₉₂);

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms; and

R₆₆ to R₆₉ are as defined in formula (10-4), provided that one set of adjacent two selected from R₆₆ and R₆₇, R₆₇ and R₆₈, and R₆₈ and R₆₉ are bonded to each other to form a substituted or unsubstituted, saturated or unsaturated ring.

In an embodiment of the invention, the compound represented by formula (10-8) is represented by formula (10-8H):

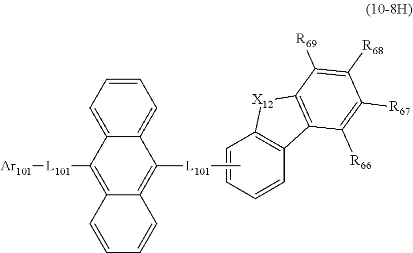

wherein

L₁₀₁ and Ar₁₀₁ are as defined in formula (10).

R₆₆ to R₆₉ are as defined in formula (10-4), provided that one set of adjacent two selected from R₆₆ and R₆₇, R₆₇ and R₆₈, and R₆₈ and R₆₉ are bonded to each other to form a substituted or unsubstituted, saturated or unsaturated ring, preferably form an unsubstituted benzene ring;

X₁₂ is O, S, or C(R₉₁)(R₉₂); and

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms.

In an embodiment of the invention, one set of adjacent two selected from R₆₆ and R₆₇, R₆₇ and R₆₈, and R₆₈ and R₆₉ of the compound represented by formula (10-7), (10-8) or (10-8H) are bonded to each other to form a ring represented by formula (10-8-1) or (10-8-2) and R₆₆ to R₆₉ not forming the ring represented by formula (10-8-1) or (10-8-2) do not form a substituted or unsubstituted, saturated or unsaturated ring:

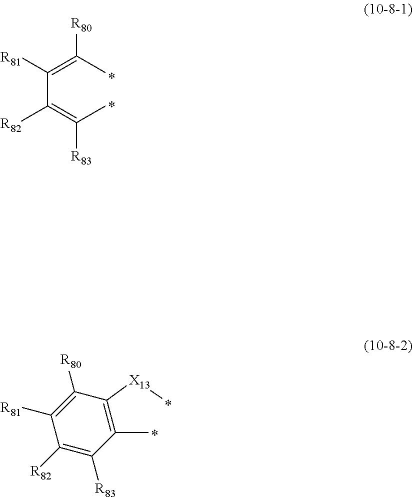

wherein:

two bonds * are bonded to one set of adjacent two selected from R₆₆ and R₆₇, R₆₇ and R₆₈, and R₆₈ and R₆₉;

R₈₀ to R₈₃ are each independently a hydrogen atom, a substituted or unsubstituted alkyl group having 1 to 50 carbon atoms, or a substituted or unsubstituted aryl group having 6 to 50 ring carbon atoms;

X₁₃ is O, S, or C(R₉₁)(R₉₂); and

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms.

In an embodiment of the invention, the compound (10) is represented by formula (10-9):

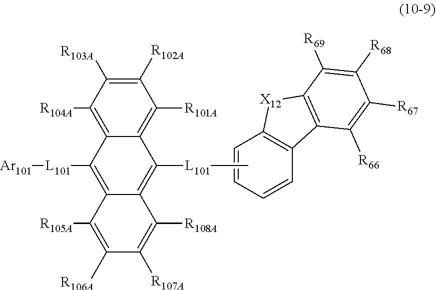

wherein:

L₁₀₁ and Ar₁₀₁ are as defined in formula (10);

R_{101A} to R_{108A} are as defined in formula (10-4);

R₆₆ to R₆₉ are as defined in formula (10-4), provided that adjacent two selected from R₆₆ and R₆₇, R₆₇ and R₆₈, and R₆₉ and R₆₇ are not bonded to each other thereby failing to form a substituted or unsubstituted, saturated or unsaturated ring;

X₁₂ is O, S, or C(R₉₁)(R₉₂); and

R₉₁ and R₉₂ are as defined above with respect to R¹ to R⁹ and preferably a hydrogen atom, a substituted or unsubstituted alkyl group having 1 to 20 carbon atoms, or a substituted or unsubstituted aryl group having 6 to 50 ring carbon atoms.

In an embodiment of the invention, the compound (10) is selected from the group consisting of the compounds represented by formulae (10-10-1) to (10-10-4):

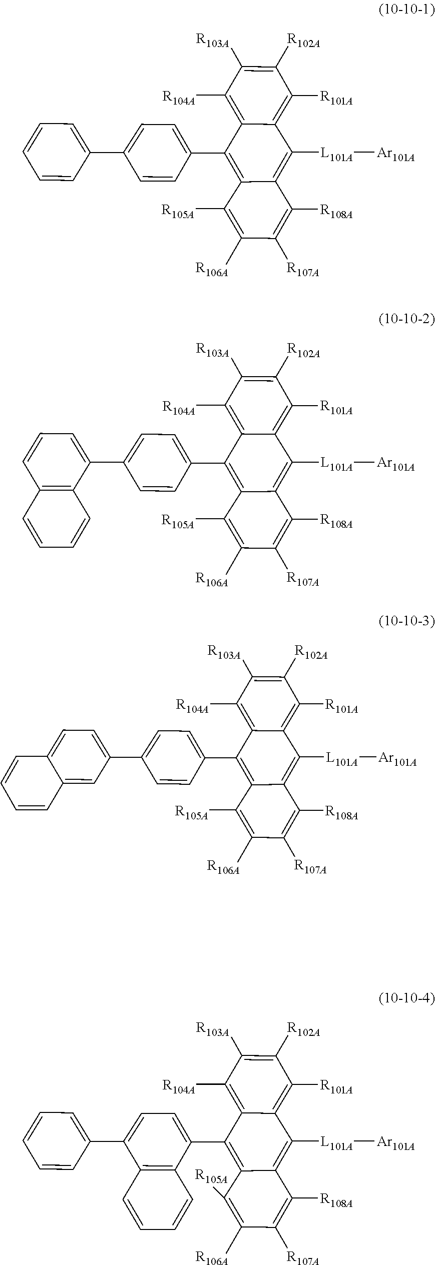

wherein L_{101A}, Ar_{101A}, and R_{101A} to R_{108A} are as defined in formula (10-3).

In an embodiment of the invention, the compounds represented by formulae (10-10-1) to (10-10-4) are represented by formulae (10-10-1H) to (10-10-4H):

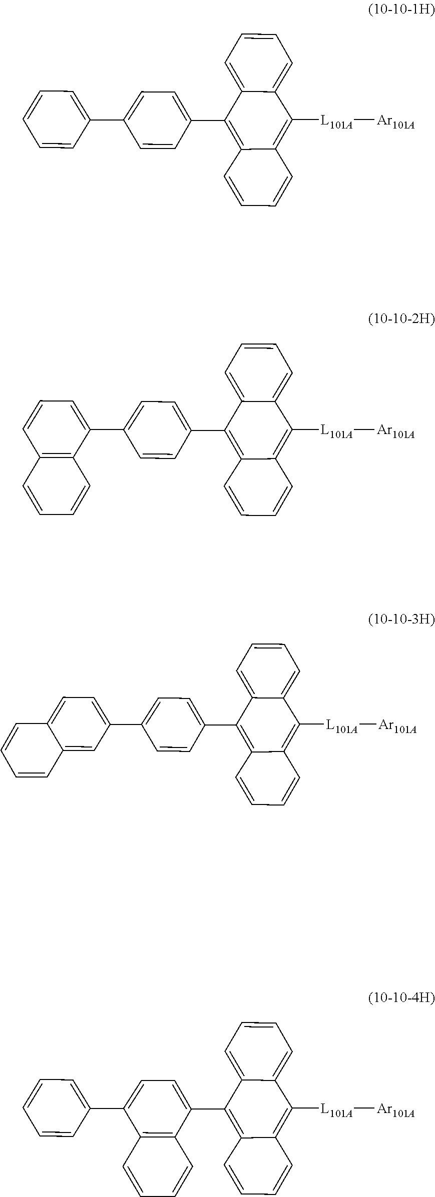

wherein L_{101A} and Ar_{101A} are as defined in formula (10-3).

In formulae (10), (10-1) to (10-4), (10-4-4A), (10-6), (10-6H), (10-6Ha), (10-6Ha-1), (10-6Ha-2), (10-7), (10-7H), (10-8), (10-8H), (10-9), (10-10-1) to (10-10-4), and (10-10-1H) to (10-10-4H), the details of each substituent and the details of the substituents referred to by “substituted or unsubstituted” are as described above in the item of “Definition.”

Examples of the compound represented by formula (10) are shown below.

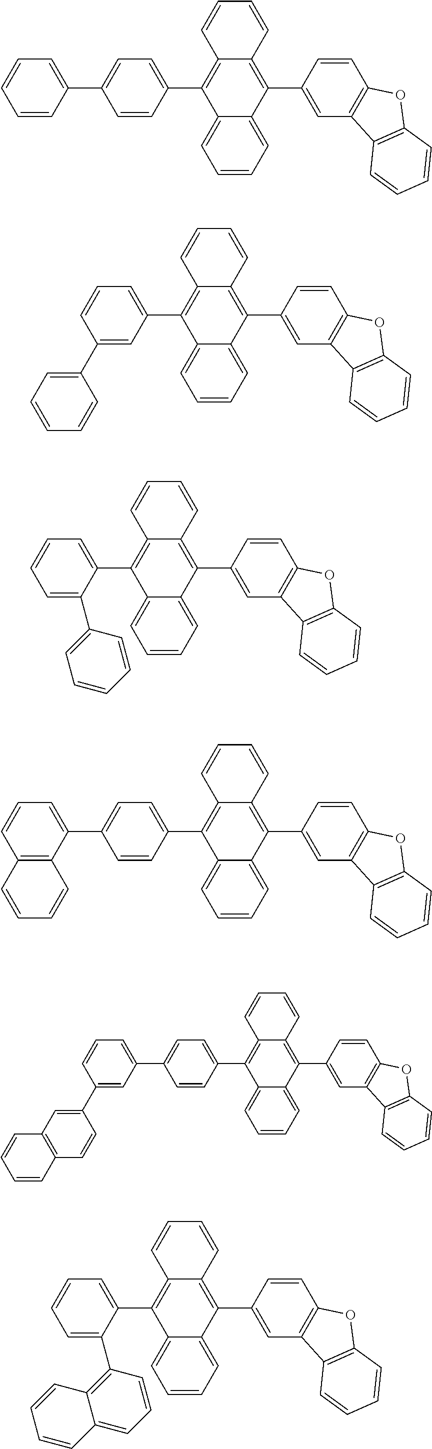 [SMILES: c1ccc(-c2ccc(-c3c4ccccc4c(-c4ccc5oc6ccccc6c5c4)c4ccccc34)cc2)cc1 ; c1ccc(-c2cccc(-c3c4ccccc4c(-c4ccc5oc6ccccc6c5c4)c4ccccc34)c2)cc1 ; c1ccc(-c2ccccc2-c2c3ccccc3c(-c3ccc4oc5ccccc5c4c3)c3ccccc23)cc1 ; c1ccc2c(-c3ccc(-c4c5ccccc5c(-c5ccc6oc7ccccc7c6c5)c5ccccc45)cc3)cccc2c1 ; c1cc(-c2ccc(-c3c4ccccc4c(-c4ccc5oc6ccccc6c5c4)c4ccccc34)cc2)cc(-c2ccc3ccccc3c2)c1 ; c1ccc(-c2c3ccccc3c(-c3ccc4oc5ccccc5c4c3)c3ccccc23)c(-c2cccc3ccccc23)c1]

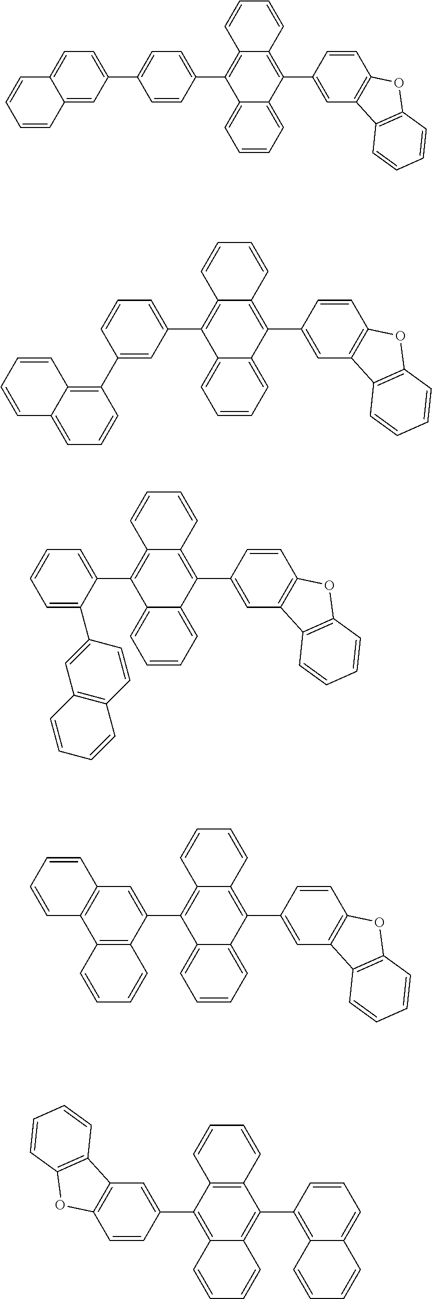 [SMILES: c1ccc2cc(-c3ccc(-c4c5ccccc5c(-c5ccc6oc7ccccc7c6c5)c5ccccc45)cc3)ccc2c1 ; c1cc(-c2cccc3ccccc23)cc(-c2c3ccccc3c(-c3ccc4oc5ccccc5c4c3)c3ccccc23)c1 ; c1ccc(-c2c3ccccc3c(-c3ccc4oc5ccccc5c4c3)c3ccccc23)c(-c2ccc3ccccc3c2)c1 ; c1ccc2c(c1)cc(-c1c3ccccc3c(-c3ccc4oc5ccccc5c4c3)c3ccccc13)c1ccccc12 ; c1ccc2c(-c3c4ccccc4c(-c4ccc5oc6ccccc6c5c4)c4ccccc34)cccc2c1]

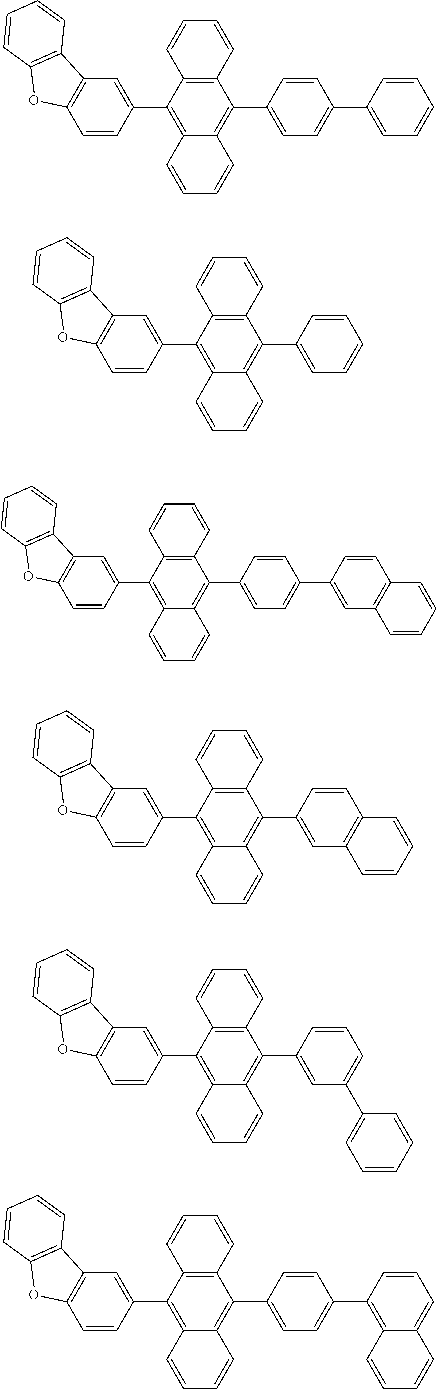 [SMILES: c1ccc(-c2ccc(-c3c4ccccc4c(-c4ccc5oc6ccccc6c5c4)c4ccccc34)cc2)cc1 ; c1ccc(-c2c3ccccc3c(-c3ccc4oc5ccccc5c4c3)c3ccccc23)cc1 ; c1ccc2cc(-c3ccc(-c4c5ccccc5c(-c5ccc6oc7ccccc7c6c5)c5ccccc45)cc3)ccc2c1 ; c1ccc2cc(-c3c4ccccc4c(-c4ccc5oc6ccccc6c5c4)c4ccccc34)ccc2c1 ; c1ccc(-c2cccc(-c3c4ccccc4c(-c4ccc5oc6ccccc6c5c4)c4ccccc34)c2)cc1 ; c1ccc2c(-c3ccc(-c4c5ccccc5c(-c5ccc6oc7ccccc7c6c5)c5ccccc45)cc3)cccc2c1]

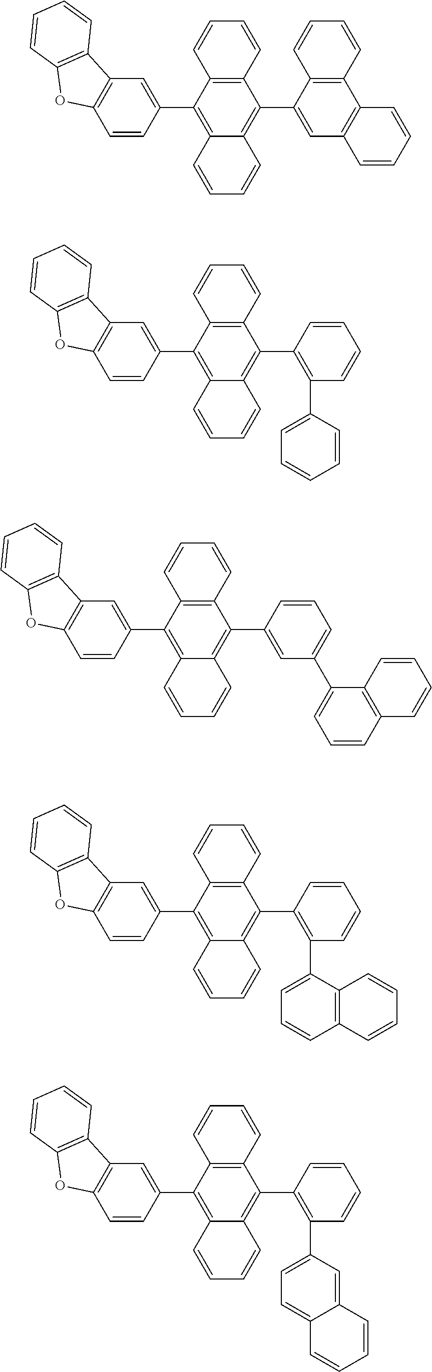 [SMILES: c1ccc2c(c1)cc(-c1c3ccccc3c(-c3ccc4oc5ccccc5c4c3)c3ccccc13)c1ccccc12 ; c1ccc(-c2ccccc2-c2c3ccccc3c(-c3ccc4oc5ccccc5c4c3)c3ccccc23)cc1 ; c1cc(-c2cccc3ccccc23)cc(-c2c3ccccc3c(-c3ccc4oc5ccccc5c4c3)c3ccccc23)c1 ; c1ccc(-c2c3ccccc3c(-c3ccc4oc5ccccc5c4c3)c3ccccc23)c(-c2cccc3ccccc23)c1 ; c1ccc(-c2c3ccccc3c(-c3ccc4oc5ccccc5c4c3)c3ccccc23)c(-c2ccc3ccccc3c2)c1]

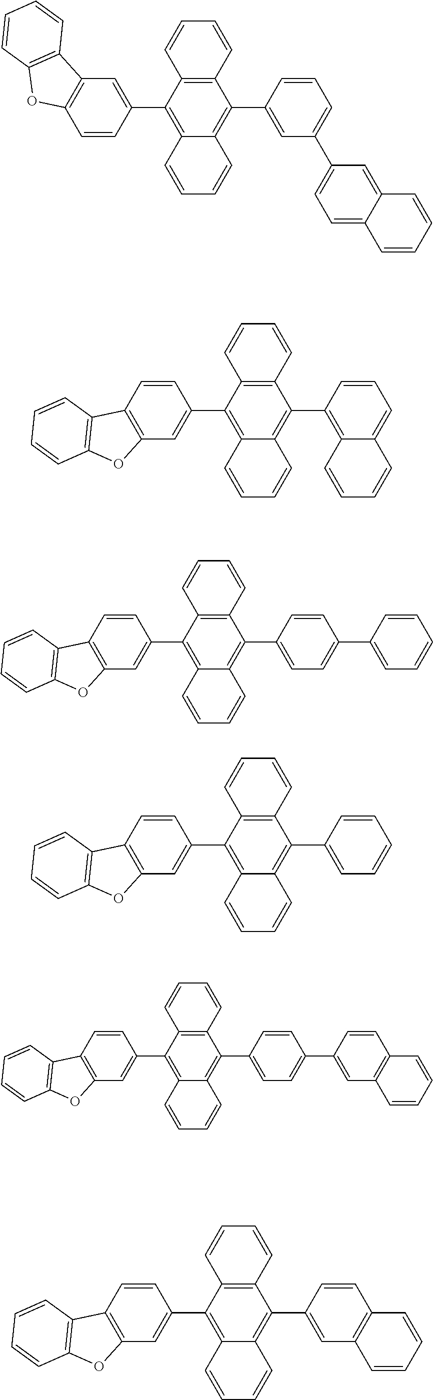

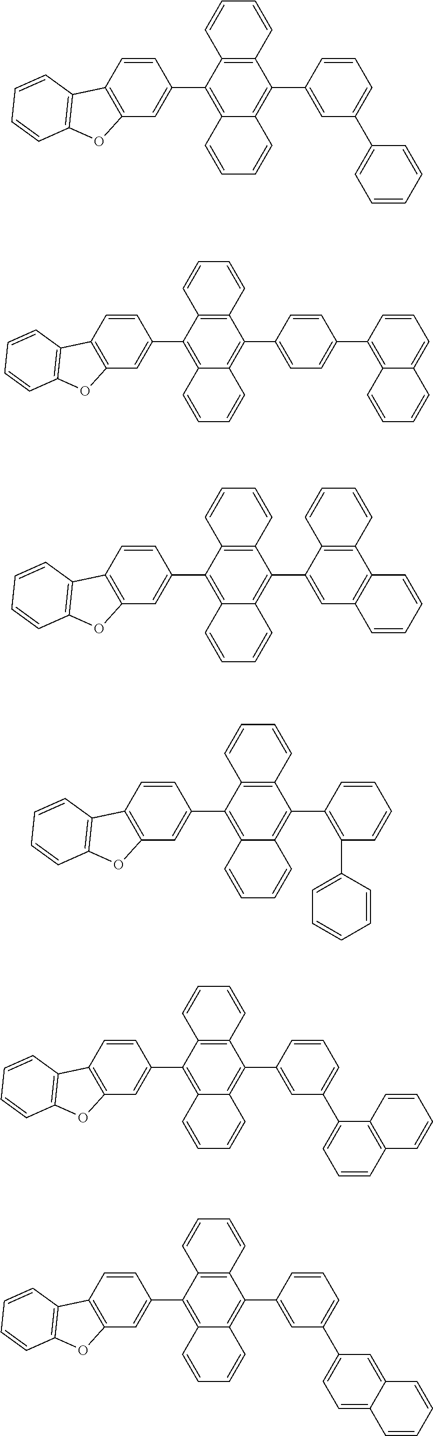 [SMILES: c1ccc(-c2cccc(-c3c4ccccc4c(-c4ccc5c(c4)oc4ccccc45)c4ccccc34)c2)cc1 ; c1ccc2c(-c3ccc(-c4c5ccccc5c(-c5ccc6c(c5)oc5ccccc56)c5ccccc45)cc3)cccc2c1 ; c1ccc2c(c1)cc(-c1c3ccccc3c(-c3ccc4c(c3)oc3ccccc34)c3ccccc13)c1ccccc12 ; c1ccc(-c2ccccc2-c2c3ccccc3c(-c3ccc4c(c3)oc3ccccc34)c3ccccc23)cc1 ; c1cc(-c2cccc3ccccc23)cc(-c2c3ccccc3c(-c3ccc4c(c3)oc3ccccc34)c3ccccc23)c1 ; c1cc(-c2ccc3ccccc3c2)cc(-c2c3ccccc3c(-c3ccc4c(c3)oc3ccccc34)c3ccccc23)c1]

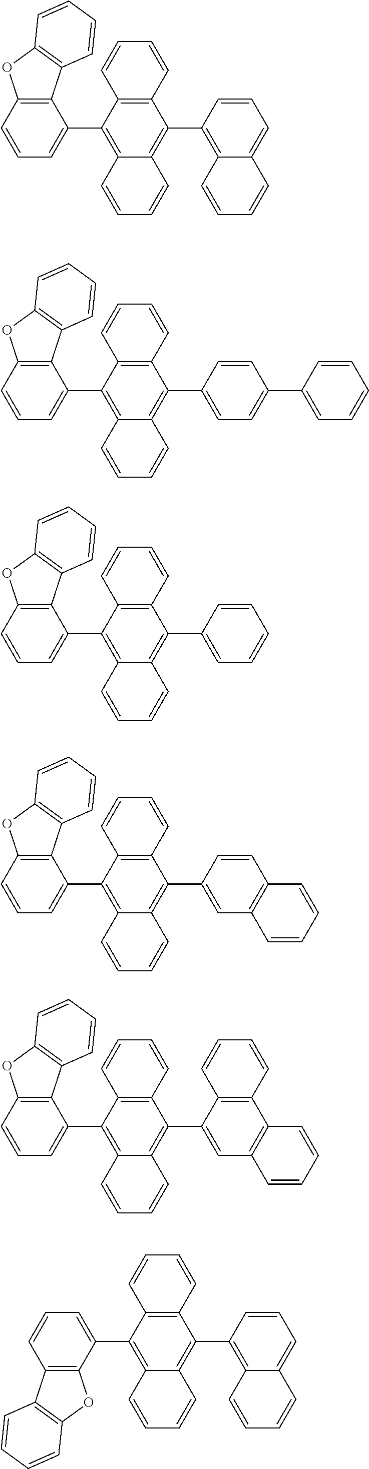

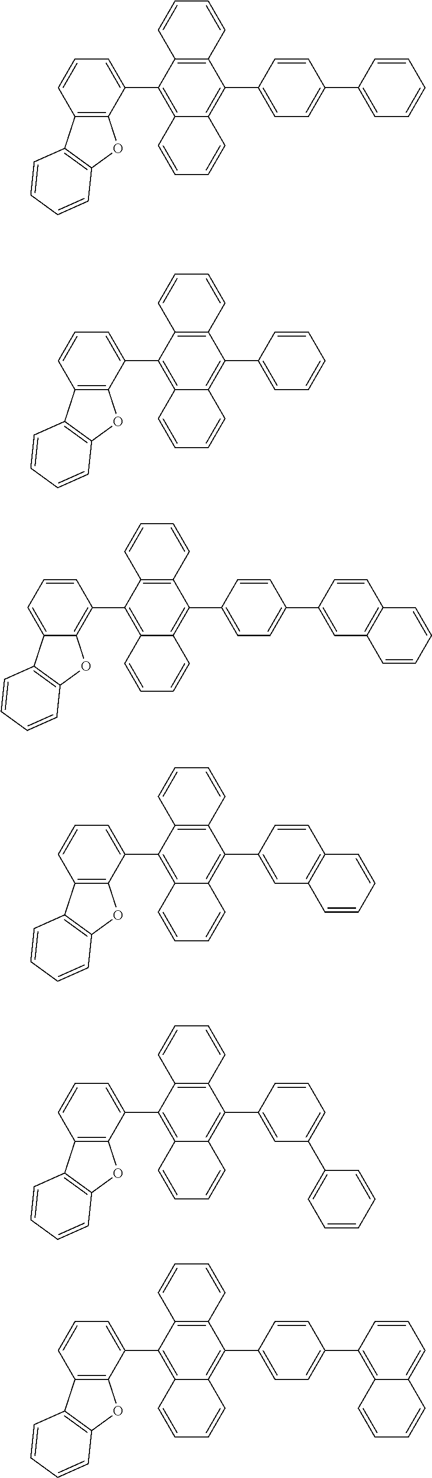 [SMILES: c1ccc(-c2ccc(-c3c4ccccc4c(-c4cccc5c4oc4ccccc45)c4ccccc34)cc2)cc1 ; c1ccc(-c2c3ccccc3c(-c3cccc4c3oc3ccccc34)c3ccccc23)cc1 ; c1ccc2cc(-c3ccc(-c4c5ccccc5c(-c5cccc6c5oc5ccccc56)c5ccccc45)cc3)ccc2c1 ; c1ccc2cc(-c3c4ccccc4c(-c4cccc5c4oc4ccccc45)c4ccccc34)ccc2c1 ; c1ccc(-c2cccc(-c3c4ccccc4c(-c4cccc5c4oc4ccccc45)c4ccccc34)c2)cc1 ; c1ccc2c(-c3ccc(-c4c5ccccc5c(-c5cccc6c5oc5ccccc56)c5ccccc45)cc3)cccc2c1]

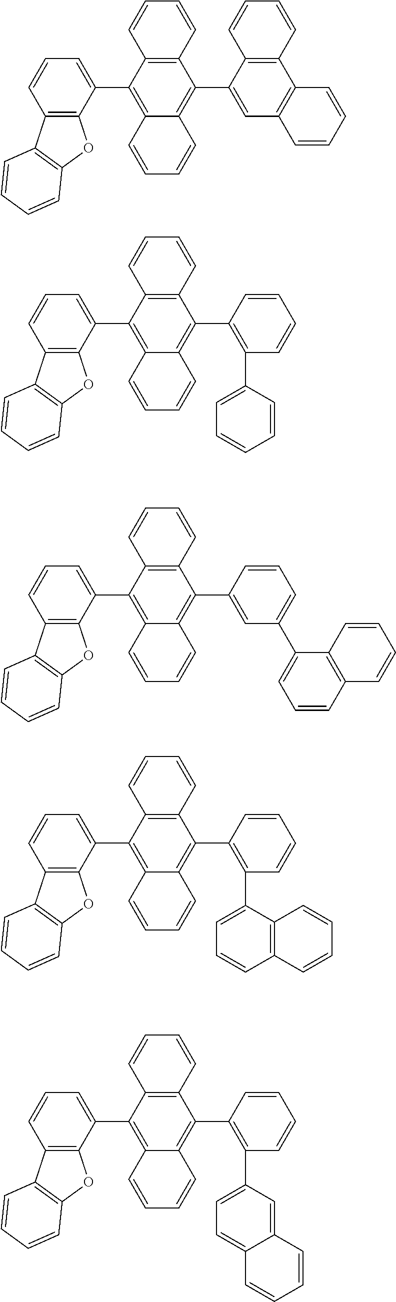 [SMILES: c1ccc2c(c1)cc(-c1c3ccccc3c(-c3cccc4c3oc3ccccc34)c3ccccc13)c1ccccc12 ; c1ccc(-c2ccccc2-c2c3ccccc3c(-c3cccc4c3oc3ccccc34)c3ccccc23)cc1 ; c1cc(-c2cccc3ccccc23)cc(-c2c3ccccc3c(-c3cccc4c3oc3ccccc34)c3ccccc23)c1 ; c1ccc(-c2c3ccccc3c(-c3cccc4c3oc3ccccc34)c3ccccc23)c(-c2cccc3ccccc23)c1 ; c1ccc(-c2c3ccccc3c(-c3cccc4c3oc3ccccc34)c3ccccc23)c(-c2ccc3ccccc3c2)c1]

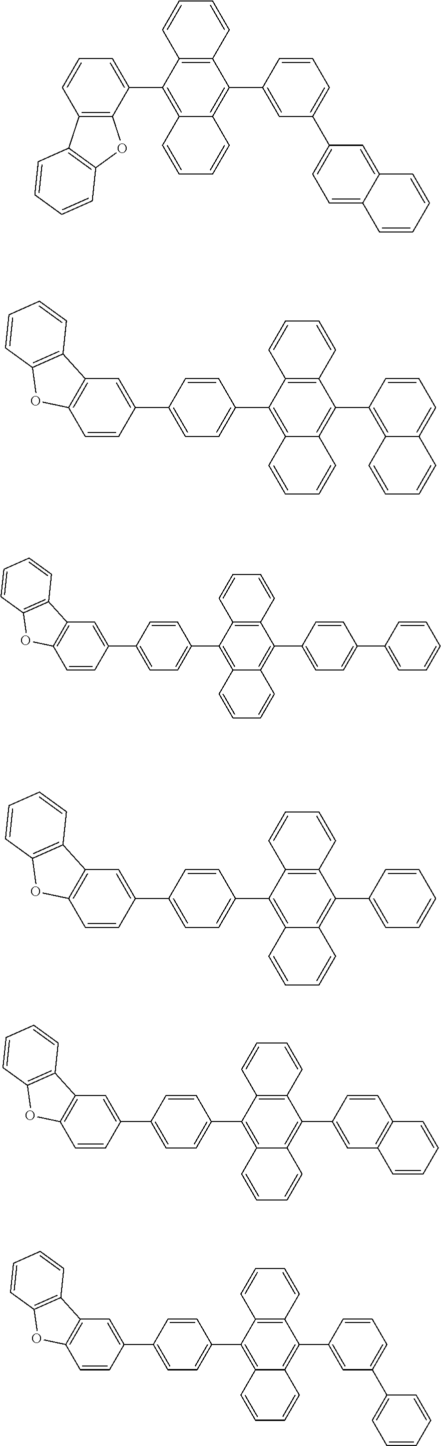 [SMILES: c1cc(-c2ccc3ccccc3c2)cc(-c2c3ccccc3c(-c3cccc4c3oc3ccccc34)c3ccccc23)c1 ; c1ccc2c(-c3c4ccccc4c(-c4ccc(-c5ccc6oc7ccccc7c6c5)cc4)c4ccccc34)cccc2c1 ; c1ccc(-c2ccc(-c3c4ccccc4c(-c4ccc(-c5ccc6oc7ccccc7c6c5)cc4)c4ccccc34)cc2)cc1 ; c1ccc(-c2c3ccccc3c(-c3ccc(-c4ccc5oc6ccccc6c5c4)cc3)c3ccccc23)cc1 ; c1ccc2cc(-c3c4ccccc4c(-c4ccc(-c5ccc6oc7ccccc7c6c5)cc4)c4ccccc34)ccc2c1 ; c1ccc(-c2cccc(-c3c4ccccc4c(-c4ccc(-c5ccc6oc7ccccc7c6c5)cc4)c4ccccc34)c2)cc1]

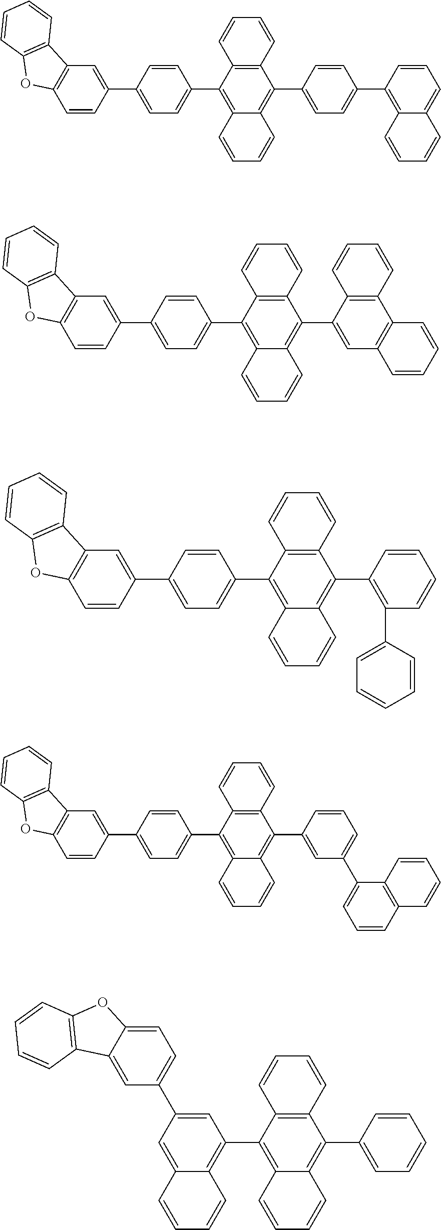 [SMILES: c1ccc2c(-c3ccc(-c4c5ccccc5c(-c5ccc(-c6ccc7oc8ccccc8c7c6)cc5)c5ccccc45)cc3)cccc2c1 ; c1ccc2c(c1)cc(-c1c3ccccc3c(-c3ccc(-c4ccc5oc6ccccc6c5c4)cc3)c3ccccc13)c1ccccc12 ; c1ccc(-c2ccccc2-c2c3ccccc3c(-c3ccc(-c4ccc5oc6ccccc6c5c4)cc3)c3ccccc23)cc1 ; c1cc(-c2cccc3ccccc23)cc(-c2c3ccccc3c(-c3ccc(-c4ccc5oc6ccccc6c5c4)cc3)c3ccccc23)c1 ; c1ccc(-c2c3ccccc3c(-c3cc(-c4ccc5oc6ccccc6c5c4)cc4ccccc34)c3ccccc23)cc1]

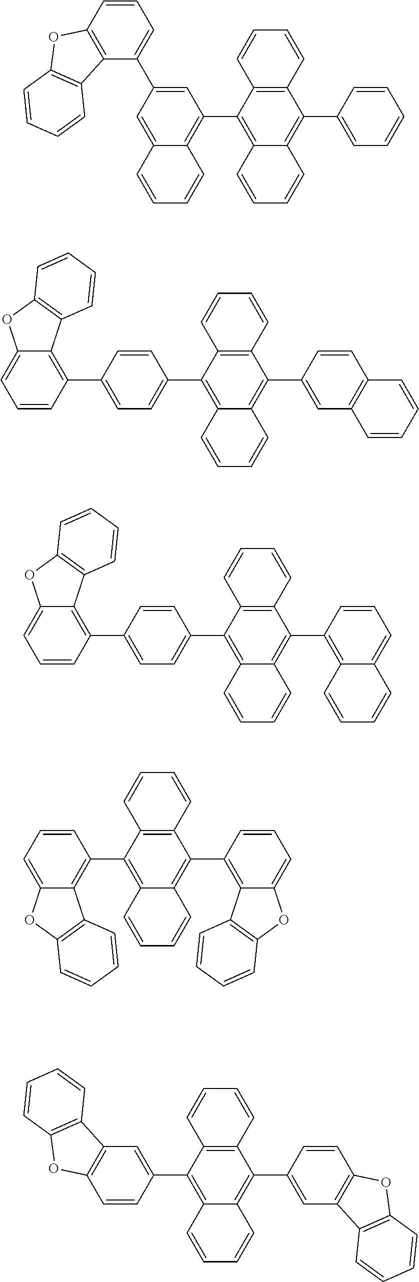 [SMILES: c1ccc(-c2c3ccccc3c(-c3cc(-c4cccc5oc6ccccc6c45)cc4ccccc34)c3ccccc23)cc1 ; c1ccc2cc(-c3c4ccccc4c(-c4ccc(-c5cccc6oc7ccccc7c56)cc4)c4ccccc34)ccc2c1 ; c1ccc2c(-c3c4ccccc4c(-c4ccc(-c5cccc6oc7ccccc7c56)cc4)c4ccccc34)cccc2c1 ; c1ccc2c(c1)oc1cccc(-c3c4ccccc4c(-c4cccc5oc6ccccc6c45)c4ccccc34)c12 ; c1ccc2c(c1)oc1ccc(-c3c4ccccc4c(-c4ccc5oc6ccccc6c5c4)c4ccccc34)cc12]

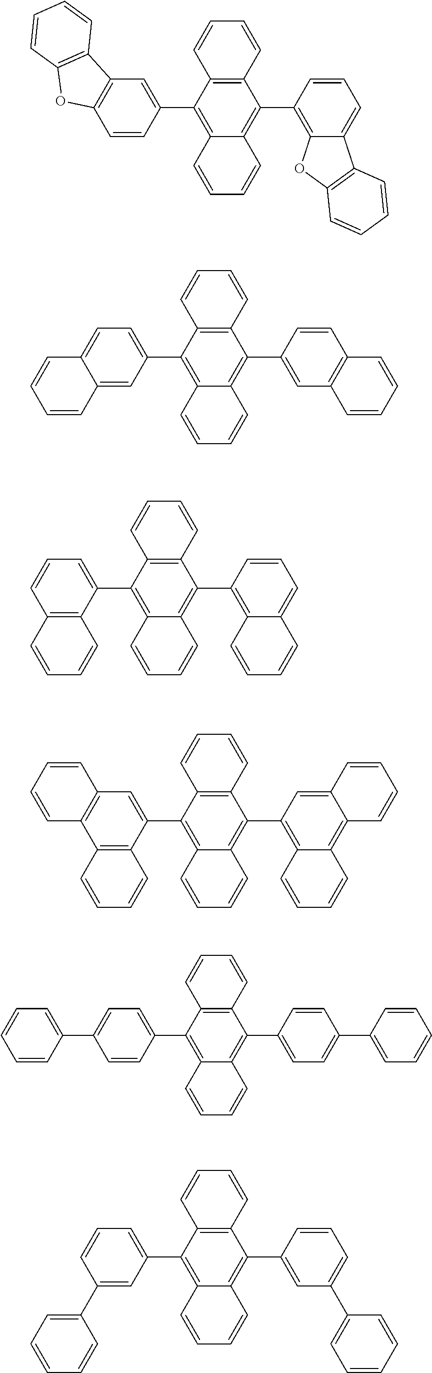

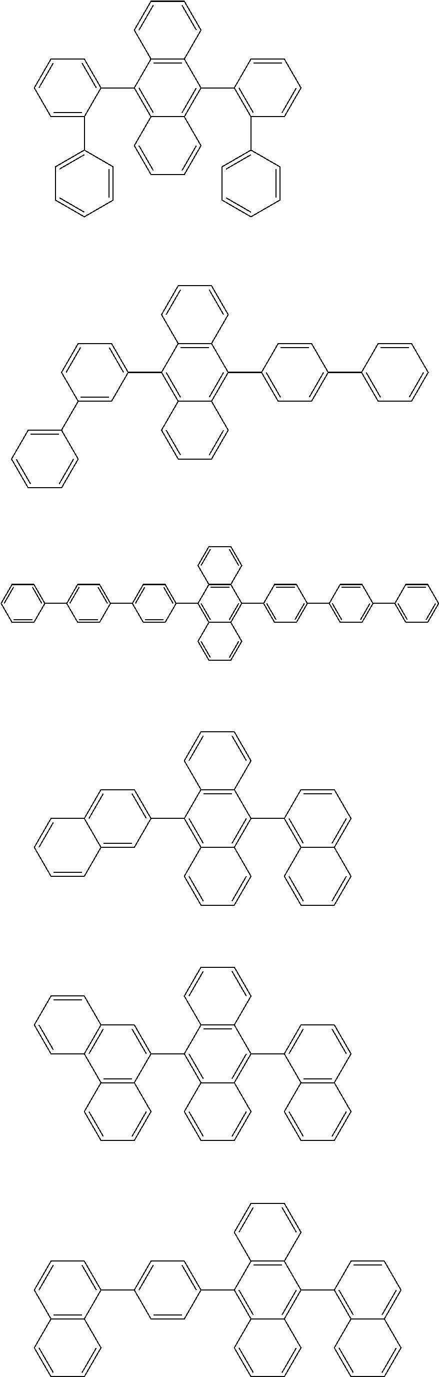

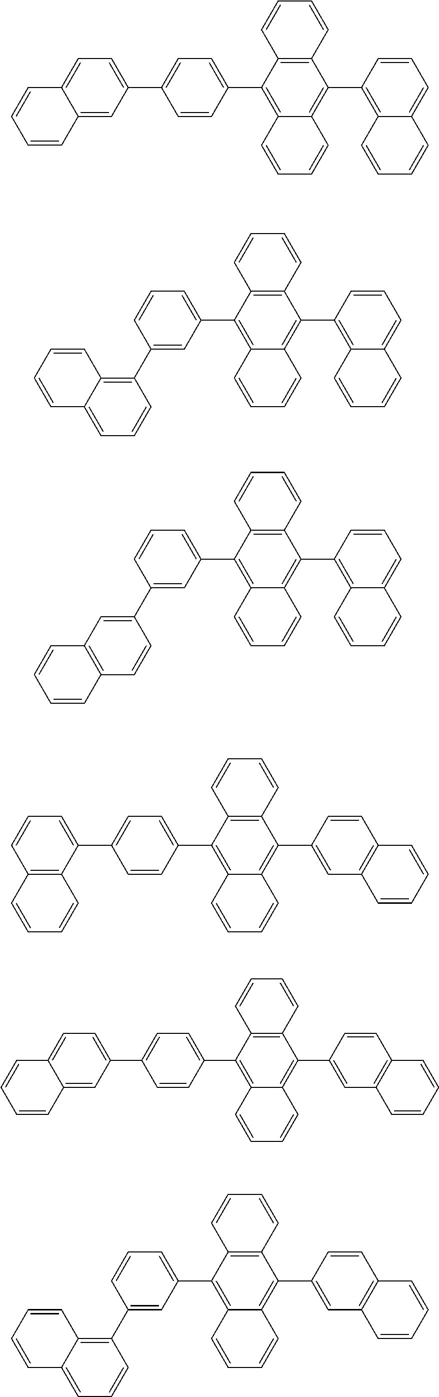

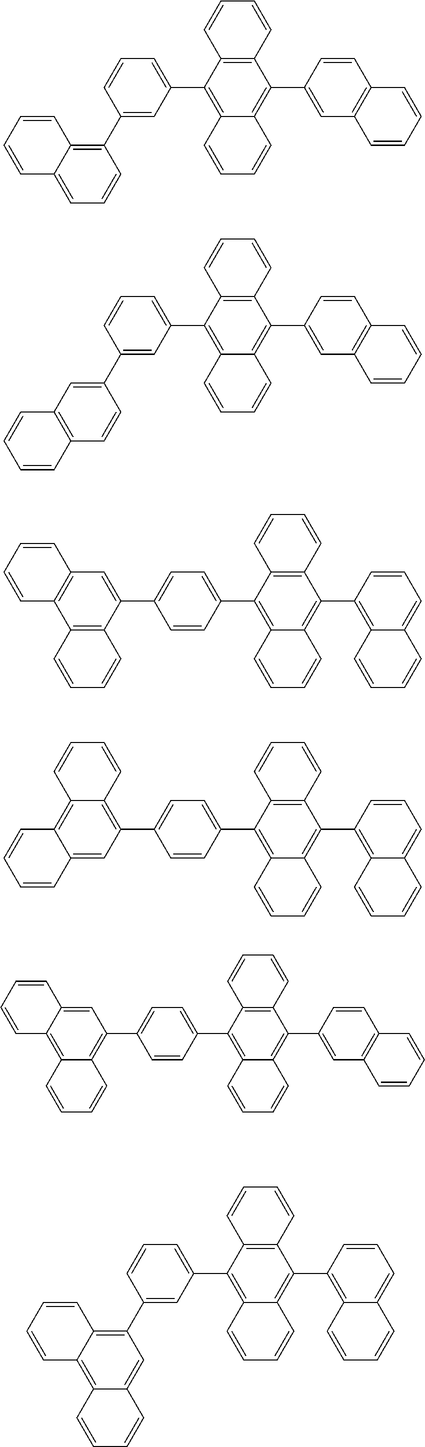

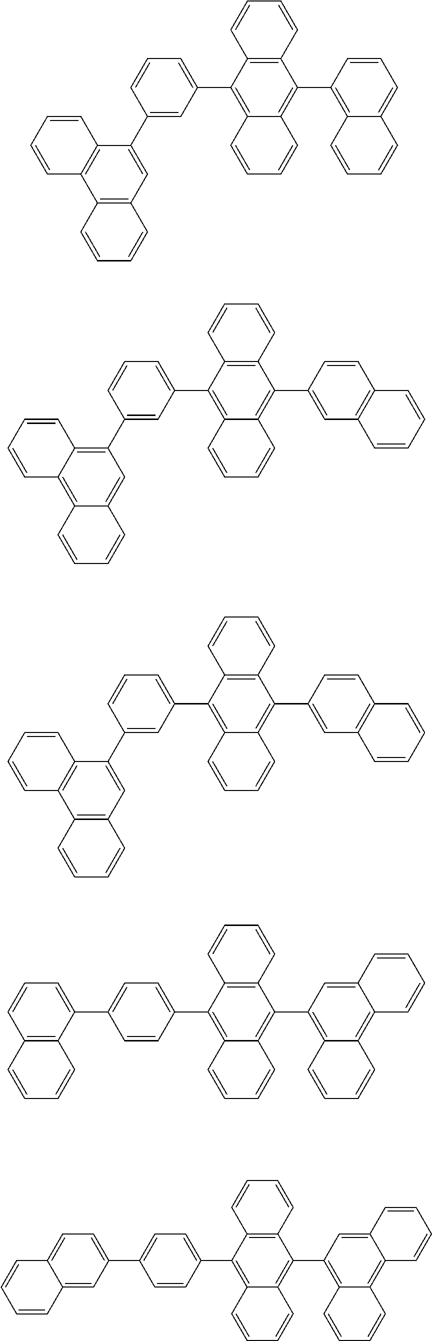

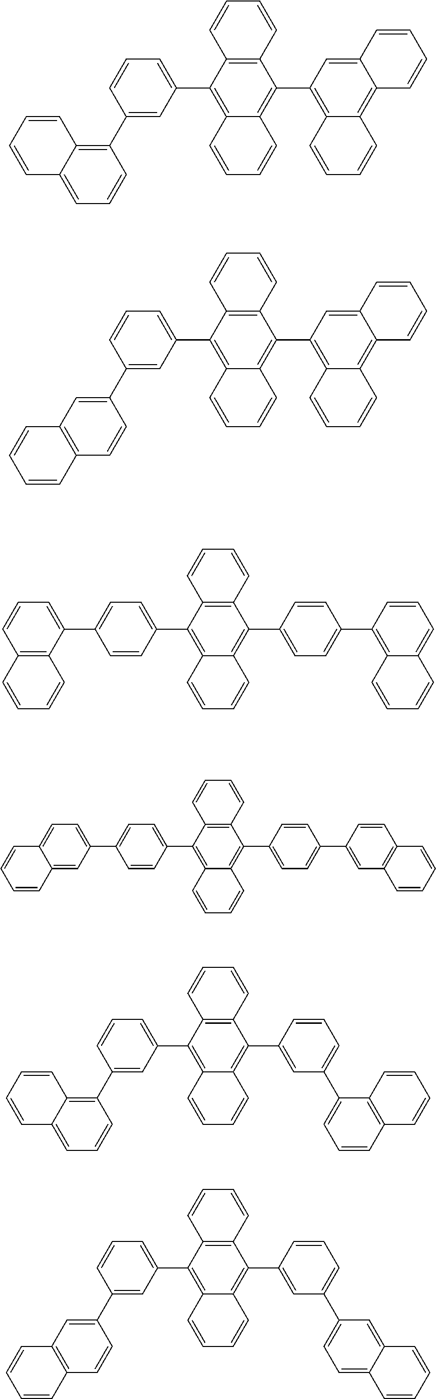

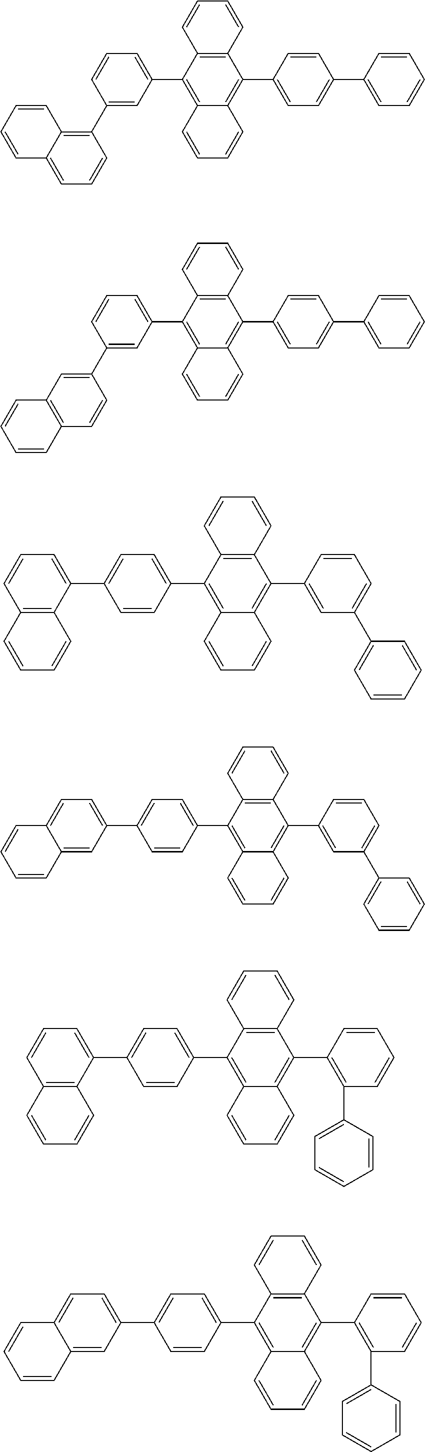

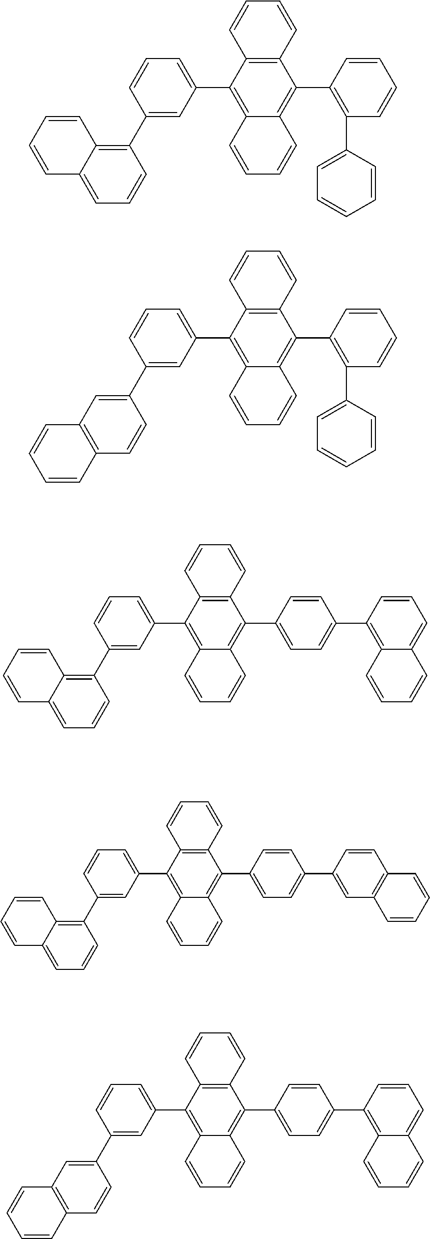

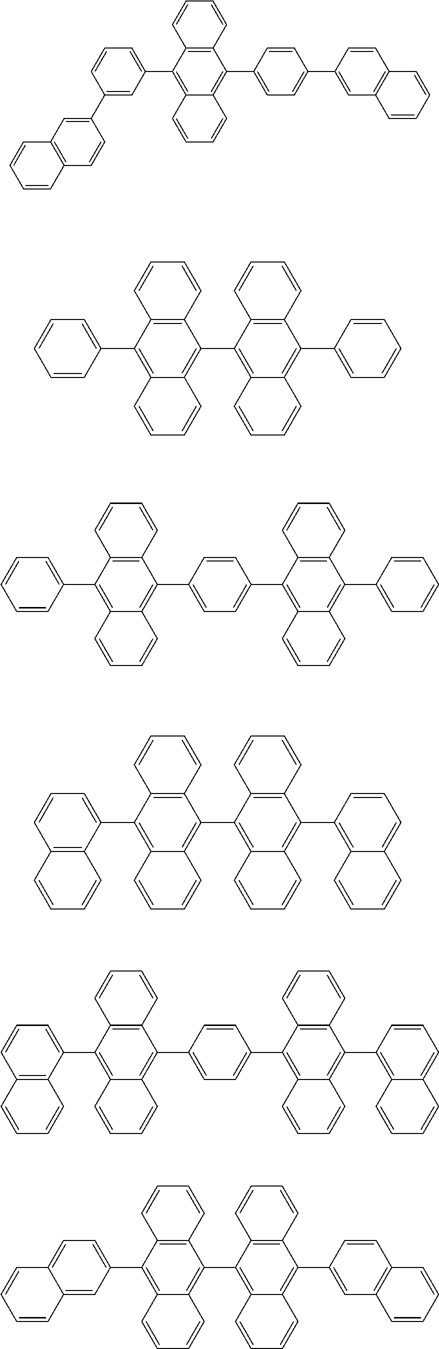

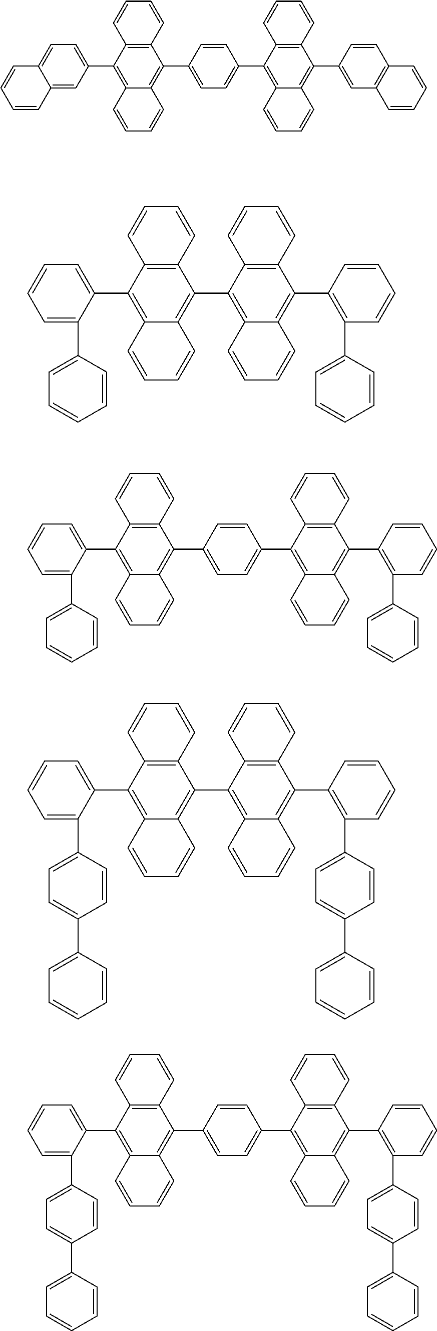

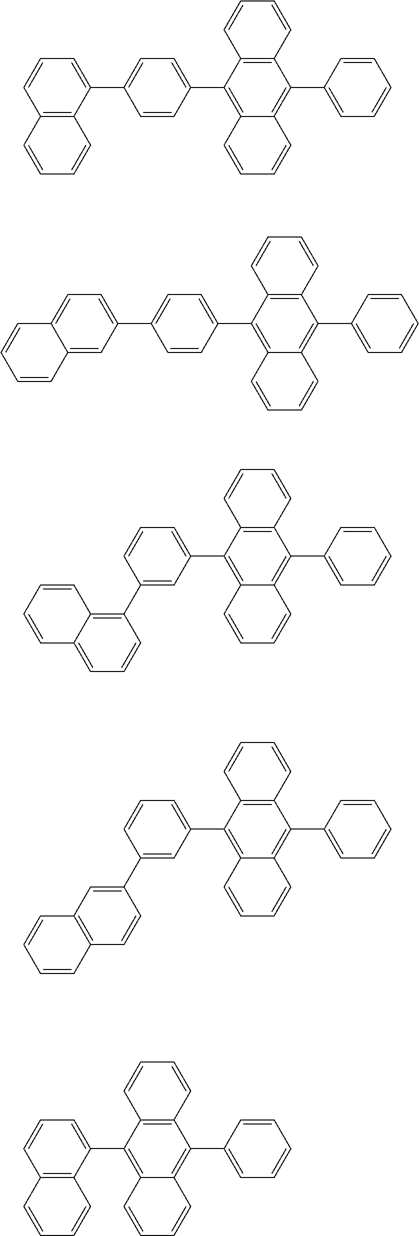

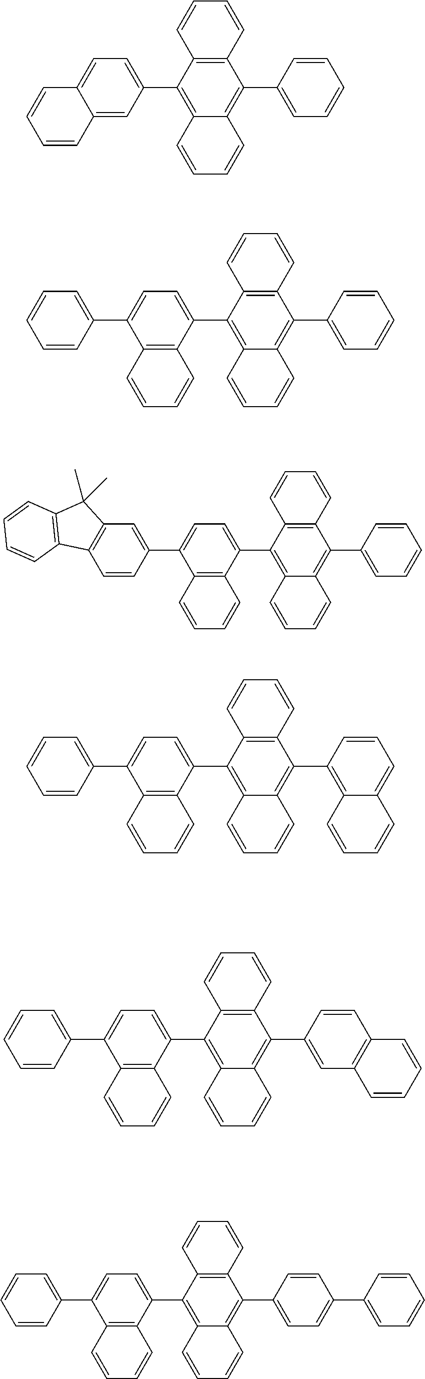

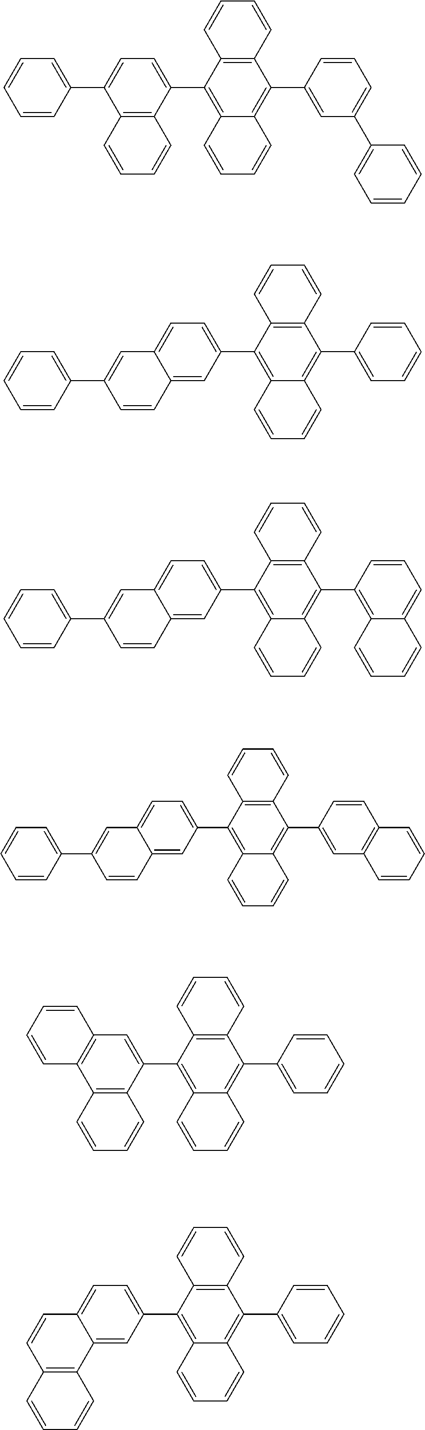

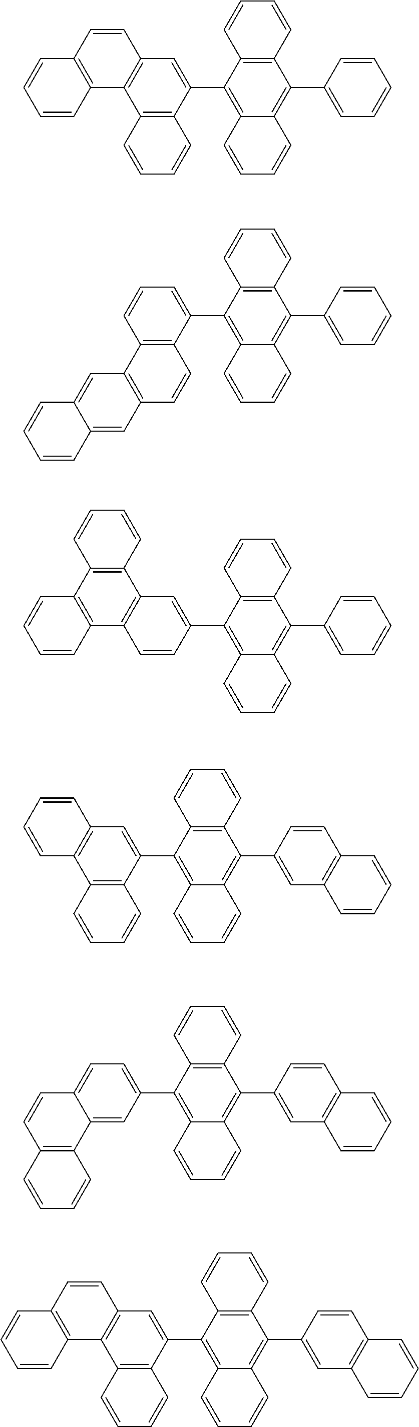

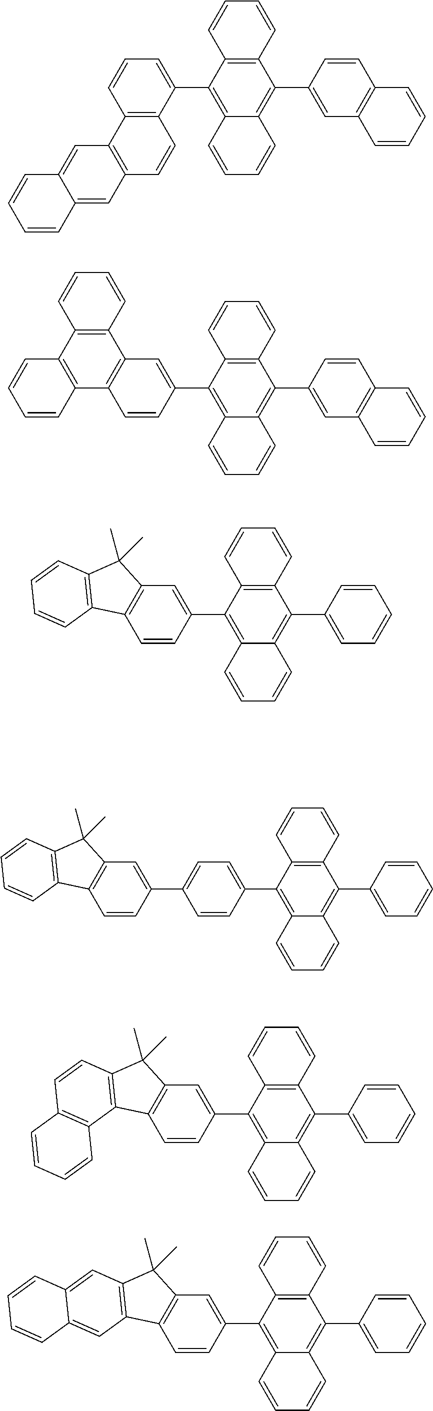

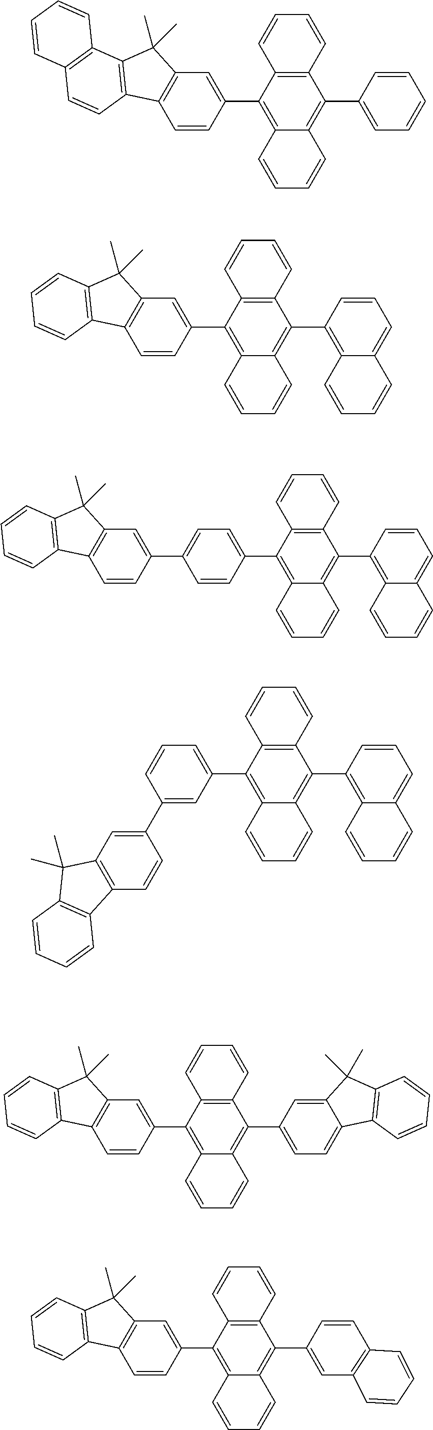

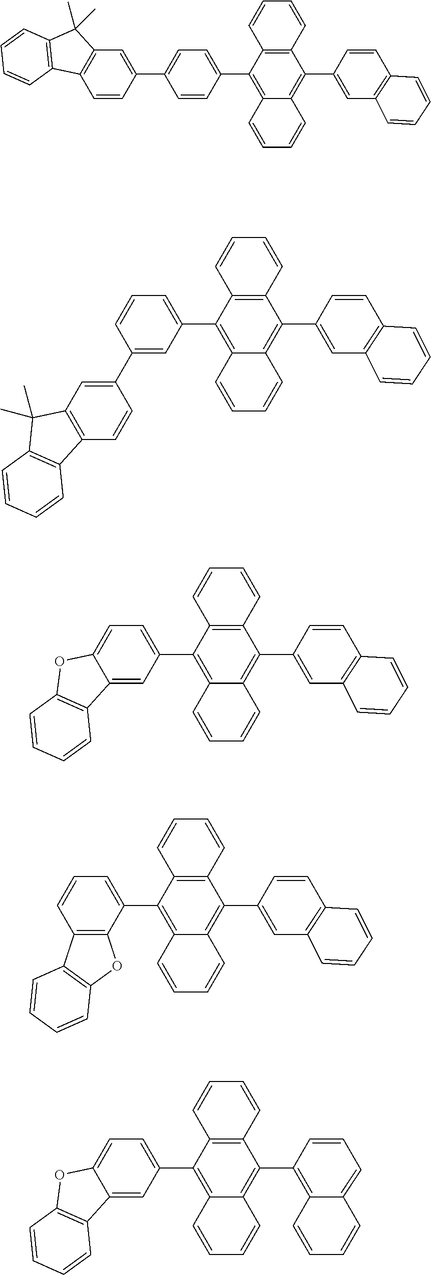

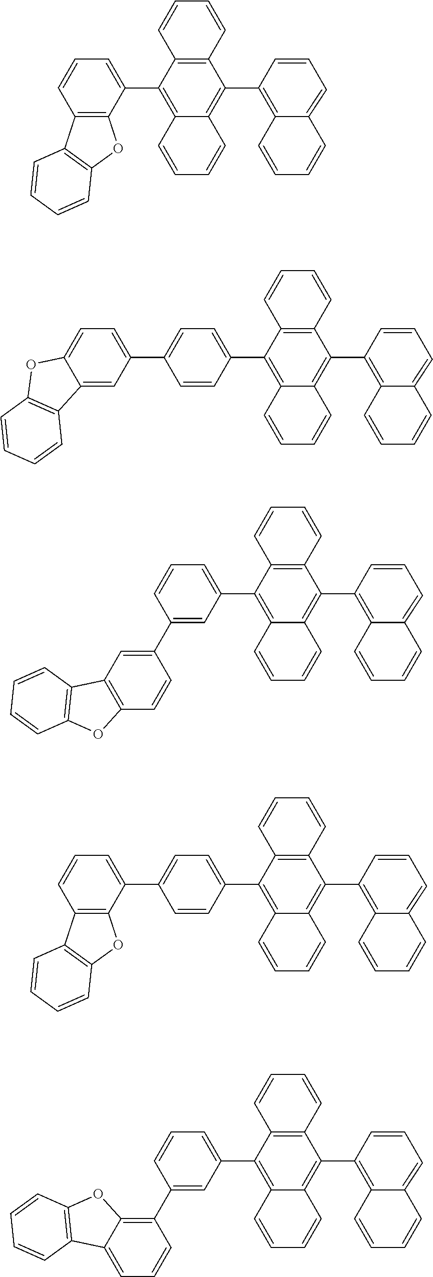 [SMILES: c1ccc2c(-c3c4ccccc4c(-c4cccc5c4oc4ccccc45)c4ccccc34)cccc2c1 ; c1ccc2c(-c3c4ccccc4c(-c4ccc(-c5ccc6oc7ccccc7c6c5)cc4)c4ccccc34)cccc2c1 ; c1cc(-c2ccc3oc4ccccc4c3c2)cc(-c2c3ccccc3c(-c3cccc4ccccc34)c3ccccc23)c1 ; c1ccc2c(-c3c4ccccc4c(-c4ccc(-c5cccc6c5oc5ccccc56)cc4)c4ccccc34)cccc2c1 ; c1cc(-c2c3ccccc3c(-c3cccc4ccccc34)c3ccccc23)cc(-c2cccc3c2oc2ccccc23)c1]

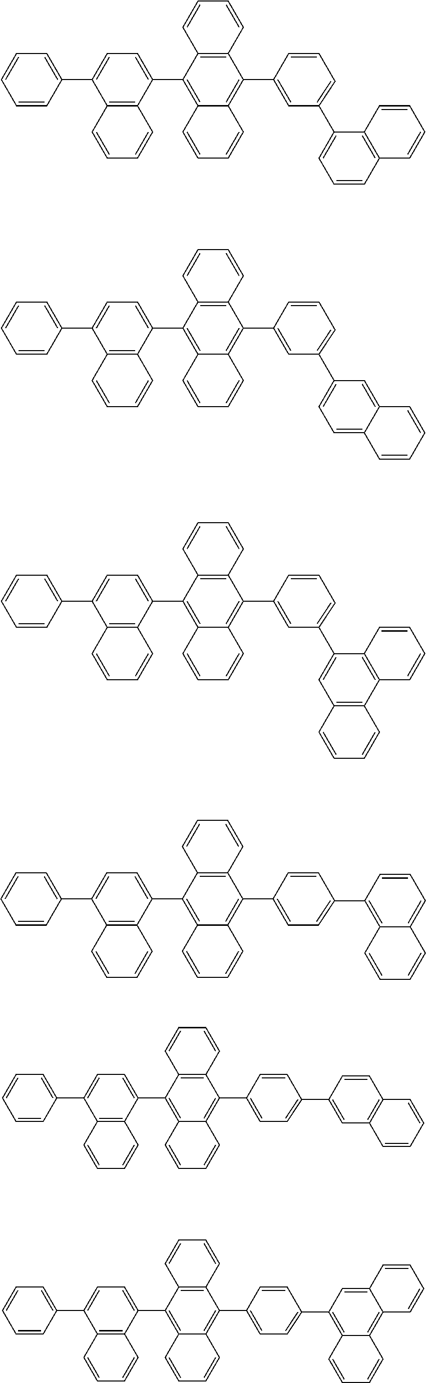

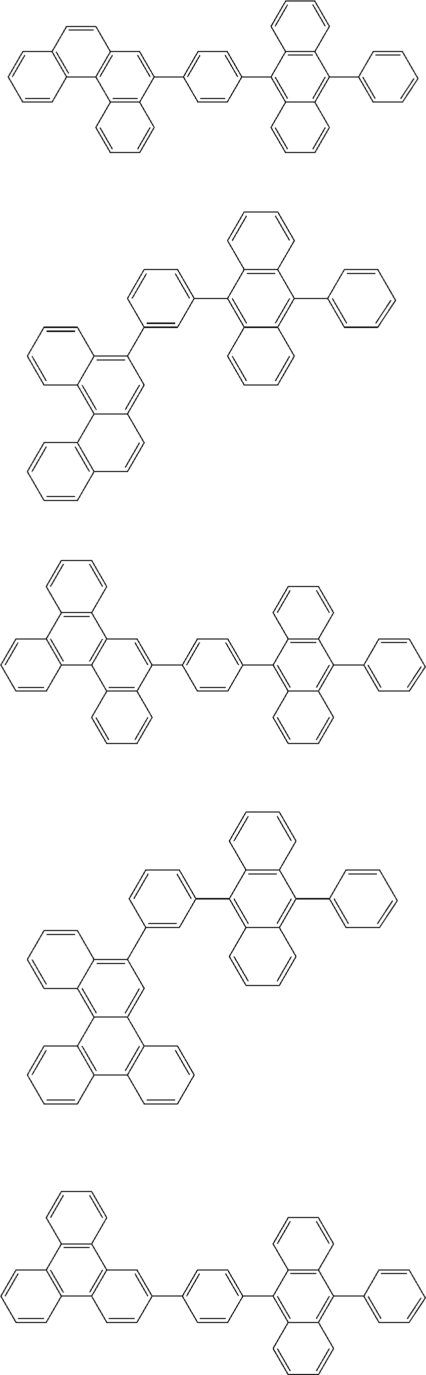

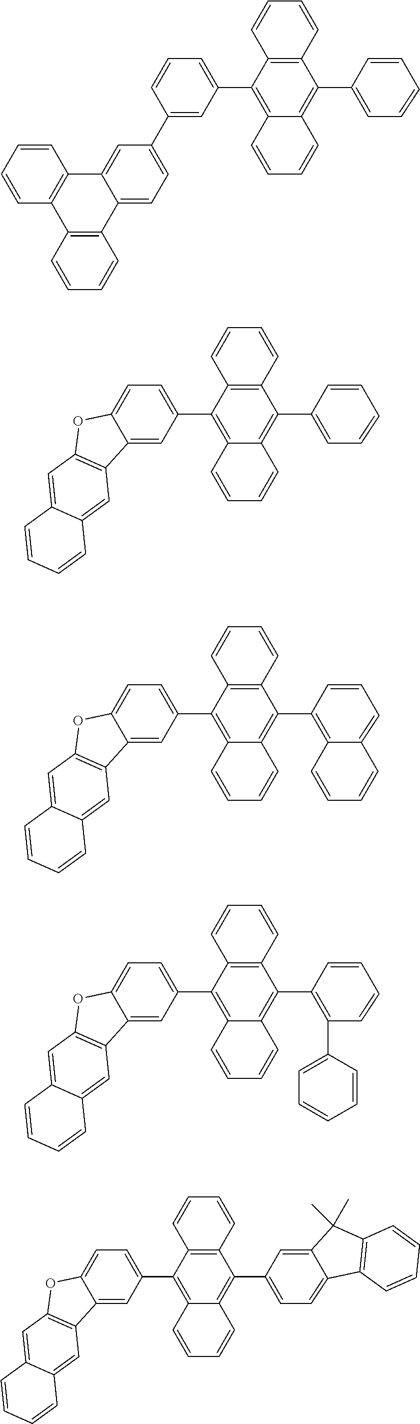

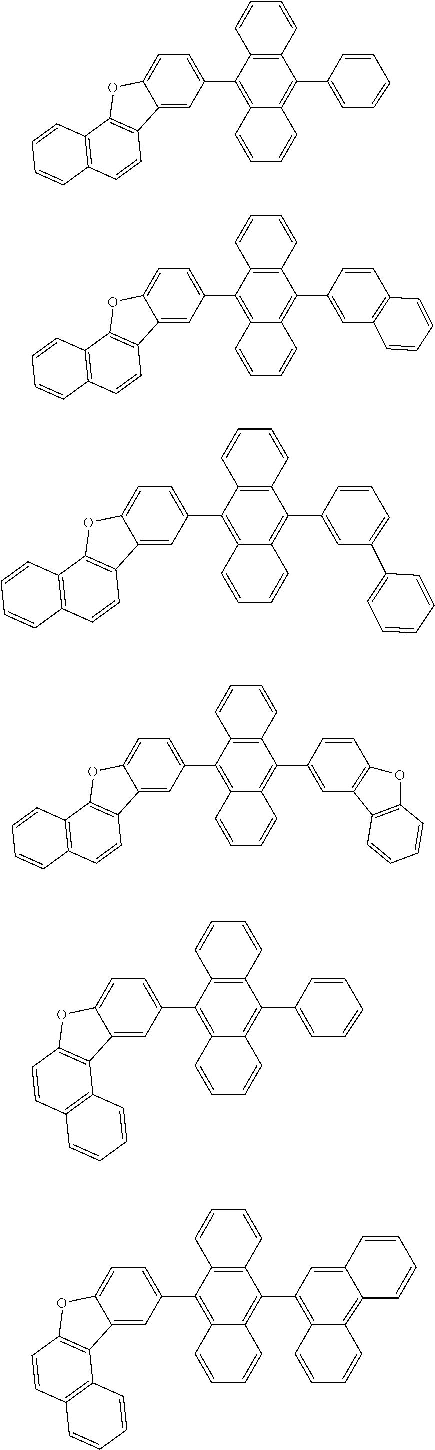 [SMILES: c1ccc(-c2c3ccccc3c(-c3ccc4oc5c6ccccc6ccc5c4c3)c3ccccc23)cc1 ; c1ccc2cc(-c3c4ccccc4c(-c4ccc5oc6c7ccccc7ccc6c5c4)c4ccccc34)ccc2c1 ; c1ccc(-c2cccc(-c3c4ccccc4c(-c4ccc5oc6c7ccccc7ccc6c5c4)c4ccccc34)c2)cc1 ; c1ccc2c(c1)ccc1c3cc(-c4c5ccccc5c(-c5ccc6oc7ccccc7c6c5)c5ccccc45)ccc3oc21 ; c1ccc(-c2c3ccccc3c(-c3ccc4oc5ccc6ccccc6c5c4c3)c3ccccc23)cc1 ; c1ccc2c(c1)cc(-c1c3ccccc3c(-c3ccc4oc5ccc6ccccc6c5c4c3)c3ccccc13)c1ccccc12]

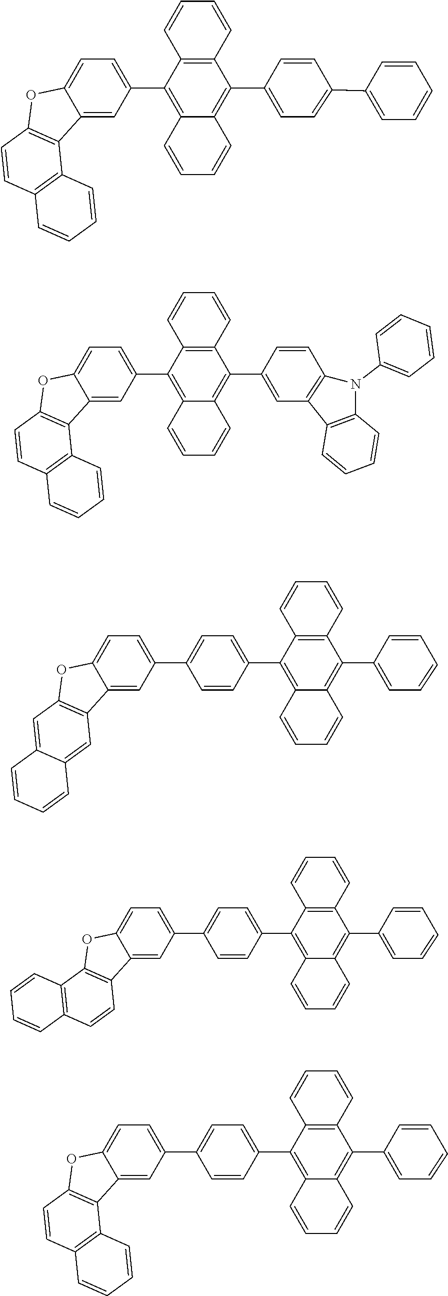 [SMILES: c1ccc(-c2ccc(-c3c4ccccc4c(-c4ccc5oc6ccc7ccccc7c6c5c4)c4ccccc34)cc2)cc1 ; c1ccc(-n2c3ccccc3c3cc(-c4c5ccccc5c(-c5ccc6oc7ccc8ccccc8c7c6c5)c5ccccc45)ccc32)cc1 ; c1ccc(-c2c3ccccc3c(-c3ccc(-c4ccc5oc6cc7ccccc7cc6c5c4)cc3)c3ccccc23)cc1 ; c1ccc(-c2c3ccccc3c(-c3ccc(-c4ccc5oc6c7ccccc7ccc6c5c4)cc3)c3ccccc23)cc1 ; c1ccc(-c2c3ccccc3c(-c3ccc(-c4ccc5oc6ccc7ccccc7c6c5c4)cc3)c3ccccc23)cc1]

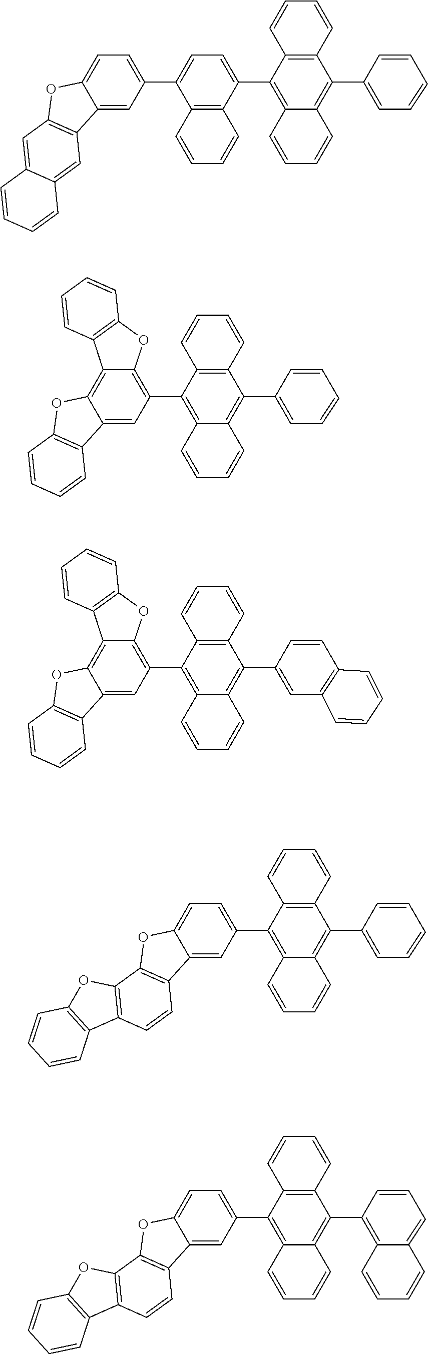 [SMILES: c1ccc(-c2c3ccccc3c(-c3ccc(-c4ccc5oc6cc7ccccc7cc6c5c4)c4ccccc34)c3ccccc23)cc1 ; c1ccc(-c2c3ccccc3c(-c3cc4c5ccccc5oc4c4c3oc3ccccc34)c3ccccc23)cc1 ; c1ccc2cc(-c3c4ccccc4c(-c4cc5c6ccccc6oc5c5c4oc4ccccc45)c4ccccc34)ccc2c1 ; c1ccc(-c2c3ccccc3c(-c3ccc4oc5c(ccc6c7ccccc7oc65)c4c3)c3ccccc23)cc1 ; c1ccc2c(-c3c4ccccc4c(-c4ccc5oc6c(ccc7c8ccccc8oc76)c5c4)c4ccccc34)cccc2c1]

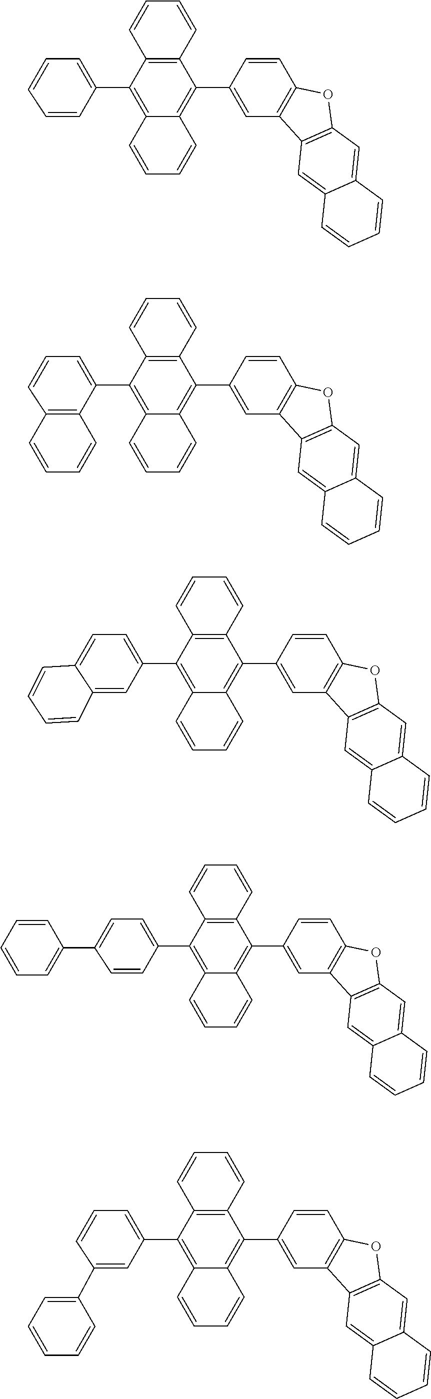 [SMILES: c1ccc(-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cccc5ccccc45)c4ccccc24)cc13 ; c1ccc2cc(-c3c4ccccc4c(-c4ccc5oc6cc7ccccc7cc6c5c4)c4ccccc34)ccc2c1 ; c1ccc(-c2ccc(-c3c4ccccc4c(-c4ccc5oc6cc7ccccc7cc6c5c4)c4ccccc34)cc2)cc1 ; c1ccc(-c2cccc(-c3c4ccccc4c(-c4ccc5oc6cc7ccccc7cc6c5c4)c4ccccc34)c2)cc1]

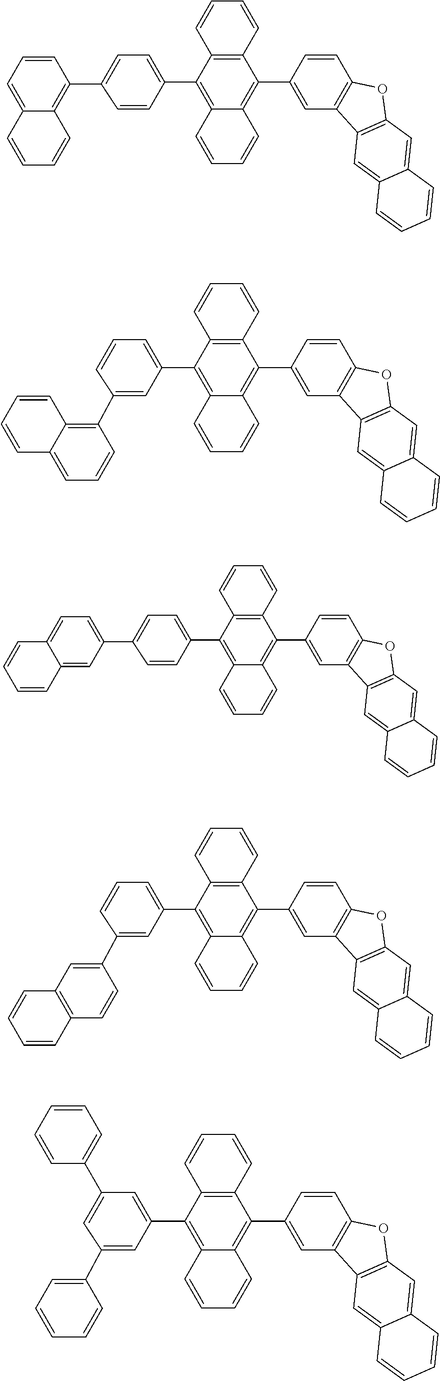 [SMILES: c1ccc2cc3c(cc2c1)oc1ccc(-c2c4ccccc4c(-c4ccc(-c5cccc6ccccc56)cc4)c4ccccc24)cc13 ; c1cc(-c2cccc3ccccc23)cc(-c2c3ccccc3c(-c3ccc4oc5cc6ccccc6cc5c4c3)c3ccccc23)c1 ; c1ccc2cc(-c3ccc(-c4c5ccccc5c(-c5ccc6oc7cc8ccccc8cc7c6c5)c5ccccc45)cc3)ccc2c1 ; c1cc(-c2ccc3ccccc3c2)cc(-c2c3ccccc3c(-c3ccc4oc5cc6ccccc6cc5c4c3)c3ccccc23)c1 ; c1ccc(-c2cc(-c3ccccc3)cc(-c3c4ccccc4c(-c4ccc5oc6cc7ccccc7cc6c5c4)c4ccccc34)c2)cc1]

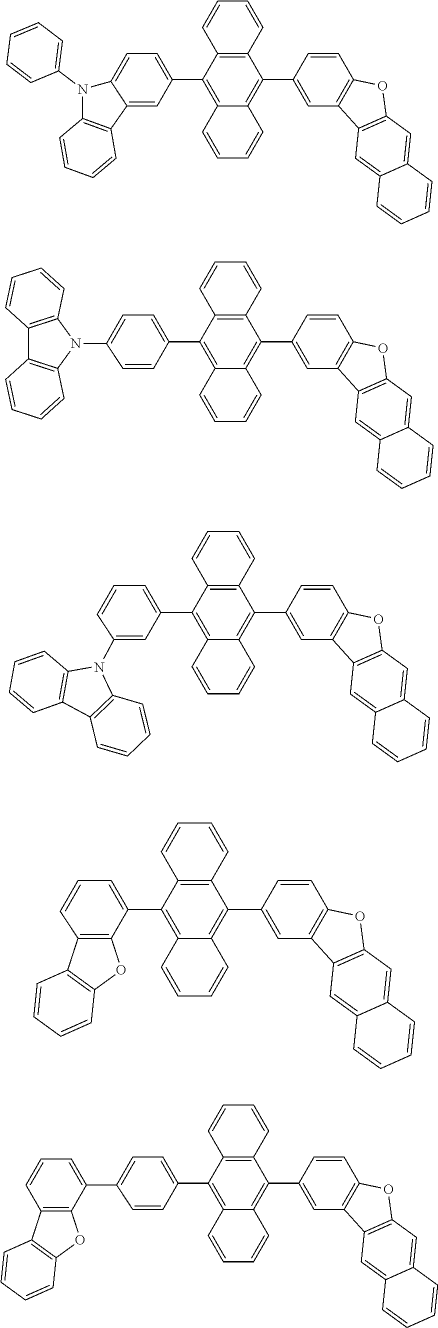 [SMILES: c1ccc(-n2c3ccccc3c3cc(-c4c5ccccc5c(-c5ccc6oc7cc8ccccc8cc7c6c5)c5ccccc45)ccc32)cc1 ; c1ccc2cc3c(cc2c1)oc1ccc(-c2c4ccccc4c(-c4ccc(-n5c6ccccc6c6ccccc65)cc4)c4ccccc24)cc13 ; c1cc(-c2c3ccccc3c(-c3ccc4oc5cc6ccccc6cc5c4c3)c3ccccc23)cc(-n2c3ccccc3c3ccccc32)c1 ; c1ccc2cc3c(cc2c1)oc1ccc(-c2c4ccccc4c(-c4cccc5c4oc4ccccc45)c4ccccc24)cc13 ; c1ccc2cc3c(cc2c1)oc1ccc(-c2c4ccccc4c(-c4ccc(-c5cccc6c5oc5ccccc56)cc4)c4ccccc24)cc13]

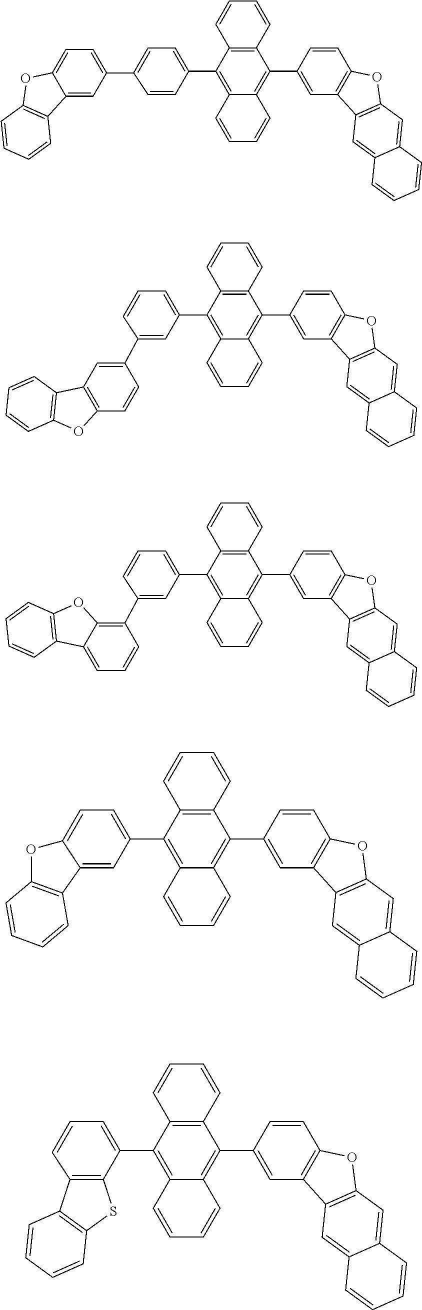 [SMILES: c1ccc2cc3c(cc2c1)oc1ccc(-c2c4ccccc4c(-c4ccc(-c5ccc6oc7ccccc7c6c5)cc4)c4ccccc24)cc13 ; c1cc(-c2ccc3oc4ccccc4c3c2)cc(-c2c3ccccc3c(-c3ccc4oc5cc6ccccc6cc5c4c3)c3ccccc23)c1 ; c1cc(-c2c3ccccc3c(-c3ccc4oc5cc6ccccc6cc5c4c3)c3ccccc23)cc(-c2cccc3c2oc2ccccc23)c1 ; c1ccc2cc3c(cc2c1)oc1ccc(-c2c4ccccc4c(-c4ccc5oc6ccccc6c5c4)c4ccccc24)cc13 ; c1ccc2cc3c(cc2c1)oc1ccc(-c2c4ccccc4c(-c4cccc5c4sc4ccccc45)c4ccccc24)cc13]

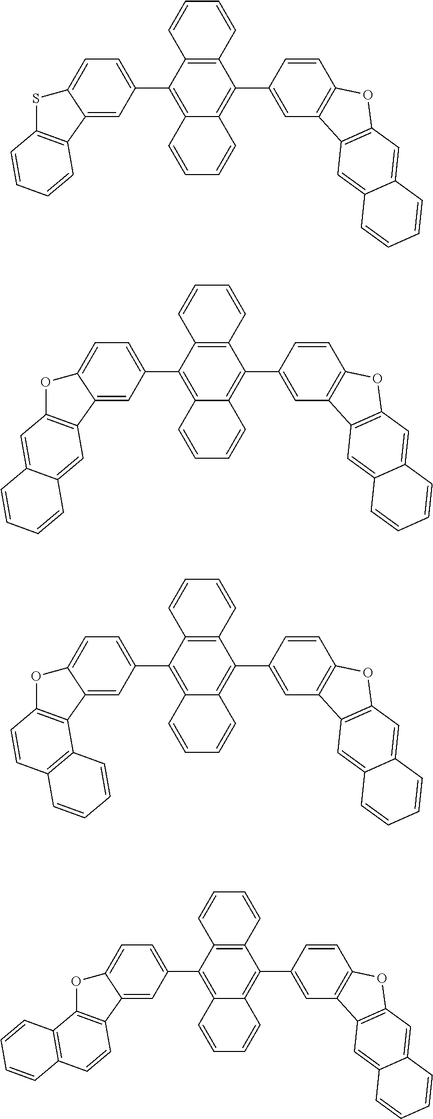 [SMILES: c1ccc2cc3c(cc2c1)oc1ccc(-c2c4ccccc4c(-c4ccc5sc6ccccc6c5c4)c4ccccc24)cc13 ; c1ccc2cc3c(cc2c1)oc1ccc(-c2c4ccccc4c(-c4ccc5oc6cc7ccccc7cc6c5c4)c4ccccc24)cc13 ; c1ccc2cc3c(cc2c1)oc1ccc(-c2c4ccccc4c(-c4ccc5oc6ccc7ccccc7c6c5c4)c4ccccc24)cc13 ; c1ccc2cc3c(cc2c1)oc1ccc(-c2c4ccccc4c(-c4ccc5oc6c7ccccc7ccc6c5c4)c4ccccc24)cc13]

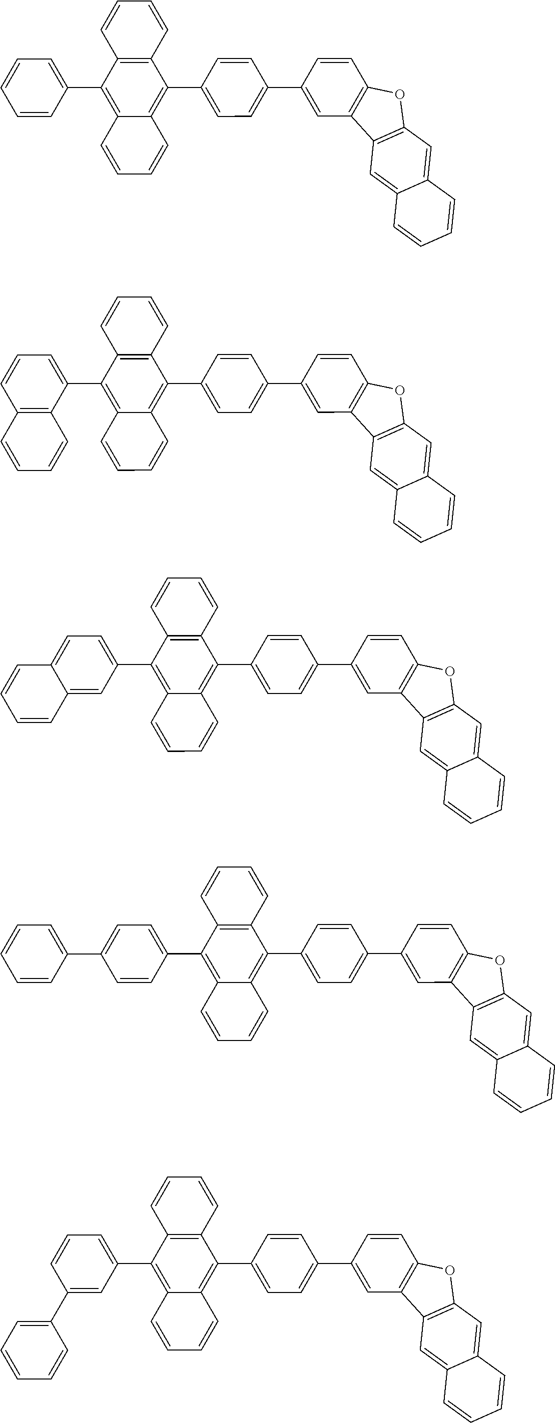 [SMILES: c1ccc(-c2c3ccccc3c(-c3ccc(-c4ccc5oc6cc7ccccc7cc6c5c4)cc3)c3ccccc23)cc1 ; c1ccc2cc3c(cc2c1)oc1ccc(-c2ccc(-c4c5ccccc5c(-c5cccc6ccccc56)c5ccccc45)cc2)cc13 ; c1ccc2cc(-c3c4ccccc4c(-c4ccc(-c5ccc6oc7cc8ccccc8cc7c6c5)cc4)c4ccccc34)ccc2c1 ; c1ccc(-c2ccc(-c3c4ccccc4c(-c4ccc(-c5ccc6oc7cc8ccccc8cc7c6c5)cc4)c4ccccc34)cc2)cc1 ; c1ccc(-c2cccc(-c3c4ccccc4c(-c4ccc(-c5ccc6oc7cc8ccccc8cc7c6c5)cc4)c4ccccc34)c2)cc1]

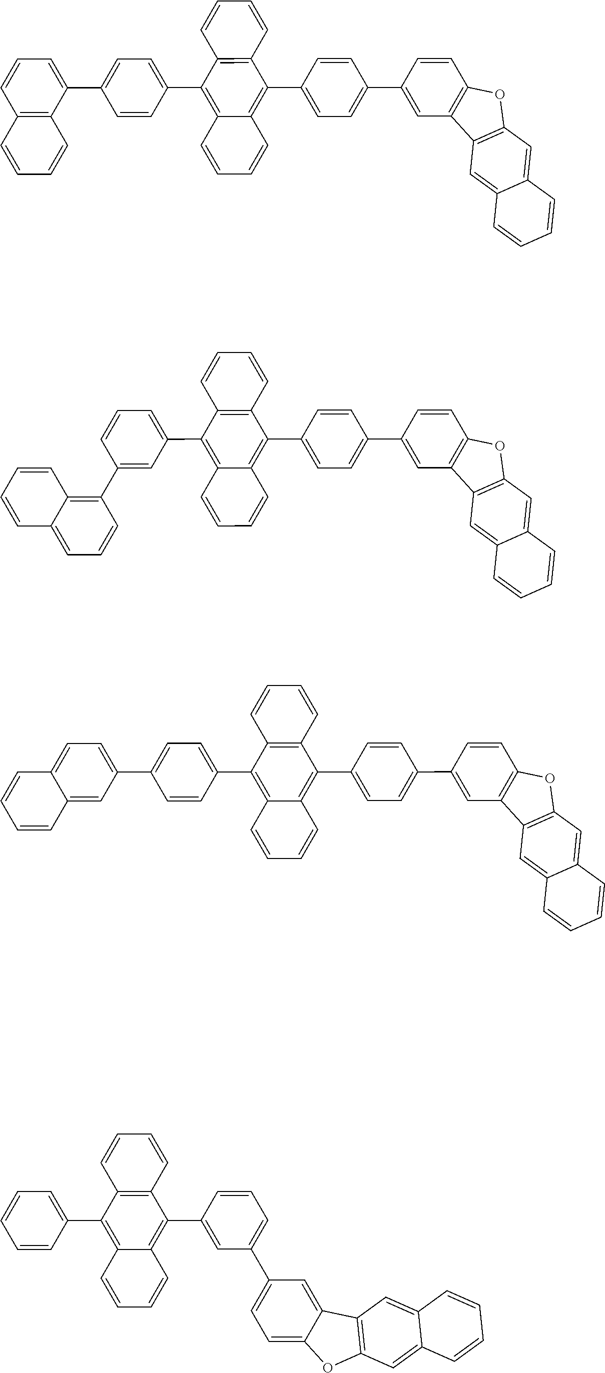 [SMILES: c1ccc2cc3c(cc2c1)oc1ccc(-c2ccc(-c4c5ccccc5c(-c5ccc(-c6cccc7ccccc67)cc5)c5ccccc45)cc2)cc13 ; c1cc(-c2cccc3ccccc23)cc(-c2c3ccccc3c(-c3ccc(-c4ccc5oc6cc7ccccc7cc6c5c4)cc3)c3ccccc23)c1 ; c1ccc2cc(-c3ccc(-c4c5ccccc5c(-c5ccc(-c6ccc7oc8cc9ccccc9cc8c7c6)cc5)c5ccccc45)cc3)ccc2c1 ; c1ccc(-c2c3ccccc3c(-c3cccc(-c4ccc5oc6cc7ccccc7cc6c5c4)c3)c3ccccc23)cc1]

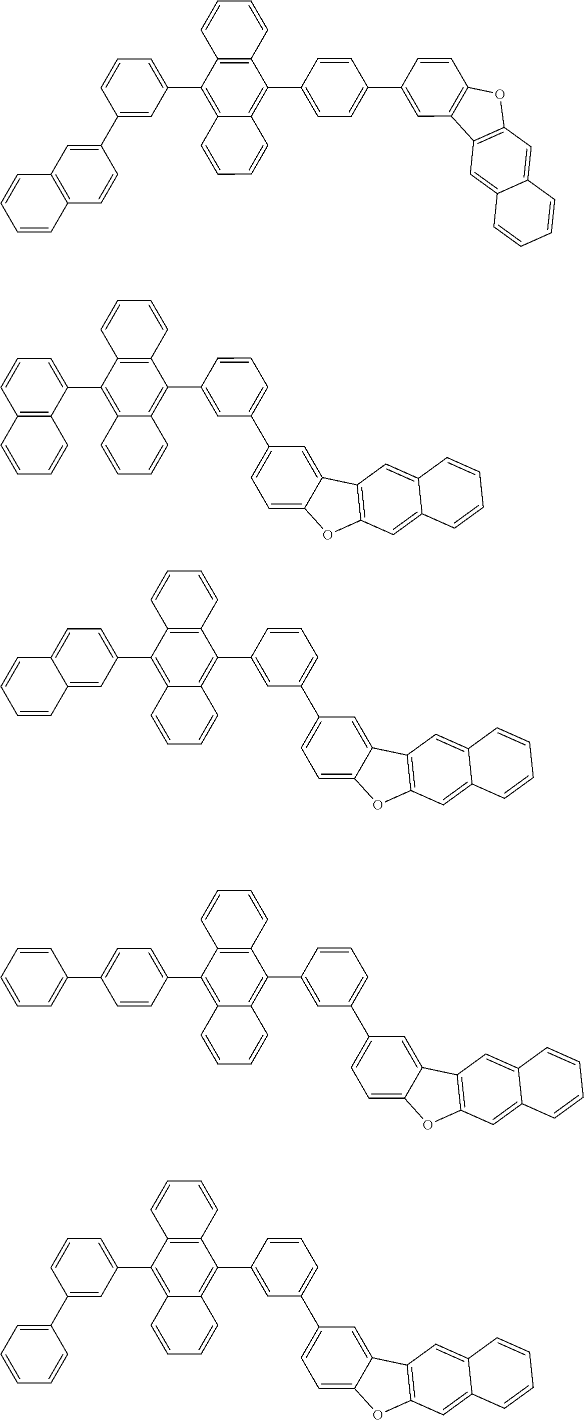 [SMILES: c1cc(-c2ccc3ccccc3c2)cc(-c2c3ccccc3c(-c3ccc(-c4ccc5oc6cc7ccccc7cc6c5c4)cc3)c3ccccc23)c1 ; c1cc(-c2ccc3oc4cc5ccccc5cc4c3c2)cc(-c2c3ccccc3c(-c3cccc4ccccc34)c3ccccc23)c1 ; c1cc(-c2ccc3oc4cc5ccccc5cc4c3c2)cc(-c2c3ccccc3c(-c3ccc4ccccc4c3)c3ccccc23)c1 ; c1ccc(-c2ccc(-c3c4ccccc4c(-c4cccc(-c5ccc6oc7cc8ccccc8cc7c6c5)c4)c4ccccc34)cc2)cc1 ; c1ccc(-c2cccc(-c3c4ccccc4c(-c4cccc(-c5ccc6oc7cc8ccccc8cc7c6c5)c4)c4ccccc34)c2)cc1]

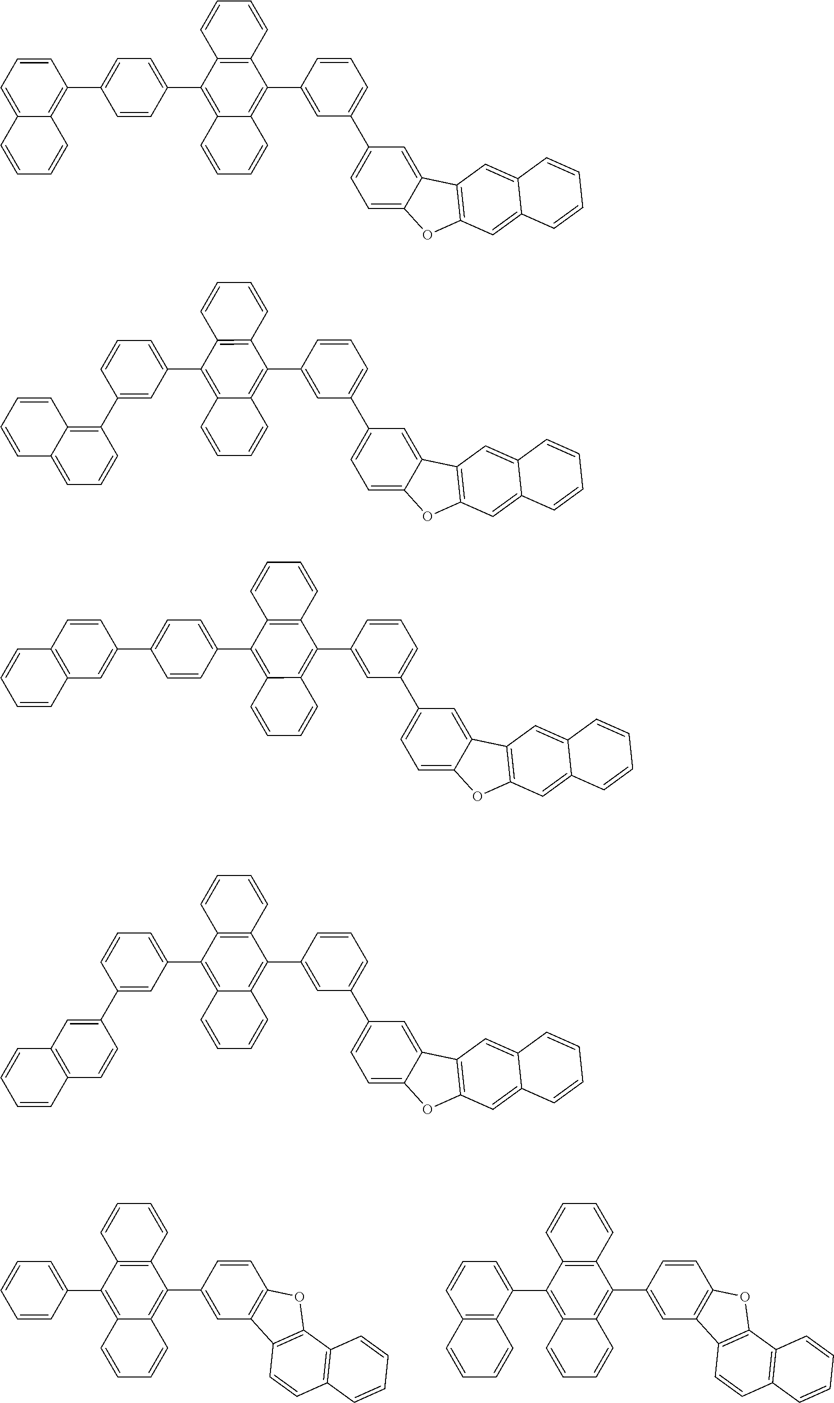 [SMILES: c1cc(-c2ccc3oc4cc5ccccc5cc4c3c2)cc(-c2c3ccccc3c(-c3ccc(-c4cccc5ccccc45)cc3)c3ccccc23)c1 ; c1cc(-c2ccc3oc4cc5ccccc5cc4c3c2)cc(-c2c3ccccc3c(-c3cccc(-c4cccc5ccccc45)c3)c3ccccc23)c1 ; c1cc(-c2ccc3oc4cc5ccccc5cc4c3c2)cc(-c2c3ccccc3c(-c3ccc(-c4ccc5ccccc5c4)cc3)c3ccccc23)c1 ; c1cc(-c2ccc3ccccc3c2)cc(-c2c3ccccc3c(-c3cccc(-c4ccc5oc6cc7ccccc7cc6c5c4)c3)c3ccccc23)c1 ; c1ccc(-c2c3ccccc3c(-c3ccc4oc5c6ccccc6ccc5c4c3)c3ccccc23)cc1 ; c1ccc2c(-c3c4ccccc4c(-c4ccc5oc6c7ccccc7ccc6c5c4)c4ccccc34)cccc2c1]

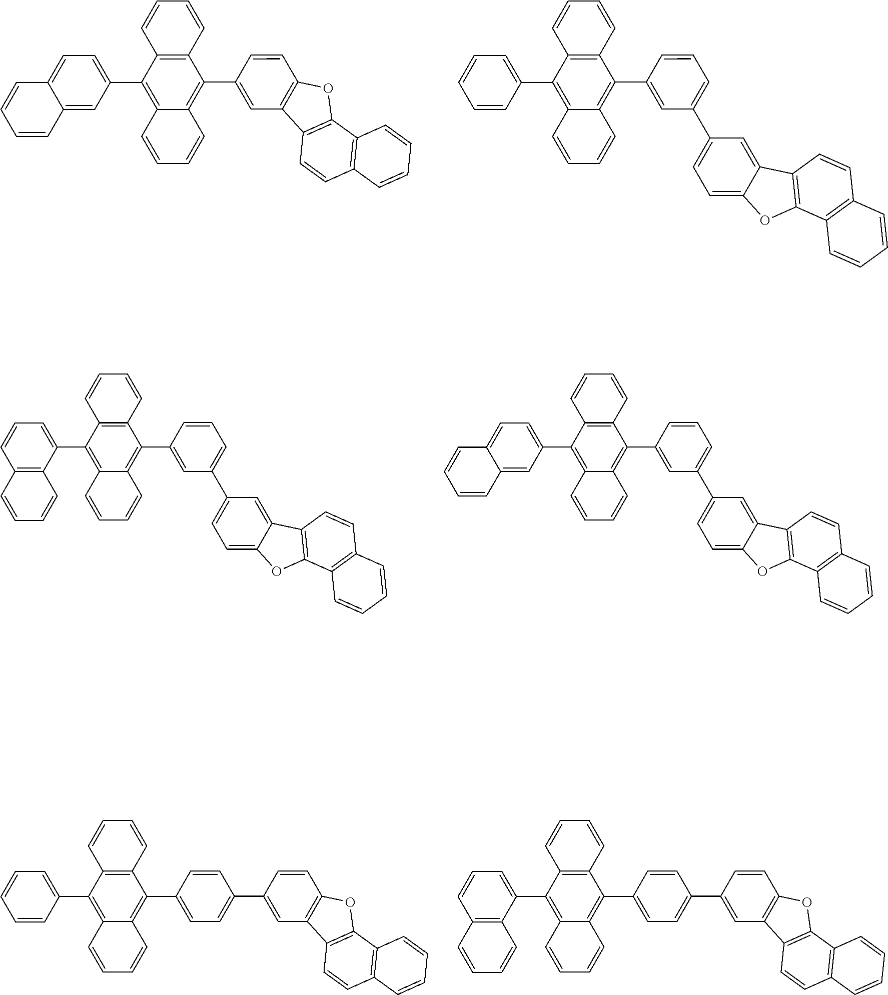

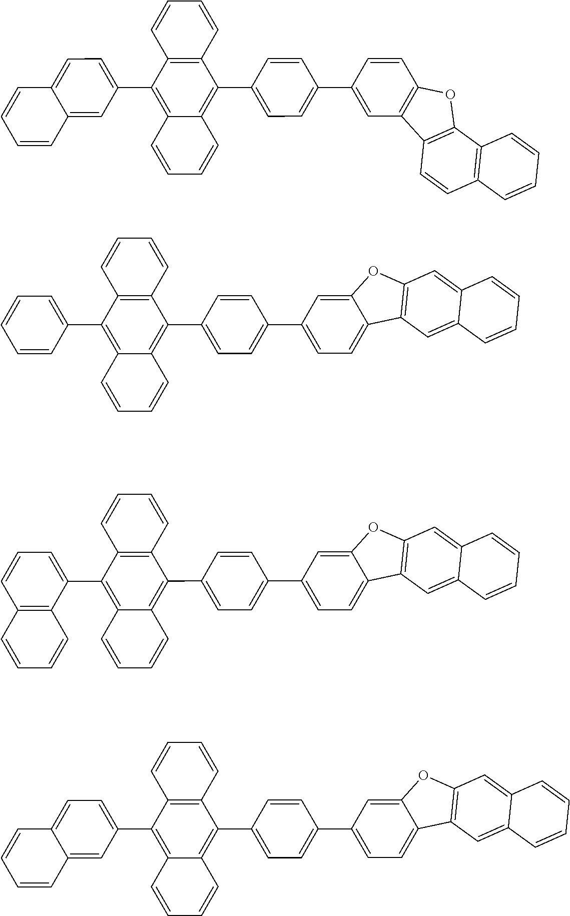 [SMILES: c1ccc2cc(-c3c4ccccc4c(-c4ccc(-c5ccc6oc7c8ccccc8ccc7c6c5)cc4)c4ccccc34)ccc2c1 ; c1ccc(-c2c3ccccc3c(-c3ccc(-c4ccc5c(c4)oc4cc6ccccc6cc45)cc3)c3ccccc23)cc1 ; c1ccc2cc3c(cc2c1)oc1cc(-c2ccc(-c4c5ccccc5c(-c5cccc6ccccc56)c5ccccc45)cc2)ccc13 ; c1ccc2cc(-c3c4ccccc4c(-c4ccc(-c5ccc6c(c5)oc5cc7ccccc7cc56)cc4)c4ccccc34)ccc2c1]

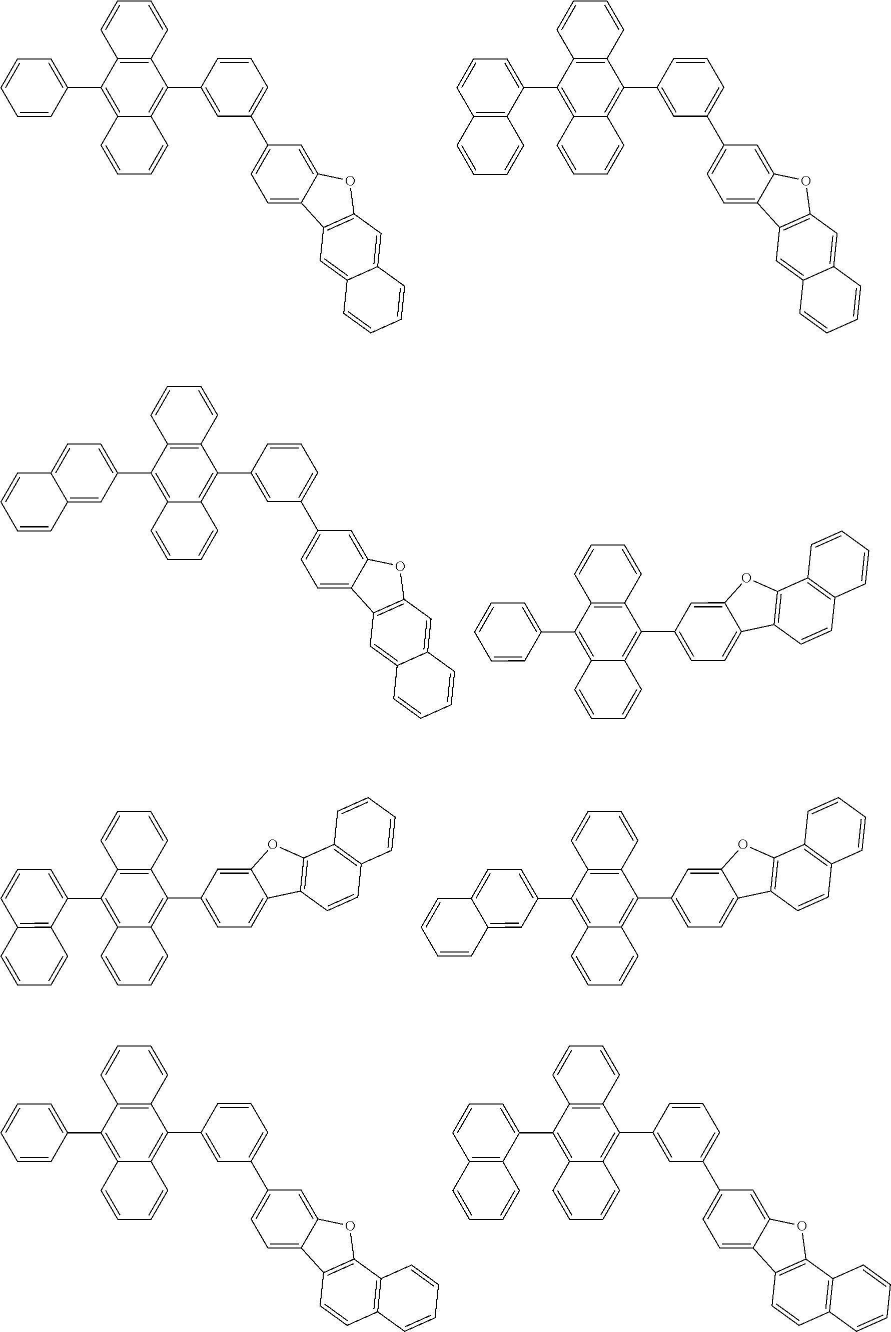

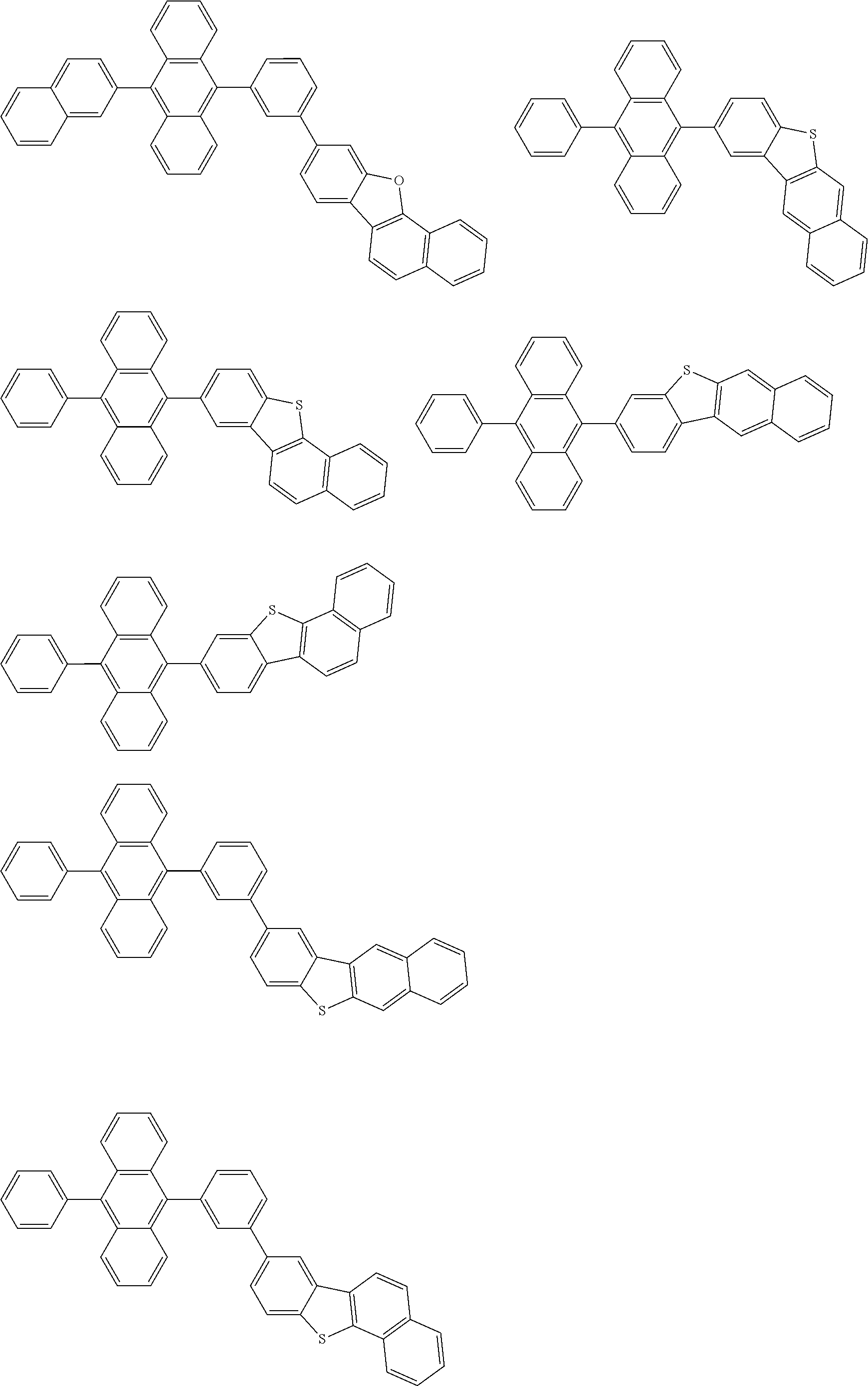

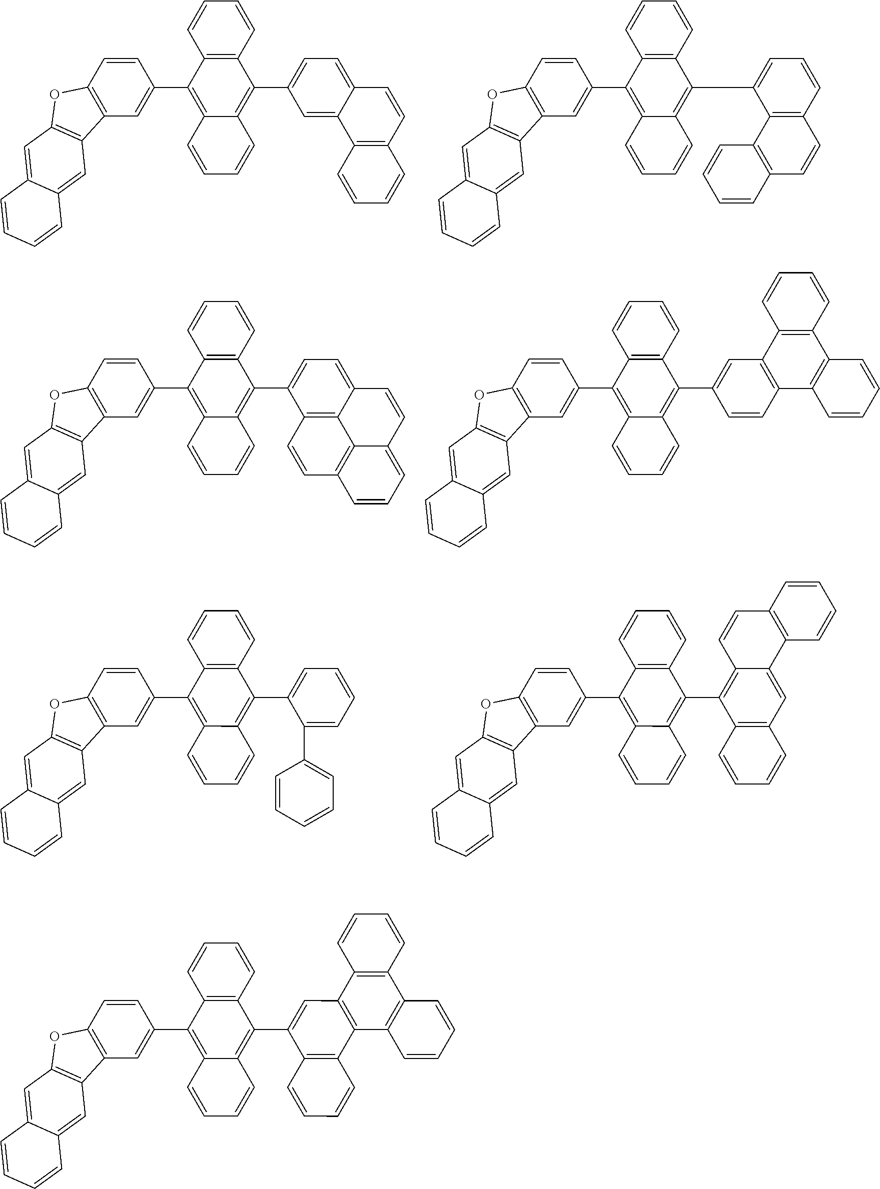

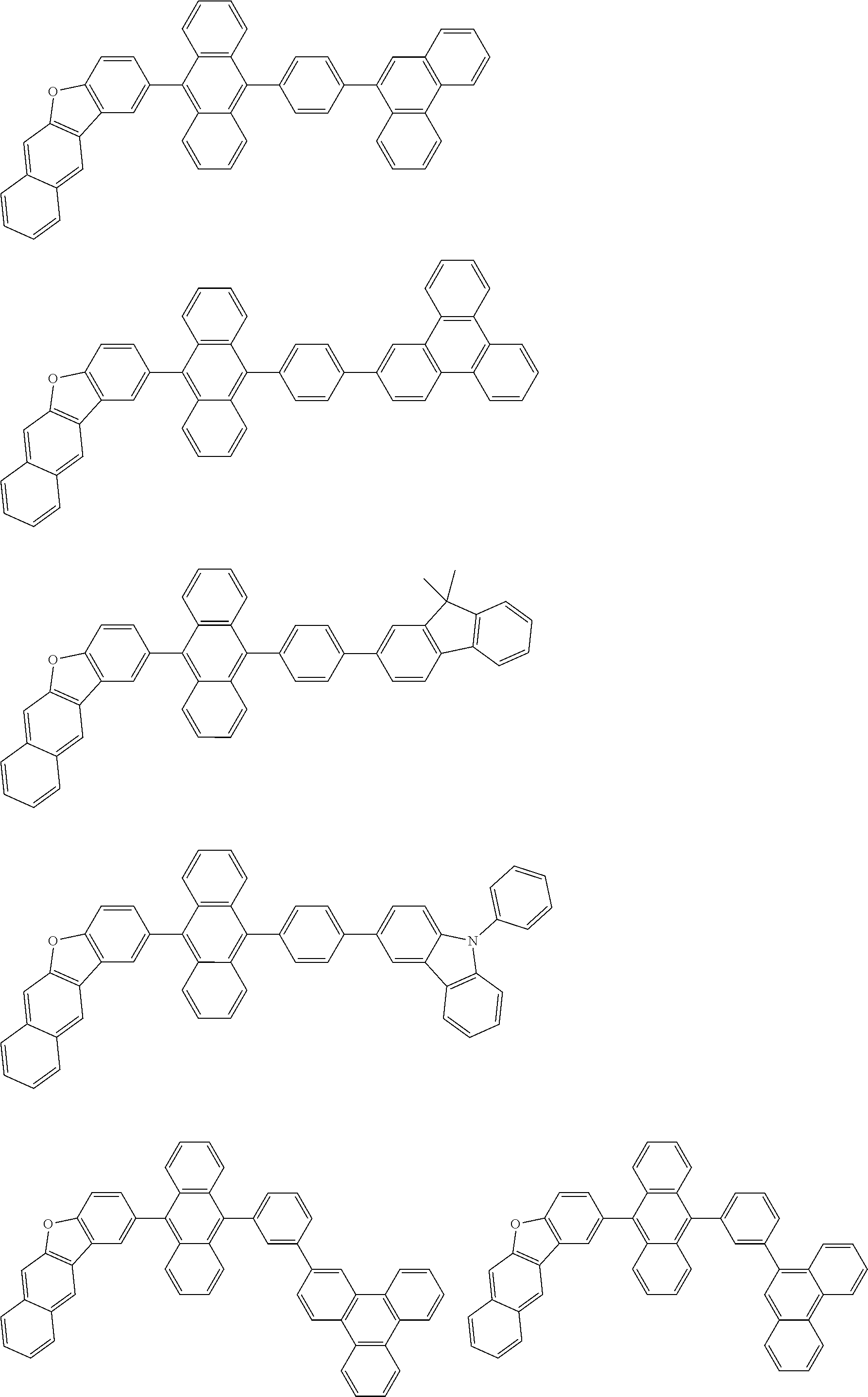

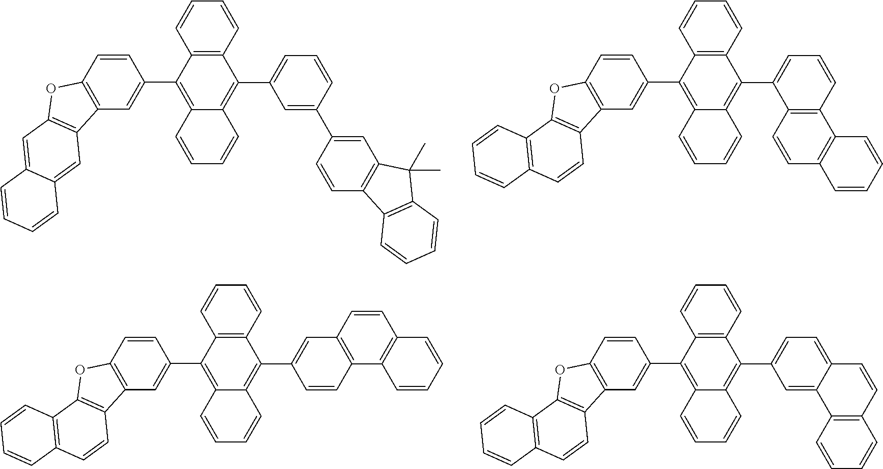

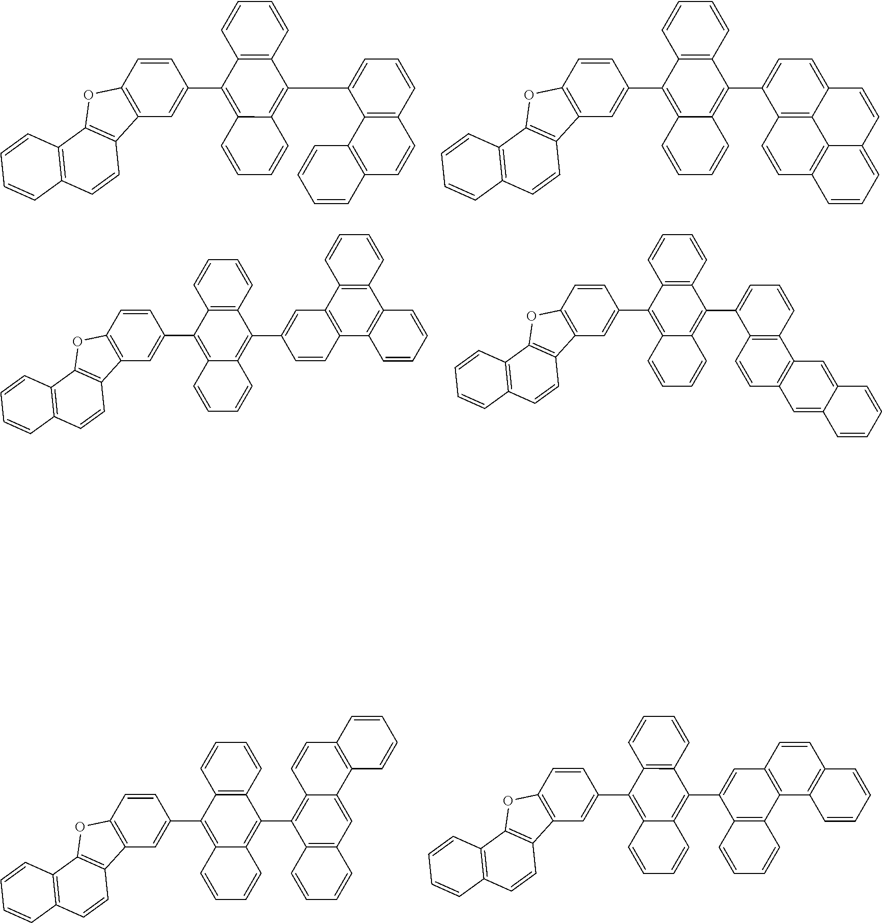

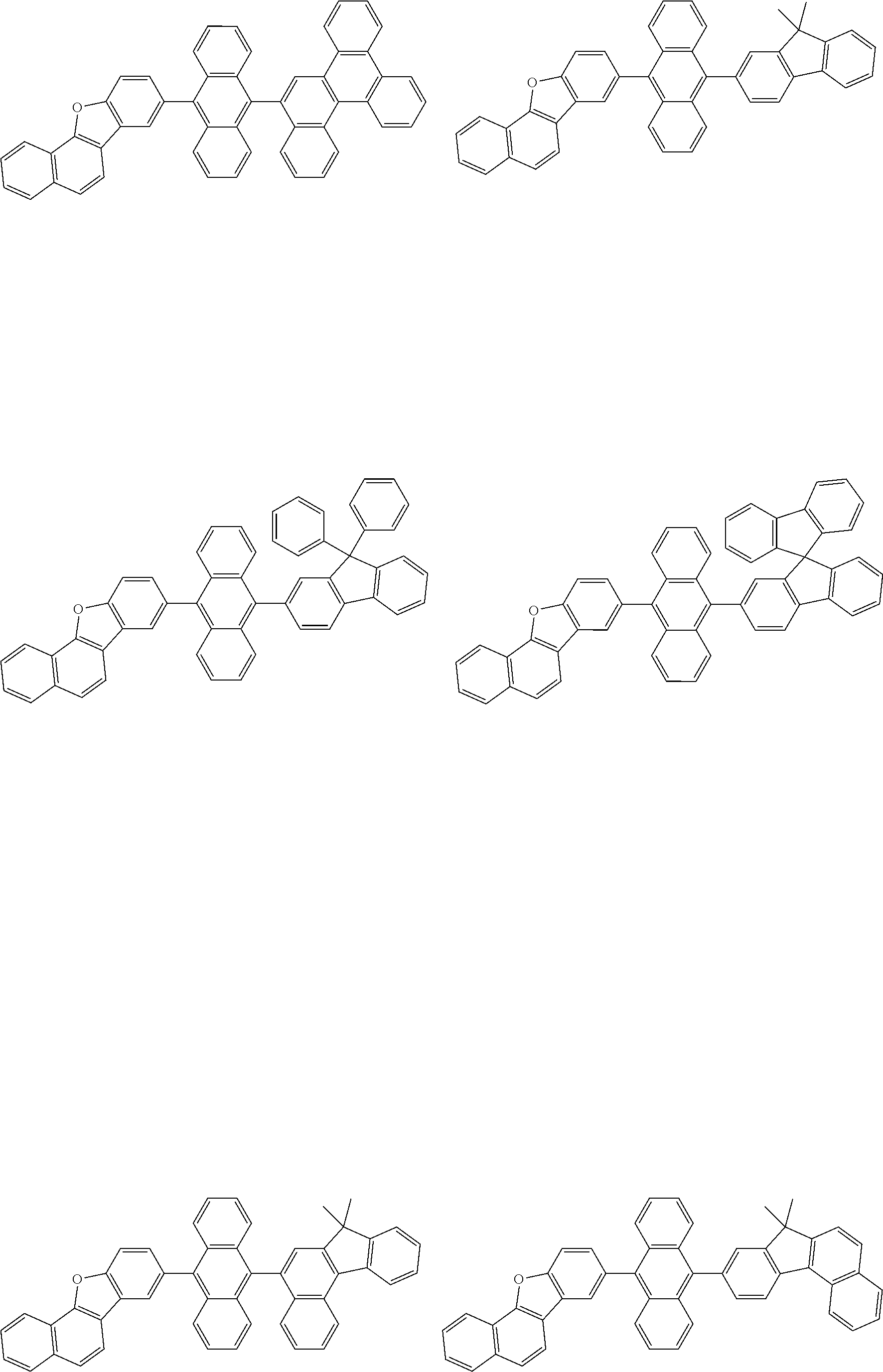

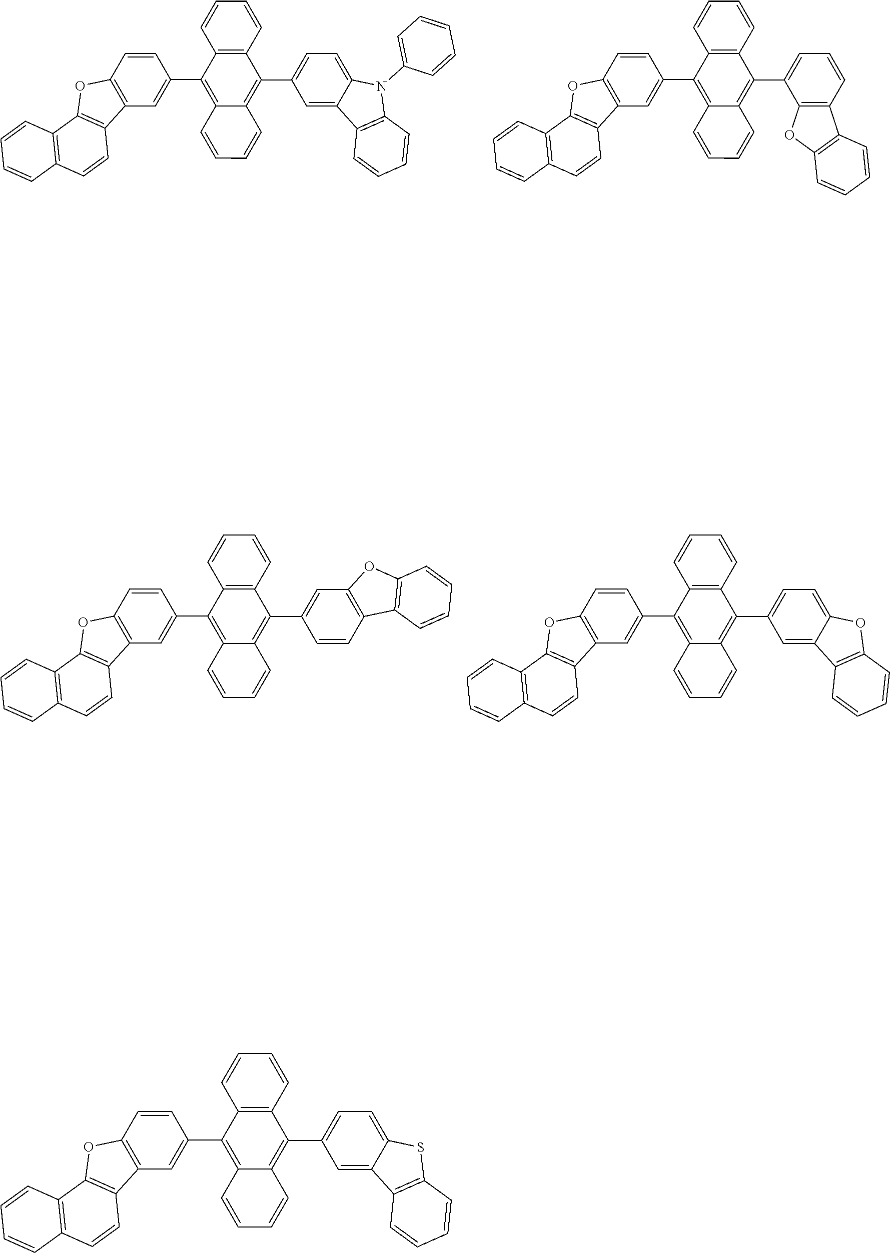 [SMILES: c1ccc(-n2c3ccccc3c3cc(-c4c5ccccc5c(-c5ccc6oc7c8ccccc8ccc7c6c5)c5ccccc45)ccc32)cc1 ; c1ccc2c(c1)ccc1c3cc(-c4c5ccccc5c(-c5cccc6c5oc5ccccc56)c5ccccc45)ccc3oc21 ; c1ccc2c(c1)ccc1c3cc(-c4c5ccccc5c(-c5ccc6c(c5)oc5ccccc56)c5ccccc45)ccc3oc21 ; c1ccc2c(c1)ccc1c3cc(-c4c5ccccc5c(-c5ccc6oc7ccccc7c6c5)c5ccccc45)ccc3oc21 ; c1ccc2c(c1)ccc1c3cc(-c4c5ccccc5c(-c5ccc6sc7ccccc7c6c5)c5ccccc45)ccc3oc21]

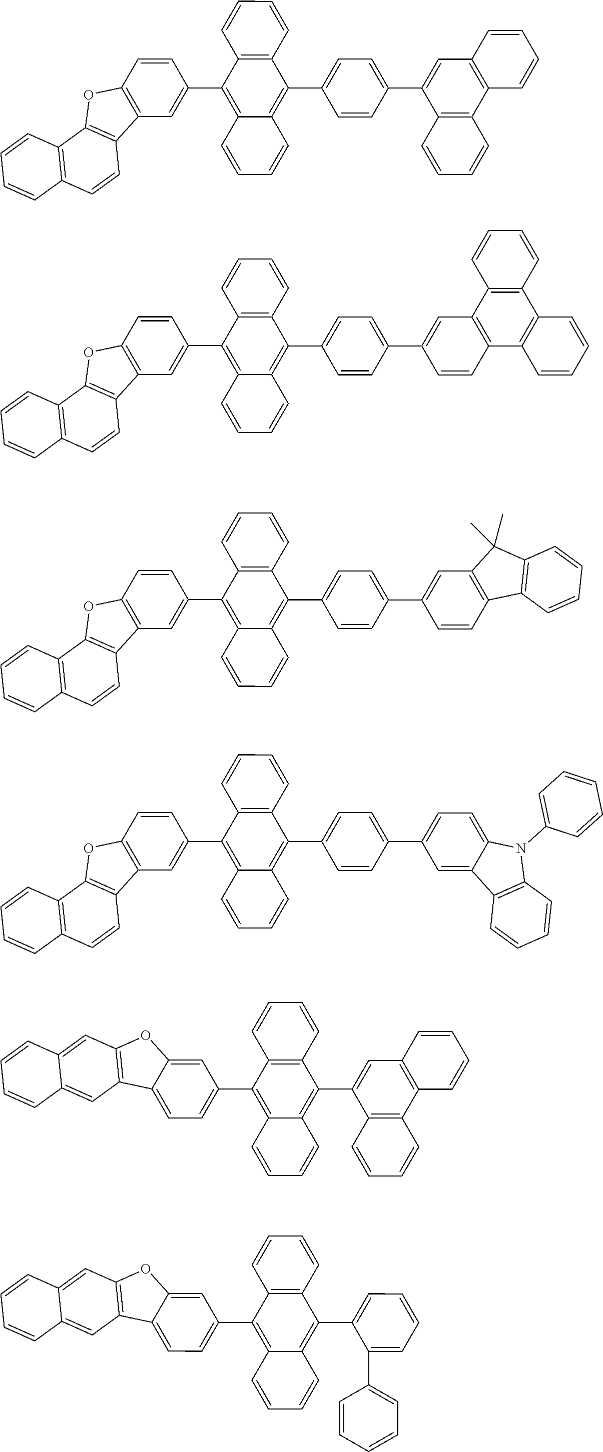

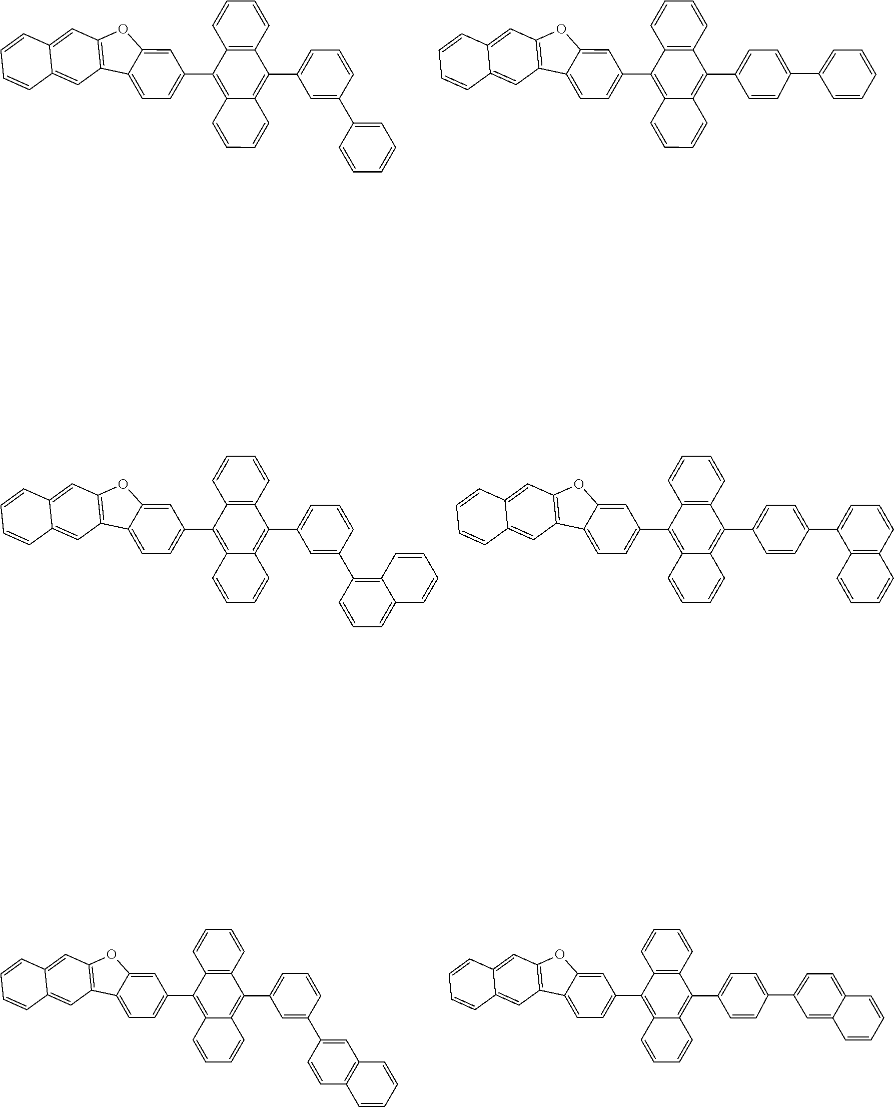 [SMILES: c1ccc(-c2cccc(-c3c4ccccc4c(-c4ccc5c(c4)oc4cc6ccccc6cc45)c4ccccc34)c2)cc1 ; c1ccc(-c2ccc(-c3c4ccccc4c(-c4ccc5c(c4)oc4cc6ccccc6cc45)c4ccccc34)cc2)cc1 ; c1cc(-c2cccc3ccccc23)cc(-c2c3ccccc3c(-c3ccc4c(c3)oc3cc5ccccc5cc34)c3ccccc23)c1 ; c1ccc2cc3c(cc2c1)oc1cc(-c2c4ccccc4c(-c4ccc(-c5cccc6ccccc56)cc4)c4ccccc24)ccc13 ; c1cc(-c2ccc3ccccc3c2)cc(-c2c3ccccc3c(-c3ccc4c(c3)oc3cc5ccccc5cc34)c3ccccc23)c1 ; c1ccc2cc(-c3ccc(-c4c5ccccc5c(-c5ccc6c(c5)oc5cc7ccccc7cc56)c5ccccc45)cc3)ccc2c1]

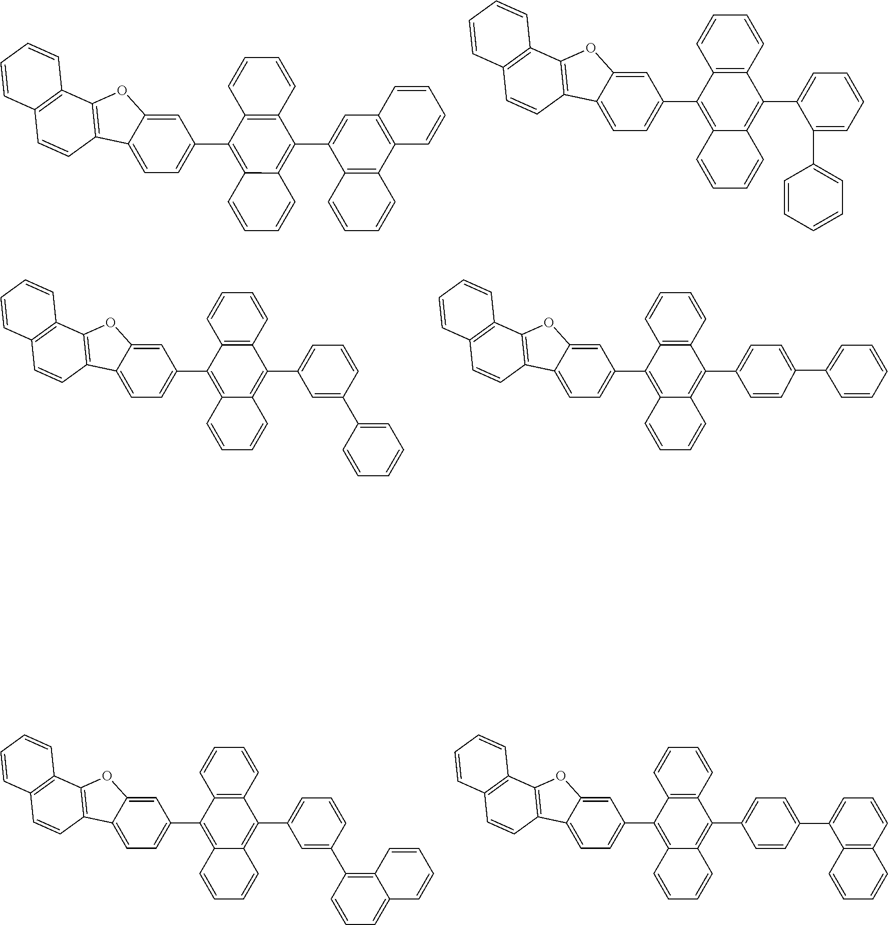 [SMILES: c1ccc2c(c1)cc(-c1c3ccccc3c(-c3ccc4c(c3)oc3c5ccccc5ccc43)c3ccccc13)c1ccccc12 ; c1ccc(-c2ccccc2-c2c3ccccc3c(-c3ccc4c(c3)oc3c5ccccc5ccc43)c3ccccc23)cc1 ; c1ccc(-c2cccc(-c3c4ccccc4c(-c4ccc5c(c4)oc4c6ccccc6ccc54)c4ccccc34)c2)cc1 ; c1ccc(-c2ccc(-c3c4ccccc4c(-c4ccc5c(c4)oc4c6ccccc6ccc54)c4ccccc34)cc2)cc1 ; c1cc(-c2cccc3ccccc23)cc(-c2c3ccccc3c(-c3ccc4c(c3)oc3c5ccccc5ccc43)c3ccccc23)c1 ; c1ccc2c(-c3ccc(-c4c5ccccc5c(-c5ccc6c(c5)oc5c7ccccc7ccc65)c5ccccc45)cc3)cccc2c1]

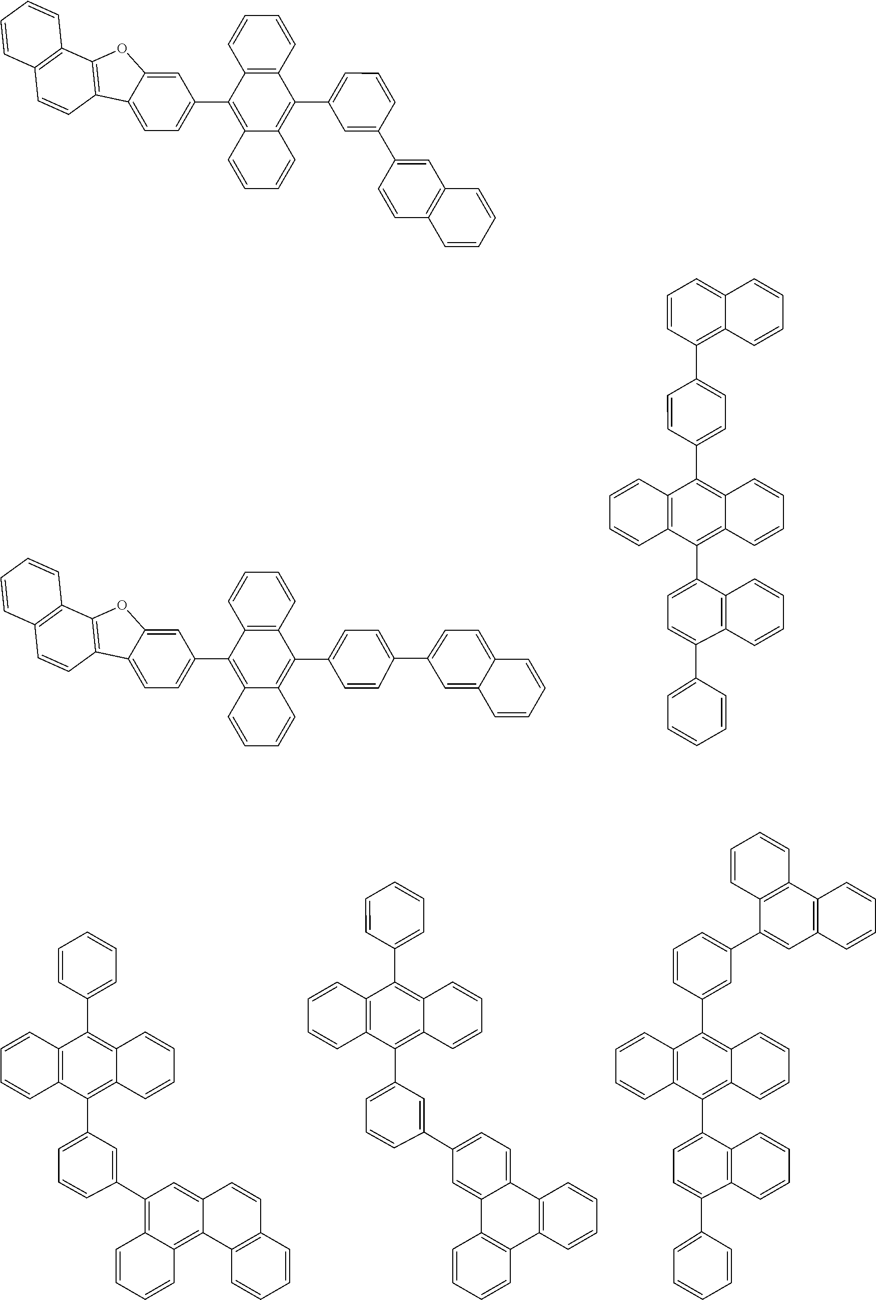

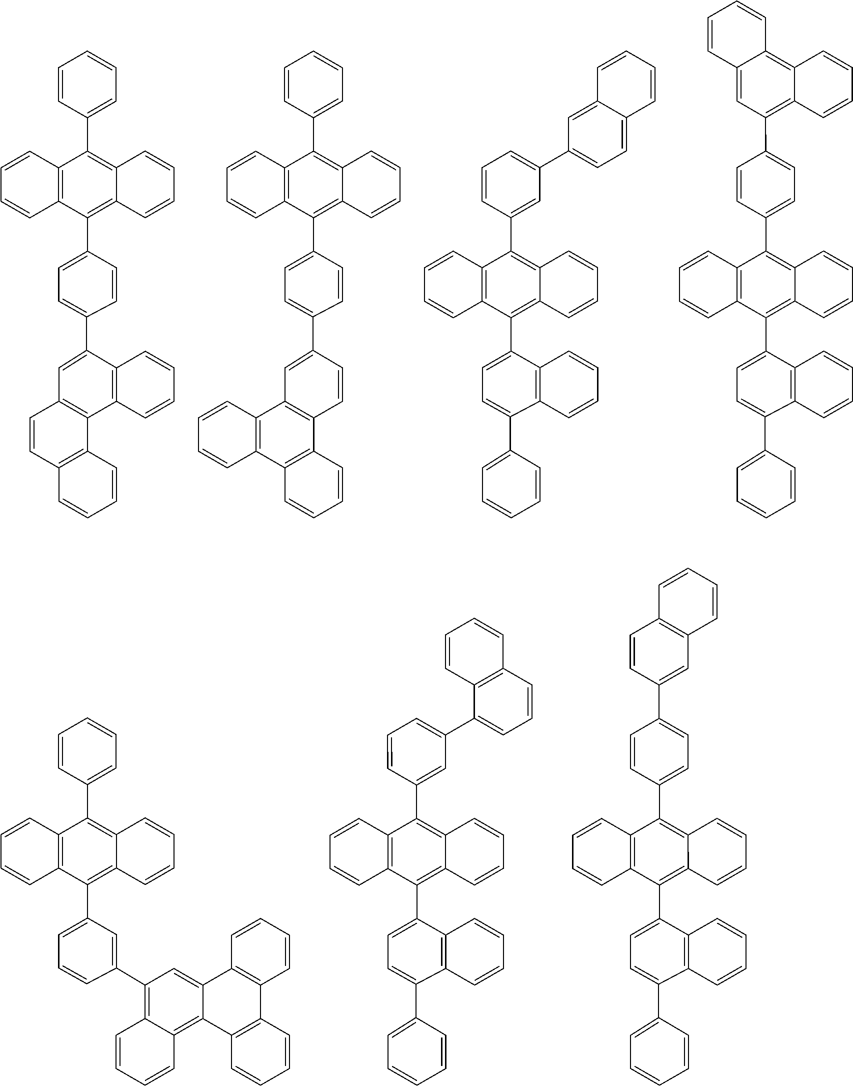

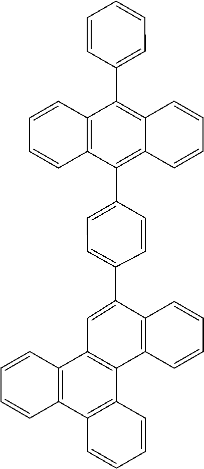 [SMILES: c1ccc(-c2c3ccccc3c(-c3ccc(-c4cc5c6ccccc6c6ccccc6c5c5ccccc45)cc3)c3ccccc23)cc1]

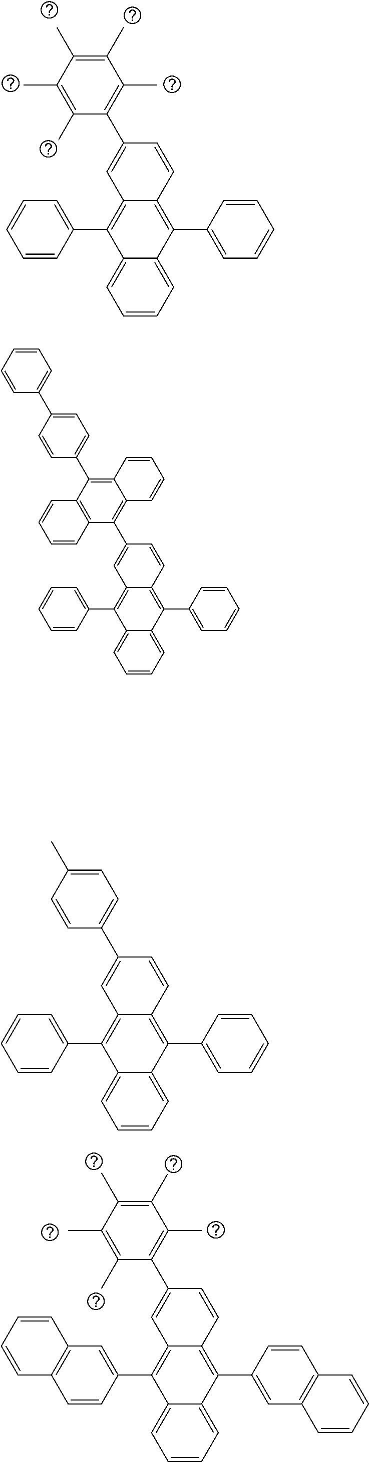

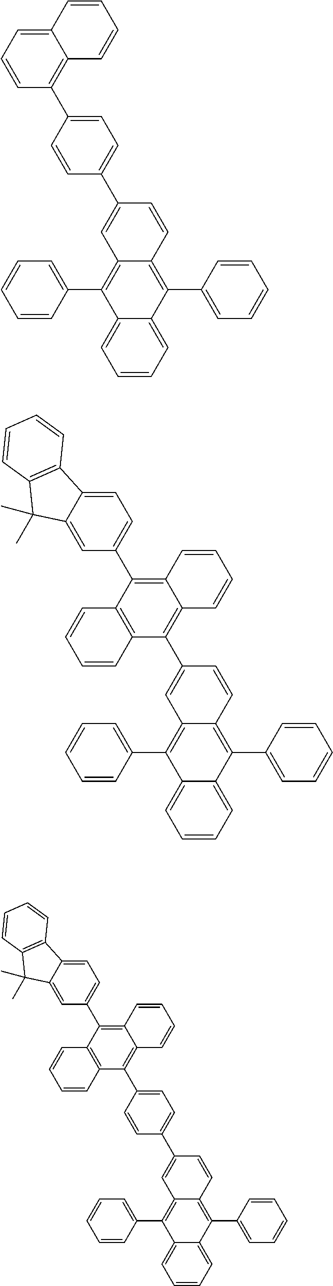

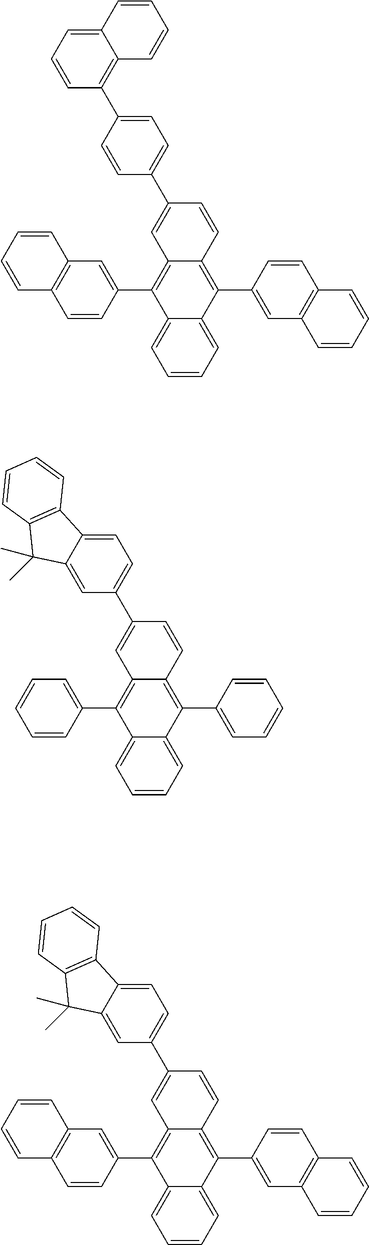

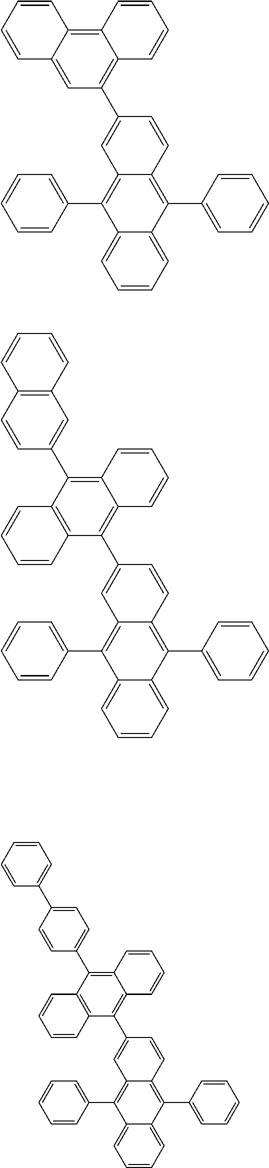

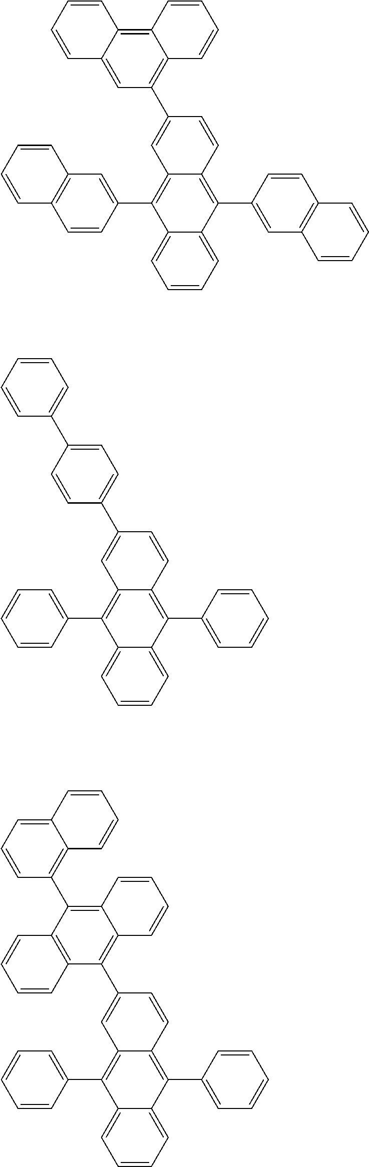

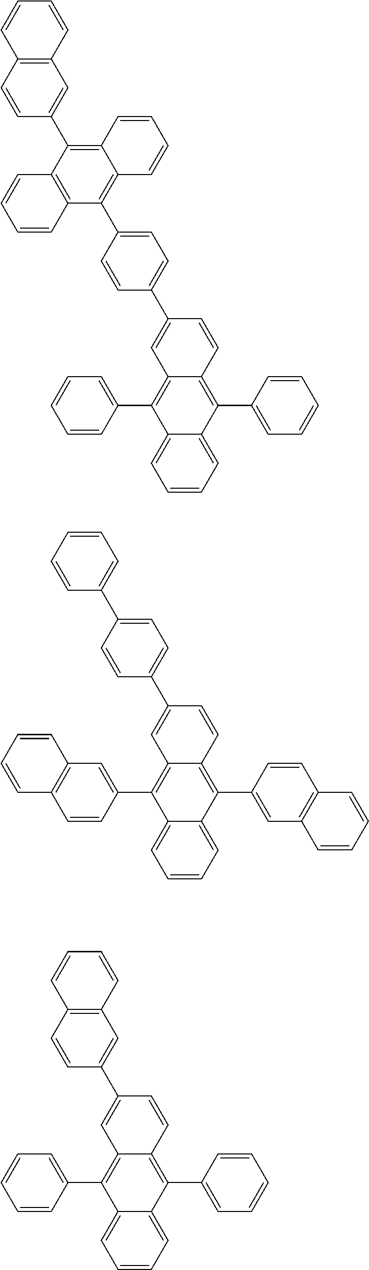

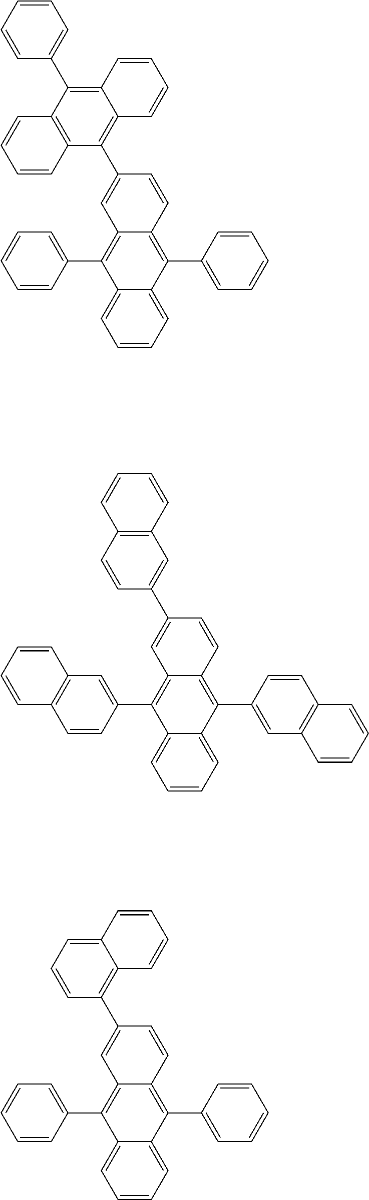

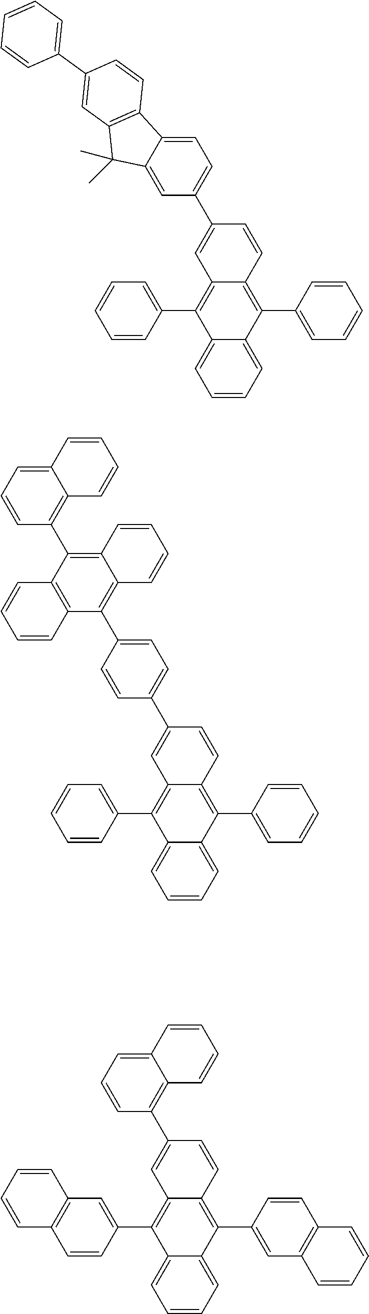

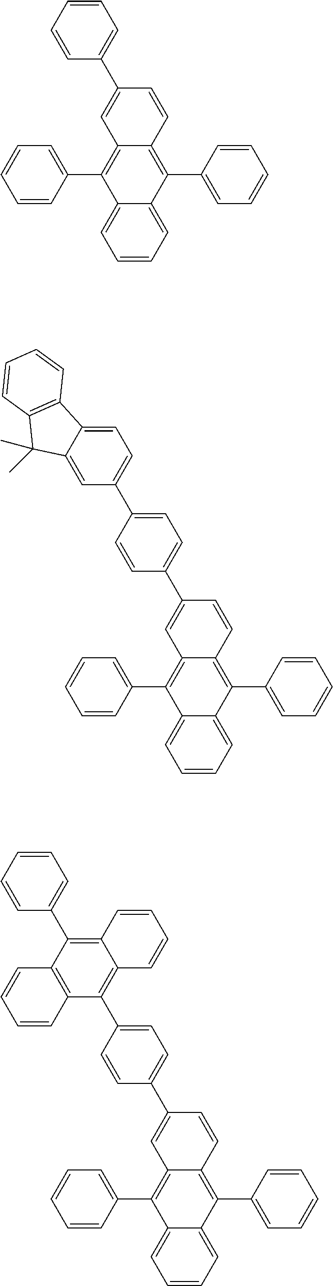

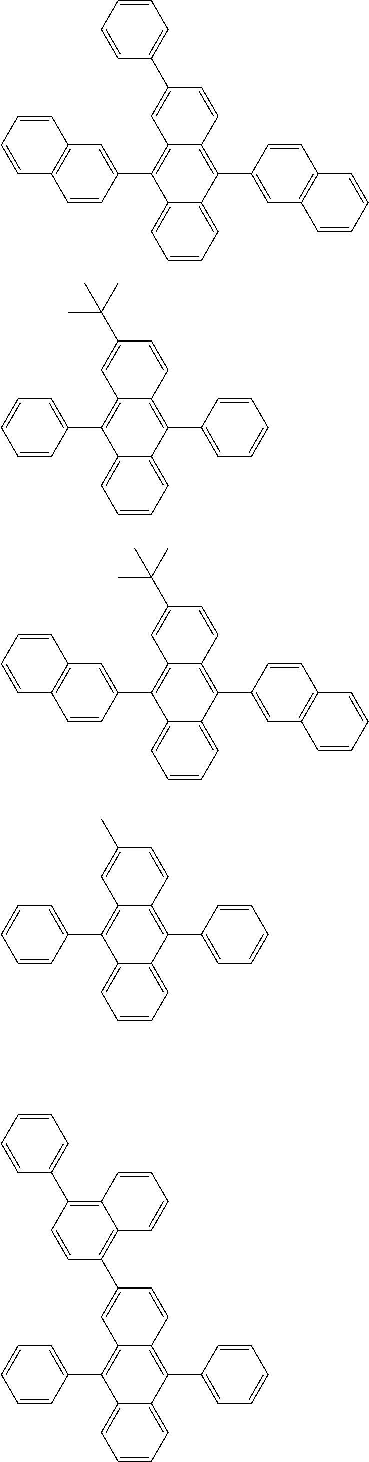

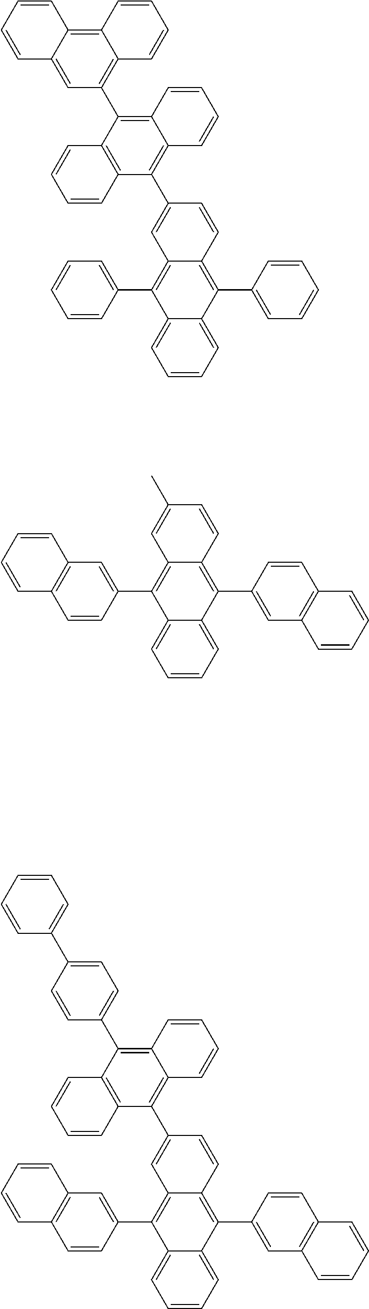

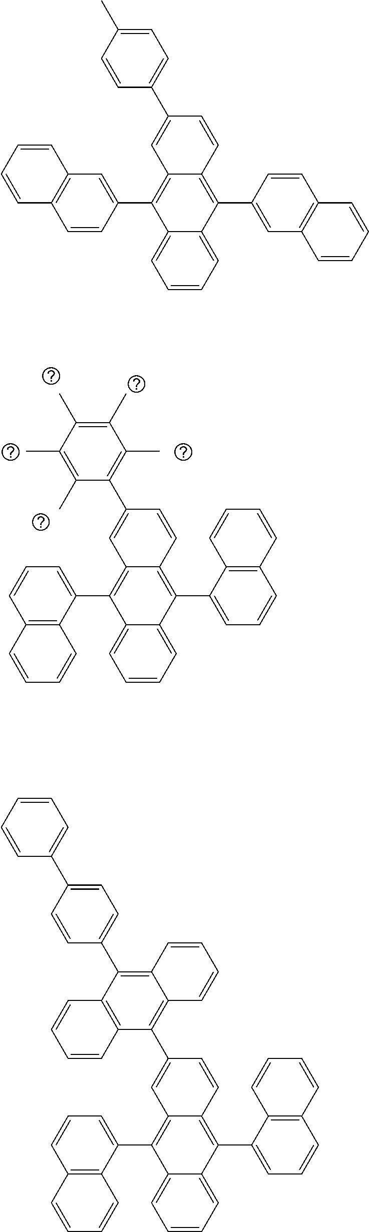

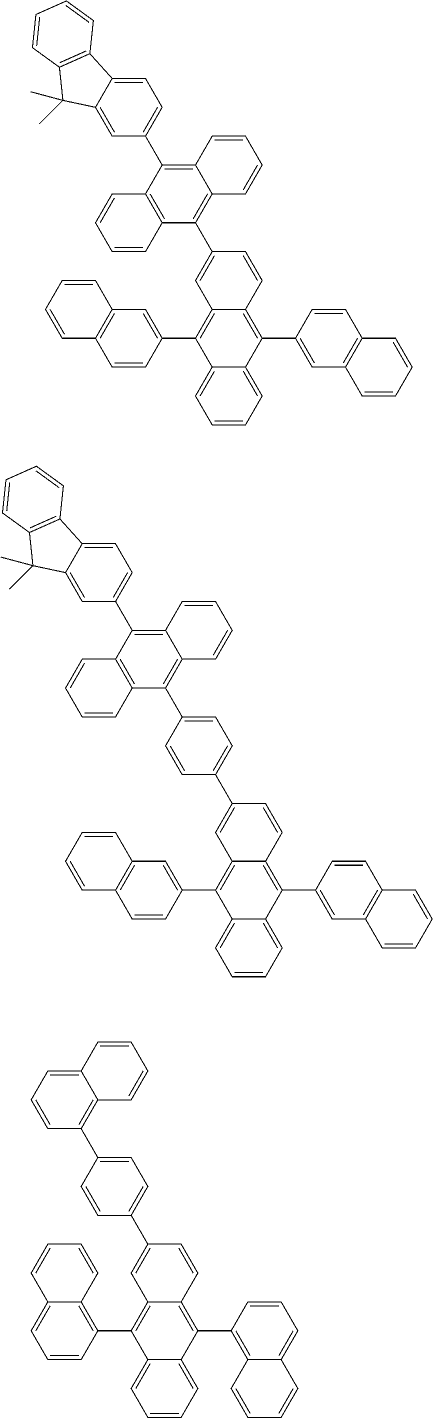

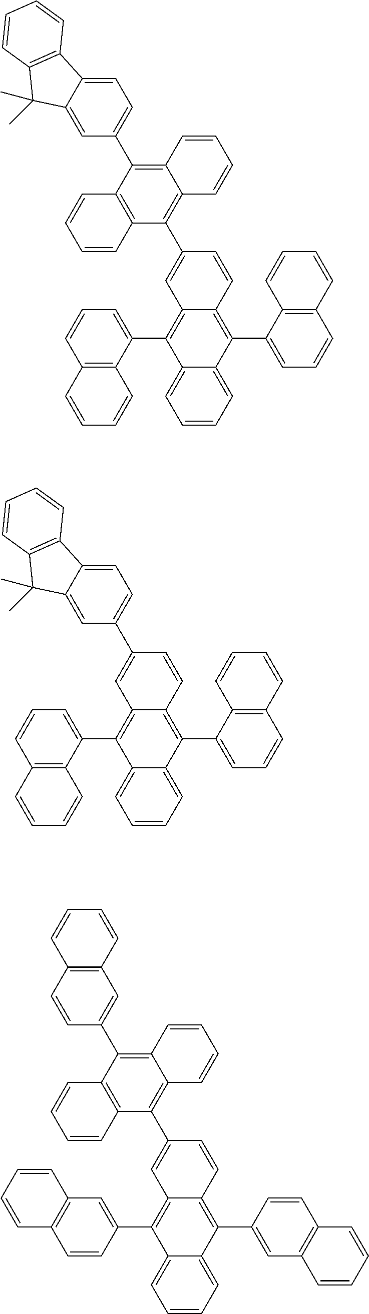

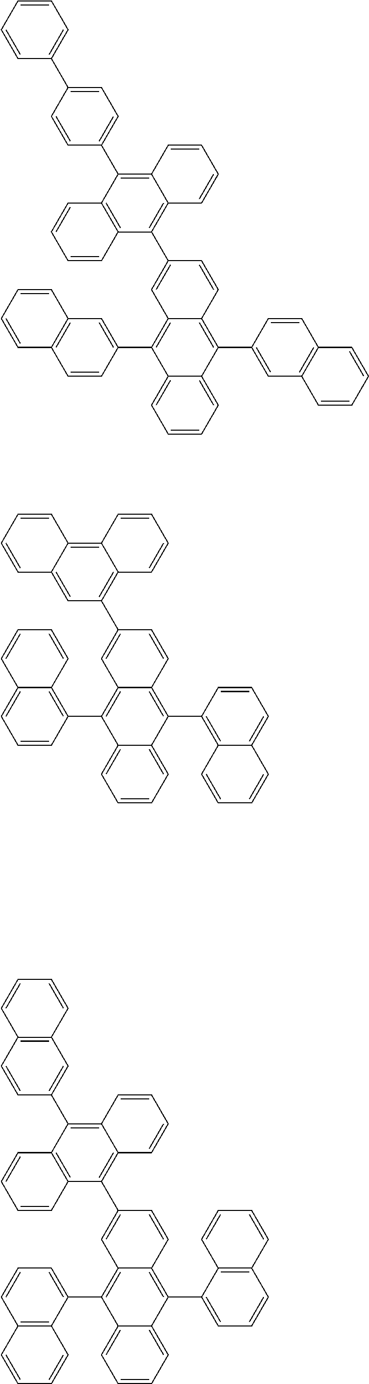

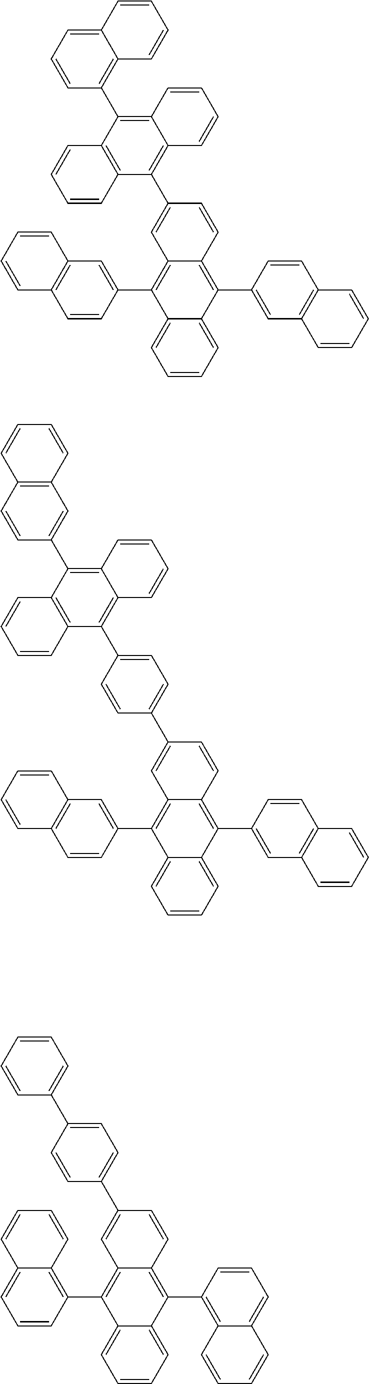

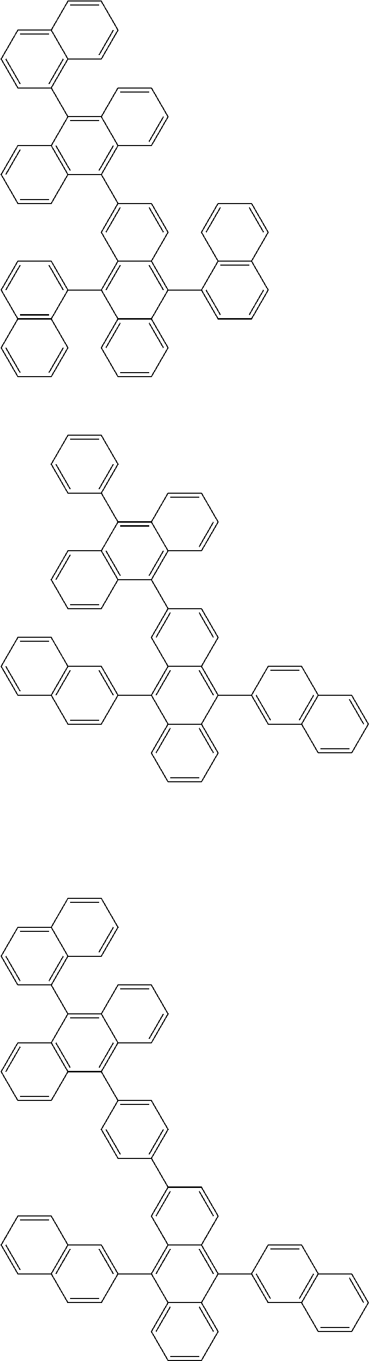

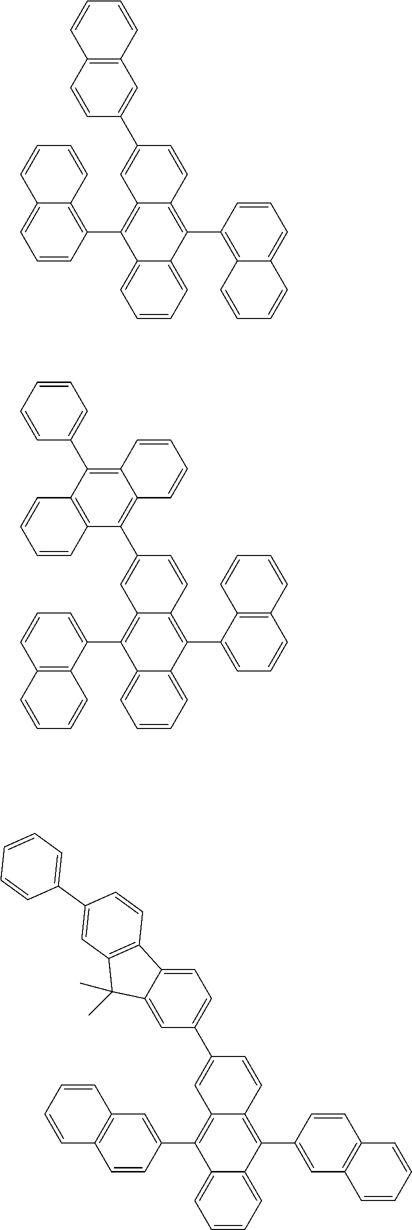

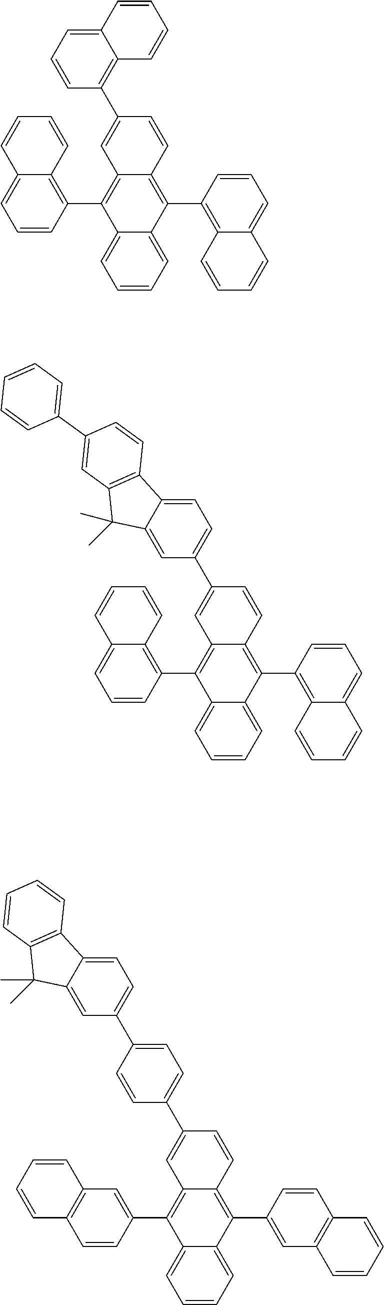

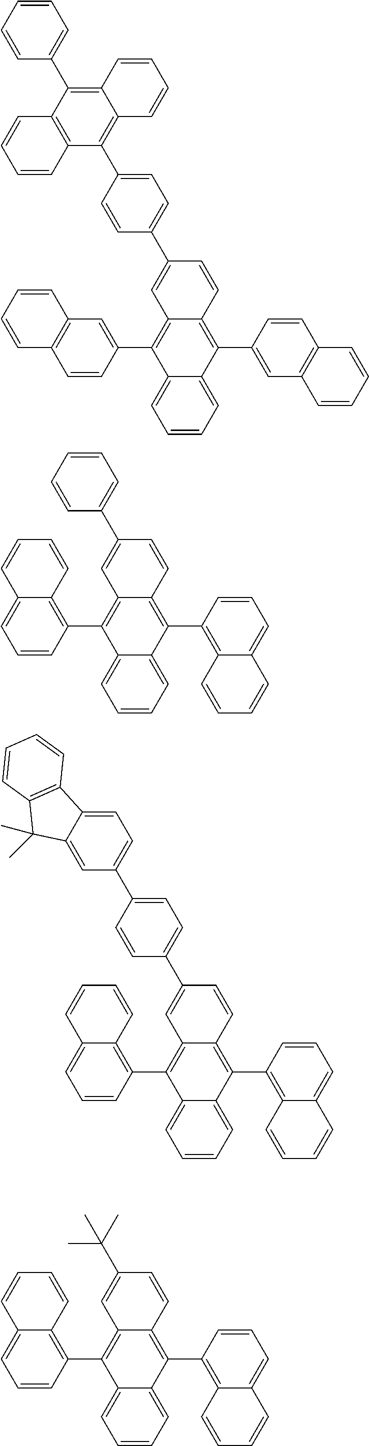

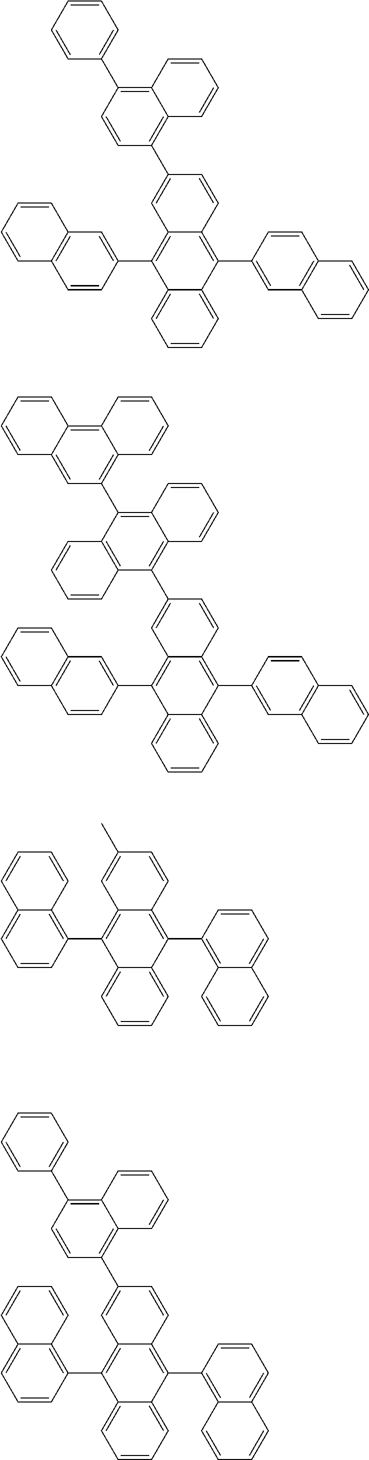

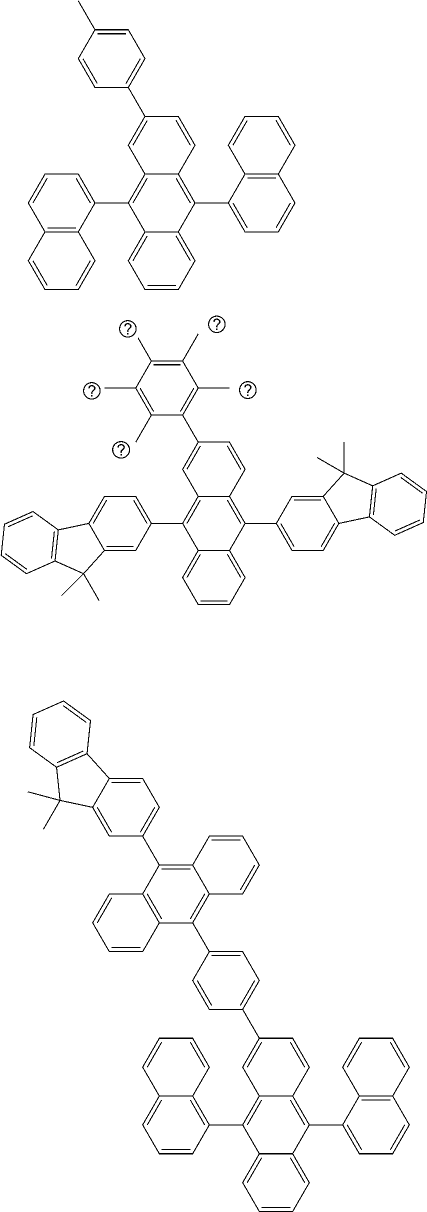

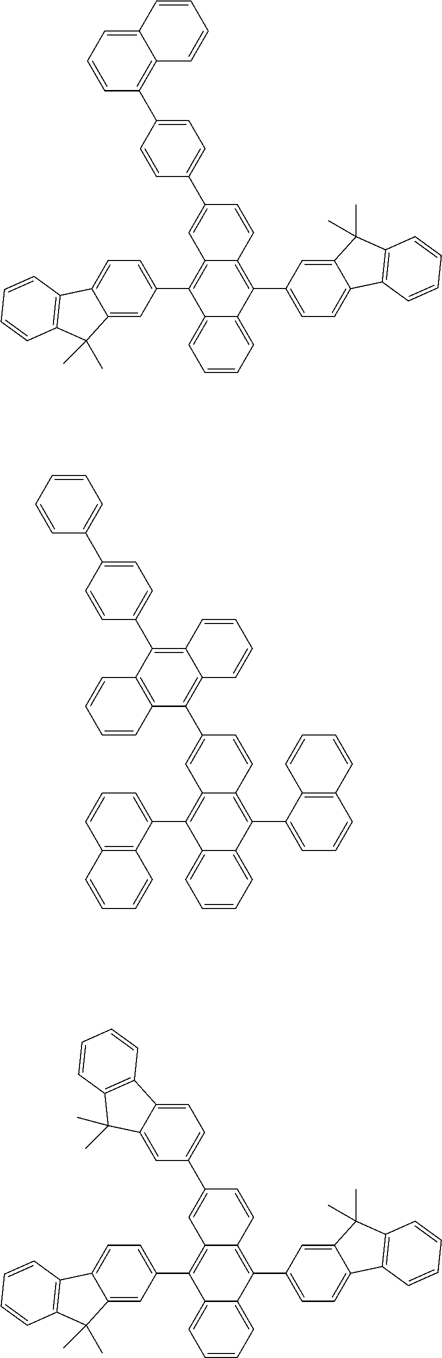

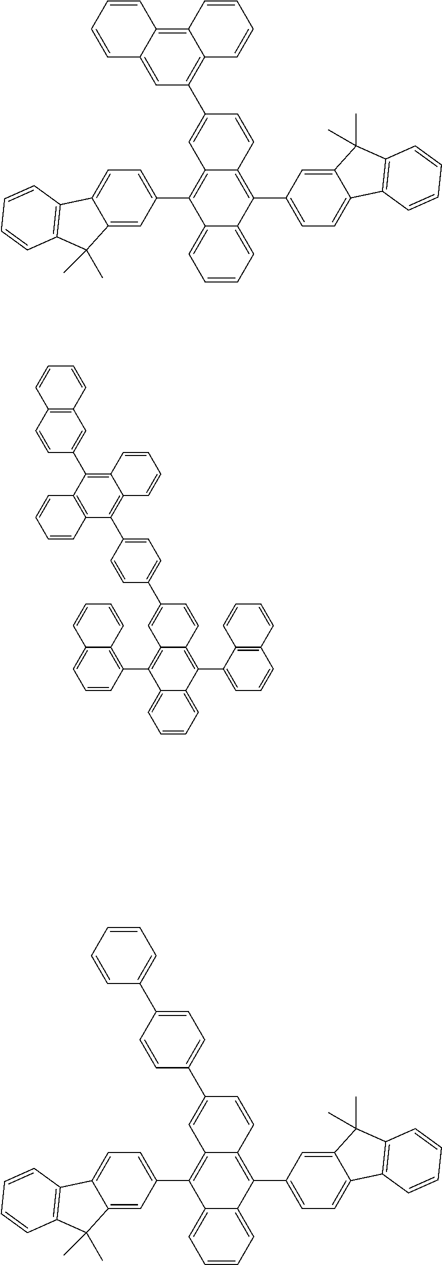

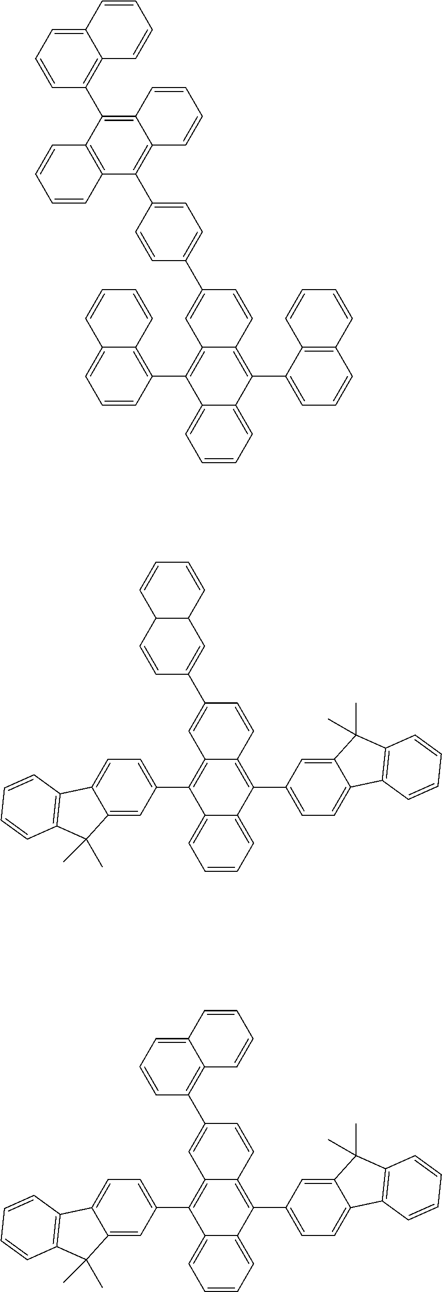

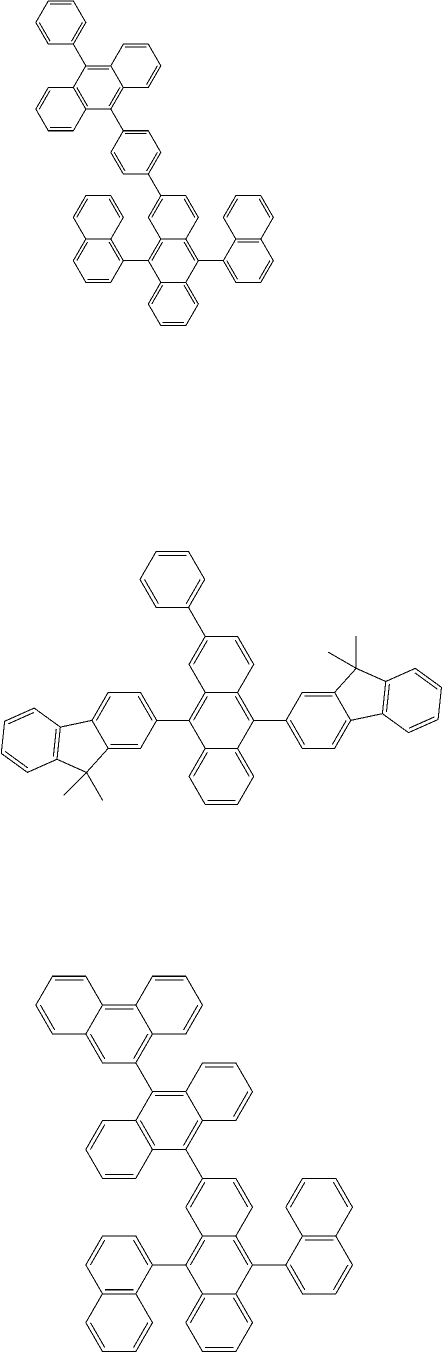

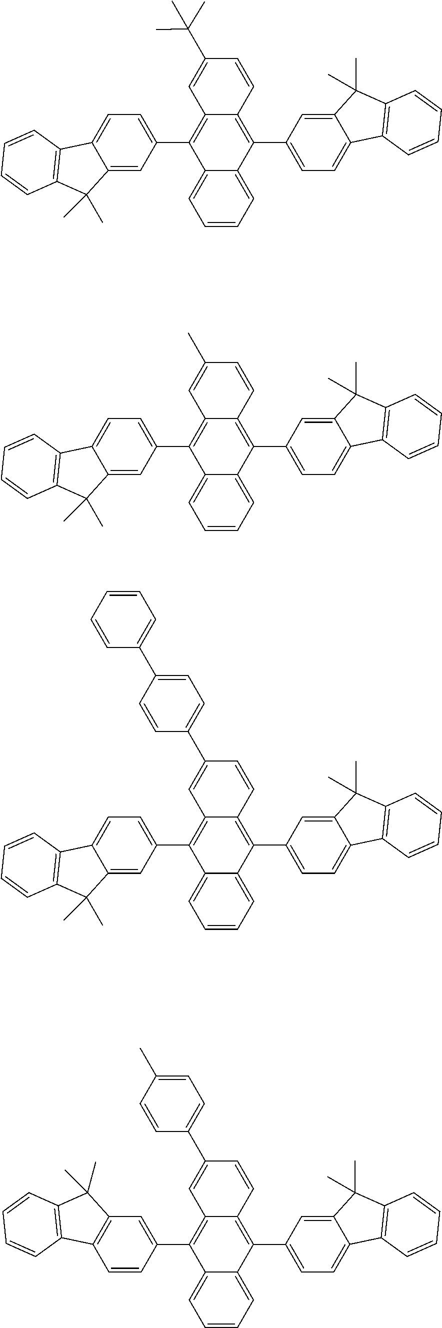

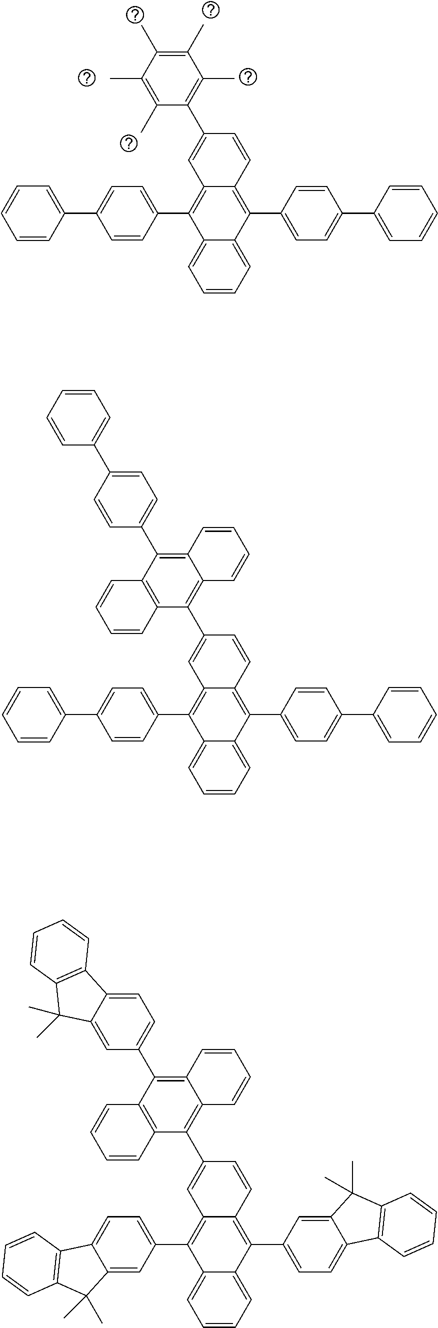

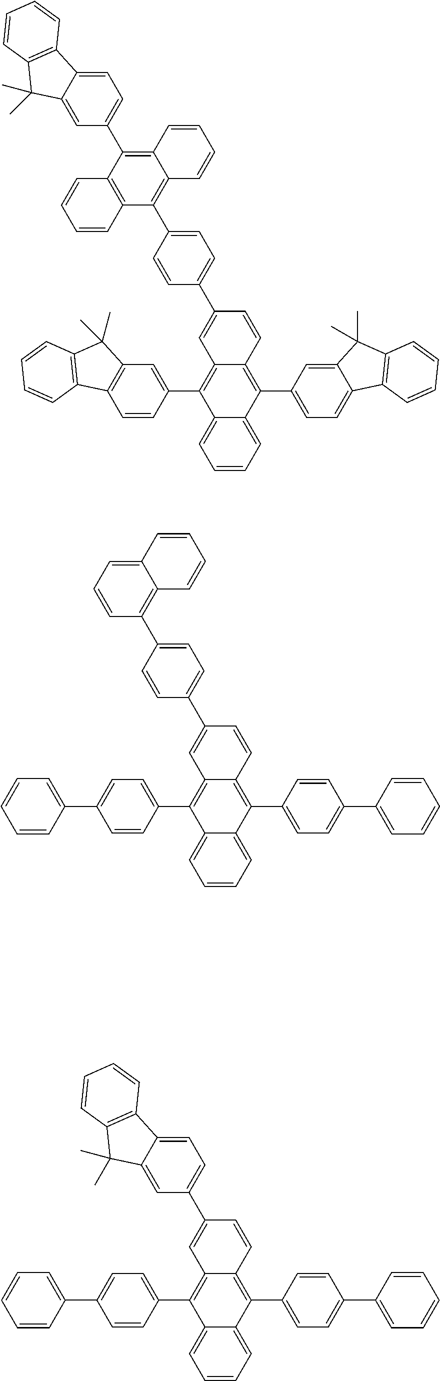

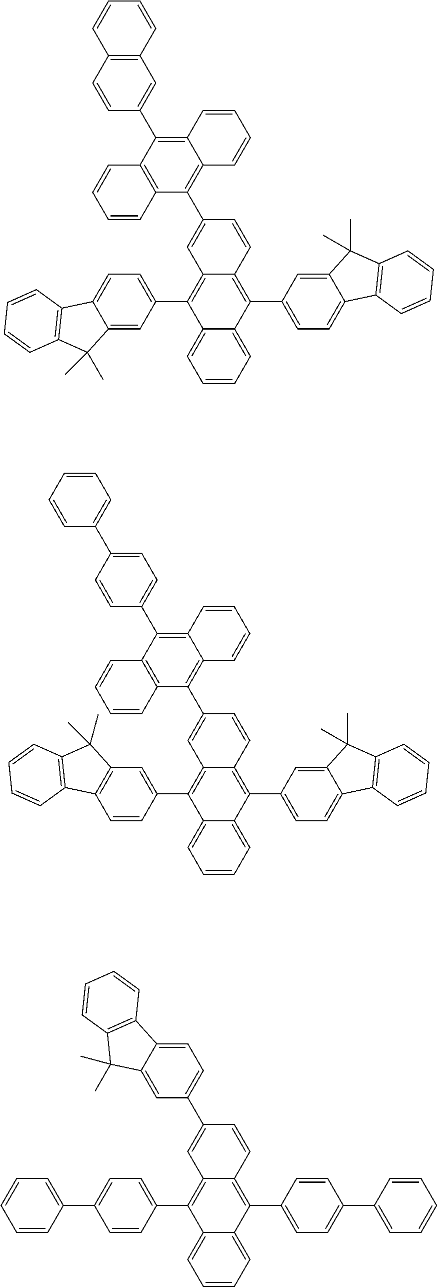

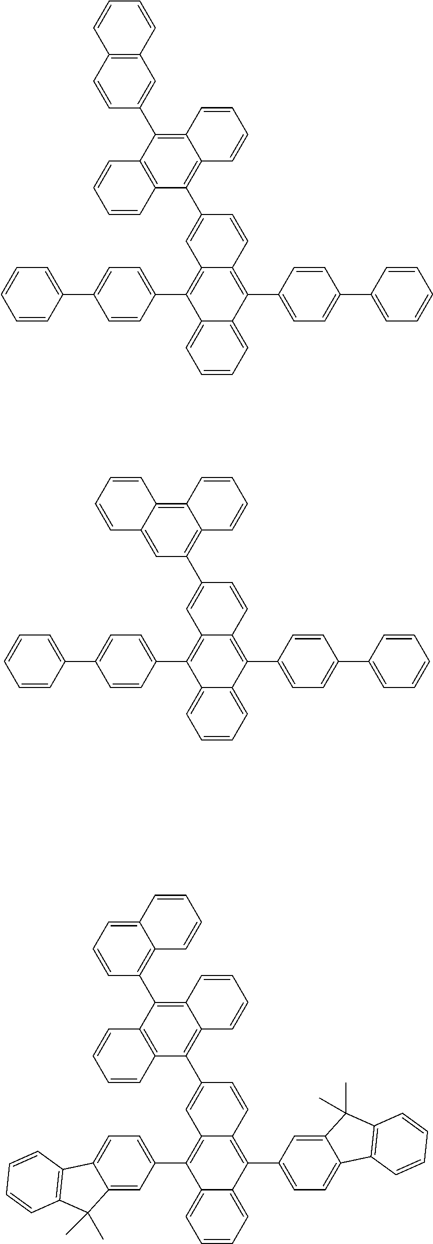

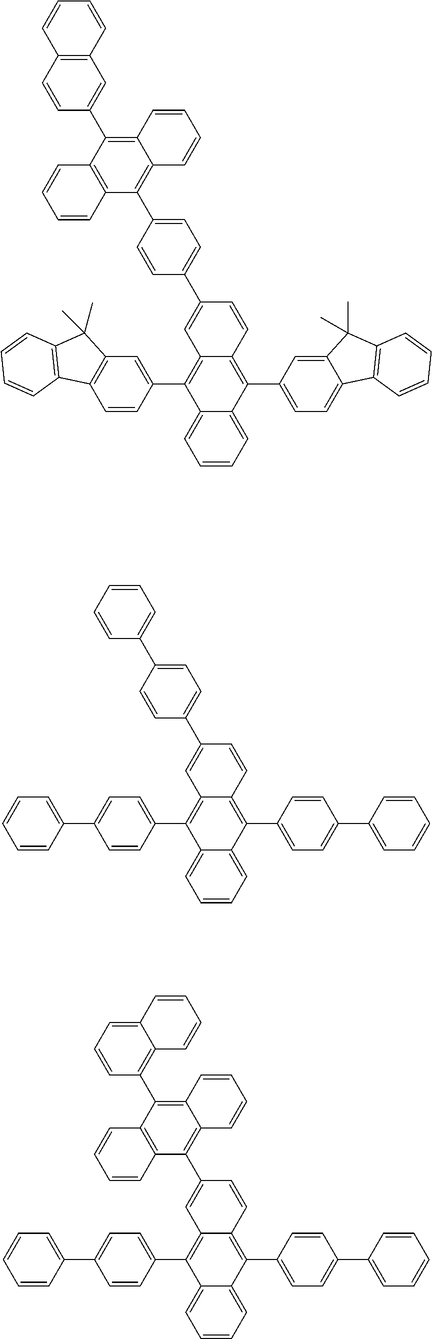

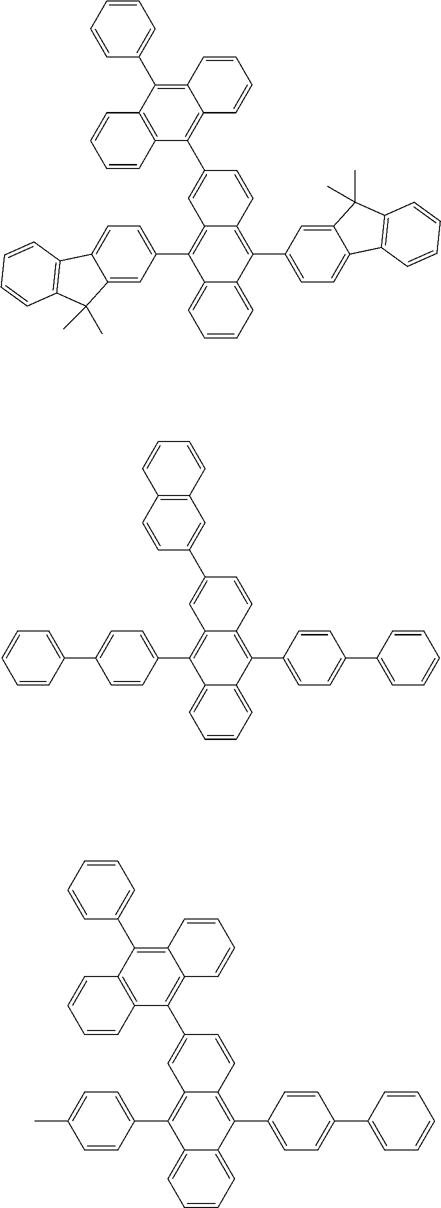

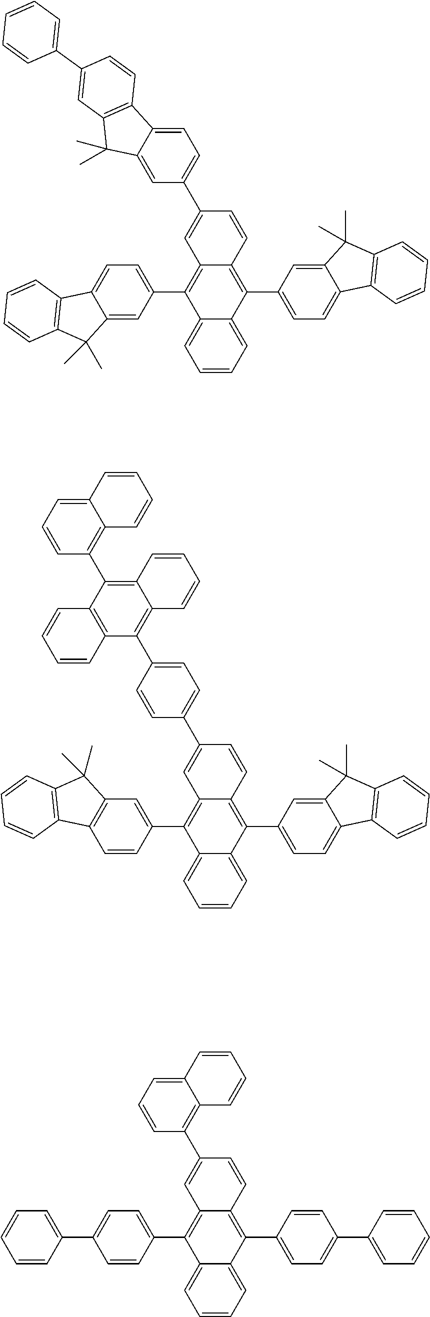

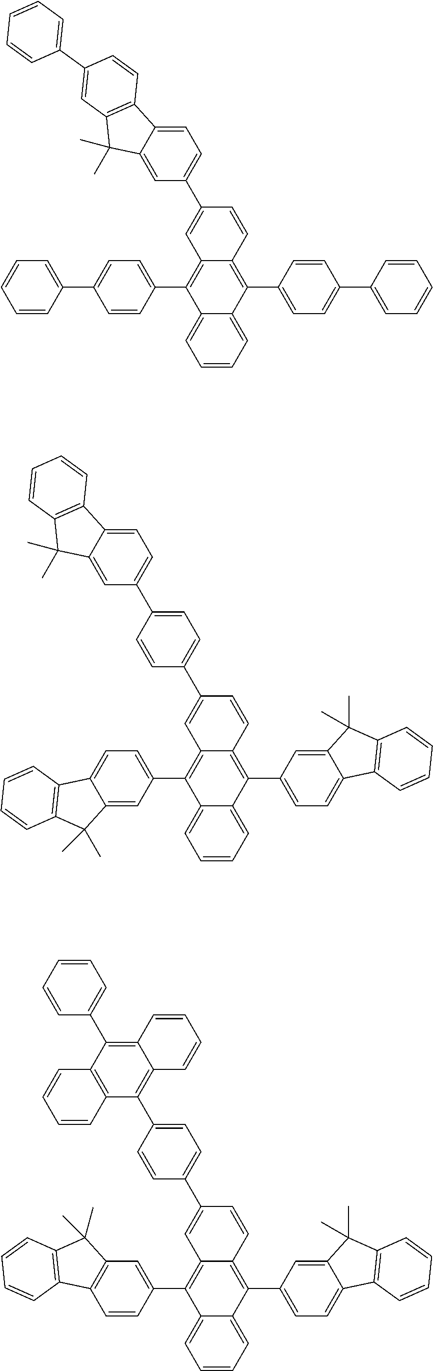

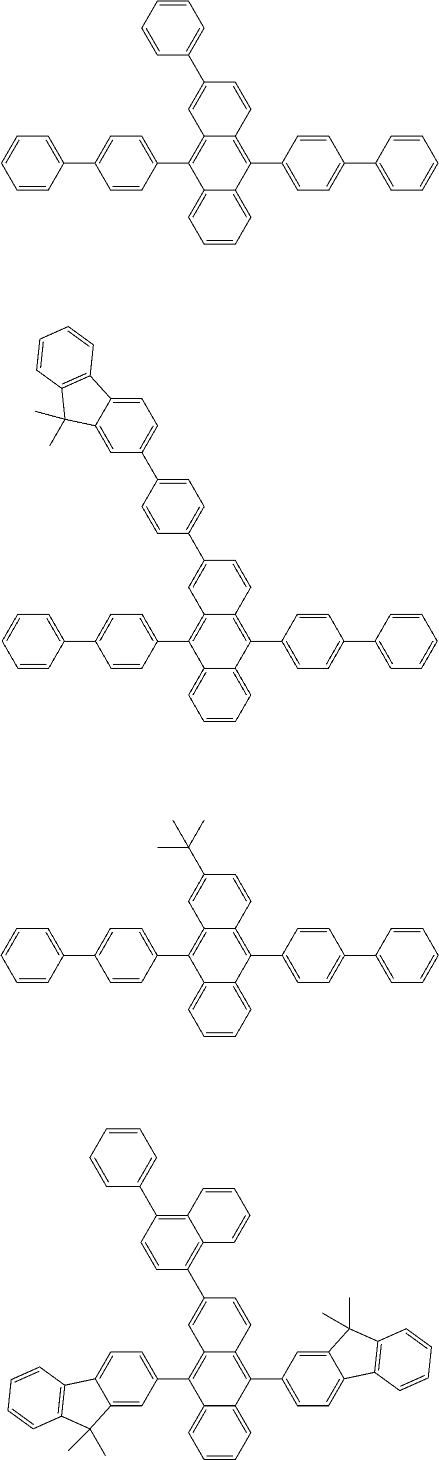

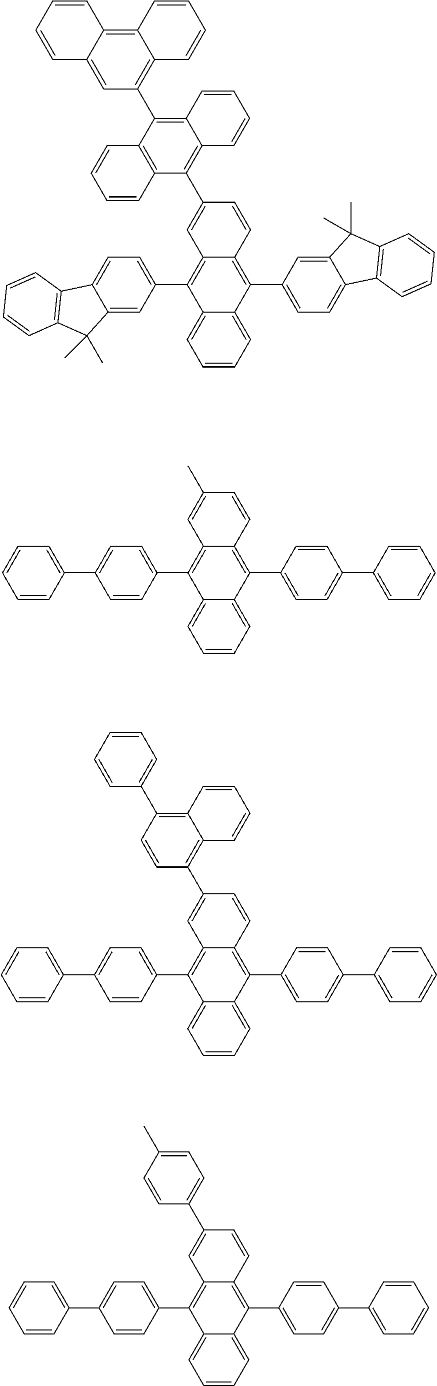

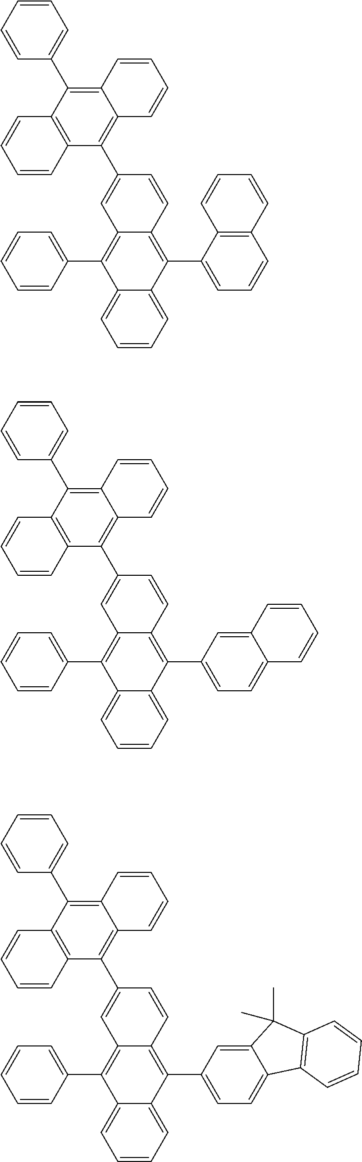

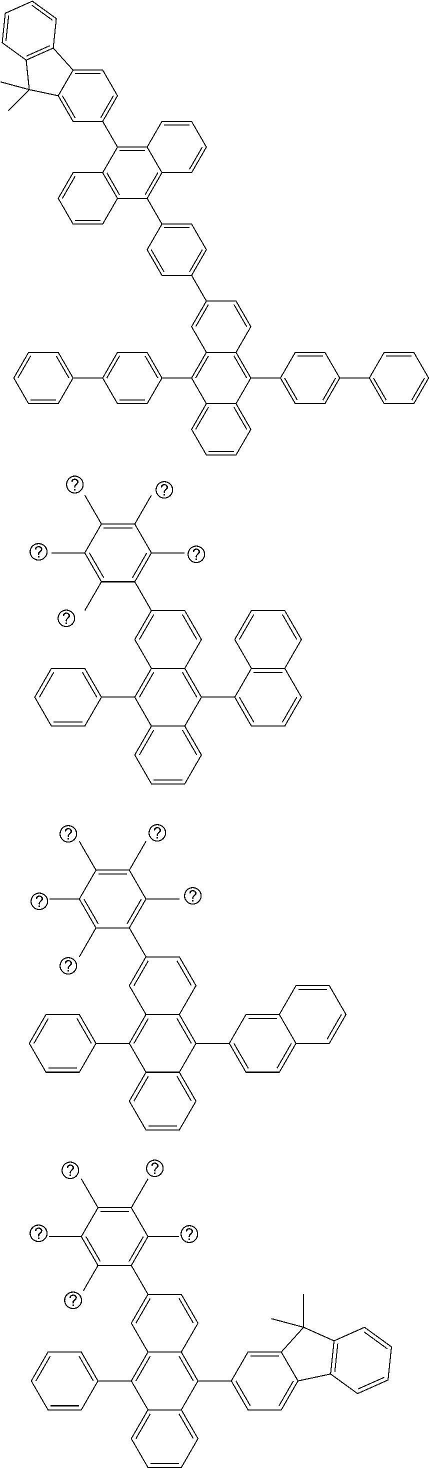

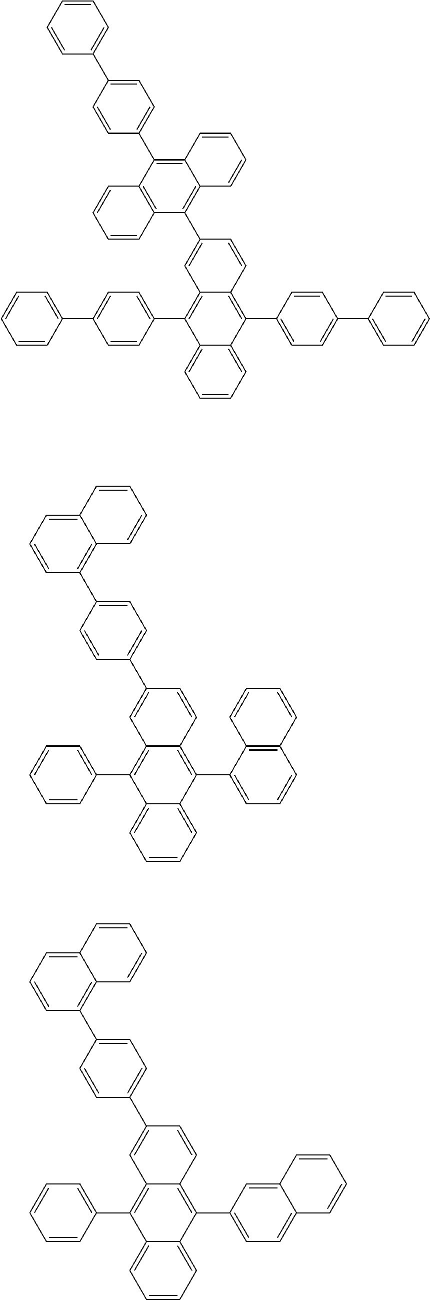

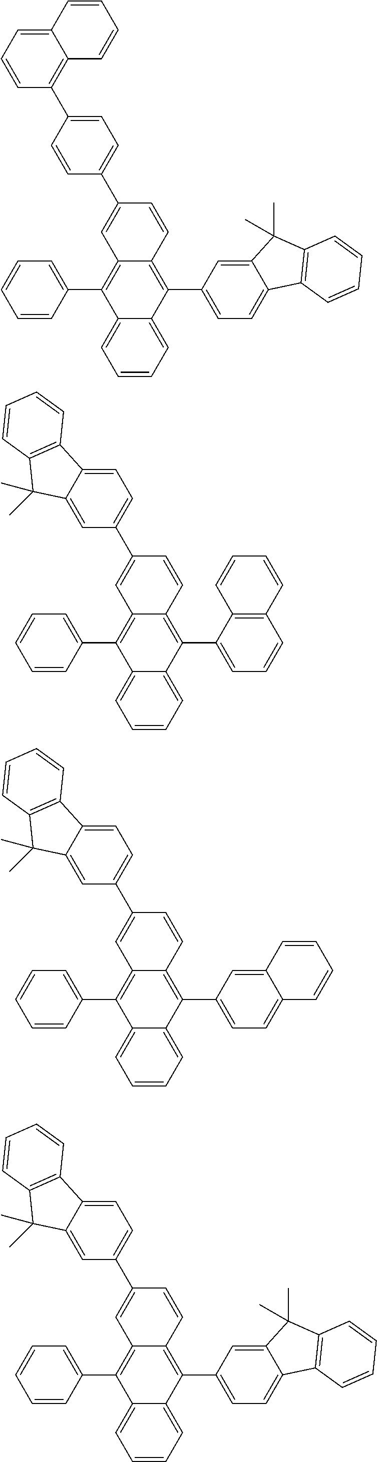

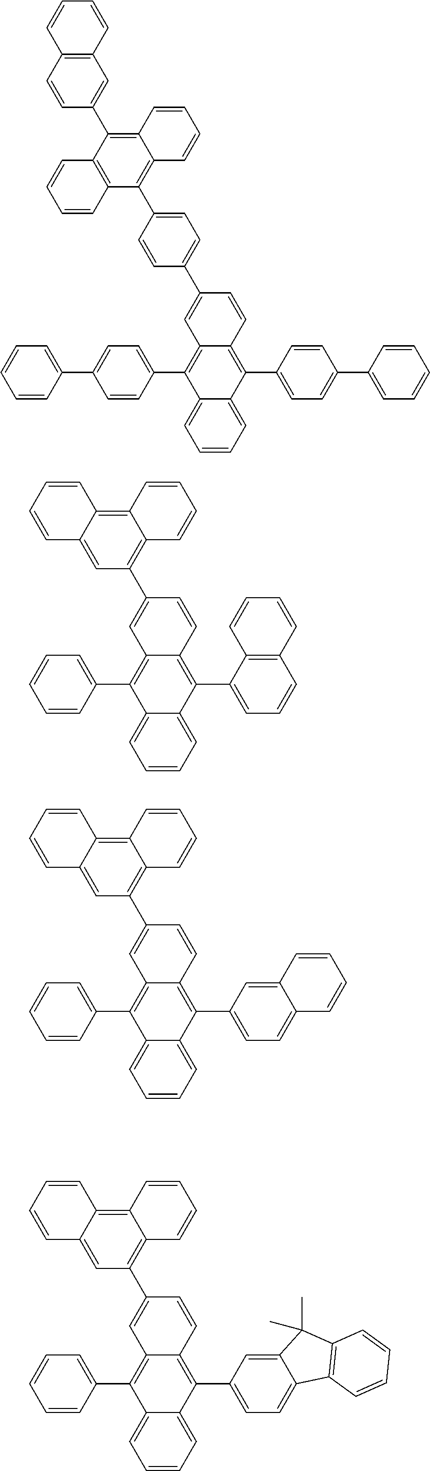

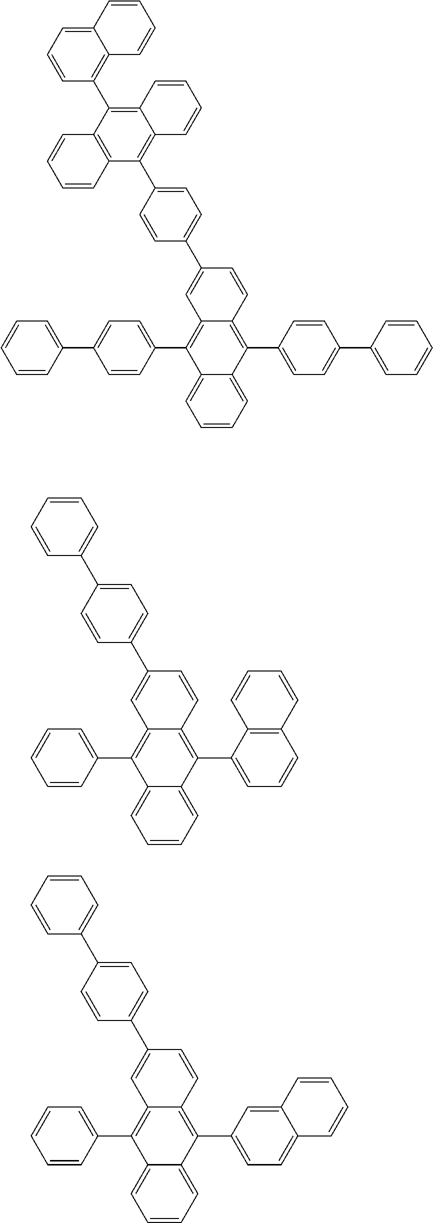

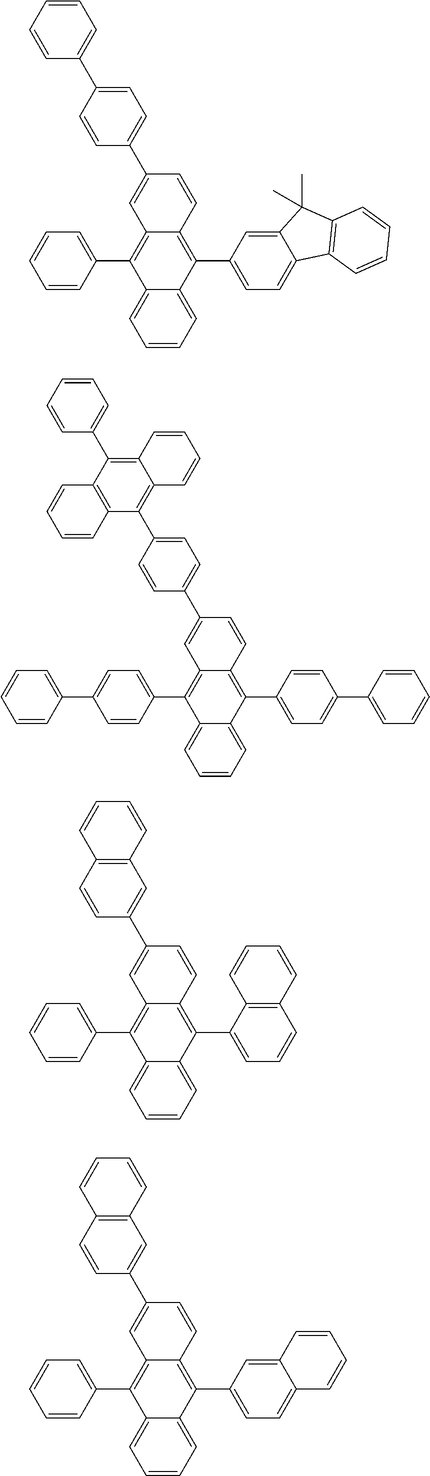

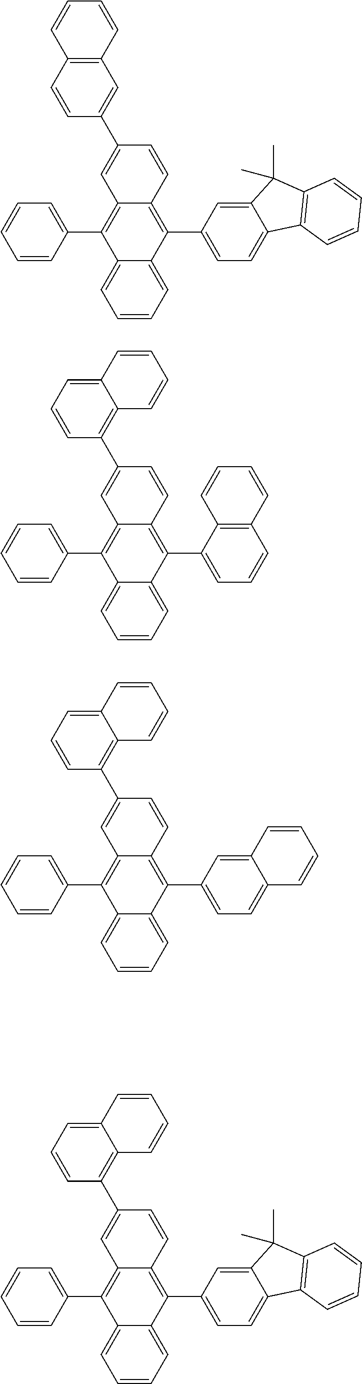

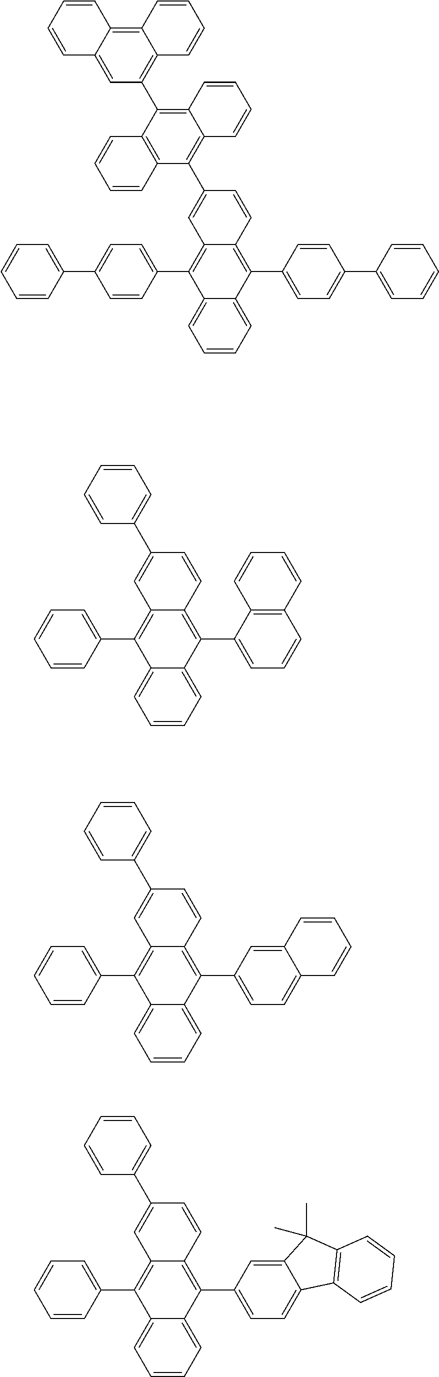

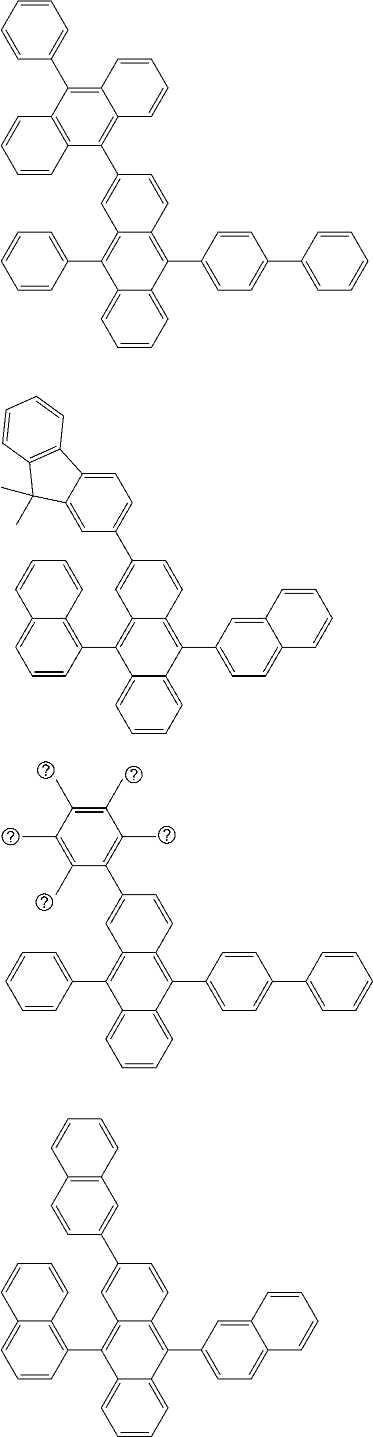

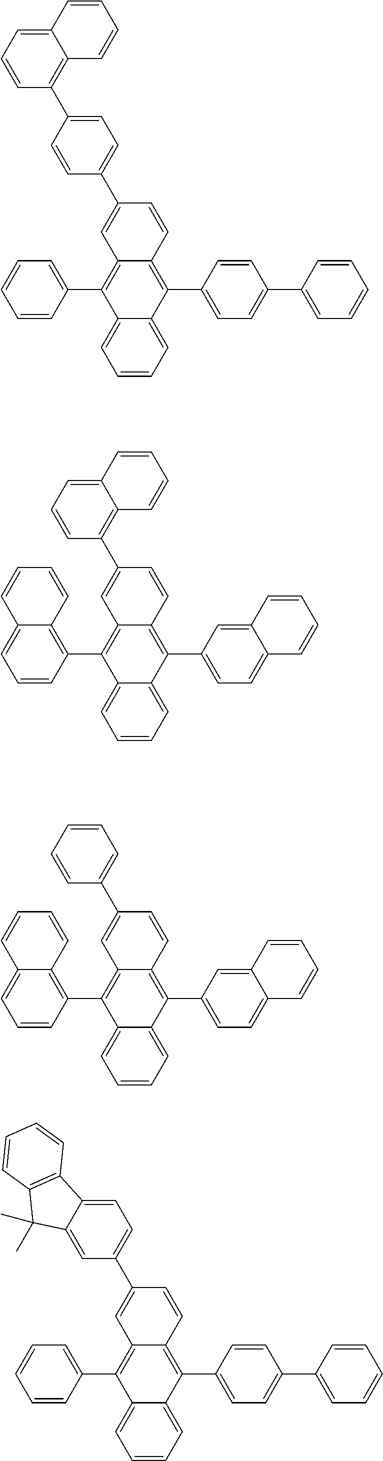

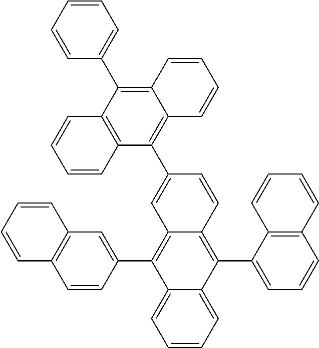 [SMILES: c1ccc(-c2c3ccccc3c(-c3ccc4c(-c5cccc6ccccc56)c5ccccc5c(-c5ccc6ccccc6c5)c4c3)c3ccccc23)cc1]

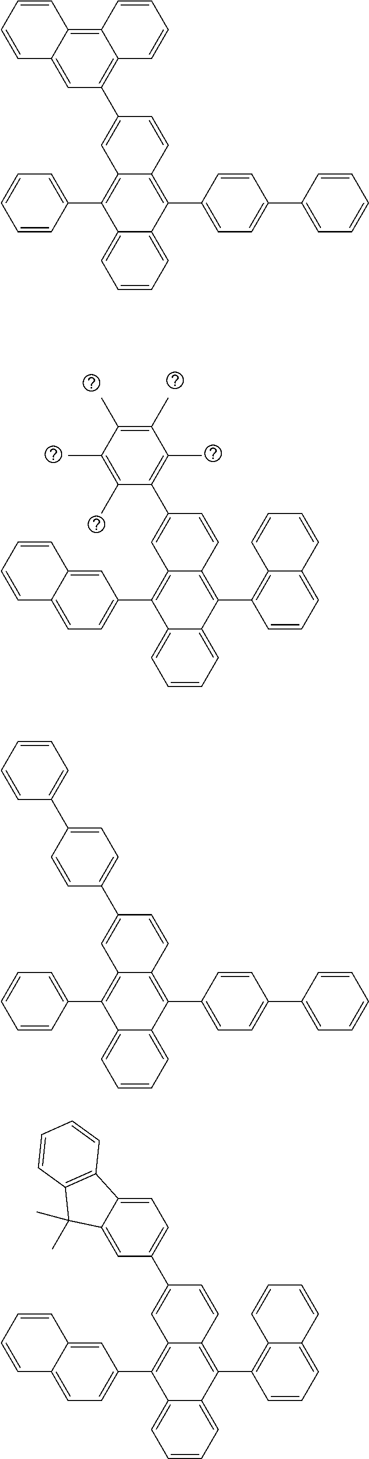

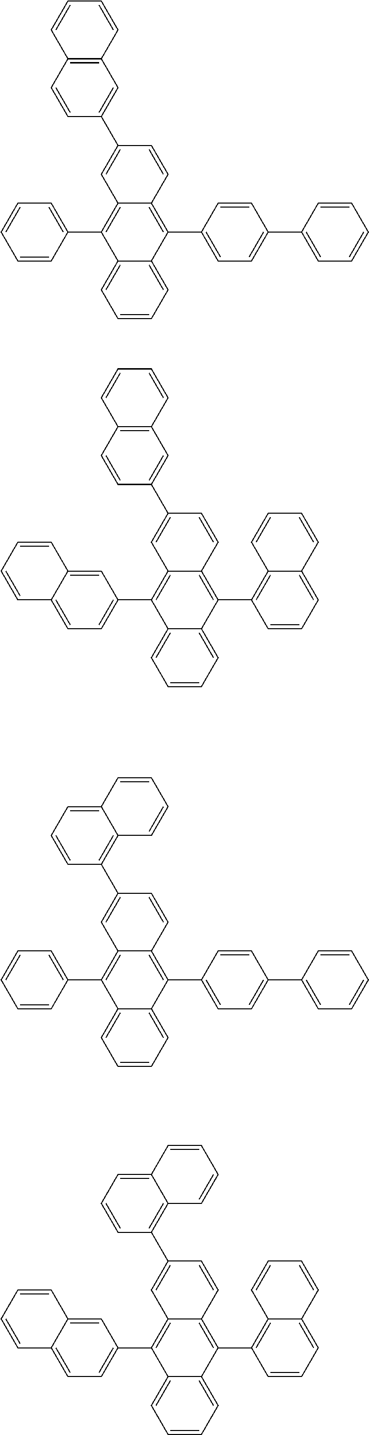

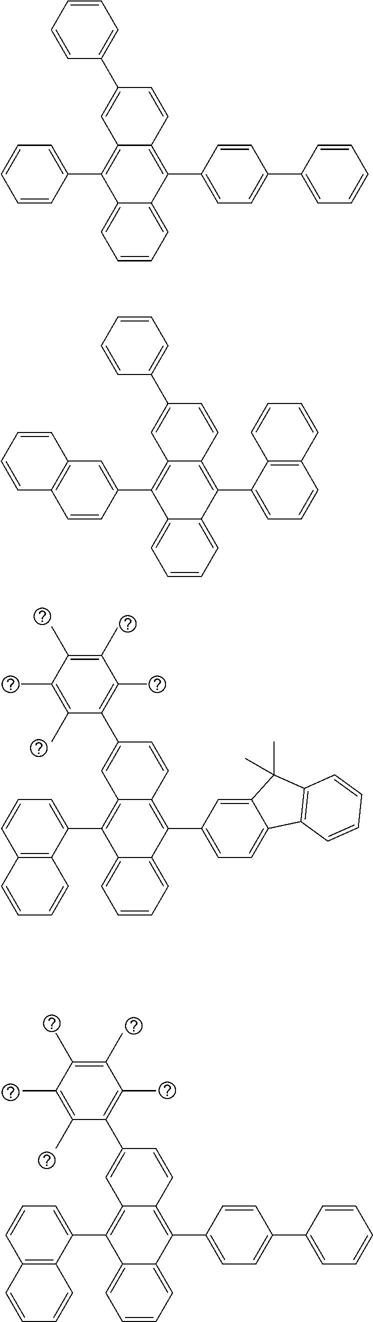

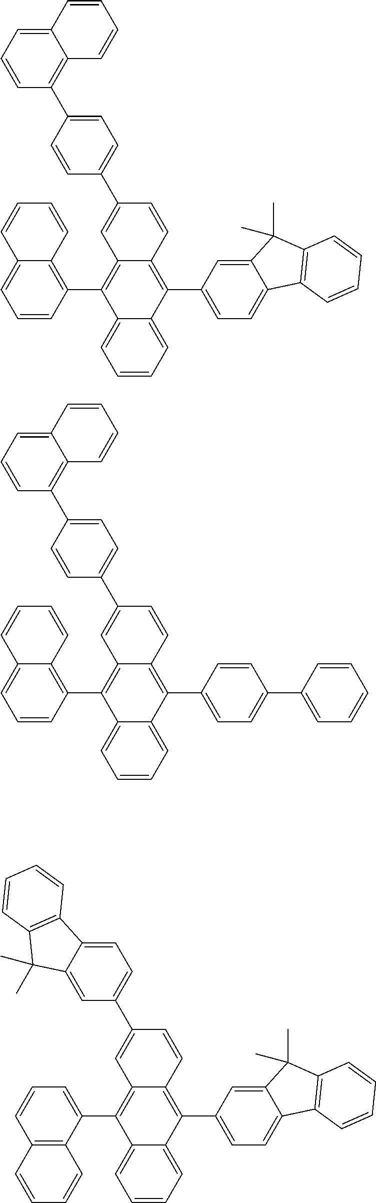

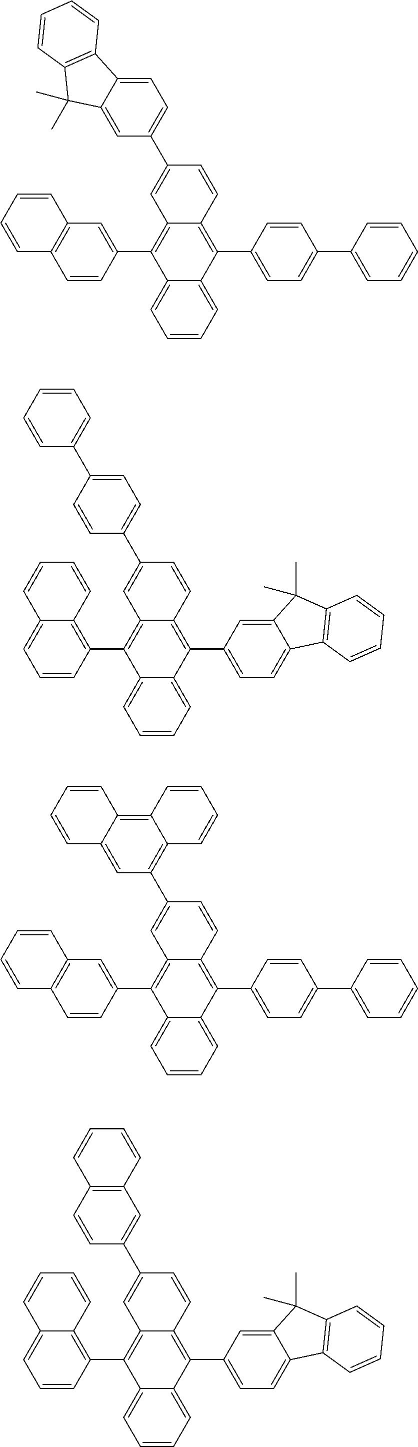

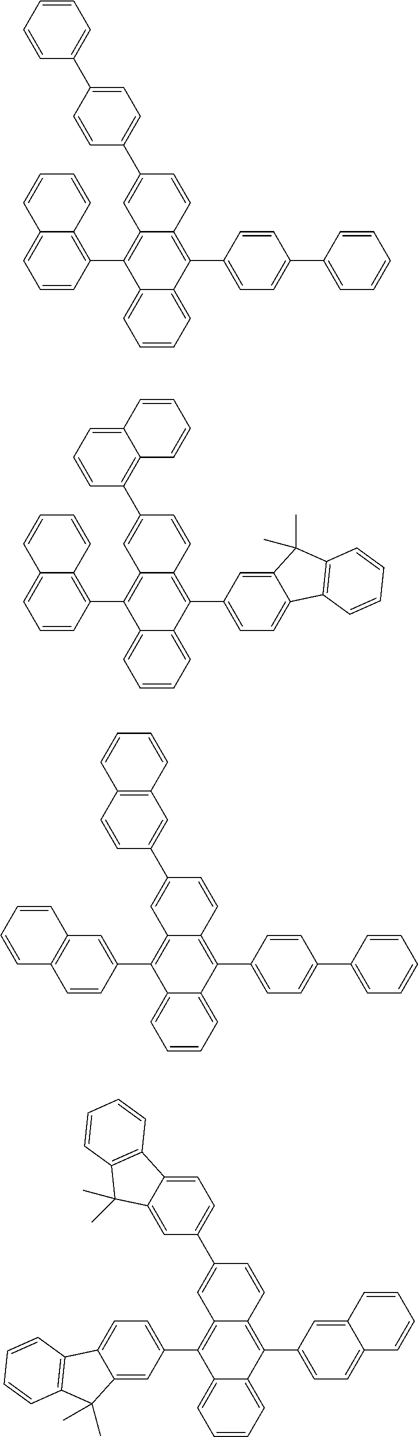

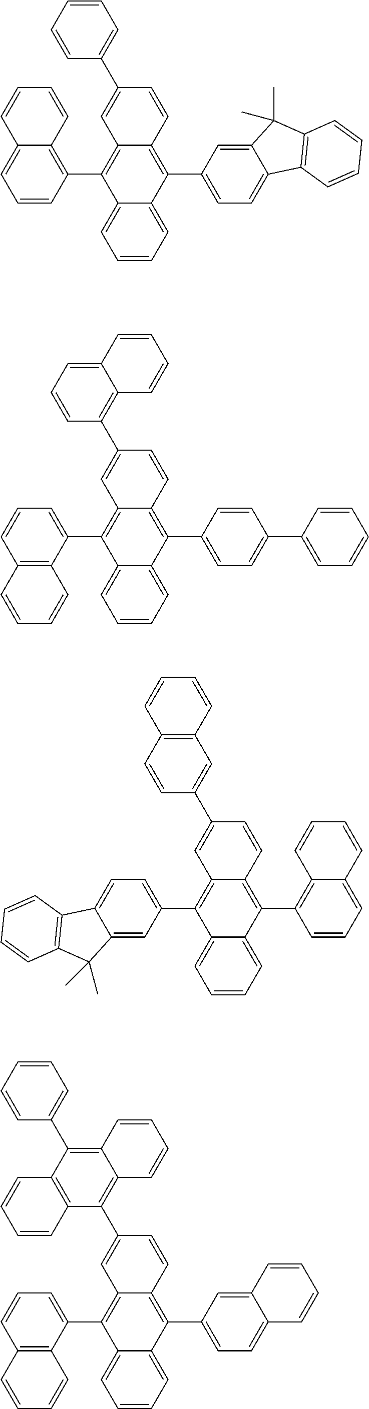

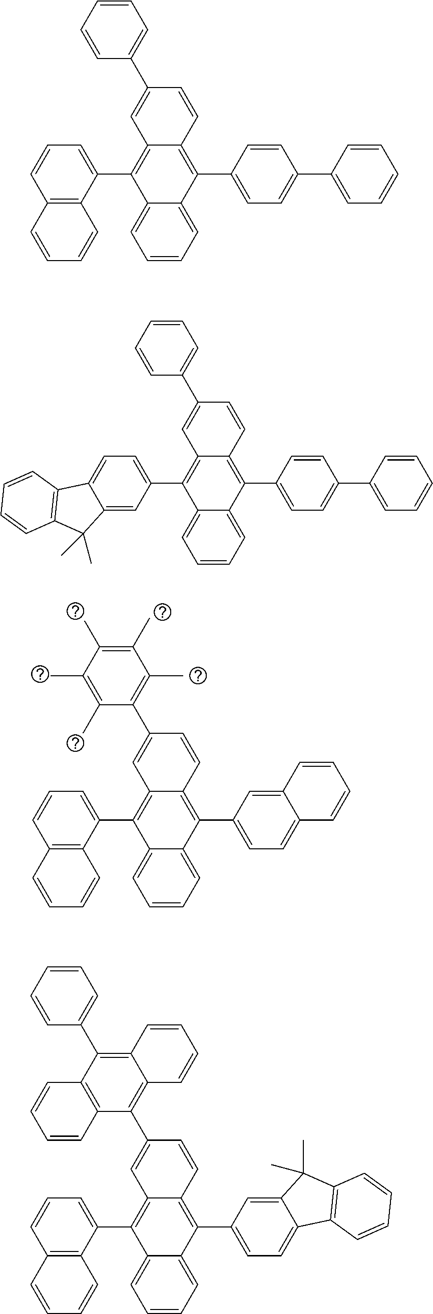

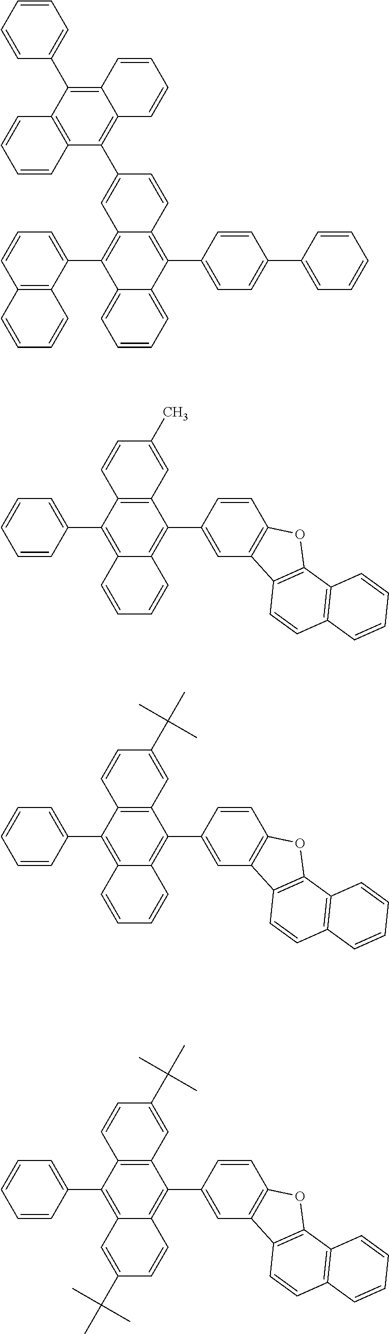

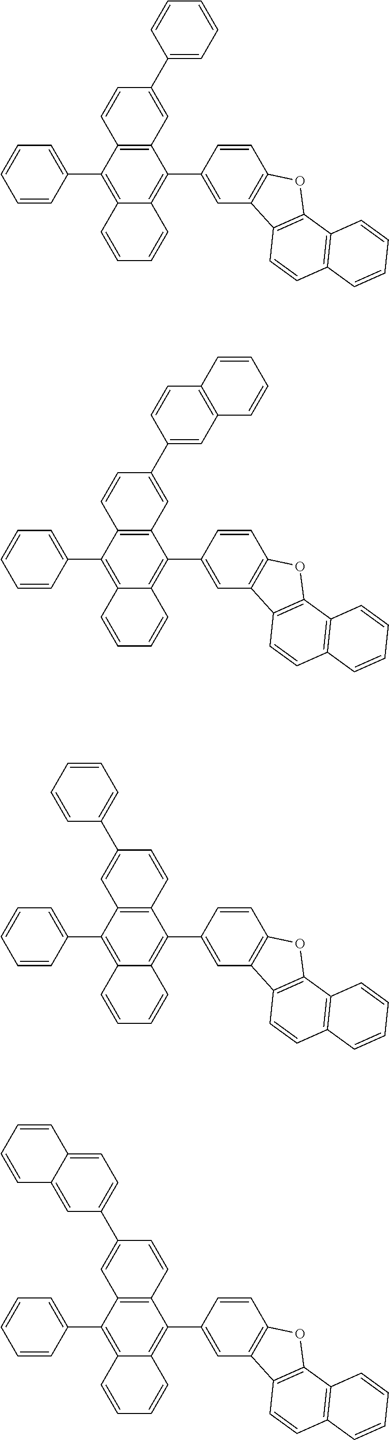 [SMILES: c1ccc(-c2ccc3c(-c4ccccc4)c4ccccc4c(-c4ccc5oc6c7ccccc7ccc6c5c4)c3c2)cc1 ; c1ccc(-c2c3ccccc3c(-c3ccc4oc5c6ccccc6ccc5c4c3)c3cc(-c4ccc5ccccc5c4)ccc23)cc1 ; c1ccc(-c2ccc3c(-c4ccc5oc6c7ccccc7ccc6c5c4)c4ccccc4c(-c4ccccc4)c3c2)cc1 ; c1ccc(-c2c3ccccc3c(-c3ccc4oc5c6ccccc6ccc5c4c3)c3ccc(-c4ccc5ccccc5c4)cc23)cc1]

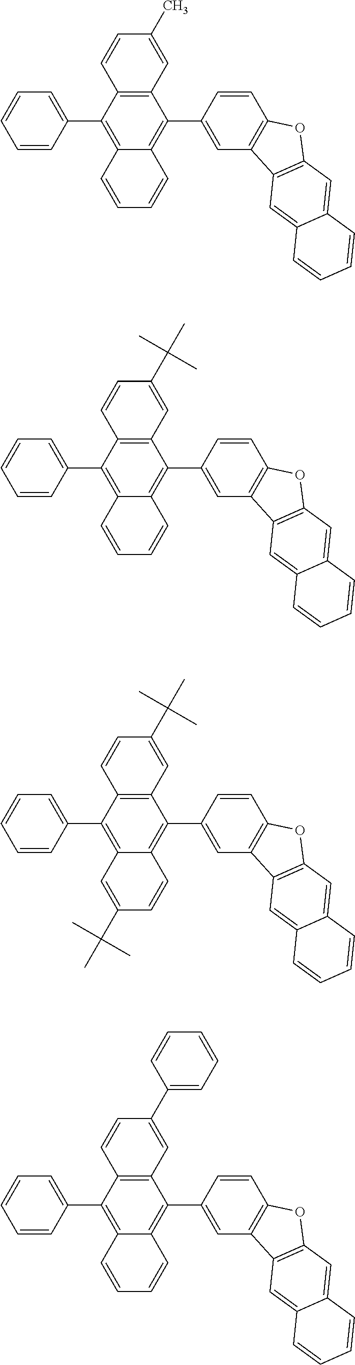 [SMILES: Cc1ccc2c(-c3ccccc3)c3ccccc3c(-c3ccc4oc5cc6ccccc6cc5c4c3)c2c1 ; CC(C)(C)c1ccc2c(-c3ccccc3)c3ccccc3c(-c3ccc4oc5cc6ccccc6cc5c4c3)c2c1 ; CC(C)(C)c1ccc2c(-c3ccc4oc5cc6ccccc6cc5c4c3)c3cc(C(C)(C)C)ccc3c(-c3ccccc3)c2c1 ; c1ccc(-c2ccc3c(-c4ccccc4)c4ccccc4c(-c4ccc5oc6cc7ccccc7cc6c5c4)c3c2)cc1]

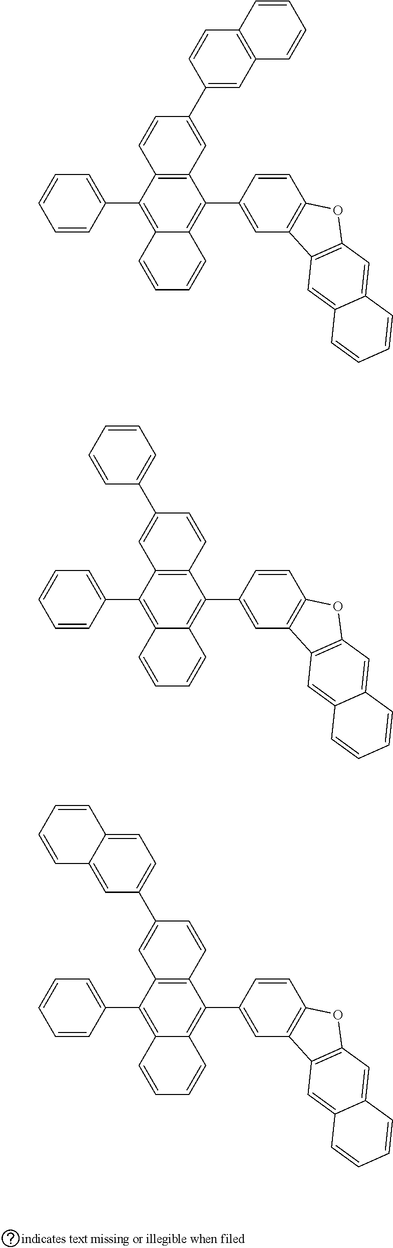 [SMILES: c1ccc(-c2c3ccccc3c(-c3ccc4oc5cc6ccccc6cc5c4c3)c3cc(-c4ccc5ccccc5c4)ccc23)cc1 ; c1ccc(-c2ccc3c(-c4ccc5oc6cc7ccccc7cc6c5c4)c4ccccc4c(-c4ccccc4)c3c2)cc1 ; c1ccc(-c2c3ccccc3c(-c3ccc4oc5cc6ccccc6cc5c4c3)c3ccc(-c4ccc5ccccc5c4)cc23)cc1]

The layered structure of the organic EL device in an aspect of the invention will be described below.

The organic EL device comprises an organic layer between a pair of electrodes consisting of a cathode and an anode. The organic layer comprises at least one layer formed from an organic compound. The organic layer may be a stack of two or more layers each formed from an organic compound. The organic layer may include an inorganic compound in addition to an organic compound.

In an embodiment of the invention, at least one layer of the organic layer is a light emitting layer.

In an embodiment of the invention, the light emitting layer comprises the compound represented by formula (1-1) and the compound represented by formula (10), wherein the content of the compound represented by formula (1-1) is 1% by mass or more and 20% by mass or less based on the total amount of the light emitting layer.

In an embodiment of the invention, the light emitting layer comprises the compound represented by formula (1-1) and the compound represented by formula (10), wherein the content of the compound represented by formula (10) is 80% by mass or more and 99% by mass or less based on the total amount of the light emitting layer.

The organic layer may be a single light emitting layer or additionally include another layer that is employed in an organic EL device, for example, a hole transporting region (a hole transporting layer, a hole injecting layer, an electron blocking layer, an exciton blocking layer, etc.) formed between an anode and a light emitting layer, a light emitting layer, a space layer, and an electron transporting region (an electron transporting layer, an electron injecting layer, a hole blocking layer, etc.) formed between a cathode and a light emitting layer, although not particularly limited thereto.

The host material other than the anthracene derivative may include, for example, a metal complex, such as an aluminum complex, a beryllium complex, and a zinc complex; a heterocyclic compound, such as an indole derivative, a pyridine derivative, a pyrimidine derivative, a triazine derivative, a quinoline derivative, an isoquinoline derivative, a quinazoline derivative, a dibenzofuran derivative, a dibenzothiophene derivative, an oxadiazole derivative, a benzimidazole derivative, and a phenanthroline derivative; a fused aromatic compound, such as a naphthalene derivative, a triphenylene derivative, a carbazole derivative, a phenanthrene derivative, a pyrene derivative, a chrysene derivative, a naphthacene derivative, and a fluoranthene derivative; and an aromatic amine compound, such as a triarylamine derivative and a fused aromatic polycyclic amine derivative. These host materials may be used in combination of two or more.

Examples of the metal complex include tris(8-quinolinolato)aluminum(III) (Alq), tris(4-methyl-8-quinolinolato)aluminum(III) (Almq₃), bis(10-hydroxybenzo[h]quinolinato)beryllium(II) (BeBq2), bis(2-methyl-8-quinolinolato)(4-phenylphenolato)aluminum(III) (BAlq), bis(8-quinolinolato)zinc(II) (Znq), bis[2-(2-benzoxazolyl)phenolato]zinc(II) (ZnPBO), and bis[2-(2-benzothiazolyl)phenolato]zinc(II) (ZnBTZ).

Examples of the heterocyclic compound include 2-(4-biphenylyl)-5-(4-tert-butylphenyl)-1,3,4-oxadiazole (PBD), 1,3-bis[5-(p-tert-butylphenyl)-1,3,4-oxadiazole-2-yl]benzene (OXD-7), 3-(4-biphenylyl)-4-phenyl-5-(4-tert-butylphenyl)-1,2,4-triazole (TAZ), 2,2′,2″-(1,3,5-benzenetriyl)tris(1-phenyl-1H-benzimidazole) (TPBI), bathophenanthroline (BPhen), and bathocuproin (BCP).

Examples of the fused aromatic compound include 9-[4-(10-phenyl-9-anthryl)phenyl]-9H-carbazole (CzPA), 3,6-diphenyl-9-[4-(10-phenyl-9-anthryl)phenyl]-9H-carbazole (DPCzPA), 9,10-bis(3,5-diphenylphenyl)anthracene (DPPA), 9,10-di(2-naphthyl)anthracene (DNA), 2-tert-butyl-9,10-di(2-naphthyl)anthracene (t-BuDNA), 9,9′-bianthryl (BANT), 9,9′-(stilbene-3,3′-diyl)diphenanthrene (DPNS), 9,9′-(stilbene-4,4′-diyl)diphenanthrene (DPNS2), 3,3′,3″-(benzene-1,3,5-triyl)tripyrene (TPB3), 9,10-diphenylanthracene (DPAnth), and 6,12-dimethoxy-5,11-diphenylchrysene.

Examples of the aromatic amine compound include N,N-diphenyl-9-[4-(10-phenyl-9-anthryl)phenyl]-9H-carbazole-3-amine (CzA1PA), 4-(10-phenyl-9-anthryl)triphenylamine (DPhPA), N,9-diphenyl-N-[4-(10-phenyl-9-anthryl)phenyl]-9H-carbazole-3-amine (PCAPA), N,9-diphenyl-N-{4-[4-(10-phenyl-9-anthryl)phenyl]phenyl}-9H-carbazole-3-amine (PCAPBA), N-(9,10-diphenyl-2-anthryl)-N,9-diphenyl-9H-carbazole-3-amine (2PCAPA), 4,4′-bis[N-(1-anthryl))-N-phenylamino]biphenyl (NPB or α-NPD), N,N′-bis(3-methylphenyl)-N,N′-diphenyl-[1,1′-biphenyl]-4,4′-diamine (TPD), 4,4′-bis[N-(99-dimethylfluorene-2-yl)-N-phenylamino]biphenyl (DFLDPBi), and 4,4′-bis[N-(spiro-9,9′-bifluorene-2-yl)-N phenylamino]biphenyl (BSPB).

A fluorescent host preferably has a singlet level higher than that of a fluorescent dopant and preferably selected from the heterocyclic compound and the fused aromatic compound, such as a pyrene compound, a chrysene compound and a naphthacene compound.

A phosphorescent host preferably has a triplet level higher than that of a phosphorescent dopant and preferably selected from the metal complex, the heterocyclic compound and the fused aromatic compound. Preferred are an indole derivative, a carbazole derivative, a pyridine derivative, a pyrimidine derivative, a triazine derivative, a quinoline derivative, an isoquinoline derivative, a quinazoline derivative, a dibenzofuran derivative, a dibenzothiophene derivative, a naphthalene derivative, a triphenylene derivative, a phenanthrene derivative, and a fluoranthene derivative.

### Electron Transporting Layer

The electron transporting layer is a layer comprising a material having a high electron transporting ability, preferably a material having an electron transporting ability of 10-6 cm²/Vs or more. Examples thereof include a metal complex, an aromatic heterocyclic compound, an aromatic hydrocarbon compound, and a polymer.

The metal complex is, for example, an aluminum complex, a beryllium complex, and a zinc complex. Examples thereof include tris(8-quinolinolato)aluminum (III) (Alq), tris(4-methyl-8-quinolinolato)aluminum (Almq₃), bis(10-hydroxybenzo[h]quinolinato)beryllium (BeBq2), bis(2-methyl-8-quinolinato)(4-phenylphenolato)aluminum (III) (BAlq), bis(8-quinolinato)zinc(II) (Znq), bis[2-(2-benzoxazolyl)phenolato]zinc(II) (ZnPBO), and bis[2-(2-benzothiazolyl)phenolato]zinc(II) (ZnBTZ).

The aromatic heterocyclic compound is, for example, an imidazole derivative, such as a benzimidazole derivative, an imidazopyridine derivative, and a benzimidazophenanthridine derivative; an azine derivative, such as a pyrimidine derivative and a triazine derivative; or a compound having a nitrogen-containing six-membered ring (inclusive of a compound having a phosphine oxide substituent on the heterocyclic ring), such as a quinoline derivative, an isoquinoline derivative, and a phenanthroline derivative. Examples thereof include 2-(4-biphenylyl)-5-(4-tert-butylphenyl)-1,3,4-oxadiazole (PBD), 1,3-bis[5-(p-tert-butylphenyl)-1,3,4-oxadiazole-2-yl]benzene (OXD-7), 3-(4-tert-butylphenyl)-4-phenyl-5-(4-biphenylyl)-1,2,4-triazole (TAZ), 3-(4-tert-butylphenyl)-4-(4-ethylphenyl)-5-(4-biphenylyl)-1,2,4-triazole (p-EtTAZ), bathophenanthroline (BPhen), bathocuproine (BCP), and 4,4′-bis(5-methylbenzoxazole-2-yl)stilbene (BzOs).

Examples of the aromatic hydrocarbon compound include an anthracene derivative and a fluoranthene derivative.

Examples of the polymer include poly[(9,9-dihexylfluorene-2,7-diyl)-co-(pyridine-3,5-diyl)] (PF-Py), and poly[(9,9-dioctylfluorene-2,7-diyl)-co-(2,2′-bipyridine-6,6′-diyl)] (PF-BPy).

A compound other than those mentioned above is also usable in the electron transporting layer if its electron transporting ability is higher than its hole transporting ability.

The electron transporting layer may be a single layer or a multi-layer of two or more layers. In a multi-layered electron transporting layer, the layer close to a light emitting layer preferably comprises a material having a high electron transporting ability and a larger energy gap.

For example, as shown in FIG. 2, the electron transporting layer may be made into a structure having an anode-side first electron transporting layer and a cathode-side second electron transporting layer.

The electron transporting layer may include, for example, a metal, such as an alkali metal, magnesium, an alkaline earth metal, and an alloy comprising two or more metals selected from the preceding metals, or a metal compound, such as an alkali metal compound, for example, 8-quinolinolatoaluminum (Liq), and an alkaline earth metal compound.

The content of the metal such as an alkali metal, magnesium, an alkaline earth metal, and an alloy comprising two or more metals selected from the preceding metals to be used in the electron transporting layer is preferably 0.1 to 50% by mass, more preferably 0.1 to 20% by mass, and still more preferably 1 to 10% by mass, although not limited thereto.

The content of the metal compound such as an alkali metal compound and an alkaline earth metal compound to be used in the electron transporting layer is preferably 1 to 99% by mass and more preferably 10 to 90% by mass.

When the electron transporting layer is a multi-layered structure, the electron transporting layer close to the light emitting layer may be formed only by the metal compound mentioned above.

### Electron Injecting Layer

The electron injecting layer is a layer comprising a material having a high electron injecting ability and has a function of effectively injecting electrons from the cathode to the light emitting layer. The material having a high electron injecting ability may be, for example, an alkali metal, magnesium, an alkaline earth metal, and a compound thereof. Examples thereof include lithium, cesium, calcium, lithium fluoride, cesium fluoride, calcium fluoride, and lithium oxide. In addition, an electron transporting material that is doped with an alkali metal, magnesium, an alkaline earth metal, or a compound thereof, for example, Alq doped with magnesium (Mg), is also usable.

A composite material comprising an organic compound and an electron donor is also usable in the electron injecting layer. Such a composite material is excellent in the electron injecting ability and the electron transporting ability, because the organic compound receives electrons from the electron donor.

The organic compound is preferably a compound excellent in transporting the received electrons. For example, the metal complex and the aromatic heterocyclic compound mentioned above with respect to the material having a high electron transporting ability are usable as such an organic compound.

Any compound capable of giving its electron to the organic compound is usable as the electron donor. Preferred examples thereof are an alkali metal, magnesium, an alkaline earth metal, and a rare earth metal, such as lithium, cesium, magnesium, calcium, erbium, and ytterbium; an alkali metal oxide and an alkaline earth metal oxide, such as lithium oxide, calcium oxide, and barium oxide; a Lewis base, such as magnesium oxide; and an organic compound, such as tetrathiafulvalene (TTF).

### Cathode

The cathode is formed preferably from a metal, an alloy, an electrically conductive compound, or a mixture thereof, each having a small work function, for example, a work function of 3.8 eV or less. Examples of the material for the cathode include an alkali metal, such as lithium and cesium, magnesium, an alkaline earth metal, such as calcium and strontium, an alloy containing these metals (for example, magnesium-silver and aluminum-lithium), a rare earth metal, such as europium and ytterbium, and an alloy containing a rare earth metal.

The cathode is generally formed by a vacuum vapor deposition or a sputtering method. A coating method and an inkjet method are usable when a silver paste is used.

When the electron injecting layer is formed, the material for the cathode is selected irrespective of whether the work function is large or small and various electroconductive materials, such as aluminum, silver, ITO, graphene, and indium oxide-tin oxide doped with silicon or silicon oxide, are usable. These electroconductive materials are made into films by a sputtering method, an inkjet method, and a spin coating method.

### Insulating Layer

Since electric field is applied to thin films of organic EL devices, the pixel defects due to leak and short circuit tends to occur. To prevent the defects, an insulating thin film layer may be interposed between the pair of electrodes.

Examples of the material for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. These materials may be used in combination. The insulating layer may be a multi-layered structure of layers each containing the above material.

### Space Layer

For example, in an organic EL device in which a fluorescent emitting layer and a phosphorescent emitting layer are stacked, a space layer is disposed between the fluorescent emitting layer and the phosphorescent emitting layer to prevent the diffusion of excitons generated in the phosphorescent emitting layer to the fluorescent emitting layer or to control the carrier balance. The space layer may be disposed between two or more phosphorescent emitting layers.

Since the space layer is disposed between the light emitting layers, a material combining the electron transporting ability and the hole transporting ability is preferably used for forming the space layer. To prevent the diffusion of triplet energy in the adjacent phosphorescent emitting layer, the triplet energy of the material for the space layer is preferably 2.6 eV or more.

The materials described above with respect to the hole transporting layer are usable as the material for the space layer.

### Electron Blocking Layer, Hole Blocking Layer and Exciton Blocking Layer

An electron blocking layer, a hole blocking layer, and an exciton (triplet) blocking layer may be provided in contact with the light emitting layer.

The electron blocking layer is a layer having a function of preventing the diffusion of electrons from the light emitting layer to the hole transporting layer. The hole blocking layer is a layer having a function of preventing the diffusion of holes from the light emitting layer to the electron transporting layer. The exciton blocking layer is a layer having a function of preventing the diffusion of excitons generated in the light emitting layer to adjacent layers and confining the excitons in the light emitting layer.

### Method for Forming Layers

The method for forming each layer of the organic EL device is not particularly limited, unless otherwise noted.

The forming method may be a known method, such as a dry film forming method and a wet film forming method. Examples of the dry film forming method include a vacuum vapor deposition method, a sputtering method, a plasma method, and an ion plating method. Examples of the wet film forming method include a spin coating method, a dipping method, a flow coating method, and an inkjet method.

### Thickness

The thickness of each layer of the organic EL device is not particularly limited, unless otherwise noted. A thickness excessively small may be easily cause defects such as pin holes and a sufficient luminance is not obtained. A thickness excessively large requires a high driving voltage to reduce the efficiency. Therefore, the thickness is preferably 5 nm to 10 μm and more preferably 10 nm to 0.2 μm.

### Electronic Device

The electronic device of the invention comprises the organic EL device described above. Examples of the electronic device include a display part, such as organic EL panel module, a display device of television set, mobile phone, smart phone, personal computer, etc., and a light emitting source of lighting equipment and vehicle lighting equipment.

### EXAMPLES

An embodiment of the invention will be described below in more details with reference to the examples. However, it should be noted that the scope of the invention is not limited thereto.

### Synthesis Example 1: Synthesis of Compound 1

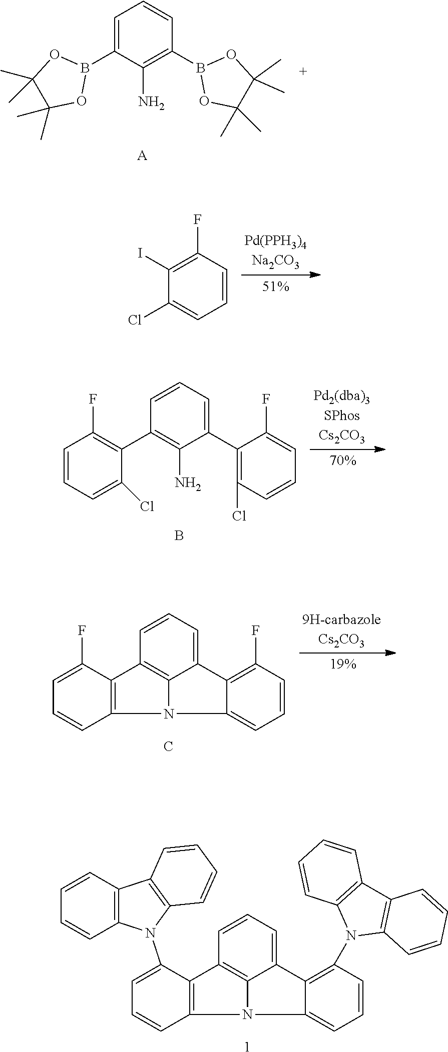

### (1) Synthesis of Compound B

Under argon atmosphere, into a suspension of chlorofluoroiodobenzene (4.04 g, 15.8 mmol, 2.1 eq), diboronic acid ester A (2.59 g, 7.5 mmol), and Pd(PPh₃)₄ (0.347 g, 0.300 mmol, 4% Pd) in dioxane (75 mL), a 2 M Na₂CO₃ aqueous solution (11.3 mL, 22.5 mmol) was added. The resultant mixture was refluxed for 8 h. The reaction mixture was extracted with dichloromethane and the solvent was evaporated off. The residue was dried under reduced pressure and purified by column chromatography to obtain a white solid (1.34 g, yield of 51%), which was identified as the aimed compound B by the result of mass spectrometric analysis (m/e=350 to the molecular weight of 350.19).

### (2) Synthesis of Compound C

Under argon atmosphere, a suspension of the compound B (0.61 g, 6.44 mmol), Pd₂(dba)₃ (64 mg, 0.070 mmol, 4% Pd), SPhos (120 mg, 0.293 mmol), and Cs₂CO₃ (3.41 g, 10.5 mmol) in anhydrous xylene (35 mL) was refluxed for 2 days. The reaction mixture was filtered. The residue was washed with water and methanol and dried under reduced pressure to obtain a dark yellow solid. The obtained solid was purified by column chromatography to obtain a white solid (0.34 g, yield of 70%), which was identified as the aimed compound C by the result of mass spectrometric analysis (m/e=277 to the molecular weight of 277.27).

### (3) Synthesis of Compound 1

Under argon atmosphere, a suspension of the compound C (50 mg, 0.180 mmol), 9H-carbazole (64 mg, 0.379 mmol), and Cs₂CO₃ (176 mg, 0.540 mmol) in DMF (15 mL) was refluxed for 10 h. The reaction mixture was extracted with dichloromethane and the solvent was evaporated off. The residue was dried under reduced pressure and purified by column chromatography to obtain a white solid (20 mg, yield of 19%), which was identified as the aimed compound 1 by the result of mass spectrometric analysis (m/e=571 to the molecular weight of 571.67).

### Synthesis Example 2: Synthesis of Compound 2

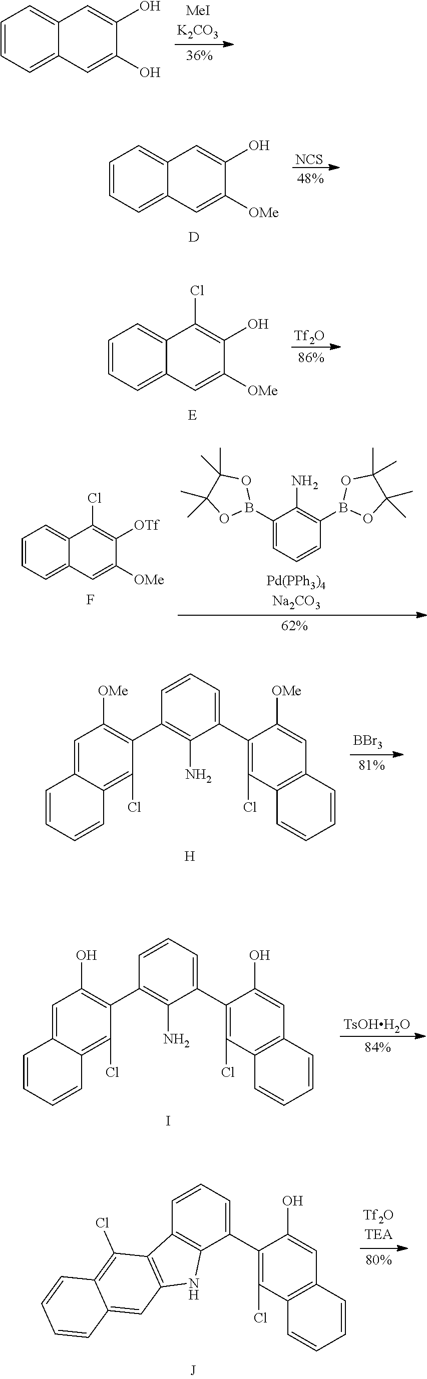

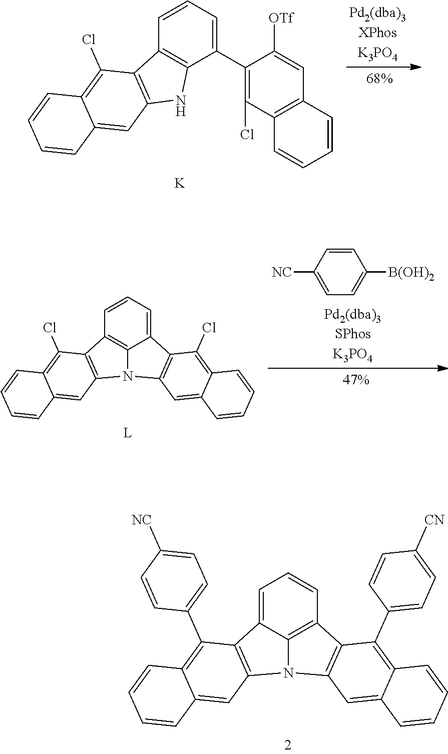

### (1) Synthesis of Compound D

Under argon atmosphere, a suspension of 2,3-naphthalenediol (475 g, 2.97 mol) and K₂CO₃ (410 g, 2.97 mol) in DMF (3 L) was stirred at 100° C. for 3 h. After allowing to cool, MeI (421 g, 2.97 mol) was added dropwise while continuing the stirring and the suspension was further stirred at room temperature for 12 h. After the reaction, the reaction mixture was extracted by adding water and ethyl acetate. The organic layer was concentrated to obtain a black oil (904 g). The obtained oil was purified by column chromatography and washed with heptane to obtain a white solid (184 g, yield of 36%), which was identified as the aimed compound D by the result of mass spectrometric analysis (m/e=174 to the molecular weight of 174.2).

### (2) Synthesis of Compound E

Under argon atmosphere, into a solution of the compound D (174 g, 1 mol) in acetonitrile (1.7 L), p-toluenesulfonic acid monohydrate (190 g, 1 mol) was added. After adding N-chlorosuccinimide (133 g, 1 mol), the mixture was stirred at room temperature for 12 h. After the reaction, the reaction mixture was extracted by adding water and ethyl acetate. The organic layer was concentrated to obtain an orange oil (904 g). The obtained oil was purified by column chromatography and washed with heptane and toluene to obtain a white solid (99 g, yield of 48%), which was identified as the aimed compound E by the result of mass spectrometric analysis (m/e=208 to the molecular weight of 208.6).

### (3) Synthesis of Compound F

Under argon atmosphere, into a solution of the compound E (99 g, 474 mmol) in chloroform (940 mL), triethylamine (79 mL, 569 mmol) was added and the resultant solution was cooled to 0° C. After adding anhydrous triflate (147 g, 522 mmol) dropwise while keeping the temperature at 0° C., the solution was stirred at room temperature for 3 h. After the reaction, the solvent was evaporated off. The residue was dried under reduced pressure to obtain a red oil (173 g). The obtained oil was purified by column chromatography to obtain a white solid (139 g, yield of 86%), which was identified as the aimed compound F by the result of mass spectrometric analysis (m/e=340 to the molecular weight of 340.7).

### (4) Synthesis of Compound H

Under argon atmosphere, into a suspension of the compound F (135 g, 396 mmol, 2.2 eq), boronic acid G (65 g, 188 mmol), Pd(PPh₃)₄ (10.9 g, 9.42 mmol, 5% Pd), and Na₂CO₃ (79.9 g, 4 eq) in 1,2-dimethoxyethane (2 L), H₂O (380 mL) was added and the resultant suspension was stirred for three days at 78° C. The reaction mixture was extracted with toluene and the extract was dried over MgSO₄. The solvent was evaporated off and the residue was dried under reduced pressure to obtain a black oil. The obtained oil was purified by column chromatography to obtain a white solid (56 g, yield of 62%), which was identified as the aimed compound H by the result of mass spectrometric analysis (m/e=474 to the molecular weight of 474.3).

### (5) Synthesis of Compound I

Under argon atmosphere, a solution of the compound H (99 g, 474 mmol) in dichloromethane (940 mL) was cooled to 0° C. BBr₃ (147 g, 522 mmol) was added dropwise while keeping the temperature at 0° C. and the resultant solution was stirred at room temperature for 12 h. After the reaction, H₂O was added dropwise. The precipitated solid was collected by filtration and washed with ethyl acetate to obtain a white solid (63 g, yield of 81%), which was identified as the aimed compound I by the result of mass spectrometric analysis (m/e=446 to the molecular weight of 446.3).

### (6) Synthesis of Compound J

Under argon atmosphere, a solution of the compound I (62 g, 139 mmol) and p-toluenesulfonic acid monohydrate (2.64 g, 13.9 mmol) in xylene (1.2 L) was stirred at 140° C. for 2 h. After the reaction, the obtained product was purified by column chromatography to obtain a white solid (50 g, yield of 84%), which was identified as the aimed compound J by the result of mass spectrometric analysis (m/e=428 to the molecular weight of 428.3).

### (7) Synthesis of Compound K

Under argon atmosphere, into a solution of the compound J (48 g, 112 mmol) in chloroform (500 mL), triethylamine (23.4 mL, 168 mmol) was added and the resultant solution was cooled to 0° C. After adding anhydrous triflate (33 g, 157 mmol) dropwise while keeping the temperature at 0° C., the solution was stirred at room temperature for 12 h. After the reaction, the obtained product was purified by column chromatography to obtain a white solid (50 g, yield of 80%), which was identified as the aimed compound K by the result of mass spectrometric analysis (m/e=560 to the molecular weight of 560.3).

### (8) Synthesis of Compound L

Under argon atmosphere, a suspension of the compound K (40.0 g, 71.4 mmol), Pd₂(dba)₃ (3.92 g, 4.28 mmol, 6% Pd), XPhos (4.08 g, 8.57 mmol), and K₃PO₄ (45.5 g, 214 mmol) in anhydrous xylene (860 mL) was refluxed for 4 h. After allowing to cool, the precipitated solid was collected by filtration, purified by silica gel column chromatography, and suspended in xylene for washing under heating to obtain a yellow solid (20 g, yield of 68%), which was identified as the aimed compound L by the result of mass spectrometric analysis (m/e=410 to the molecular weight of 410.3).

### (9) Synthesis of Compound 2

Under argon atmosphere, a suspension of 4-cyanophenylboronic acid (6.45 g, 43.9 mmol), the compound L (3.00 g, 7.31 mmol), Pd₂(dba)₃ (536 mg, 0.585 mmol), SPhos (961 mg, 2.34 mmol), and K₃PO₄ (31 g, 146 mmol) in DMF (360 mL) was stirred at 100° C. for 3.5 h. After the reaction, the solvent was evaporated off. The residue was purified by silica gel column chromatography to obtain a yellow solid (1.86 g, yield of 47%), which was identified as the aimed compound 2 by the result of mass spectrometric analysis (m/e=543 to the molecular weight of 543.6).

### Synthesis Example 3: Synthesis of Compound 3

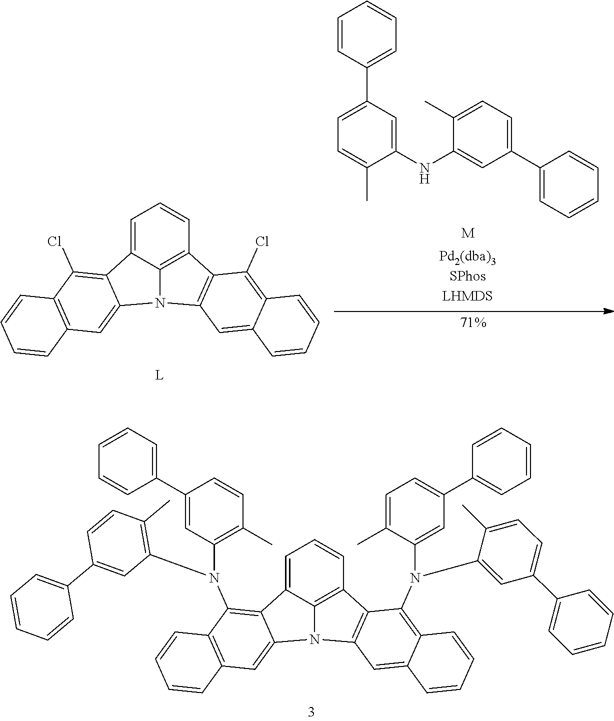

Under argon atmosphere, a suspension of the compound L (1.00 g, 2.44 mmol), amine M (4.26 g, 12.2 mmol), Pd₂(dba)₃ (0.045 g, 0.049 mmol), and SPhos (0.080 g, 0.195 mmol) in toluene (120 mL) was heated to 100° C. under stirring. Thereafter, a 1 M lithium hexamethyldisilazide/THF solution (12.2 mL, 12.2 mmol) was added and the suspension was refluxed for 5 h. The reaction mixture was filtered through celite and the solvent was evaporated off. The residue was dried under reduced pressure and purified by column chromatography to obtain a yellow solid (1.78 g, yield of 71%), which was identified as the aimed compound 3 by the result of mass spectrometric analysis (m/e=1036 to the molecular weight of 1036.31).

### Examples 1 to 3

### Measurement of Absorption Peak Wavelength

Each of the compounds 1 to 3 obtained in Synthesis Example 1 to 3 was measured for the absorption peak wavelength by using Spectrophotometer U-3310 manufactured by Hitachi High-Tech Science Corporation.

### Measurement of Orientation

The compounds 1 to 3 obtained in Synthesis Examples 1 to 3 were measured for the orientation in the manner described below.

Each of the compounds 1 to 3 was vapor-deposited on a glass substrate into a thickness of 50 nm. The deposited film was measured for the values of Psi (V) and Delta (Δ) by using a spectroscopic ellipsometer M-2000 manufactured by J.A. Woollam company under the following conditions:

angle of incident light: 45 to 75°;
wavelength: 235 to 1680 nm;
uniaxial anisotropy model: data were fitted to make the mean squared error (MSE) to 2.0 or less.

From the obtained values, the extinction coefficients in the horizontal direction and the normal direction (kₒ, kₑₓ) were calculated. The orientation parameter S′ of each of the compounds 1 to 3 was determined by using kₒ and kₑₓ at the absorption peak wavelength (S1).

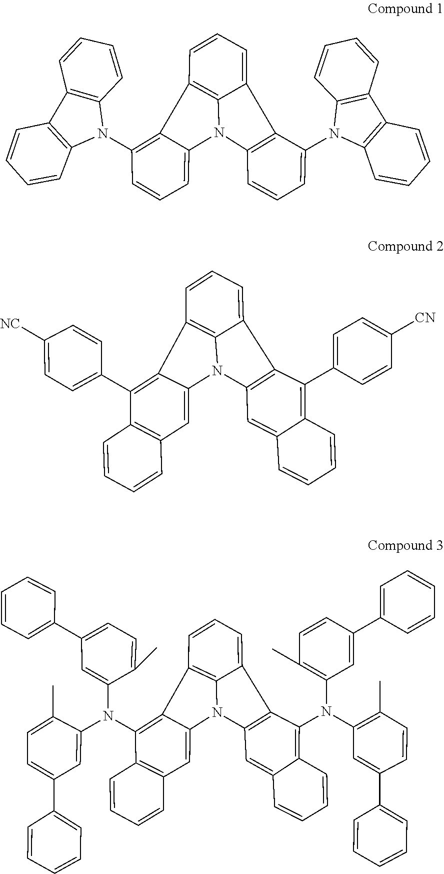 [SMILES: c1ccc2c(c1)c1ccccc1n2-c1cccc2c1c1cccc3c4c(-n5c6ccccc6c6ccccc65)cccc4n2c13 ; N#Cc1ccc(-c2c3ccccc3cc3c2c2cccc4c5c(-c6ccc(C#N)cc6)c6ccccc6cc5n3c24)cc1 ; Cc1ccc(-c2ccccc2)cc1N(c1cc(-c2ccccc2)ccc1C)c1c2ccccc2cc2c1c1cccc3c4c(N(c5cc(-c6ccccc6)ccc5C)c5cc(-c6ccccc6)ccc5C)c5ccccc5cc4n2c13]

### Comparative Example 1

The comparative compound 1 was measured for the absorption peak wavelength and the orientation in the same manner as in Examples 1 to 3. The results are shown in Table 1.

### Comparative Example 2

The comparative compound 2 was measured for the absorption peak wavelength and the orientation in the same manner as in Examples 1 to 3. The results are shown in Table 1.

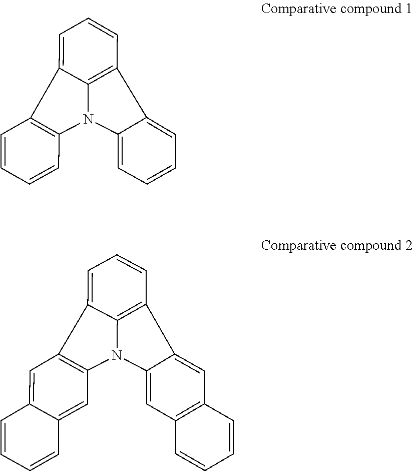 [SMILES: c1ccc2c(c1)c1cccc3c4ccccc4n2c13 ; c1ccc2cc3c(cc2c1)c1cccc2c4cc5ccccc5cc4n3c12]

TABLE 1

| | | Absorption peak | Orientation |
|---|---|---|---|
| | | wavelength λ (nm) | parameter S′ |
| Example 1 | Compound 1 | 373 | 0.74 |
| Example 2 | Compound 2 | 432 | 0.81 |
| Example 3 | Compound 3 | 455 | 0.90 |
| Comparative | Comparative | 364 | 0.67 |
| Example 1 | compound 1 | | |
| Comparative | Comparative | 423 | 0.68 |
| Example 2 | compound 2 | | |

Table 1 shows that the compounds 1 to 3 used in Examples 1 to 3, as compared with the comparative compounds 1 and 2 used in Comparative Examples 1 and 2, have a higher orientation and the organic EL devices comprising the compounds of the invention have a higher emission efficiency as described below.

Production of Organic EL Device Each organic EL device was produced in a manner described below.

### Device Example 1

A 25 mm×75 mm×1.1 mm thickness glass substrate having ITO transparent electrode (anode) (product of Geomatec Company) was ultrasonically cleaned in isopropyl alcohol for 5 min and then UV/ozone cleaned for 30 min. The thickness of ITO transparent electrode was 130 nm.

The cleaned glass substrate was mounted to a substrate holder of a vacuum vapor deposition apparatus. First, the compound HI-1 was vapor-deposited on the surface having the transparent electrode so as to cover the transparent electrode to form a hole injecting layer with a thickness of 5 nm.

On the hole injecting layer, the compound HT-1 was vapor-deposited to form a first hole transporting layer with a thickness of 80 nm.

Then, on the first hole transporting layer, the compound HT-2 was vapor-deposited to form a second hole transporting layer with a thickness of 10 nm.

Successively after forming the second hole transporting layer, the compound BH-1 and the compound 2 (dopant material) were vapor co-deposited to form a light emitting layer with a thickness of 25 nm. The concentration of the compound 2 (dopant material) in the light emitting layer was 4% by mass.

On the light emitting layer, the compound ET-1 was vapor-deposited to form a first electron transporting layer with a thickness of 10 nm.

Successively after forming the first electron transporting layer, the compound ET-2 was vapor-deposited to form a second electron transporting layer with a thickness of 15 nm.

Then, on the second electron transporting layer, lithium fluoride (LiF) was vapor-deposited to form an electron injecting electrode with a thickness of 1 nm.

Then, metallic aluminum (Al) was vapor-deposited on the electron injecting electrode to form a metallic cathode with a thickness of 80 nm.

The layered structure of the organic EL device is shown below.

ITO (130)/HI-1 (5)/HT-1 (80)/HT-2 (10)/BH-1:compound 2 (25:4% by mass)/ET-1 (10)/ET-2 (15)/LiF (1)/Al (80)

The numeral in each parenthesis is the thickness (nm).

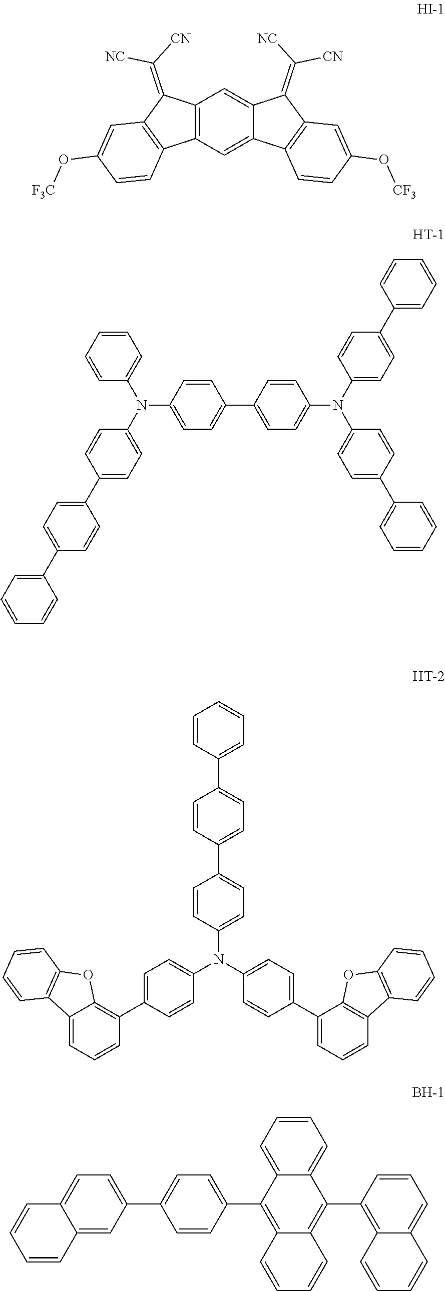

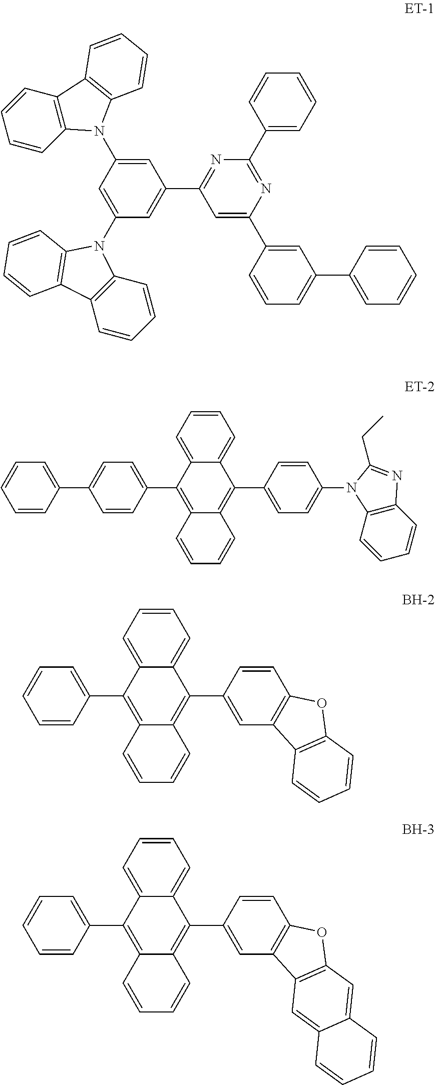 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1 ; c1ccc(-c2c3ccccc3c(-c3ccc4oc5ccccc5c4c3)c3ccccc23)cc1 ; c1ccc(-c2c3ccccc3c(-c3ccc4oc5cc6ccccc6cc5c4c3)c3ccccc23)cc1]

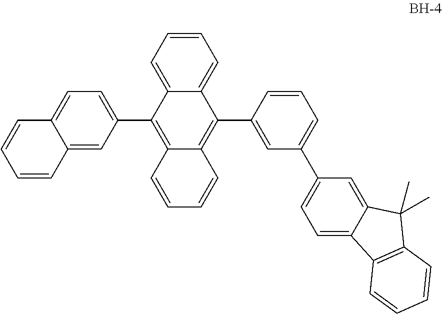 [SMILES: CC1(C)c2ccccc2-c2ccc(-c3cccc(-c4c5ccccc5c(-c5ccc6ccccc6c5)c5ccccc45)c3)cc21]

### Evaluation of Organic EL Device

The organic EL device thus produced was operated by applying a voltage so as to obtain a current density of 10 mA/cm² and EL emission spectrum was measured by using a spectroradiometer CS-1000 manufactured by Konica Minolta. The external quantum efficiency (EQE (%)) was calculated from the obtained spectral radiance spectrum. The results are shown in Table 2.

### Device Example 2

An organic EL device was produced in the same manner as in Device Example 1 except for using the compound 3 as the dopant material in place of the compound 2 and evaluated in the same manner as in Device Example 1. The result is shown in Table 2.

### Device Example 3

An organic EL device was produced in the same manner as in Device Example 1 except for using the compound BH-2 as the host material in place of the compound BH-1 and evaluated in the same manner as in Device Example 1. The result is shown in Table 2.

### Device Example 4

An organic EL device was produced in the same manner as in Device Example 2 except for using the compound BH-2 as the host material in place of the compound BH-1 and evaluated in the same manner as in Device Example 2. The result is shown in Table 2.

### Device Example 5

An organic EL device was produced in the same manner as in Device Example 1 except for using the compound BH-3 as the host material in place of the compound BH-1 and evaluated in the same manner as in Device Example 1. The result is shown in Table 2.

### Device Example 6

An organic EL device was produced in the same manner as in Device Example 2 except for using the compound BH-3 as the host material in place of the compound BH-1 and evaluated in the same manner as in Device Example 2. The result is shown in Table 2.

### Device Example 7

An organic EL device was produced in the same manner as in Device Example 2 except for using the compound BH-4 as the host material in place of the compound BH-1 and evaluated in the same manner as in Device Example 2. The result is shown in Table 2.

### Device Comparative Example 1

An organic EL device was produced in the same manner as in Device Example 1 except for using the comparative compound 2 as the dopant material in place of the compound 2 and evaluated in the same manner as in Device Example 1. The result is shown in Table 2.

### Device Comparative Example 2

An organic EL device was produced in the same manner as in Device Example 5 except for using the comparative compound 2 as the dopant material in place of the compound 2 and evaluated in the same manner as in Device Example 5. The result is shown in Table 2.

TABLE 2

| | BH | BD | EQE (%) |
|---|---|---|---|
| Device example 1 | BH-1 | Compound 2 | 7.2 |
| Device example 2 | BH-1 | Compound 3 | 8.2 |
| Device example 3 | BH-2 | Compound 2 | 7.0 |
| Device example 4 | BH-2 | Compound 3 | 7.7 |
| Device example 5 | BH-3 | Compound 2 | 7.0 |
| Device example 6 | BH-3 | Compound 3 | 7.5 |
| Device example 7 | BH-4 | Compound 3 | 7.3 |
| Device comparative | BH-1 | Comparative | 6.4 |
| example 1 | | compound 2 | |
| Device comparative | BH-3 | Comparative | 4.8 |
| example 2 | | compound 2 | |

Table 2 shows that as compared with Device Comparative Examples 1 and 2 wherein the light emitting layer includes the comparative compound 2, higher emission efficiencies are obtained in Device Examples 1 to 7 wherein the light emitting layer includes the compound 2 or the compound 3.

In addition, it can be found that as compared with Device Comparative Example 1 wherein the light emitting layer includes the compound BH-1 and the comparative compound 2, higher emission efficiencies are obtained in Device Example 1 wherein the light emitting layer includes the compound BH-1 and the compound 2 and Device Example 2 wherein the light emitting layer includes the compound BH-1 and the compound 3.

Similarly, as compared with Device Comparative Example 2 wherein the light emitting layer includes the compound BH-3 and the comparative compound 2, higher emission efficiencies are obtained in Device Example 5 wherein the light emitting layer includes the compound BH-3 and the compound 2 and Device Example 6 wherein the light emitting layer includes the compound BH-3 and the compound 3.

### Examples 4 and 5

### Measurement of PLQY of the Compounds Used in Device Examples 1 and 2

A toluene solution (5 μmol/mL) of each of the compounds 2 and 3 was measured for PLQY by using an absolute PL quantum yield spectrometer (Quantaurus-QY) manufactured by Hamamatsu Photonics K.K.

The results are shown in Table 3.

TABLE 3

| | | PLQY |
|---|---|---|
| Example 4 | Compound 2 | 0.86 |
| Example 5 | Compound 3 | 0.86 |

### REFERENCE SIGNS LIST

1, 11: Organic EL device
2: Substrate
3: Anode
4: Cathode
5: Light emitting layer
6: Hole transporting region (hole transporting layer)
6a: First hole transporting layer
6b: Second hole transporting layer
7: Electron transporting region (electron transporting layer)
7a: First electron transporting layer
7b: Second electron transporting layer
10, 20: Emission unit

1. A compound represented by formula (1): 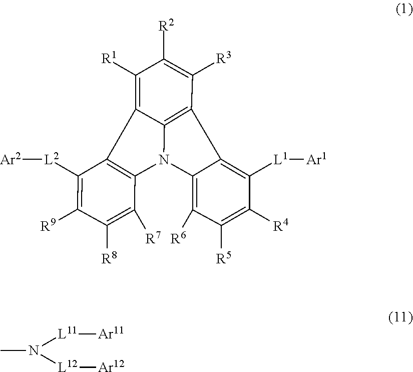
wherein:
R¹ to R⁹ are each independently a hydrogen atom, a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³), a group represented by —N(R¹⁰⁴)(R¹⁰⁵), or a substituted or unsubstituted heterocyclic group having 5 to 50 ring atoms,
or
adjacent two selected from R¹ to R³, adjacent two selected from R⁴ to R⁶, or adjacent two selected from R⁷ to R⁹ form a substituted or unsubstituted ring structure;
R¹⁰¹ to R¹⁰⁵ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heterocyclic group having 5 to 50 ring atoms;
Ar¹ and Ar² are each independently a substituted or unsubstituted aryl group having 6 to 50 ring carbon atoms, a substituted or unsubstituted heterocyclic group having 5 to 50 ring atoms, or a group represented by formula (11);
Ar¹¹ and Ar¹¹ are each independently a substituted or unsubstituted aryl group having 6 to 50 ring carbon atoms or a substituted or unsubstituted heterocyclic group having 5 to 50 ring atoms and Ar¹¹ and Ar¹¹ may be bonded to each other via a single bond; and
L¹, L², L¹¹ and L¹² are each independently a single bond, a substituted or unsubstituted arylene group having 6 to 50 ring carbon atoms, or a substituted or unsubstituted divalent heterocyclic group having 5 to 50 ring atoms.

2. The compound according to claim 1, wherein Ar¹ and Ar² are each independently a substituted or unsubstituted aryl group having 10 to 50 ring carbon atoms, a substituted or unsubstituted heterocyclic group having 5 to 50 ring atoms, a group represented by formula (11), or a group represented by formula (21): 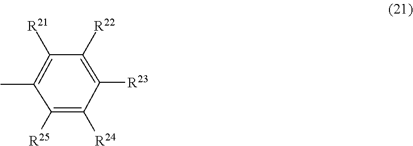
wherein:
R²¹ to R²⁵ are each independently a hydrogen atom, a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³) wherein R¹⁰¹ to R¹⁰³ are as defined above, a group represented by —N(R¹⁰⁴)(R¹⁰⁵) wherein R¹⁰⁴ and R¹⁰⁵ are as defined above, or a substituted or unsubstituted heterocyclic group having 5 to 50 ring atoms;
provided that at least one of R²¹ to R²⁵ is a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³) wherein R¹⁰¹ to R¹⁰³ are as defined above, a group represented by —N(R¹⁰⁴)(R¹⁰⁵) wherein R¹⁰⁴ and R¹⁰⁵ are as defined above, or a substituted or unsubstituted heterocyclic group having 5 to 50 ring atoms.

3. The compound according to claim 1, wherein the compound is represented by formula (2): 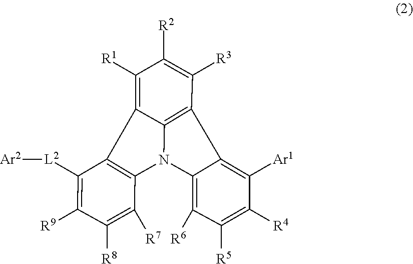
wherein R¹ to R⁹, Ar¹ to Ar², and L² are as defined above.

4. The compound according to claim 1, wherein the compound is represented by formula (3): 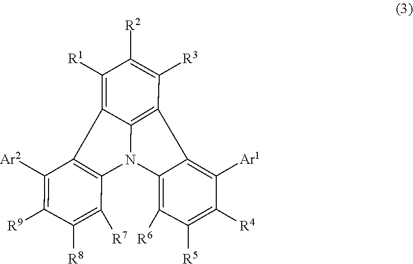
wherein R¹ to R⁹ and Ar¹ to Ar² are as defined above.

5. The compound according to claim 1, wherein the compound is represented by formula (4): 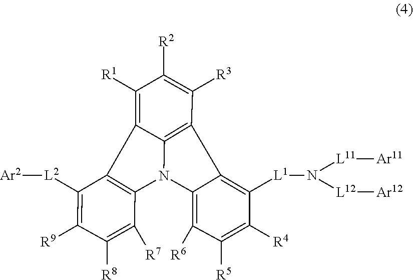
wherein R¹ to R⁹, Ar², Ar¹¹ to Ar¹², L¹ to L², and L¹¹ to L¹² are as defined above.

6. The compound according to claim 1, wherein the compound is represented by formula (5): 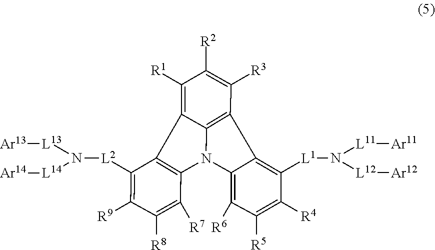
wherein:
R¹ to R⁹, Ar², Ar¹¹ to Ar¹², L¹ to L², and L¹¹ to L¹² are as defined above;
L¹³ to L¹⁴ are as defined above with respect to L¹¹ to L¹²; and
Ar¹³ to Ar¹⁴ are as defined above with respect to Ar¹¹ to Ar¹².

7. The compound according to claim 1, wherein the compound is represented by formula (6): 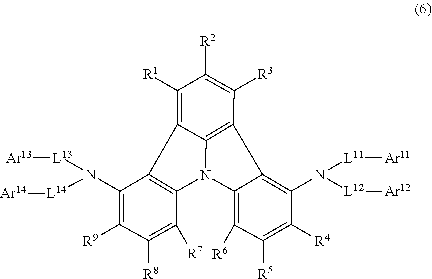
wherein:
R¹ to R⁹, Ar², Ar¹¹ to Ar¹², and L¹¹ to L¹² are as defined above;
L¹³ to L¹⁴ are as defined above with respect to L¹¹ to L¹²; and
Ar¹³ to Ar¹⁴ are as defined above with respect to Ar¹¹ to Ar¹².

8. The compound according to claim 1, wherein the compound is represented by formula (7): 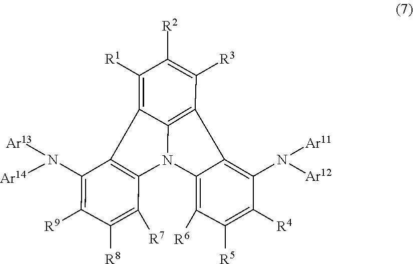
wherein:
R¹ to R⁹, Ar², and Ar¹¹ to Ar¹² are as defined above; and
Ar¹³ to Ar¹⁴ are as defined above with respect to Ar¹¹ to Ar¹².

9. The compound according to claim 1, wherein Ar¹ and Ar² are each independently a substituted aryl group having 6 to 50 ring carbon atoms.

10. The compound according to claim 1, wherein the compound is represented by formula (8): 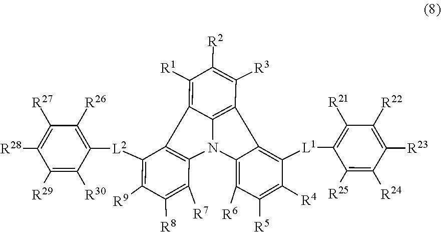
wherein:
R¹ to R⁹, L¹ to L² and R²¹ to R²⁵ are as defined above; and
R²⁶ to R³⁰ areas defined above with respect to R²¹ to R²⁵.

11. The compound according to claim 1, wherein the compound is represented by formula (9): 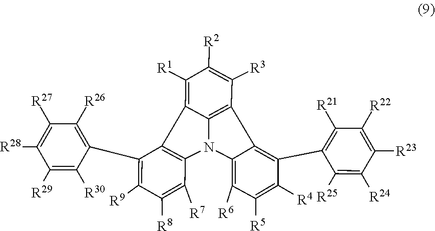
wherein:
R¹ to R⁹ and R²¹ to R²⁵ are as defined above; and
R²⁶ to R³⁰ are as defined above with respect to R²¹ to R²⁵.

12. The compound according to claim 10, wherein at least one of R²¹ to R²⁵ is a cyano group and at least one of R²⁶ to R³⁰ is a cyano group.

13. The compound according to any one of claim 10, wherein R²³ and R²⁸ are both cyano groups.

14. The compound according to claim 1, wherein the compound is represented by formula (1-1): 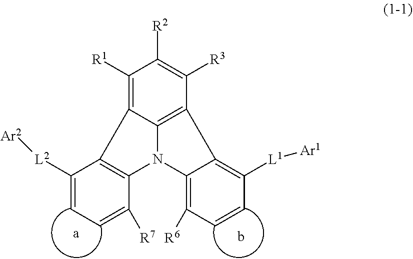
wherein:
R¹ to R³, R⁶ to R⁷, L¹, L², Ar¹, and Ar² are as defined above; and
each of ring a and ring b is a substituted or unsubstituted aromatic hydrocarbon ring having 6 to 30 ring carbon atoms or a substituted or unsubstituted aromatic heterocyclic ring having 5 to 30 ring atoms.

15. The compound according to claim 1, wherein the compound is represented by formula (1-1-1): 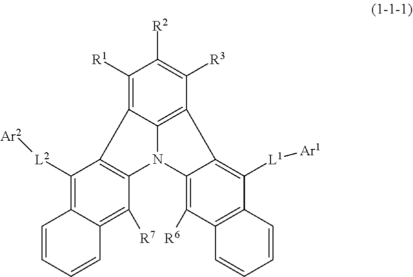
wherein R¹ to R³, R⁶ to R⁷, L¹, L², Ar¹, and Ar² are as defined above.

16. The compound according to claim 1, wherein formula (11) is represented by formula (31): 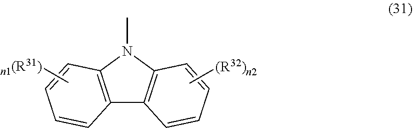
wherein:
R³¹ to R³² are each independently a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³) wherein R¹⁰¹ to R¹⁰³ are as defined above, a group represented by —N(R¹⁰⁴)(R¹⁰⁵) wherein R¹⁰⁴ and R¹⁰⁵ are as defined above, or a substituted or unsubstituted heterocyclic group having 5 to 50 ring atom,
or
adjacent two selected from R³¹ and R³² form a ring structure; and
n1 and n2 are each independently an integer of 0 to 4.

17. The compound according to claim 1, wherein -L¹-Ar¹ and -L²-Ar² are the same.

18. The compound according to claim 1, wherein adjacent two selected from R¹ to R³, adjacent two selected from R⁴ to R⁶, and adjacent two selected from R⁷ to R⁹ do not form a ring structure.

19. The compound according to claim 1, wherein R¹ to R⁹ are each independently a hydrogen atom, a cyano group, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, a group represented by —N(R¹⁰⁴)(R¹⁰⁵), or a substituted or unsubstituted heterocyclic group having 5 to 50 ring atoms.

20. The compound according to claim 1, wherein the substituent referred by “substituted or unsubstituted” is selected from the group consisting of a halogen atom, a cyano group, an alkyl group, an alkenyl group, an alkynyl group, a cycloalkyl group, an alkoxy group, an alkylthio group, an aryl group, an aralkyl group, an aryloxy group, an arylthio group, a group represented by —Si(R^{1O1})(R¹⁰²)(R¹⁰³), a group represented by —N(R¹⁰⁴)(R¹⁰⁵), a heterocyclic group, a nitro group, a hydroxyl group, a carboxyl group, a vinyl group, a carbonyl group having a substituent selected from an alkyl group and aryl group, a sulfonyl group having a substituent selected from an alkyl group and aryl group, a di-substituted phosphoryl group having a substituent selected from an alkyl group and aryl group, an alkylcarbonyloxy group, an arylcarbonyloxy group, an alkylsulfonyloxy group, an arylsulfonyloxy group, and a (meth)acryloyl group.

21. The compound according to claim 1, wherein the substituent referred by “substituted or unsubstituted” is selected from the group consisting of a halogen atom, a cyano group, an alkyl group, a cycloalkyl group, an aryl group, and a heterocyclic group.

22. A compound represented by formula (1): 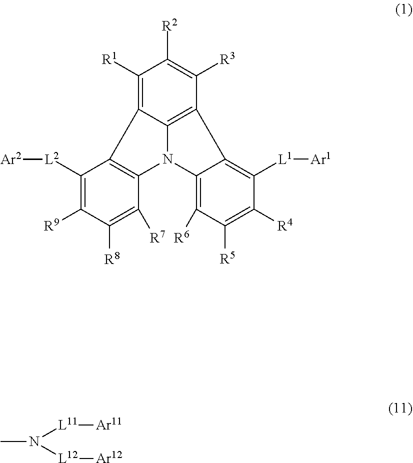
wherein:
R¹ to R⁹ are each independently a hydrogen atom, a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted alkylthio group having 1 to 20 carbon atoms, a substituted or unsubstituted aryl group having 6 to 50 ring carbon atoms, a substituted or unsubstituted aralkyl group having 7 to 51 carbon atoms, a substituted or unsubstituted aryloxy group having 6 to 50 ring carbon atoms, a substituted or unsubstituted arylthio group having 6 to 50 ring carbon atoms, a group represented by —Si(R¹⁰¹)(R¹⁰²)(R¹⁰³), a group represented by —N(R¹⁰⁴)(R¹⁰⁵), or a substituted or unsubstituted heterocyclic group having 5 to 50 ring atoms,
or
adjacent two selected from R¹ to R³, adjacent two selected from R⁴ to R⁶, or adjacent two selected from R⁷ to R⁹ form a substituted or unsubstituted ring structure;
R¹⁰¹ to R¹⁰⁵ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heterocyclic group having 5 to 50 ring atom;
Ar¹ and Ar² are each independently a substituted or unsubstituted aryl group having 6 to 50 ring carbon atoms, a substituted or unsubstituted heterocyclic group having 5 to 50 ring atoms, or a group represented by formula (11);
Ar¹¹ and Ar¹¹ are each independently a substituted or unsubstituted aryl group having 6 to 50 ring carbon atoms or a substituted or unsubstituted heterocyclic group having 5 to 50 ring atoms and Ar¹¹ and Ar¹¹ may be bonded to each other via a single bond or may form a substituted or unsubstituted ring structure together with R⁴; and
L¹, L², L¹¹ and L¹² are each independently a single bond, a substituted or unsubstituted arylene group having 6 to 50 ring carbon atoms, or a substituted or unsubstituted divalent heterocyclic group having 5 to 50 ring atoms.

23. A material for organic electroluminescence device which comprises the compound according to claim 1.

24. An organic electroluminescence device comprising a cathode, an anode, and an organic layer disposed between the cathode and the anode, wherein the organic layer comprises a light emitting layer and at least one layer of the organic layer comprises the compound according to claim 1.

25. The organic electroluminescence device according to claim 24, wherein the light emitting layer comprises the compound.

26. The organic electroluminescence device according to claim 24, wherein at least one layer of the organic layer comprises the compound and a compound represented by formula (10): 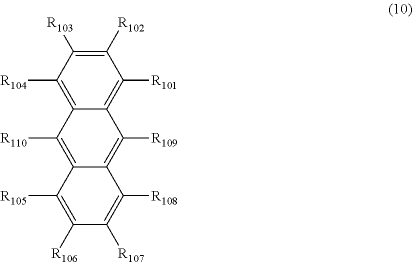
wherein:
at least one of R₁₀₁ to R₁₁₀ is a group represented by formula (31);
two or more groups represented by formula (31), if present, may be the same or different;
-L₁₀₁-Ar₁₀₁ (31)
wherein:
L₁₀₁ is a single bond, a substituted or unsubstituted arylene group having 6 to 30 ring carbon atoms, or a substituted or unsubstituted divalent heterocyclic group having 5 to 30 ring atoms;
Ar₁₀₁ is a substituted or unsubstituted aryl group having 6 to 50 ring carbon atoms or a substituted or unsubstituted monovalent heterocyclic group having 5 to 50 ring atoms;
one or more sets of adjacent two selected from R₁₀₁ to R₁₁₀ not representing the group represented by formula (31) form a substituted or unsubstituted, saturated or unsaturated ring or do not form a ring;
R₁₀₁ to R₁₁₀ not representing the group represented by formula (31) and not forming the ring are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a substituted or unsubstituted aryl group having 6 to 50 ring carbon atoms, or a substituted or unsubstituted monovalent heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted monovalent heterocyclic group having 5 to 50 ring atoms; and
two or more R₉₀₁ to R₉₀₇, if present, may be the same or different.

27. The organic electroluminescence device according to claim 26, wherein the compound represented by formula (10) is represented by formula (10-1) or (10-2): 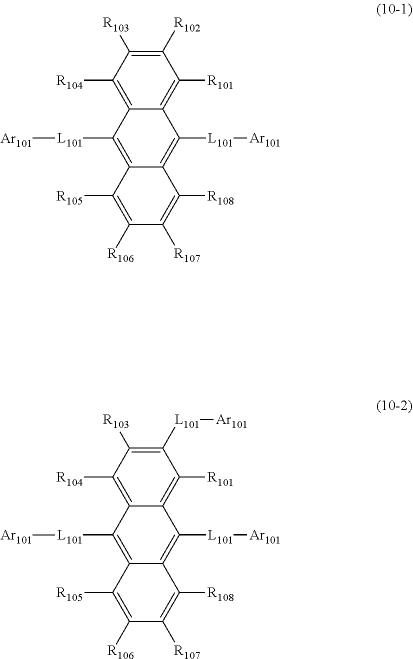
wherein:
R₁₀₁ to R₁₀₈, L₁₀₁, and Ar₁₀₁ of formula (10-1) are as defined in formula (10); and
R₁₀₁, R₁₀₃ to R₁₀₈, L₁₀₁, and Ar₁₀₁ of formula (10-2) are as defined in formula (10).

28. The organic electroluminescence device according to claim 26, wherein the compound represented by formula (10) is represented by formula (10-3): 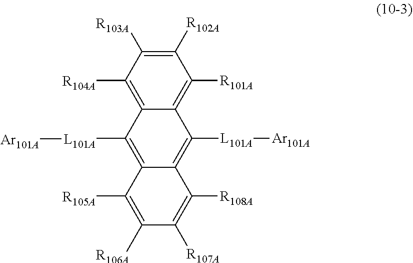
wherein:
R_{101A} to R_{108A} are each independently a hydrogen atom or a substituted or unsubstituted aryl group having 6 to 50 ring carbon atoms;
L_{101A} is a single bond or a substituted or unsubstituted arylene group having 6 to 30 ring carbon atoms and two L_{101A} may be the same or different; and
Ar_{101A} is a substituted or unsubstituted aryl group having 6 to 50 ring carbon atoms and two Ar_{101A} may be the same or different.

29. The organic electroluminescence device according to claim 26, wherein the compound represented by formula (10) is represented by formula (10-4): 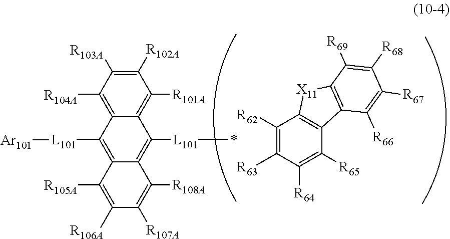
wherein:
L₁₀₁ and Ar₁₀₁ are as defined in formula (10);
R_{101A} to R_{108A} are each independently a hydrogen atom or a substituted or unsubstituted aryl group having 6 to 50 ring carbon atoms;
X₁₁ is O, S, C(R₉₁)(R₉₂), or N(R₆₁);
R₉₁ and R₉₂ are as defined above with respect to R¹ to R⁹;
R₆₁ is a hydrogen atom, a substituted or unsubstituted alkyl group having 1 to 50 carbon atoms, or a substituted or unsubstituted aryl group having 6 to 50 ring carbon atoms;
at least one of R₆₂ to R₆₉ is a bond bonded to L₁₀₁;
one or more sets of adjacent two selected from R₆₂ to R₆₉ not bonded to L₁₀₁ form a substituted or unsubstituted, saturated or unsaturated ring or do not form a ring;
R₆₂ to R₆₉ not bonded to L₁₀₁ and not form the ring are each independently a hydrogen atom, a substituted or unsubstituted alkyl group having 1 to 50 carbon atoms, or a substituted or unsubstituted aryl group having 6 to 50 ring carbon atoms.

30. The organic electroluminescence device according to claim 29, wherein the compound represented by formula (10) is represented by formula (10-6): 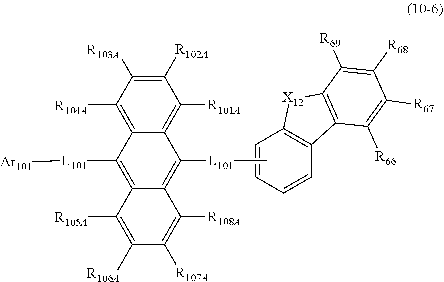
wherein:
L₁₀₁ and Ar₁₀₁ are as defined in formula (10);
R_{101A} to R_{108A} are as defined in formula (10-4);
R₆₆ to R₆₉ are as defined in formula (10-4);
X₁₂ is O, S, or C(R₉₁)(R₉₂); and
R₉₁ and R₉₂ are as defined above with respect to R¹ to R⁹.

31. The organic electroluminescence device according to claim 29, wherein the compound represented by formula (10) is represented by formula (10-7): 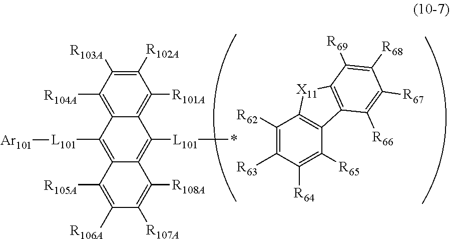
wherein:
L₁₀₁ and Ar₁₀₁ are as defined in formula (10);
R_{101A} to R_{108A} are as defined in formula (10-4);
X₁₁ is as defined in formula (10-4); and
R₆₂ to R₆₉ are as defined in formula (10-4), provided that one set of adjacent two selected from R₆₆ and R₆₇, R₆₇ and R₆₈, and R₆₈ and R₆₉ are bonded to each other to form a substituted or unsubstituted, saturated or unsaturated ring.

32. The organic electroluminescence device according to claim 26, wherein the compound represented by formula (10) is represented by formula (10-8): 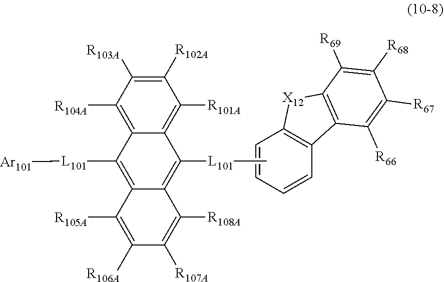
wherein:
L₁₀₁ and Ar₁₀₁ are as defined in formula (10);
R_{101A} to R_{108A} are as defined in formula (10-4);
X₁₂ is O, S, or C(R₉₁)(R₉₂);
R₉₁ and R₉₂ are as defined above with respect to R¹ to R⁹; and
R₆₆ to R₆₉ are as defined in formula (10-4), provided that one set of adjacent two selected from R₆₆ and R₆₇, R₆₇ and R₆₈, and R₆₈ and R₆₉ are bonded to each other to form a substituted or unsubstituted, saturated or unsaturated ring.

33. The organic electroluminescence device according to claim 31, wherein:
one set of adjacent two selected from R₆₆ and R₆₇, R₆₇ and R₆₈, and R₆₈ and R₆₉ are bonded to each other to form a ring represented by formula (10-8-1) or (10-8-2); and
R₆₆ to R₆₉ not forming the ring represented by formula (10-8-1) or (10-8-2) do not form a substituted or unsubstituted, saturated or unsaturated ring, 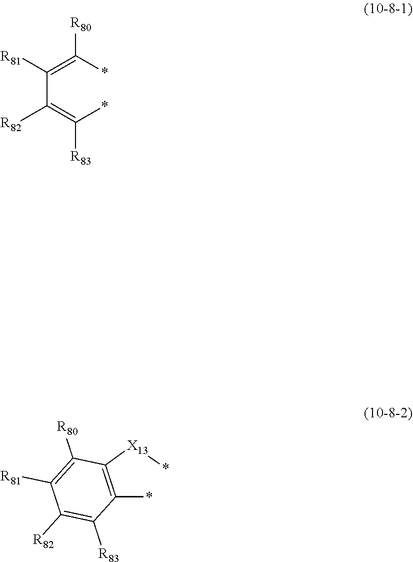
wherein:
two bonds * are bonded to one set of adjacent two selected from R₆₆ and R₆₇, R₆₇ and R₆₈, and R₆₈ and R₆₉;
R₈₀ to R₈₃ are each independently a hydrogen atom, a substituted or unsubstituted alkyl group having 1 to 50 carbon atoms, or a substituted or unsubstituted aryl group having 6 to 50 ring carbon atoms;
X₁₃ is O, S, or C(R₉₁)(R₉₂); and
R₉₁ and R₉₂ are as defined above with respect to R¹ to R⁹.

34. The organic electroluminescence device according to claim 29, wherein the compound represented by formula (10) is represented by formula (10-9): 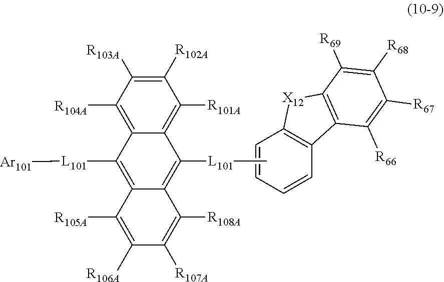
wherein:
L₁₀₁ and Ar₁₀₁ are as defined in formula (10);
R_{101A} to R_{108A} are as defined in formula (10-4);
R₆₆ to R₆₉ are as defined in formula (10-4), provided that one set of adjacent two selected from R₆₆ and R₆₇, R₆₇ and R₆₈, and R₆₉ and R₆₇ are not bonded to each other thereby failing to form a substituted or unsubstituted, saturated or unsaturated ring;
X₁₂ is O, S, or C(R₉₁)(R₉₂); and
R₉₁ and R₉₂ are as defined above with respect to R¹ to R⁹.

35. The organic electroluminescence device according to claim 26, wherein the compound represented by formula (10) is represented by formula (10-4A): 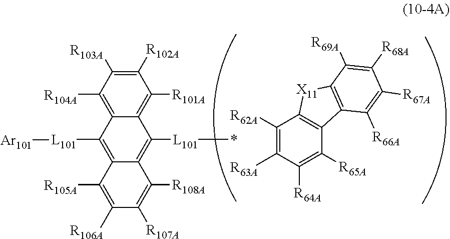
wherein:
L₁₀₁ and Ar₁₀₁ are as defined in formula (10);
R_{101A} to R_{108A} are each independently a hydrogen atom or a substituted or unsubstituted aryl group having 6 to 50 ring carbon atoms;
X₁₁ is O, S, C(R₉₁)(R₉₂), or N(R₆₁);
R₉₁ and R₉₂ are as defined above with respect to R¹ to R⁹;
R₆₁ is a hydrogen atom, a substituted or unsubstituted alkyl group having 1 to 50 carbon atoms, or a substituted or unsubstituted aryl group having 6 to 50 ring carbon atoms;
one set of adjacent two selected from R_{62A} to R_{69A} form a ring represented by formula (10-4A-1): 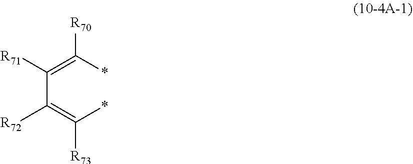
wherein:
two bonds * are bonded to adjacent two selected from R_{62A} to R_{69A};
one of R₇₀ to R₇₃ is a bond bonded to L₁₀₁; and
R₇₀ to R₇₃ not bonded to L₁₀₁ are each independently a hydrogen atom, a substituted or unsubstituted alkyl group having 1 to 50 carbon atoms, or a substituted or unsubstituted aryl group having 6 to 50 ring carbon atoms;
one or more sets of adjacent two selected from R_{62A} to R_{69A} not forming the ring represented by formula (10-4A-1) form a substituted or unsubstituted, saturated or unsaturated ring or do not form a ring; and
R_{62A} to R_{69A} not forming the ring represented by formula (10-4A-1) and a substituted or unsubstituted, saturated or unsaturated ring are each independently a hydrogen atom, a substituted or unsubstituted alkyl group having 1 to 50 carbon atoms, or a substituted or unsubstituted aryl group having 6 to 50 ring carbon atoms.

36. An electronic device comprising the organic electroluminescence device according to claim 24.
